SEQUENCE LISTING (422 sequences)

SEQ ID NO: 1 - protein, 71 aa, Cardiovirus B
  source 1..71: note = Cardiovirus Theiler's-like cardiovirus
MACKHGYPLMCPLCTALDKTSDGLFTLLFDNEWYPTDLLTVDLEDEVFYPDDPHMEWTDLPLIQDIEMEPQ

SEQ ID NO: 2 - protein, 67 aa, Cardiovirus A
  source 1..67: note = Cardiovirus Encephalomyocarditis virus
MATTMEQETCAHSLTFEECPKCSALQYRNGFYLLKYDEEWYPEELLTDGEDDVFDPELDMEVVFELQ

SEQ ID NO: 3 - protein, 127 aa, Enterovirus C
NYHLATQDDLQNAVNVMWSRDLLVTESRAQGTDSIARCNCNAGVYYCESRRKYYPVSFVGPTFQYMEANNYYPARYQSHMLIGHGFASPGDCGGILRCHHGVIGIITAGGEGLVAFSDIRDLYAYEE

SEQ ID NO: 4 - protein, 219 aa, Erbovirus A
  source 1..219: note = Erbovirus Equine rhinitis B virus 1
MVTMAGNMICNVFAGLATEICSPKQGPLLDNELPLPLELAEFPNKDNNCWVAALSHYYTLCDVTNHVTKVTPTTSGIRYYLTAWQSILQTDLFNGYYPAAFAVETGLCHGPFPMQQHGYVRNATSHPYNFCLCSEPVPGEDYWHAVVKVDLSRTEARVDKWLCIDDDRMYLSGPPTRVKLASSYKIPTWIESLAQFCLQLHPVQHRRTLANSLRNEQCR

SEQ ID NO: 5 - protein, 67 aa, unidentified
  source 1..67: note = Cardiovirus Encephalomyocarditis virus
MATTMEQEICAHSMTFEECPKCSALQYRNGFYLLKYDEEWYPEESLTDGEDDVFDPDLDMEVVFETQ

SEQ ID NO: 6 - protein, 71 aa, Cardiovirus B
MACKHGYPFLCPLCTAIDTTHDGSFTLLIDNEWYPTDLLTVDLDDDVFHPDDSVMEWTDLPLIQDVVMEPQ

SEQ ID NO: 7 - protein, 149 aa, Enterovirus C
GFGHQNKAVYTAGYKICNYHLATQDDLQNAVNVMWSRDLLVTESRAQGTDSIARCNCNAGVYYCESRRKYYPVSFVGPTFQYMEANNYYPARYQSHMLIGHGFASPGDCGGILRCHHGVIGIITAGGEGLVAFSDIRDLYAYEEEAMEQ

SEQ ID NO: 8 - protein, 182 aa, unidentified
  source 1..182: note = Enterovirus Human rhinovirus 16
GPEEEFGMSIIKNNTCVVTTTNGKFTGLGIYDRILILPTHADPGSEIQVNGIHTKVLDSYDLFNKEGVKLEITVLKLDRNEKFRDIRKYIPESEDDYPECNLALVANQTEPTIIKVGDVVSYGNILLSGTQTARMLKYNYPTKSGYCGGVLYKIGQILGIHVGGNGRDGFSSMLLRSYFTEQ

SEQ ID NO: 9 - protein, 229 aa, Vesiculovirus indiana
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHTHCEGRAYLPHRMGKTPPMLNVPEHFRRPFNIGLYKGTIELTMTIYDDESLEAAPMIWDHFNSSKFSDFREKALMFGLIVEKKASGAWVLDSISHFK

SEQ ID NO: 10 - protein, 230 aa, Alphainfluenzavirus influenzae
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 11 - protein, 512 aa, unidentified
  REGION 1..512: note = Simplexvirus sp.
MATDIDMLIDLGLDLSDSDLDEDPPEPAESRRDDLESDSSGECSSSDEDMEDPHGEDGPEPILDAARPAVRPSRPEDPGVPSTQTPRPTERQGPNDPQPAPHSVWSRLGARRPSCSPEQHGGKVARLQPPPTKAQPARGGRRGRRRGRGRGGPGAADGLSDPRRRAPRTNRNPGGPRPGAGWTDGPGAPHGEAWRGSEQPDPPGGQRTRGVRQAPPPLMTLAIAPPPADPRAPAPERKAPAADTIDATTRLVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTTAAVLDNLATRLRPFLQCYLKARGLCGLDELCSRRRLADIKDIASFVFVILARLANRVERGVAEIDYATLGVGVGEKMHFYLPGACMAGLIEILDTHRQECSSRVCELTASHIVAPPYVHGKYFYCNSLF

SEQ ID NO: 12 - protein, 700 aa, synthetic construct
  REGION 1..700: note = DRD1-GFP
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPTGGGGSGGGGSGGGGSMVSKGEELFTGVVPILVELDGDVNGHKFSVSGEGEGDATYGKLTLKFICTTGKLPVPWPTLVTTLTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSKLSKDPNEKRDHMVLLEFVTAAGITLGMDELYK

SEQ ID NO: 13 - protein, 239 aa, synthetic construct
  REGION 1..239: note = GFP
MVSKGEELFTGVVPILVELDGDVNGHKFSVSGEGEGDATYGKLTLKFICTTGKLPVPWPTLVTTLTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSKLSKDPNEKRDHMVLLEFVTAAGITLGMDELYK

SEQ ID NO: 14 - protein, 460 aa, synthetic construct
  REGION 1..460: note = DRD1-Strep
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPTTGTRPLWSHPQFEK

SEQ ID NO: 15 - protein, 635 aa, synthetic construct
  REGION 1..635: note = ITK
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDRESRQRWVLALKEETRNNNSLVPKYHPNFWMDGKWRCCSQLEKLATGCAQYDPTKNASKKPLPPTPEDNRRPLWEPEETVVIALYDYQTNDPQELALRRNEEYCLLDSSEIHWWRVQDRNGHEGYVPSSYLVEKSPNNLETYEWYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPVCFGRQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGLGGGGGGGGHHHHHHV

SEQ ID NO: 16 - protein, 500 aa, synthetic construct
  REGION 1..500: note = C1 Inhibitor
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 17 - protein, 883 aa, synthetic construct
  REGION 1..883: note = T7 RNA polymerase
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 18 - protein, 1480 aa, synthetic construct
  REGION 1..1480: note = CFTR
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECLFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 19 - protein, 550 aa, synthetic construct
  REGION 1..550: note = Luciferase insert sequence
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 20 - DNA, 2237 bp, synthetic construct
  misc_feature 1..2237: note = Luciferase insert sequence
  source 1..2237: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 21 - protein, 67 aa, synthetic construct; identical to SEQ ID NO: 2
  REGION 1..67: note = L1 insert sequence

SEQ ID NO: 22 - DNA, 204 bp, synthetic construct
  misc_feature 1..204: note = L1 insert sequence
  source 1..204: mol_type = other DNA
atggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaa

SEQ ID NO: 23 - DNA, 564 bp, synthetic construct
  misc_feature 1..564: note = IRES insert sequence
  source 1..564: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 24 - protein, 76 aa, Cardiovirus B
MACKHGYPDVCPICTAVDATPGFEYLLMADGEWYPTDLLCVDLDDDVFWPSDTSNQSQTMDWTDVPLIRDIVMEPQ

SEQ ID NO: 25 - DNA, 46 bp, synthetic construct
  misc_feature 1..46: note = primer
  source 1..46: mol_type = other DNA
gcccgggatccaccggtcgccaccatggtgagcaagggcgaggagc

SEQ ID NO: 26 - DNA, 53 bp, synthetic construct
  misc_feature 1..53: note = primer
  source 1..53: mol_type = other DNA
agatggctggcaactagaaggcacagttacttgtacagctcgtccatgccgag

SEQ ID NO: 27 - DNA, 59 bp, synthetic construct
  misc_feature 1..59: note = primer
  source 1..59: mol_type = other DNA
cactctcggcatggacgagctgtacaagtaactgtgccttctagttgccagccatctgt

SEQ ID NO: 28 - DNA, 44 bp, synthetic construct
  misc_feature 1..44: note = primer
  source 1..44: mol_type = other DNA
cagctcctcgcccttgctcaccatggtggcgaccggtggatccc

SEQ ID NO: 29 - DNA, 53 bp, synthetic construct
  misc_feature 1..53: note = primer
  source 1..53: mol_type = other DNA
cggccagtaacgttaggggggggggattacttgtacagctcgtccatgccgag

SEQ ID NO: 30 - DNA, 57 bp, synthetic construct
  misc_feature 1..57: note = primer
  source 1..57: mol_type = other DNA
cggtaccgcgggcccgggatccaccggtcgccaccatggtgagcaagggcgaggagc

SEQ ID NO: 31 - DNA, 50 bp, synthetic construct
  misc_feature 1..50: note = primer
  source 1..50: mol_type = other DNA
ctcggcatggacgagctgtacaagtaatcccccccccctaacgttactgg

SEQ ID NO: 32 - DNA, 81 bp, synthetic construct
  misc_feature 1..81: note = primer
  source 1..81: mol_type = other DNA
acgggggaggggcaaacaacagatggctggcaactagaaggcacagctgtaactcgaaaacgacttccatgtctaattcgg

SEQ ID NO: 33 - DNA, 72 bp, synthetic construct
  misc_feature 1..72: note = primer
  source 1..72: mol_type = other DNA
cgcgggcccgggatccaccggtcgccaccatgaacaccatcaatattgccaagaacgacttttctgacatcg

SEQ ID NO: 34 - DNA, 59 bp, synthetic construct
  misc_feature 1..59: note = primer
  source 1..59: mol_type = other DNA
agatggctggcaactagaaggcacagttagggtcaggcaaatgcgaaatcggactccag

SEQ ID NO: 35 - DNA, 62 bp, synthetic construct
  misc_feature 1..62: note = primer
  source 1..62: mol_type = other DNA
cctggagtccgatttcgcatttgcctgaccctaactgtgccttctagttgccagccatctgt

SEQ ID NO: 36 - DNA, 53 bp, synthetic construct
  misc_feature 1..53: note = primer
  source 1..53: mol_type = other DNA
cgttcttggcaatattgatggtgttcatggtggcgaccggtggatcccgggcc

SEQ ID NO: 37 - DNA, 47 bp, synthetic construct
  misc_feature 1..47: note = primer
  source 1..47: mol_type = other DNA
accttggccgactctggtaatggtaatacgactcactataggaaaaa

SEQ ID NO: 38 - DNA, 45 bp, synthetic construct
  misc_feature 1..45: note = primer
  source 1..45: mol_type = other DNA
agtcagtgagcgaggaagcccaaaaaacccctcaagacccgttta

SEQ ID NO: 39 - DNA, 43 bp, synthetic construct
  misc_feature 1..43: note = primer
  source 1..43: mol_type = other DNA
aaacgggtcttgaggggttttttgggcttcctcgctcactgac

SEQ ID NO: 40 - DNA, 38 bp, synthetic construct
  misc_feature 1..38: note = primer
  source 1..38: mol_type = other DNA
tagtgagtcgtattaccattaccagagtcggccaaggt

SEQ ID NO: 41 - DNA, 60 bp, synthetic construct
  misc_feature 1..60: note = primer
  source 1..60: mol_type = other DNA
gcccgggatccaccggtcgccacctcgccaccatgaggactctgaacacctctgccatgg

SEQ ID NO: 42 - DNA, 67 bp, synthetic construct
  misc_feature 1..67: note = primer
  source 1..67: mol_type = other DNA
cttttcgaactgcgggtggctccagagcggccgcgttcccgtggttgggtgctgaccgttttgtgtg

SEQ ID NO: 43 - DNA, 60 bp, synthetic construct
  misc_feature 1..60: note = primer
  source 1..60: mol_type = other DNA
acgcggccgctctggagccacccgcagttcgaaaagtaaagcggccgcgactctagatca

SEQ ID NO: 44 - DNA, 35 bp, synthetic construct
  misc_feature 1..35: note = primer
  source 1..35: mol_type = other DNA
gtgttcagagtcctcatggtggcgaggtggcgacc

SEQ ID NO: 45 - DNA, 45 bp, synthetic construct
  misc_feature 1..45: note = primer
  source 1..45: mol_type = other DNA
atccaccggtcgccaccatgaggactctgaacacctctgccatgg

SEQ ID NO: 46 - DNA, 60 bp, synthetic construct
  misc_feature 1..60: note = primer
  source 1..60: mol_type = other DNA
tgtggtatggctgattatgatttactgtaactcgaaaacgacttccatgtctaattcggg

SEQ ID NO: 47 - DNA, 60 bp, synthetic construct
  misc_feature 1..60: note = primer
  source 1..60: mol_type = other DNA
gttttcgagttacagtaaatcataatcagccataccacatttgtagaggttttacttgct

SEQ ID NO: 48 - DNA, 40 bp, synthetic construct
  misc_feature 1..40: note = primer
  source 1..40: mol_type = other DNA
tggcagaggtgttcagagtcctcatggtggcgaccggtgg

SEQ ID NO: 49 - DNA, 49 bp, synthetic construct
  misc_feature 1..49: note = primer
  source 1..49: mol_type = other DNA
caccatcaccatcaccatgttatggccacaaccatggaacaagagactt

SEQ ID NO: 50 - DNA, 60 bp, synthetic construct
  misc_feature 1..60: note = primer
  source 1..60: mol_type = other DNA
tcttgatgagctgttcttccaggaggataaagttgttcatggtggcgaccggtggatccc

SEQ ID NO: 51 - DNA, 60 bp, synthetic construct
  misc_feature 1..60: note = primer
  source 1..60: mol_type = other DNA
cgggcccgggatccaccggtcgccaccatgaacaactttatcctcctggaagaacagctc

SEQ ID NO: 52 - DNA, 49 bp, synthetic construct
  misc_feature 1..49: note = primer
  source 1..49: mol_type = other DNA
aagtctcttgttccatggttgtggccataacatggtgatggtgatggtg

SEQ ID NO: 53 - DNA, 26 bp, synthetic construct
  misc_feature 1..26: note = primer
  source 1..26: mol_type = other DNA
ctctcggcatggacgagctgtacaag

SEQ ID NO: 54 - DNA, 29 bp, synthetic construct
  misc_feature 1..29: note = primer
  source 1..29: mol_type = other DNA
ttacttgtacagctcgtccatgccgagag

SEQ ID NO: 55 - DNA, 55 bp, synthetic construct
  misc_feature 1..55: note = primer
  source 1..55: mol_type = other DNA
tgcgcgcaagtctcttgttccatggttgtggccatggtggcgaccggtggatccc

SEQ ID NO: 56 - DNA, 36 bp, synthetic construct
  misc_feature 1..36: note = primer
  source 1..36: mol_type = other DNA
cccgaattagacatggaagtcgttttcgagttacag

SEQ ID NO: 57 - DNA, 49 bp, synthetic construct
  misc_feature 1..49: note = primer
  source 1..49: mol_type = other DNA
gggatccaccggtcgccaccatggccacaaccatggaacaagagacttg

SEQ ID NO: 58 - DNA, 36 bp, synthetic construct
  misc_feature 1..36: note = primer
  source 1..36: mol_type = other DNA
ctgtaactcgaaaacgacttccatgtctaattcggg

SEQ ID NO: 59 - DNA, 40 bp, synthetic construct
  misc_feature 1..40: note = primer
  source 1..40: mol_type = other DNA
tctcttgttccatggttgtggccatggtggcgaccggtgg

SEQ ID NO: 60 - DNA, 60 bp, synthetic construct
  misc_feature 1..60: note = primer
  source 1..60: mol_type = other DNA
acgtggttttcctttgaaaaacacgatgataaatgaggactctgaacacctctgccatgg

SEQ ID NO: 61 - DNA, 54 bp, synthetic construct
  misc_feature 1..54: note = primer
  source 1..54: mol_type = other DNA
gcagaggtgttcagagtcctcatttatcatcgtgtttttcaaaggaaaaccacg

SEQ ID NO: 62 - DNA, 45 bp, synthetic construct
  misc_feature 1..45: note = primer
  source 1..45: mol_type = other DNA
agtcgttttcgagttacagtaatcccccccccctaacgttactgg

SEQ ID NO: 63 - DNA, 52 bp, synthetic construct
  misc_feature 1..52: note = primer
  source 1..52: mol_type = other DNA
ccagtaacgttaggggggggggattactgtaactcgaaaacgacttccatgt

SEQ ID NO: 64 - DNA, 39 bp, synthetic construct
  misc_feature 1..39: note = primer
  source 1..39: mol_type = other DNA
ccaccggtcgccaccatggccacaaccatggaacaagag

SEQ ID NO: 65 - DNA, 60 bp, synthetic construct
  misc_feature 1..60: note = primer
  source 1..60: mol_type = other DNA
gatggtgtcccccgccacctccgccacctccaagtcctgattctgcaatttcagccagtt

SEQ ID NO: 66 - DNA, 90 bp, synthetic construct
  misc_feature 1..90: note = primer
  source 1..90: mol_type = other DNA
aattgcagaatcaggacttggaggtggcggaggtggcgggggacaccatcaccatcaccatgtttaatcccccccccctaacgttactgg

SEQ ID NO: 67 - DNA, 50 bp, synthetic construct
  misc_feature 1..50: note = primer
  source 1..50: mol_type = other DNA
ttataggcggacagcagcagggtcagcaccatggtggcgaggtggcgacc

SEQ ID NO: 68 - DNA, 60 bp, synthetic construct
  misc_feature 1..60: note = primer
  source 1..60: mol_type = other DNA
cggccgctcgattacaaggatgacgacgataaggtttaaagcggccgcgactctagatca

SEQ ID NO: 69 - DNA, 60 bp, synthetic construct
  misc_feature 1..60: note = primer
  source 1..60: mol_type = other DNA
taaaccttatcgtcgtcatccttgtaatcgagcggccgcgttgtagggcccatgggggcg

SEQ ID NO: 70 - DNA, 60 bp, synthetic construct
  misc_feature 1..60: note = primer
  source 1..60: mol_type = other DNA
gcgggcccgggatccaccggtcgccacctcgccaccatggtgctgaccctgctgctgtcc

SEQ ID NO: 71 - DNA, 55 bp, synthetic construct
  misc_feature 1..55: note = primer
  source 1..55: mol_type = other DNA
ccctgtcttcatggggcgagtatatgaccccagggccggaggtggcggaggtggc

SEQ ID NO: 72 - DNA, 50 bp, synthetic construct
  misc_feature 1..50: note = primer
  source 1..50: mol_type = other DNA
ggagggtcagcagggtcagcctggaggccatggtggcgaccggtggatcc

SEQ ID NO: 73 - DNA, 56 bp, synthetic construct
  misc_feature 1..56: note = primer
  source 1..56: mol_type = other DNA
cggtaccgcgggcccgggatccaccggtcgccaccatggcctccaggctgaccctg

SEQ ID NO: 74 - DNA, 52 bp, synthetic construct
  misc_feature 1..52: note = primer
  source 1..52: mol_type = other DNA
tggtgtcccccgccacctccgccacctccggccctggggtcatatactcgcc

SEQ ID NO: 75 - DNA, 47 bp, synthetic construct
  misc_feature 1..47: note = primer
  source 1..47: mol_type = other DNA
aattgcagaatcaggacttggaggtggcggaggtggcgggggacacc

SEQ ID NO: 76 - DNA, 39 bp, synthetic construct
  misc_feature 1..39: note = primer
  source 1..39: mol_type = other DNA
cataatcagccataccacatttgtagaggttttacttgc

SEQ ID NO: 77 - DNA, 28 bp, synthetic construct
  misc_feature 1..28: note = primer
  source 1..28: mol_type = other DNA
tacttgtacagctcgtccatgccgagag

SEQ ID NO: 78 - DNA, 28 bp, synthetic construct
  misc_feature 1..28: note = primer
  source 1..28: mol_type = other DNA
ctctcggcatggacgagctgtacaagta

SEQ ID NO: 79 - DNA, 39 bp, synthetic construct
  misc_feature 1..39: note = primer
  source 1..39: mol_type = other DNA
gcaagtaaaacctctacaaatgtggtatggctgattatg

SEQ ID NO: 80 - DNA, 63 bp, synthetic construct
  misc_feature 1..63: note = primer
  source 1..63: mol_type = other DNA
tcctctctgcttctagaataaatcataatcagccataccacatttgtagaggttttacttgct

SEQ ID NO: 81 - DNA, 66 bp, synthetic construct
  misc_feature 1..66: note = primer
  source 1..66: mol_type = other DNA
tgtcatgaatcagtaggtccgcaaagtaaccagcgtagtgcttgtacagctcgtccatgccgagag

SEQ ID NO: 82 - DNA, 75 bp, synthetic construct
  misc_feature 1..75: note = primer
  source 1..75: mol_type = other DNA
actttgcggacctactgattcatgacattgagacaaatccagggatgaactttctacgtaagatagtgaaaaatt

SEQ ID NO: 83 - DNA, 60 bp, synthetic construct
  misc_feature 1..60: note = primer
  source 1..60: mol_type = other DNA
acctctacaaatgtggtatggctgattatgatttattctagaagcagagaggaatctttg

SEQ ID NO: 84 - DNA, 85 bp, synthetic construct
  misc_feature 1..85: note = primer
  source 1..85: mol_type = other DNA
gctggttactttgcggacctactgattcatgacattgagacaaatccagggggattcggacaccaaaacaaagcggtgtacactg

SEQ ID NO: 85 - DNA, 70 bp, synthetic construct
  misc_feature 1..70: note = primer
  source 1..70: mol_type = other DNA
aaacctctacaaatgtggtatggctgattatgatttgttccatggcttcttcttcgtaggcatacaagtc

SEQ ID NO: 86 - DNA, 81 bp, synthetic construct
  misc_feature 1..81: note = primer
  source 1..81: mol_type = other DNA
tgtctcaatgtcatgaatcagtaggtccgcaaagtaaccagcgtagtgcttgtacagctcgtccatgccgagagtgatccc

SEQ ID NO: 87 - DNA, 80 bp, synthetic construct
  misc_feature 1..80: note = primer
  source 1..80: mol_type = other DNA
gagacttgtatgcctacgaagaagaagccatggaacaaatcataatcagccataccacatttgtagaggttttacttgct

SEQ ID NO: 88 - DNA, 73 bp, synthetic construct
  misc_feature 1..73: note = primer
  source 1..73: mol_type = other DNA
catggcagaggtgttcagagtcctcatggtggcgaccggtggattcacgacacctgaaatggaagaaaaaaac

SEQ ID NO: 89 - DNA, 53 bp, synthetic construct
  misc_feature 1..53: note = primer
  source 1..53: mol_type = other DNA
attaccgccatgcattagttattaggctccggtgcccgtcagtgggcagagcg

SEQ ID NO: 90 - DNA, 67 bp, synthetic construct
  misc_feature 1..67: note = primer
  source 1..67: mol_type = other DNA
agtttttttcttccatttcaggtgtcgtgaatccaccggtcgccaccatgaggactctgaacacctc

SEQ ID NO: 91 - DNA, 56 bp, synthetic construct
  misc_feature 1..56: note = primer
  source 1..56: mol_type = other DNA
gtgcgctctgcccactgacgggcaccggagcctaataactaatgcatggcggtaat

SEQ ID NO: 92 - DNA, 67 bp, synthetic construct
  misc_feature 1..67: note = primer
  source 1..67: mol_type = other DNA
gaggccgaggccgcctcggcctctgagctaatccaccggtcgccaccatgaggactctgaacacctc

SEQ ID NO: 93 - DNA, 67 bp, synthetic construct
  misc_feature 1..67: note = primer
  source 1..67: mol_type = other DNA
ataaccgtattaccgccatgcattagttattaggtgtggaaagtccccaggctccccagcaggcaga

SEQ ID NO: 94 - DNA, 62 bp, synthetic construct
  misc_feature 1..62: note = primer
  source 1..62: mol_type = other DNA
ttcagagtcctcatggtggcgaccggtggattagctcagaggccgaggcggcctcggcctct

SEQ ID NO: 95 - DNA, 66 bp, synthetic construct
  misc_feature 1..66: note = primer
  source 1..66: mol_type = other DNA
tctgcctgctggggagcctggggactttccacacctaataactaatgcatggcggtaatacggtta

SEQ ID NO: 96 - DNA, 38 bp, synthetic construct
  misc_feature 1..38: note = primer
  source 1..38: mol_type = other DNA
ggaggtggcggaggtggcgggggacaccatcaccatca

SEQ ID NO: 97 - DNA, 64 bp, synthetic construct
  misc_feature 1..64: note = primer
  source 1..64: mol_type = other DNA
agacaacgctggccttttccagaggcgacctctgcatggtggcgaccggtggatcccgggcccg

SEQ ID NO: 98 - DNA, 65 bp, synthetic construct
  misc_feature 1..65: note = primer
  source 1..65: mol_type = other DNA
cgggcccgggatccaccggtcgccaccatgcagaggtcgcctctggaaaaggccagcgttgtctc

SEQ ID NO: 99 - DNA, 56 bp, synthetic construct
  misc_feature 1..56: note = primer
  source 1..56: mol_type = other DNA
ccccgccacctccgccacctccaagccttgtatcttgcacctcttcttctgtctcc

SEQ ID NO: 100 - DNA, 7808 bp, synthetic construct
  misc_feature 1..7808: note = synthetic construct
  source 1..7808: mol_type = other DNA
cgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccacgatgaagtgggtgaccttcatcagcctgctgttcctgttttcttccgcctacagcgaagtgcagctggttgaaagcggaggcggactggtccagccaggcagaagcctgagactgtcttgtgccgcctctggcttcacctttgacgactacgccatgcactgggtgcggcaggcccctggcaagggactcgagtgggtcagcgccatcacctggaatagcggccacatcgactacgcagatagcgttgaaggcagattcaccatctccagggacaacgccaagaattctctgtacctgcagatgaacagcctgcgggccgaggataccgctgtgtactactgcgccaaagtgtcctacctgagcaccgccagctccctggactactggggccagggcaccctggtgacagtgagctctgctagcacaaaaggacctagcgtgtttcccctggcccctagcagcaaaagcaccagcggcggaaccgccgctctgggttgtctggtgaaggactatttccctgaacctgtgaccgtgtcctggaactctggcgccctgactagcggcgtgcataccttccctgccgtgctgcaaagctctggcctgtatagcctttcttctgtggtgaccgtgcctagcagctctctgggcacacagacatacatctgcaatgtgaaccacaagccctccaacaccaaggtggacaaaaaggtggaacccaagagctgcgacaagacccacacctgtcctccgtgccccgctcctgagctgctgggcggcccttctgtgttcctgttcccccccaaacctaaagacacactgatgatcagccggacccctgaggtgacctgcgtggtggtggacgtgagccacgaggaccccgaggtgaagttcaactggtacgtggacggcgtggaggtccacaacgccaagaccaaacctagagaggaacaatacaacagcacatatagagtggtgtctgtgctgacagtgctccaccaggactggctgaacggaaaggaatacaagtgcaaggtgtccaacaaggccctccctgctccaatcgagaagaccattagcaaggccaagggccaacctagagagccccaggtctacaccctgccaccaagtagagatgagctgaccaagaaccaggtgagcctaacatgcctggtgaagggcttttaccccagcgacatcgccgtggaatgggagagcaacggccagcctgagaacaactacaagacaacacctcctgttctggattctgatggcagcttcttcctgtacagcaagctgacagtggataagagccggtggcagcagggcaacgtgttcagctgctccgttatgcacgaggccctgcataatcactacacccagaagagcctgtctctgagccctggcaaggaagtgcagctggttgaaagcggaggcggactggtccagccaggcagaagcctgagactgtcttgtgccgcctctggcttcacctttgacgactacgccatgcactgggtgcggcaggcccctggcaagggactcgagtgggtcagcgccatcacctggaatagcggccacatcgactacgcagatagcgttgaaggcagattcaccatctccagggacaacgccaagaattctctgtacctgcagatgaacagcctgcgggccgaggataccgctgtgtactactgcgccaaagtgtcctacctgagcaccgccagctccctggactactggggccagggcaccctggtgacagtgagctctgctagcacaaaaggacctagcgtgtttcccctggcccctagcagcaaaagcaccagcggcggaaccgccgctctgggttgtctggtgaaggactatttccctgaacctgtgaccgtgtcctggaactctggcgccctgactagcggcgtgcataccttccctgccgtgctgcaaagctctggcctgtatagcctttcttctgtggtgaccgtgcctagcagctctctgggcacacagacatacatctgcaatgtgaaccacaagccctccaacaccaaggtggacaaaaaggtggaacccaagagctgcgacaagacccacacctgtcctccgtgccccgctcctgagctgctgggcggcccttctgtgttcctgttcccccccaaacctaaagacacactgatgatcagccggacccctgaggtgacctgcgtggtggtggacgtgagccacgaggaccccgaggtgaagttcaactggtacgtggacggcgtggaggtccacaacgccaagaccaaacctagagaggaacaatacaacagcacatatagagtggtgtctgtgctgacagtgctccaccaggactggctgaacggaaaggaatacaagtgcaaggtgtccaacaaggccctccctgctccaatcgagaagaccattagcaaggccaagggccaacctagagagccccaggtctacaccctgccaccaagtagagatgagctgaccaagaaccaggtgagcctaacatgcctggtgaagggcttttaccccagcgacatcgccgtggaatgggagagcaacggccagcctgagaacaactacaagacaacacctcctgttctggattctgatggcagcttcttcctgtacagcaagctgacagtggataagagccggtggcagcagggcaacgtgttcagctgctccgttatgcacgaggccctgcataatcactacacccagaagagcctgtctctgagccctggcaagcaagcgaaaacggcgcggaagcggagctactaacttcagcctgctgaagcaggctggagatgtggaggagaaccctggacctgatatccagatgacccagtctccatctagcctgagcgccagcgtgggagatagagtgaccatcacctgtagagcctctcaaggcatccggaactacctggcctggtatcagcagaaacctggcaaggctcctaagctgctgatctacgccgcttccaccctgcagagcggcgttccttctagattcagcggcagcggctccggaacagacttcaccctgacaattagctccctgcaacctgaagatgtggctacatactactgccagagatacaatcgggccccttacacctttggacagggcaccaaggtggaaatcaagcggaccgtggccgccccatctgtgttcatcttcccccccagcgacgagcagctgaaaagcggcacagccagcgtggtgtgcctgctgaacaacttctaccccagggaagccaaggtgcagtggaaggtggacaatgccctgcagagcggcaacagccaggagagcgtgaccgagcaggacagcaaggacagcacctacagcctgagcagcaccctcacactgtctaaagccgactacgagaagcacaaggtctacgcctgcgaggtgacccaccagggcctgtcctcccctgtgacaaagagctttaacagaggcgagtgctaagatatccagatgacccagtctccatctagcctgagcgccagcgtgggagatagagtgaccatcacctgtagagcctctcaaggcatccggaactacctggcctggtatcagcagaaacctggcaaggctcctaagctgctgatctacgccgcttccaccctgcagagcggcgttccttctagattcagcggcagcggctccggaacagacttcaccctgacaattagctccctgcaacctgaagatgtggctacatactactgccagagatacaatcgggccccttacacctttggacagggcaccaaggtggaaatcaagcggaccgtggccgccccatctgtgttcatcttcccccccagcgacgagcagctgaaaagcggcacagccagcgtggtgtgcctgctgaacaacttctaccccagggaagccaaggtgcagtggaaggtggacaatgccctgcagagcggcaacagccaggagagcgtgaccgagcaggacagcaaggacagcacctacagcctgagcagcaccctcacactgtctaaagccgactacgagaagcacaaggtctacgcctgcgaggtgacccaccagggcctgtcctcccctgtgacaaagagctttaacagaggcgagtgctaacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaaatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactctt

SEQ ID NO: 101 - DNA, 28 bp, synthetic construct
  misc_feature 1..28: note = synthetic construct
  source 1..28: mol_type = other DNA
cgcgatgtacgggccagatatacgcgtt

SEQ ID NO: 102 - DNA, 380 bp, synthetic construct
  misc_feature 1..380: note = CMV enhancer
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 103 - DNA, 204 bp, synthetic construct
  misc_feature 1..204: note = CMV promoter
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 104 - DNA, 110 bp, synthetic construct
  misc_feature 1..110: note = synthetic construct
  source 1..110: mol_type = other DNA
ggtttagtgaaccgtcagatccgctagcgctaccggactcagatctcgagctcaagcttcgaattctgcagtcgacggtaccgcgggcccgggatccaccggtcgccacg

SEQ ID NO: 105 - DNA, 54 bp, synthetic construct
  misc_feature 1..54: note = Albumin signal peptide (codon optimized)
  source 1..54: mol_type = other DNA
atgaagtgggtgaccttcatcagcctgctgttcctgttttcttccgcctacagc

SEQ ID NO: 106 - DNA, 363 bp, synthetic construct
  misc_feature 1..363: note = Adalimumab Heavy Chain (variable region)
  source 1..363: mol_type = other DNA
gaagtgcagctggttgaaagcggaggcggactggtccagccaggcagaagcctgagactgtcttgtgccgcctctggcttcacctttgacgactacgccatgcactgggtgcggcaggcccctggcaagggactcgagtgggtcagcgccatcacctggaatagcggccacatcgactacgcagatagcgttgaaggcagattcaccatctccagggacaacgccaagaattctctgtacctgcagatgaacagcctgcgggccgaggataccgctgtgtactactgcgccaaagtgtcctacctgagcaccgccagctccctggactactggggccagggcaccctggtgacagtgagctct

SEQ ID NO: 107 - DNA, 990 bp, Homo sapiens
  source 1..990: mol_type = genomic DNA
gctagcacaaaaggacctagcgtgtttcccctggcccctagcagcaaaagcaccagcggcggaaccgccgctctgggttgtctggtgaaggactatttccctgaacctgtgaccgtgtcctggaactctggcgccctgactagcggcgtgcataccttccctgccgtgctgcaaagctctggcctgtatagcctttcttctgtggtgaccgtgcctagcagctctctgggcacacagacatacatctgcaatgtgaaccacaagccctccaacaccaaggtggacaaaaaggtggaacccaagagctgcgacaagacccacacctgtcctccgtgccccgctcctgagctgctgggcggcccttctgtgttcctgttcccccccaaacctaaagacacactgatgatcagccggacccctgaggtgacctgcgtggtggtggacgtgagccacgaggaccccgaggtgaagttcaactggtacgtggacggcgtggaggtccacaacgccaagaccaaacctagagaggaacaatacaacagcacatatagagtggtgtctgtgctgacagtgctccaccaggactggctgaacggaaaggaatacaagtgcaaggtgtccaacaaggccctccctgctccaatcgagaagaccattagcaaggccaagggccaacctagagagccccaggtctacaccctgccaccaagtagagatgagctgaccaagaaccaggtgagcctaacatgcctggtgaagggcttttaccccagcgacatcgccgtggaatgggagagcaacggccagcctgagaacaactacaagacaacacctcctgttctggattctgatggcagcttcttcctgtacagcaagctgacagtggataagagccggtggcagcagggcaacgtgttcagctgctccgttatgcacgaggccctgcataatcactacacccagaagagcctgtctctgagccctggcaag

SEQ ID NO: 108 - DNA, 16 bp, synthetic construct
  misc_feature 1..16: note = Furin cleavage site
  source 1..16: mol_type = other DNA
caagcgaaaacggcgc

SEQ ID NO: 109 - skipped

SEQ ID NO: 110 - DNA, 57 bp, synthetic construct
  misc_feature 1..57: note = P2A self-cleaving peptide
  source 1..57: mol_type = other DNA
gctactaacttcagcctgctgaagcaggctggagatgtggaggagaaccctggacct

SEQ ID NO: 111 - DNA, 321 bp, synthetic construct
  misc_feature 1..321: note = Adalimumab Light Chain (variable region)
  source 1..321: mol_type = other DNA
gatatccagatgacccagtctccatctagcctgagcgccagcgtgggagatagagtgaccatcacctgtagagcctctcaaggcatccggaactacctggcctggtatcagcagaaacctggcaaggctcctaagctgctgatctacgccgcttccaccctgcagagcggcgttccttctagattcagcggcagcggctccggaacagacttcaccctgacaattagctccctgcaacctgaagatgtggctacatactactgccagagatacaatcgggccccttacacctttggacagggcaccaaggtggaaatcaag

SEQ ID NO: 112 - DNA, 324 bp, Homo sapiens
  source 1..324: mol_type = genomic DNA
cggaccgtggccgccccatctgtgttcatcttcccccccagcgacgagcagctgaaaagcggcacagccagcgtggtgtgcctgctgaacaacttctaccccagggaagccaaggtgcagtggaaggtggacaatgccctgcagagcggcaacagccaggagagcgtgaccgagcaggacagcaaggacagcacctacagcctgagcagcaccctcacactgtctaaagccgactacgagaagcacaaggtctacgcctgcgaggtgacccaccagggcctgtcctcccctgtgacaaagagctttaacagaggcgagtgctaa

SEQ ID NO: 113 - DNA, 12 bp, synthetic construct
  misc_feature 1..12: note = synthetic construct
  source 1..12: mol_type = other DNA
ccccccccccta

SEQ ID NO: 114 - DNA, 552 bp, synthetic construct
  misc_feature 1..552: note = IRES
  source 1..552: mol_type = other DNA
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 115 - DNA, 204 bp, synthetic construct; identical to SEQ ID NO: 22
  misc_feature 1..204: note = Enhancer peptide
  source 1..204: mol_type = other DNA

SEQ ID NO: 116 - DNA, 105 bp, synthetic construct
  misc_feature 1..105: note = synthetic construct
  source 1..105: mol_type = other DNA
atcataatcagccataccacatttgtagaggttttacttgctttaaaaaacctcccacacctccccctgaacctgaaacataaaatgaatgcaattgttgttgtt

SEQ ID NO: 117 - DNA, 122 bp, synthetic construct
  misc_feature 1..122: note = SV40 poly(A) signal
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 118 - DNA, 178 bp, synthetic construct
  misc_feature 1..178: note = Linker
  source 1..178: mol_type = other DNA
aggcgtcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgc

SEQ ID NO: 119 - DNA, 795 bp, synthetic construct
  misc_feature 1..795: note = Neomycin/Kanamycin resistance gene
  source 1..795: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 120 - DNA, 325 bp, synthetic construct
  misc_feature 1..325: note = synthetic construct
  source 1..325: mol_type = other DNA
attattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 121 - DNA, 589 bp, synthetic construct
  misc_feature 1..589: note = Origin of replication
  source 1..589: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 122 - DNA, 72 bp, synthetic construct
  misc_feature 1..72: note = synthetic construct
  source 1..72: mol_type = other DNA
aacgccagcaacgcggcctttttacggttcctgggcttttgctggccttttgctcacatgttcttgactctt

SEQ ID NO: 123 - protein, 18 aa, synthetic construct
  REGION 1..18: note = Albumin signal peptide
MKWVTFISLLFLFSSAYS

SEQ ID NO: 124 - protein, 121 aa, synthetic construct [sequence omitted]

SEQ ID NO: 125 - protein, 330 aa, Homo sapiens [sequence omitted]

SEQ ID NO: 126 - protein, 4 aa, synthetic construct
  REGION 1..4: note = Furin cleavage site
RKRR

SEQ ID NO: 127 - skipped

SEQ ID NO: 128 - protein, 19 aa, synthetic construct
  REGION 1..19: note = P2A self-cleaving peptide
ATNFSLLKQAGDVEENPGP

SEQ ID NO: 129 - protein, 107 aa, synthetic construct [sequence omitted]

SEQ ID NO: 130 - protein, 107 aa, Homo sapiens [sequence omitted]

SEQ ID NO: 131 - protein, 67 aa, synthetic construct; identical to SEQ ID NO: 2
  REGION 1..67: note = Enhancer peptide

SEQ ID NO: 132 - protein, 451 aa, synthetic construct
  REGION 1..451: note = Albumin complete heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 214 aa, synthetic construct
  REGION 1..214: note = Albumin complete light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - DNA, 1353 bp, synthetic construct
  misc_feature 1..1353: note = Albumin Heavy Chain complete
  source 1..1353: mol_type = other DNA
gaagtgcagctggttgaaagcggaggcggactggtccagccaggcagaagcctgagactgtcttgtgccgcctctggcttcacctttgacgactacgccatgcactgggtgcggcaggcccctggcaagggactcgagtgggtcagcgccatcacctggaatagcggccacatcgactacgcagatagcgttgaaggcagattcaccatctccagggacaacgccaagaattctctgtacctgcagatgaacagcctgcgggccgaggataccgctgtgtactactgcgccaaagtgtcctacctgagcaccgccagctccctggactactggggccagggcaccctggtgacagtgagctctgctagcacaaaaggacctagcgtgtttcccctggcccctagcagcaaaagcaccagcggcggaaccgccgctctgggttgtctggtgaaggactatttccctgaacctgtgaccgtgtcctggaactctggcgccctgactagcggcgtgcataccttccctgccgtgctgcaaagctctggcctgtatagcctttcttctgtggtgaccgtgcctagcagctctctgggcacacagacatacatctgcaatgtgaaccacaagccctccaacaccaaggtggacaaaaaggtggaacccaagagctgcgacaagacccacacctgtcctccgtgccccgctcctgagctgctgggcggcccttctgtgttcctgttcccccccaaacctaaagacacactgatgatcagccggacccctgaggtgacctgcgtggtggtggacgtgagccacgaggaccccgaggtgaagttcaactggtacgtggacggcgtggaggtccacaacgccaagaccaaacctagagaggaacaatacaacagcacatatagagtggtgtctgtgctgacagtgctccaccaggactggctgaacggaaaggaatacaagtgcaaggtgtccaacaaggccctccctgctccaatcgagaagaccattagcaaggccaagggccaacctagagagccccaggtctacaccctgccaccaagtagagatgagctgaccaagaaccaggtgagcctaacatgcctggtgaagggcttttaccccagcgacatcgccgtggaatgggagagcaacggccagcctgagaacaactacaagacaacacctcctgttctggattctgatggcagcttcttcctgtacagcaagctgacagtggataagagccggtggcagcagggcaacgtgttcagctgctccgttatgcacgaggccctgcataatcactacacccagaagagcctgtctctgagccctggcaag

SEQ ID NO: 135 - DNA, 645 bp, synthetic construct
  misc_feature 1..645: note = Albumin Light Chain complete
  source 1..645: mol_type = other DNA
gatatccagatgacccagtctccatctagcctgagcgccagcgtgggagatagagtgaccatcacctgtagagcctctcaaggcatccggaactacctggcctggtatcagcagaaacctggcaaggctcctaagctgctgatctacgccgcttccaccctgcagagcggcgttccttctagattcagcggcagcggctccggaacagacttcaccctgacaattagctccctgcaacctgaagatgtggctacatactactgccagagatacaatcgggccccttacacctttggacagggcaccaaggtggaaatcaagcggaccgtggccgccccatctgtgttcatcttcccccccagcgacgagcagctgaaaagcggcacagccagcgtggtgtgcctgctgaacaacttctaccccagggaagccaaggtgcagtggaaggtggacaatgccctgcagagcggcaacagccaggagagcgtgaccgagcaggacagcaaggacagcacctacagcctgagcagcaccctcacactgtctaaagccgactacgagaagcacaaggtctacgcctgcgaggtgacccaccagggcctgtcctcccctgtgacaaagagctttaacagaggcgagtgctaa

SEQ ID NO: 136 - DNA, 5096 bp, synthetic construct
  misc_feature 1..5096: note = synthetic construct
  source 1..5096: mol_type = other DNA
cgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccacgatgaagtgggtgaccttcatcagcctgctgttcctgttttcttccgcctacagcgaagtgcagctggttgaaagcggaggcggactggtccagccaggcagaagcctgagactgtcttgtgccgcctctggcttcacctttgacgactacgccatgcactgggtgcggcaggcccctggcaagggactcgagtgggtcagcgccatcacctggaatagcggccacatcgactacgcagatagcgttgaaggcagattcaccatctccagggacaacgccaagaattctctgtacctgcagatgaacagcctgcgggccgaggataccgctgtgtactactgcgccaaagtgtcctacctgagcaccgccagctccctggactactggggccagggcaccctggtgacagtgagctctgctagcacaaaaggacctagcgtgtttcccctggcccctagcagcaaaagcaccagcggcggaaccgccgctctgggttgtctggtgaaggactatttccctgaacctgtgaccgtgtcctggaactctggcgccctgactagcggcgtgcataccttccctgccgtgctgcaaagctctggcctgtatagcctttcttctgtggtgaccgtgcctagcagctctctgggcacacagacatacatctgcaatgtgaaccacaagccctccaacaccaaggtggacaaaaaggtggaacccaagagctgcgacaagacccacacctgtcctccgtgccccgctcctgagctgctgggcggcccttctgtgttcctgttcccccccaaacctaaagacacactgatgatcagccggacccctgaggtgacctgcgtggtggtggacgtgagccacgaggaccccgaggtgaagttcaactggtacgtggacggcgtggaggtccacaacgccaagaccaaacctagagaggaacaatacaacagcacatatagagtggtgtctgtgctgacagtgctccaccaggactggctgaacggaaaggaatacaagtgcaaggtgtccaacaaggccctccctgctccaatcgagaagaccattagcaaggccaagggccaacctagagagccccaggtctacaccctgccaccaagtagagatgagctgaccaagaaccaggtgagcctaacatgcctggtgaagggcttttaccccagcgacatcgccgtggaatgggagagcaacggccagcctgagaacaactacaagacaacacctcctgttctggattctgatggcagcttcttcctgtacagcaagctgacagtggataagagccggtggcagcagggcaacgtgttcagctgctccgttatgcacgaggccctgcataatcactacacccagaagagcctgtctctgagccctggcaagcaagcgaaaacggcgcggaagcggagctactaacttcagcctgctgaagcaggctggagatgtggaggagaaccctggacctatgaagtgggtgaccttcatcagcctgctgttcctgttttcttccgcctacagcgatatccagatgacccagtctccatctagcctgagcgccagcgtgggagatagagtgaccatcacctgtagagcctctcaaggcatccggaactacctggcctggtatcagcagaaacctggcaaggctcctaagctgctgatctacgccgcttccaccctgcagagcggcgttccttctagattcagcggcagcggctccggaacagacttcaccctgacaattagctccctgcaacctgaagatgtggctacatactactgccagagatacaatcgggccccttacacctttggacagggcaccaaggtggaaatcaagcggaccgtggccgccccatctgtgttcatcttcccccccagcgacgagcagctgaaaagcggcacagccagcgtggtgtgcctgctgaacaacttctaccccagggaagccaaggtgcagtggaaggtggacaatgccctgcagagcggcaacagccaggagagcgtgaccgagcaggacagcaaggacagcacctacagcctgagcagcaccctcacactgtctaaagccgactacgagaagcacaaggtctacgcctgcgaggtgacccaccagggcctgtcctcccctgtgacaaagagctttaacagaggcgagtgctaaatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactctt

SEQ ID NO: 137 - protein, 9 aa, synthetic construct
  REGION 1..9: note = CDR
  VARIANT 9: note = T or A
QRYNRAPYX

SEQ ID NO: 138 - protein, 12 aa, synthetic construct
  REGION 1..12: note = CDR
  VARIANT 12: note = Y or N
VSYLSTASSLDX

SEQ ID NO: 139 - protein, 7 aa, synthetic construct
  REGION 1..7: note = CDR
AASTLQS

SEQ ID NO: 140 - protein, 17 aa, synthetic construct
  REGION 1..17: note = CDR
AITWNSGHIDYADSVEG

SEQ ID NO: 141 - protein, 11 aa, synthetic construct
  REGION 1..11: note = CDR
RASQGIRNYLA

SEQ ID NO: 142 - protein, 5 aa, synthetic construct
  REGION 1..5: note = CDR
DYAMH

SEQ ID NO: 143 - protein, 9 aa, synthetic construct
  REGION 1..9: note = CDR
QKYNSAPYA

SEQ ID NO: 144 - protein, 9 aa, synthetic construct
  REGION 1..9: note = CDR
QKYNRAPYA

SEQ ID NO: 145 - protein, 9 aa, synthetic construct
  REGION 1..9: note = CDR
QKYQRAPYT

SEQ ID NO: 146 - protein, 9 aa, synthetic construct
  REGION 1..9: note = CDR
QKYSSAPYT

SEQ ID NO: 147 - protein, 9 aa, synthetic construct
  REGION 1..9: note = CDR
QKYNSAPYT

SEQ ID NO: 148 - protein, 9 aa, synthetic construct
  REGION 1..9: note = CDR
QKYNRAPYT

SEQ ID NO: 149 - protein, 9 aa, synthetic construct
  REGION 1..9: note = CDR
QKYNSAPYY

SEQ ID NO: 150 - protein, 9 aa, synthetic construct
  REGION 1..9: note = CDR
QKYNSAPYN

SEQ ID NO: 151 - protein, 9 aa, synthetic construct
  REGION 1..9: note = CDR
QKYTSAPYT

SEQ ID NO: 152 - protein, 9 aa, synthetic construct
  REGION 1..9: note = CDR
QKYNRAPYN

SEQ ID NO: 153 - protein, 9 aa, synthetic construct
  REGION 1..9: note = CDR
QKYNSAAYS

SEQ ID NO: 154 - protein, 9 aa, synthetic construct
  REGION 1..9: note = CDR
QQYNSAPDT

SEQ ID NO: 155 - protein, 9 aa, synthetic construct
  REGION 1..9: note = CDR
QKYNSDPYT

SEQ ID NO: 156 - protein, 9 aa, synthetic construct
  REGION 1..9: note = CDR
QKYISAPYT

SEQ ID NO: 157 - protein, 9 aa, synthetic construct
  REGION 1..9: note = CDR
QKYNRPPYT

SEQ ID NO: 158 - protein, 9 aa, synthetic construct
  REGION 1..9: note = CDR
QRYNRAPYA

SEQ ID NO: 159 - protein, 12 aa, synthetic construct
  REGION 1..12: note = CDR
ASYLSTSSSLDN

SEQ ID NO: 160 - protein, 12 aa, synthetic construct
  REGION 1..12: note = CDR
ASYLSTSSSLDK

SEQ ID NO: 161 - protein, 12 aa, synthetic construct
  REGION 1..12: note = CDR
ASYLSTSSSLDY

SEQ ID NO: 162 - protein, 12 aa, synthetic construct
  REGION 1..12: note = CDR
ASYLSTSSSLDD

SEQ ID NO: 163 - protein, 12 aa, synthetic construct
  REGION 1..12: note = CDR
ASYLSTSFSLDY

SEQ ID NO: 164 - protein, 12 aa, synthetic construct
  REGION 1..12: note = CDR
ASYLSTSSSLHY

SEQ ID NO: 165 - protein, 12 aa, synthetic construct
  REGION 1..12: note = CDR
ASFLSTSSSLEY

SEQ ID NO: 166 - protein, 12 aa, synthetic construct
  REGION 1..12: note = CDR
ASYLSTASSLEY

SEQ ID NO: 167 - protein, 12 aa, synthetic construct
  REGION 1..12: note = CDR
VSYLSTASSLDN

SEQ ID NO: 168 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 169 - DNA, 380 bp, synthetic construct; identical to SEQ ID NO: 102
  misc_feature 1..380: note = CMV Enhancer
  source 1..380: mol_type = other DNA

SEQ ID NO: 170 - DNA, 204 bp, synthetic construct; identical to SEQ ID NO: 103
  misc_feature 1..204: note = CMV promoter
  source 1..204: mol_type = other DNA

SEQ ID NO: 171 - DNA, 22 bp, synthetic construct
  misc_feature 1..22: note = Linker region
  source 1..22: mol_type = other DNA
ggtttagtgaaccgtcagatcc

SEQ ID NO: 172 - DNA, 81 bp, synthetic construct
  misc_feature 1..81: note = Multiple cloning site (MSC)
  source 1..81: mol_type = other DNA
gctagcgctaccggactcagatctcgagctcaagcttcgaattctgcagtcgacggtaccgcgggcccgggatccaccggt

SEQ ID NO: 173 - skipped

SEQ ID NO: 174 - DNA, 54 bp, synthetic construct; identical to SEQ ID NO: 105
  misc_feature 1..54: note = Albumin signal peptide
  source 1..54: mol_type = other DNA

SEQ ID NO: 175 - DNA, 363 bp, synthetic construct; identical to SEQ ID NO: 106
  misc_feature 1..363: note = Adalimumab variable heavy (VH) domain
  source 1..363: mol_type = other DNA

SEQ ID NO: 176 - DNA, 990 bp, synthetic construct; identical to SEQ ID NO: 107
  misc_feature 1..990: note = hIgG1 constant heavy (CH) domain
  source 1..990: mol_type = other DNA

SEQ ID NO: 177 - DNA, 12 bp, synthetic construct
  misc_feature 1..12: note = Furin cleavage site
  source 1..12: mol_type = other DNA
cgaaaacggcgc

SEQ ID NO: 178 - skipped

SEQ ID NO: 179 - DNA, 57 bp, synthetic construct; identical to SEQ ID NO: 110
  misc_feature 1..57: note = P2A cleavage site
  source 1..57: mol_type = other DNA

SEQ ID NO: 180 - DNA, 54 bp, synthetic construct; identical to SEQ ID NO: 105
  misc_feature 1..54: note = Albumin signal peptide
  source 1..54: mol_type = other DNA

SEQ ID NO: 181 - DNA, 321 bp, synthetic construct; identical to SEQ ID NO: 111
  misc_feature 1..321: note = Adalimumab variable light (VL) chain
  source 1..321: mol_type = other DNA

SEQ ID NO: 182 - DNA, 321 bp, synthetic construct
  misc_feature 1..321: note = hIgG kappa constant light (CL) chain
  source 1..321: mol_type = other DNA
cggaccgtggccgccccatctgtgttcatcttcccccccagcgacgagcagctgaaaagcggcacagccagcgtggtgtgcctgctgaacaacttctaccccagggaagccaaggtgcagtggaaggtggacaatgccctgcagagcggcaacagccaggagagcgtgaccgagcaggacagcaaggacagcacctacagcctgagcagcaccctcacactgtctaaagccgactacgagaagcacaaggtctacgcctgcgaggtgacccaccagggcctgtcctcccctgtgacaaagagctttaacagaggcgagtgc

SEQ ID NO: 183 - skipped

SEQ ID NO: 184 - DNA, 105 bp, synthetic construct; identical to SEQ ID NO: 116
  misc_feature 1..105: note = Linker region
  source 1..105: mol_type = other DNA

SEQ ID NO: 185 - DNA, 122 bp, synthetic construct; identical to SEQ ID NO: 117
  misc_feature 1..122: note = SV40 polyadenylation signal
  source 1..122: mol_type = other DNA

SEQ ID NO: 186 - DNA, 178 bp, synthetic construct; identical to SEQ ID NO: 118
  misc_feature 1..178: note = Linker region
  source 1..178: mol_type = other DNA

SEQ ID NO: 187 - DNA, 795 bp, synthetic construct; identical to SEQ ID NO: 119
  misc_feature 1..795: note = Neomycin/Kanamycin resistance gene
  source 1..795: mol_type = other DNA

SEQ ID NO: 188 - DNA, 325 bp, synthetic construct; identical to SEQ ID NO: 120
  misc_feature 1..325: note = Linker region
  source 1..325: mol_type = other DNA

SEQ ID NO: 189 - DNA, 589 bp, synthetic construct; identical to SEQ ID NO: 121
  misc_feature 1..589: note = Ori
  source 1..589: mol_type = other DNA

SEQ ID NO: 190 - DNA, 100 bp, synthetic construct
  misc_feature 1..100: note = Linker region
  source 1..100: mol_type = other DNA
aacgccagcaacgcggcctttttacggttcctgggcttttgctggccttttgctcacatgttcttgactcttcgcgatgtacgggccagatatacgcgtt

SEQ ID NO: 191 - DNA, 380 bp, synthetic construct; identical to SEQ ID NO: 102
  misc_feature 1..380: note = CMV Enhancer
  source 1..380: mol_type = other DNA

SEQ ID NO: 192 - DNA, 204 bp, synthetic construct; identical to SEQ ID NO: 103
  misc_feature 1..204: note = CMV promoter
  source 1..204: mol_type = other DNA

SEQ ID NO: 193 - DNA, 22 bp, synthetic construct
  misc_feature 1..22: note = Linker region
  source 1..22: mol_type = other DNA
ggtttagtgaaccgtcagatcc

SEQ ID NO: 194 - DNA, 81 bp, synthetic construct; identical to SEQ ID NO: 172
  misc_feature 1..81: note = Multiple cloning site (MSC)
  source 1..81: mol_type = other DNA

SEQ ID NO: 195 - skipped

SEQ ID NO: 196 - DNA, 54 bp, synthetic construct; identical to SEQ ID NO: 105
  misc_feature 1..54: note = Albumin signal peptide
  source 1..54: mol_type = other DNA

SEQ ID NO: 197 - DNA, 363 bp, synthetic construct; identical to SEQ ID NO: 106
  misc_feature 1..363: note = Adalimumab variable heavy (VH) domain
  source 1..363: mol_type = other DNA

SEQ ID NO: 198 - DNA, 990 bp, synthetic construct; identical to SEQ ID NO: 107
  misc_feature 1..990: note = hIgG1 constant heavy (CH) domain
  source 1..990: mol_type = other DNA

SEQ ID NO: 199 - DNA, 12 bp, synthetic construct
  misc_feature 1..12: note = Furin cleavage site
  source 1..12: mol_type = other DNA
cgaaaacggcgc

SEQ ID NO: 200 - skipped

SEQ ID NO: 201 - DNA, 57 bp, synthetic construct; identical to SEQ ID NO: 110
  misc_feature 1..57: note = P2A cleavage site
  source 1..57: mol_type = other DNA

SEQ ID NO: 202 - DNA, 54 bp, synthetic construct; identical to SEQ ID NO: 105
  misc_feature 1..54: note = Albumin signal peptide
  source 1..54: mol_type = other DNA

SEQ ID NO: 203 - DNA, 321 bp, synthetic construct; identical to SEQ ID NO: 111
  misc_feature 1..321: note = Adalimumab variable light (VL) chain
  source 1..321: mol_type = other DNA

SEQ ID NO: 204 - DNA, 321 bp, synthetic construct; identical to SEQ ID NO: 182
  misc_feature 1..321: note = hIgG kappa constant light (CL) chain
  source 1..321: mol_type = other DNA

SEQ ID NO: 205 - skipped

SEQ ID NO: 206 - DNA, 12 bp, synthetic construct
  misc_feature 1..12: note = Linker region
  source 1..12: mol_type = other DNA
ccccccccccta

SEQ ID NO: 207 - DNA, 552 bp, synthetic construct; identical to SEQ ID NO: 114
  misc_feature 1..552: note = Internal ribosome entry site (IRES) (non-coding)
  source 1..552: mol_type = other DNA

SEQ ID NO: 208 - DNA, 201 bp, synthetic construct
  misc_feature 1..201: note = Enhancer protein L
  source 1..201: mol_type = other DNA
atggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacag

SEQ ID NO: 209 - skipped

SEQ ID NO: 210 - DNA, 105 bp, synthetic construct; identical to SEQ ID NO: 116
  misc_feature 1..105: note = Linker region
  source 1..105: mol_type = other DNA

SEQ ID NO: 211 - DNA, 122 bp, synthetic construct; identical to SEQ ID NO: 117
  misc_feature 1..122: note = SV40 polyadenylation signal
  source 1..122: mol_type = other DNA

SEQ ID NO: 212 - DNA, 178 bp, synthetic construct; identical to SEQ ID NO: 118
  misc_feature 1..178: note = Linker region
  source 1..178: mol_type = other DNA

SEQ ID NO: 213 - DNA, 795 bp, synthetic construct; identical to SEQ ID NO: 119
  misc_feature 1..795: note = Neomycin/Kanamycin resistance gene
  source 1..795: mol_type = other DNA

SEQ ID NO: 214 - DNA, 325 bp, synthetic construct; identical to SEQ ID NO: 120
  misc_feature 1..325: note = Linker region
  source 1..325: mol_type = other DNA

SEQ ID NO: 215 - DNA, 589 bp, synthetic construct; identical to SEQ ID NO: 121
  misc_feature 1..589: note = Ori
  source 1..589: mol_type = other DNA

SEQ ID NO: 216 - DNA, 100 bp, synthetic construct; identical to SEQ ID NO: 190
  misc_feature 1..100: note = Linker region
  source 1..100: mol_type = other DNA

SEQ ID NO: 217 - DNA, 141 bp, synthetic construct
  misc_feature 1..141: note = 5' inverted terminal repeat (ITR)
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 218 - DNA, 27 bp, synthetic construct
  misc_feature 1..27: note = Linker region
  source 1..27: mol_type = other DNA
tctagacaactttgtatagaaaagttg

SEQ ID NO: 219 - DNA, 589 bp, synthetic construct
  misc_feature 1..589: note = CMV enhancer and promoter
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 220 - DNA, 30 bp, synthetic construct
  misc_feature 1..30: note = Linker region
  source 1..30: mol_type = other DNA
caagtttgtacaaaaaagcaggctgccacc

SEQ ID NO: 221 - DNA, 54 bp, synthetic construct; identical to SEQ ID NO: 105
  misc_feature 1..54: note = Albumin signal peptide
  source 1..54: mol_type = other DNA

SEQ ID NO: 222 - DNA, 363 bp, synthetic construct; identical to SEQ ID NO: 106
  misc_feature 1..363: note = Adalimumab variable heavy (VH) domain
  source 1..363: mol_type = other DNA

SEQ ID NO: 223 - DNA, 990 bp, synthetic construct; identical to SEQ ID NO: 107
  misc_feature 1..990: note = hIgG1 constant heavy (CH) domain
  source 1..990: mol_type = other DNA

SEQ ID NO: 224 - DNA, 12 bp, synthetic construct
  misc_feature 1..12: note = Furin cleavage site
  source 1..12: mol_type = other DNA
cgaaaacggcgc

SEQ ID NO: 225 - skipped

SEQ ID NO: 226 - DNA, 57 bp, synthetic construct; identical to SEQ ID NO: 110
  misc_feature 1..57: note = P2A cleavage site
  source 1..57: mol_type = other DNA

SEQ ID NO: 227 - DNA, 54 bp, synthetic construct; identical to SEQ ID NO: 105
  misc_feature 1..54: note = Albumin signal peptide
  source 1..54: mol_type = other DNA

SEQ ID NO: 228 - DNA, 321 bp, synthetic construct; identical to SEQ ID NO: 111
  misc_feature 1..321: note = Adalimumab variable light (VL) chain
  source 1..321: mol_type = other DNA

SEQ ID NO: 229 - DNA, 321 bp, synthetic construct; identical to SEQ ID NO: 182
  misc_feature 1..321: note = hIgG kappa constant light (CL) chain
  source 1..321: mol_type = other DNA

SEQ ID NO: 230 - skipped

SEQ ID NO: 231 - DNA, 54 bp, synthetic construct
  misc_feature 1..54: note = Linker region
  source 1..54: mol_type = other DNA
acccagctttcttgtacaaagtgggaattcctagagctcgctgatcagcctcga

SEQ ID NO: 232 - DNA, 208 bp, synthetic construct
  misc_feature 1..208: note = BGH polyadenylation signal
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 233 - skipped

SEQ ID NO: 234 - DNA, 141 bp, synthetic construct
  misc_feature 1..141: note = 3' inverted terminal repeat (ITR)
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 235 - DNA, 74 bp, synthetic construct
  misc_feature 1..74: note = Linker region
  source 1..74: mol_type = other DNA
ggcgcctgatgcggtattttctccttacgcatctgtgcggtatttcacaccgcatacgtcaaagcaaccatagt

SEQ ID NO: 236 - DNA, 456 bp, synthetic construct
  misc_feature 1..456: note = F1 ori
  source 1..456: mol_type = other DNA
acgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattt

SEQ ID NO: 237 - DNA, 281 bp, synthetic construct
  misc_feature 1..281: note = Linker region
  source 1..281: mol_type = other DNA
tatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtg

SEQ ID NO: 238 - DNA, 105 bp, synthetic construct
  misc_feature 1..105: note = AmpR promoter
  source 1..105: mol_type = other DNA
cgcggaacccctatttgtttatttttctaaatacattcaaatatgtatccgctcatgagacaataaccctgataaatgcttcaataatattgaaaaaggaagagt

SEQ ID NO: 239 - DNA, 861 bp, synthetic construct
  misc_feature 1..861: note = AmpR gene
  source 1..861: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 240 - DNA, 170 bp, synthetic construct
  misc_feature 1..170: note = Linker region
  source 1..170: mol_type = other DNA
ctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 241 - DNA, 589 bp, synthetic construct
  misc_feature 1..589: note = pUC ori
  source 1..589: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 242 - DNA, 61 bp, synthetic construct
  misc_feature 1..61: note = Linker region
  source 1..61: mol_type = other DNA
aacgccagcaacgcggcctttttacggttcctggccttttgctggccttttgctcacatgt

SEQ ID NO: 243 - DNA, 141 bp, synthetic construct; identical to SEQ ID NO: 217
  misc_feature 1..141: note = 5' inverted terminal repeat (ITR)
  source 1..141: mol_type = other DNA

SEQ ID NO: 244 - DNA, 27 bp, synthetic construct
  misc_feature 1..27: note = Linker region
  source 1..27: mol_type = other DNA
tctagacaactttgtatagaaaagttg

SEQ ID NO: 245 - DNA, 589 bp, synthetic construct; identical to SEQ ID NO: 219
  misc_feature 1..589: note = CMV enhancer and promoter
  source 1..589: mol_type = other DNA

SEQ ID NO: 246 - DNA, 30 bp, synthetic construct; identical to SEQ ID NO: 220
  misc_feature 1..30: note = Linker region
  source 1..30: mol_type = other DNA

SEQ ID NO: 247 - DNA, 54 bp, synthetic construct; identical to SEQ ID NO: 105
  misc_feature 1..54: note = Albumin signal peptide
  source 1..54: mol_type = other DNA

SEQ ID NO: 248 - DNA, 363 bp, synthetic construct; identical to SEQ ID NO: 106
  misc_feature 1..363: note = Adalimumab variable heavy (VH) domain
  source 1..363: mol_type = other DNA

SEQ ID NO: 249 - DNA, 990 bp, synthetic construct; identical to SEQ ID NO: 107
  misc_feature 1..990: note = hIgG1 constant heavy (CH) domain
  source 1..990: mol_type = other DNA

SEQ ID NO: 250 - DNA, 12 bp, synthetic construct
  misc_feature 1..12: note = Furin cleavage site
  source 1..12: mol_type = other DNA
cgaaaacggcgc

SEQ ID NO: 251 - skipped

SEQ ID NO: 252 - DNA, 57 bp, synthetic construct; identical to SEQ ID NO: 110
  misc_feature 1..57: note = P2A cleavage site
  source 1..57: mol_type = other DNA

SEQ ID NO: 253 - DNA, 54 bp, synthetic construct; identical to SEQ ID NO: 105
  misc_feature 1..54: note = Albumin signal peptide
  source 1..54: mol_type = other DNA

SEQ ID NO: 254 - DNA, 321 bp, synthetic construct; identical to SEQ ID NO: 111
  misc_feature 1..321: note = Adalimumab variable light (VL) chain
  source 1..321: mol_type = other DNA

SEQ ID NO: 255 - DNA, 321 bp, synthetic construct; identical to SEQ ID NO: 182
  misc_feature 1..321: note = hIgG kappa constant light (CL) chain
  source 1..321: mol_type = other DNA

SEQ ID NO: 256 - skipped

SEQ ID NO: 257 - DNA, 12 bp, synthetic construct
  misc_feature 1..12: note = Linker region
  source 1..12: mol_type = other DNA
ccccccccccta

SEQ ID NO: 258 - DNA, 552 bp, synthetic construct; identical to SEQ ID NO: 114
  misc_feature 1..552: note = Internal ribosome entry site (IRES) (non-coding)
  source 1..552: mol_type = other DNA

SEQ ID NO: 259 - DNA, 201 bp, synthetic construct; identical to SEQ ID NO: 208
  misc_feature 1..201: note = Enhancer protein L
  source 1..201: mol_type = other DNA

SEQ ID NO: 260 - skipped

SEQ ID NO: 261 - DNA, 54 bp, synthetic construct; identical to SEQ ID NO: 231
  misc_feature 1..54: note = Linker region
  source 1..54: mol_type = other DNA

SEQ ID NO: 262 - DNA, 208 bp, synthetic construct; identical to SEQ ID NO: 232
  misc_feature 1..208: note = BGH polyadenylation signal
  source 1..208: mol_type = other DNA

SEQ ID NO: 263 - skipped

SEQ ID NO: 264 - DNA, 141 bp, synthetic construct; identical to SEQ ID NO: 234
  misc_feature 1..141: note = 3' inverted terminal repeat (ITR)
  source 1..141: mol_type = other DNA

SEQ ID NO: 265 - DNA, 74 bp, synthetic construct; identical to SEQ ID NO: 235
  misc_feature 1..74: note = Linker region
  source 1..74: mol_type = other DNA

SEQ ID NO: 266 - DNA, 456 bp, synthetic construct; identical to SEQ ID NO: 236
  misc_feature 1..456: note = F1 ori
  source 1..456: mol_type = other DNA

SEQ ID NO: 267 - DNA, 281 bp, synthetic construct; identical to SEQ ID NO: 237
  misc_feature 1..281: note = Linker region
  source 1..281: mol_type = other DNA

SEQ ID NO: 268 - DNA, 105 bp, synthetic construct; identical to SEQ ID NO: 238
  misc_feature 1..105: note = AmpR promoter
  source 1..105: mol_type = other DNA

SEQ ID NO: 269 - DNA, 861 bp, synthetic construct; identical to SEQ ID NO: 239
  misc_feature 1..861: note = AmpR gene
  source 1..861: mol_type = other DNA

SEQ ID NO: 270 - DNA, 170 bp, synthetic construct; identical to SEQ ID NO: 240
  misc_feature 1..170: note = Linker region
  source 1..170: mol_type = other DNA

SEQ ID NO: 271 - DNA, 589 bp, synthetic construct; identical to SEQ ID NO: 241
  misc_feature 1..589: note = pUC ori
  source 1..589: mol_type = other DNA

SEQ ID NO: 272 - DNA, 61 bp, synthetic construct; identical to SEQ ID NO: 242
  misc_feature 1..61: note = Linker region
  source 1..61: mol_type = other DNA

SEQ ID NO: 273 - DNA, 304 bp, synthetic construct
  misc_feature 1..304: note = CMV enhancer
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 274 - DNA, 204 bp, synthetic construct; identical to SEQ ID NO: 103
  misc_feature 1..204: note = CMV promoter
  source 1..204: mol_type = other DNA

SEQ ID NO: 275 - DNA, 22 bp, synthetic construct
  misc_feature 1..22: note = Linker region
  source 1..22: mol_type = other DNA
ggtttagtgaaccgtcagatcc

SEQ ID NO: 276 - DNA, 81 bp, synthetic construct; identical to SEQ ID NO: 172
  misc_feature 1..81: note = Multiple cloning site (MSC)
  source 1..81: mol_type = other DNA

SEQ ID NO: 277 - skipped

SEQ ID NO: 278 - DNA, 117 bp, synthetic construct
  misc_feature 1..117: note = GBA signal peptide
  source 1..117: mol_type = other DNA
atggagttttcaagtccttccagagaggaatgtcccaagcctttgagtagggtaagcatcatggctggcagcctcacaggattgcttctacttcaggcagtgtcgtgggcatcaggt

SEQ ID NO: 279 - DNA, 1491 bp, synthetic construct
  misc_feature 1..1491: note = GBA mature protein
  source 1..1491: mol_type = other DNA
gcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccag

SEQ ID NO: 280 - DNA, 12 bp, synthetic construct
  misc_feature 1..12: note = GSSG linker
  source 1..12: mol_type = other DNA
ggctcgagcggc

SEQ ID NO: 281 - DNA, 510 bp, synthetic construct
  misc_feature 1..510: note = NanoLuc luciferase
  source 1..510: mol_type = other DNA
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 282 - skipped

SEQ ID NO: 283 - DNA, 126 bp, synthetic construct
  misc_feature 1..126: note = Linker region
  source 1..126: mol_type = other DNA
ttacagcggccgcgactctagatcataatcagccataccacatttgtagaggttttacttgctttaaaaaacctcccacacctccccctgaacctgaaacataaaatgaatgcaattgttgttgtt

SEQ ID NO: 284 - DNA, 122 bp, synthetic construct; identical to SEQ ID NO: 117
  misc_feature 1..122: note = SV40 polyadenylation signal
  source 1..122: mol_type = other DNA

SEQ ID NO: 285 - skipped

SEQ ID NO: 286 - DNA, 456 bp, synthetic construct
  misc_feature 1..456: note = F1 ori
  source 1..456: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgt

SEQ ID NO: 287 - DNA, 26 bp, synthetic construct
  misc_feature 1..26: note = Linker region
  source 1..26: mol_type = other DNA
caggtggcacttttcggggaaatgtg

SEQ ID NO: 288 - DNA, 105 bp, synthetic construct; identical to SEQ ID NO: 238
  misc_feature 1..105: note = AmpR promoter
  source 1..105: mol_type = other DNA

SEQ ID NO: 289 - DNA, 358 bp, synthetic construct
  misc_feature 1..358: note = SV40 promoter
  source 1..358: mol_type = other DNA
ctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 290 - DNA, 34 bp, synthetic construct
  misc_feature 1..34: note = Linker region
  source 1..34: mol_type = other DNA
gatcgatcaagagacaggatgaggatcgtttcgc

SEQ ID NO: 291 - DNA, 795 bp, synthetic construct; identical to SEQ ID NO: 119
  misc_feature 1..795: note = NeoR/KanR resistance gene
  source 1..795: mol_type = other DNA

SEQ ID NO: 292 - DNA, 231 bp, synthetic construct
  misc_feature 1..231: note = Linker region
  source 1..231: mol_type = other DNA
gcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatga

SEQ ID NO: 293 - DNA, 48 bp, synthetic construct
  misc_feature 1..48: note = HSV TK polyadenylation signal
  source 1..48: mol_type = other DNA
cggcaataaaaagacagaataaaacgcacggtgttgggtcgtttgttc

SEQ ID NO: 294 - DNA, 328 bp, synthetic construct
  misc_feature 1..328: note = Linker region
  source 1..328: mol_type = other DNA
ataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 295 - DNA, 589 bp, synthetic construct; identical to SEQ ID NO: 121
  misc_feature 1..589: note = Ori
  source 1..589: mol_type = other DNA

SEQ ID NO: 296 - DNA, 174 bp, synthetic construct
  misc_feature 1..174: note = Linker region
  source 1..174: mol_type = other DNA
aacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccg

SEQ ID NO: 297 - DNA, 304 bp, synthetic construct; identical to SEQ ID NO: 273
  misc_feature 1..304: note = CMV enhancer
  source 1..304: mol_type = other DNA

SEQ ID NO: 298 - DNA, 204 bp, synthetic construct; identical to SEQ ID NO: 103
  misc_feature 1..204: note = CMV promoter
  source 1..204: mol_type = other DNA

SEQ ID NO: 299 - DNA, 22 bp, synthetic construct
  misc_feature 1..22: note = Linker region
  source 1..22: mol_type = other DNA
ggtttagtgaaccgtcagatcc

SEQ ID NO: 300 - DNA, 81 bp, synthetic construct; identical to SEQ ID NO: 172
  misc_feature 1..81: note = Multiple cloning site (MSC)
  source 1..81: mol_type = other DNA

SEQ ID NO: 301 - skipped

SEQ ID NO: 302 - DNA, 117 bp, synthetic construct; identical to SEQ ID NO: 278
  misc_feature 1..117: note = GBA signal peptide
  source 1..117: mol_type = other DNA

SEQ ID NO: 303 - DNA, 1491 bp, synthetic construct; identical to SEQ ID NO: 279
  misc_feature 1..1491: note = GBA mature protein
  source 1..1491: mol_type = other DNA

SEQ ID NO: 304 - DNA, 12 bp, synthetic construct
  misc_feature 1..12: note = GSSG linker
  source 1..12: mol_type = other DNA
ggctcgagcggc

SEQ ID NO: 305 - DNA, 510 bp, synthetic construct; identical to SEQ ID NO: 281
  misc_feature 1..510: note = NanoLuc luciferase
  source 1..510: mol_type = other DNA

SEQ ID NO: 306 - skipped

SEQ ID NO: 307 - DNA, 20 bp, synthetic construct
  misc_feature 1..20: note = Linker region
  source 1..20: mol_type = other DNA
ttactaatccccccccccta

SEQ ID NO: 308 - DNA, 552 bp, synthetic construct; identical to SEQ ID NO: 114
  misc_feature 1..552: note = Internal ribosome entry site (IRES) (non-coding)
  source 1..552: mol_type = other DNA

SEQ ID NO: 309 - DNA, 201 bp, synthetic construct; identical to SEQ ID NO: 208
  misc_feature 1..201: note = Enhancer protein L
  source 1..201: mol_type = other DNA

SEQ ID NO: 310 - skipped

SEQ ID NO: 311 - DNA, 105 bp, synthetic construct; identical to SEQ ID NO: 116
  misc_feature 1..105: note = Linker region
  source 1..105: mol_type = other DNA

SEQ ID NO: 312 - DNA, 122 bp, synthetic construct; identical to SEQ ID NO: 117
  misc_feature 1..122: note = SV40 polyadenylation signal
  source 1..122: mol_type = other DNA

SEQ ID NO: 313 - skipped

SEQ ID NO: 314 - DNA, 456 bp, synthetic construct; identical to SEQ ID NO: 286
  misc_feature 1..456: note = F1 ori
  source 1..456: mol_type = other DNA

SEQ ID NO: 315 - DNA, 26 bp, synthetic construct
  misc_feature 1..26: note = Linker region
  source 1..26: mol_type = other DNA
caggtggcacttttcggggaaatgtg

SEQ ID NO: 316 - DNA, 105 bp, synthetic construct; identical to SEQ ID NO: 238
  misc_feature 1..105: note = AmpR promoter
  source 1..105: mol_type = other DNA

SEQ ID NO: 317 - DNA, 358 bp, synthetic construct; identical to SEQ ID NO: 289
  misc_feature 1..358: note = SV40 promoter
  source 1..358: mol_type = other DNA

SEQ ID NO: 318 - DNA, 34 bp, synthetic construct; identical to SEQ ID NO: 290
  misc_feature 1..34: note = Linker region
  source 1..34: mol_type = other DNA

SEQ ID NO: 319 - DNA, 795 bp, synthetic construct; identical to SEQ ID NO: 119
  misc_feature 1..795: note = NeoR/KanR resistance gene
  source 1..795: mol_type = other DNA

SEQ ID NO: 320 - DNA, 231 bp, synthetic construct; identical to SEQ ID NO: 292
  misc_feature 1..231: note = Linker region
  source 1..231: mol_type = other DNA

SEQ ID NO: 321 - DNA, 48 bp, synthetic construct; identical to SEQ ID NO: 293
  misc_feature 1..48: note = HSV TK polyadenylation signal
  source 1..48: mol_type = other DNA

SEQ ID NO: 322 - DNA, 328 bp, synthetic construct; identical to SEQ ID NO: 294
  misc_feature 1..328: note = Linker region
  source 1..328: mol_type = other DNA

SEQ ID NO: 323 - DNA, 589 bp, synthetic construct; identical to SEQ ID NO: 121
  misc_feature 1..589: note = Ori
  source 1..589: mol_type = other DNA

SEQ ID NO: 324 - DNA, 174 bp, synthetic construct; identical to SEQ ID NO: 296
  misc_feature 1..174: note = Linker region
  source 1..174: mol_type = other DNA

SEQ ID NO: 325 - protein, 18 aa, synthetic construct
  REGION 1..18: note = Albumin signal peptide
MKWVTFISLLFLFSSAYS

SEQ ID NO: 326 - protein, 121 aa, synthetic construct [sequence omitted]

SEQ ID NO: 327 - protein, 330 aa, synthetic construct [sequence omitted]

SEQ ID NO: 328 - protein, 4 aa, synthetic construct
  REGION 1..4: note = Furin cleavage site
RKRR

SEQ ID NO: 329 - skipped

SEQ ID NO: 330 - protein, 19 aa, synthetic construct
  REGION 1..19: note = P2A cleavage site
ATNFSLLKQAGDVEENPGP

SEQ ID NO: 331 - protein, 18 aa, synthetic construct
  REGION 1..18: note = Albumin signal peptide
MKWVTFISLLFLFSSAYS

SEQ ID NO: 332 - protein, 107 aa, synthetic construct [sequence omitted]

SEQ ID NO: 333 - protein, 107 aa, synthetic construct [sequence omitted]

SEQ ID NO: 334 - protein, 18 aa, synthetic construct
  REGION 1..18: note = Albumin signal peptide
MKWVTFISLLFLFSSAYS

SEQ ID NO: 335 - protein, 121 aa, synthetic construct [sequence omitted]

SEQ ID NO: 336 - protein, 330 aa, synthetic construct [sequence omitted]

SEQ ID NO: 337 - protein, 4 aa, synthetic construct
  REGION 1..4: note = Furin cleavage site
RKRR

SEQ ID NO: 338 - skipped

SEQ ID NO: 339 - protein, 19 aa, synthetic construct
  REGION 1..19: note = P2A cleavage site
ATNFSLLKQAGDVEENPGP

SEQ ID NO: 340 - protein, 18 aa, synthetic construct
  REGION 1..18: note = Albumin signal peptide
MKWVTFISLLFLFSSAYS

SEQ ID NO: 341 - protein, 107 aa, synthetic construct [sequence omitted]

SEQ ID NO: 342 - protein, 107 aa, synthetic construct [sequence omitted]

SEQ ID NO: 343 - protein, 67 aa, synthetic construct; identical to SEQ ID NO: 2
  REGION 1..67: note = Enhancer protein L

SEQ ID NO: 344 - protein, 18 aa, synthetic construct
  REGION 1..18: note = Albumin signal peptide
MKWVTFISLLFLFSSAYS

SEQ ID NO: 345 - protein, 121 aa, synthetic construct [sequence omitted]

SEQ ID NO: 346 - protein, 330 aa, synthetic construct [sequence omitted]

SEQ ID NO: 347 - protein, 4 aa, synthetic construct
  REGION 1..4: note = Furin cleavage site
RKRR

SEQ ID NO: 348 - skipped

SEQ ID NO: 349 - protein, 19 aa, synthetic construct
  REGION 1..19: note = P2A cleavage site
ATNFSLLKQAGDVEENPGP

SEQ ID NO: 350 - protein, 18 aa, synthetic construct
  REGION 1..18: note = Albumin signal peptide
MKWVTFISLLFLFSSAYS

SEQ ID NO: 351 - protein, 107 aa, synthetic construct [sequence omitted]

SEQ ID NO: 352 - protein, 107 aa, synthetic construct [sequence omitted]

SEQ ID NO: 353 - protein, 18 aa, synthetic construct
  REGION 1..18: note = Albumin signal peptide
MKWVTFISLLFLFSSAYS

SEQ ID NO: 354 - protein, 121 aa, synthetic construct [sequence omitted]

SEQ ID NO: 355 - protein, 330 aa, synthetic construct [sequence omitted]

SEQ ID NO: 356 - protein, 4 aa, synthetic construct
  REGION 1..4: note = Furin cleavage site
RKRR

SEQ ID NO: 357 - skipped

SEQ ID NO: 358 - protein, 19 aa, synthetic construct
  REGION 1..19: note = P2A cleavage site
ATNFSLLKQAGDVEENPGP

SEQ ID NO: 359 - protein, 18 aa, synthetic construct
  REGION 1..18: note = Albumin signal peptide
MKWVTFISLLFLFSSAYS

SEQ ID NO: 360 - protein, 107 aa, synthetic construct [sequence omitted]

SEQ ID NO: 361 - protein, 107 aa, synthetic construct [sequence omitted]

SEQ ID NO: 362 - protein, 67 aa, synthetic construct; identical to SEQ ID NO: 2
  REGION 1..67: note = Enhancer protein L

SEQ ID NO: 363 - protein, 39 aa, synthetic construct
  REGION 1..39: note = GBA signal peptide
MEFSSPSREECPKPLSRVSIMAGSLTGLLLLQAVSWASG

SEQ ID NO: 364 - protein, 497 aa, synthetic construct
  REGION 1..497: note = Mature GBA protein
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 365 - protein, 4 aa, synthetic construct
  REGION 1..4: note = GSSG linker
GSSG

SEQ ID NO: 366 - protein, 170 aa, synthetic construct
  REGION 1..170: note = NanoLuc luciferase
VFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 367 - protein, 264 aa, synthetic construct
  REGION 1..264: note = NeoR/KanR resistance gene
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 368 - protein, 39 aa, synthetic construct; identical to SEQ ID NO: 363
  REGION 1..39: note = GBA signal peptide

SEQ ID NO: 369 - protein, 497 aa, synthetic construct; identical to SEQ ID NO: 364
  REGION 1..497: note = Mature GBA protein

SEQ ID NO: 370 - protein, 4 aa, synthetic construct
  REGION 1..4: note = GSSG linker
GSSG

SEQ ID NO: 371 - protein, 170 aa, synthetic construct; identical to SEQ ID NO: 366
  REGION 1..170: note = NanoLuc luciferase

SEQ ID NO: 372 - protein, 67 aa, synthetic construct; identical to SEQ ID NO: 2
  REGION 1..67: note = Enhancer protein L

SEQ ID NO: 373 - protein, 264 aa, synthetic construct; identical to SEQ ID NO: 367
  REGION 1..264: note = NeoR/KanR resistance gene

SEQ ID NO: 374 - protein, 120 aa, synthetic construct [sequence omitted]

SEQ ID NO: 375 - protein, 111 aa, synthetic construct [sequence omitted]

SEQ ID NO: 376 - protein, 113 aa, synthetic construct [sequence omitted]

SEQ ID NO: 377 - protein, 107 aa, synthetic construct [sequence omitted]

SEQ ID NO: 378 - protein, 120 aa, synthetic construct [sequence omitted]

SEQ ID NO: 379 - protein, 107 aa, synthetic construct [sequence omitted]

SEQ ID NO: 380 - protein, 123 aa, synthetic construct [sequence omitted]

SEQ ID NO: 381 - protein, 107 aa, synthetic construct [sequence omitted]

SEQ ID NO: 382 - protein, 119 aa, synthetic construct
  REGION 1..119: note = Ustekinumab VH
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 383 - protein, 107 aa, synthetic construct
  REGION 1..107: note = Ustekinumab VL
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 384 - protein, 122 aa, synthetic construct [sequence omitted]

SEQ ID NO: 385 - protein, 106 aa, synthetic construct [sequence omitted]

SEQ ID NO: 386 - protein, 127 aa, synthetic construct [sequence omitted]

SEQ ID NO: 387 - protein, 108 aa, synthetic construct [sequence omitted]

SEQ ID NO: 388 - protein, 121 aa, synthetic construct [sequence omitted]

SEQ ID NO: 389 - protein, 112 aa, synthetic construct [sequence omitted]

SEQ ID NO: 390 - protein, 122 aa, synthetic construct
  REGION 1..122: note = Ibalizumab VH
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSS

SEQ ID NO: 391 - protein, 112 aa, synthetic construct
  REGION 1..112: note = Ibalizumab VL
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIK

SEQ ID NO: 392 - protein, 126 aa, synthetic construct [sequence omitted]

SEQ ID NO: 393 - protein, 107 aa, synthetic construct [sequence omitted]

SEQ ID NO: 394 - protein, 116 aa, synthetic construct
  REGION 1..116: note = Atoltivimab VH
QVQLVESGGGVVQPGRSLRLSCAASGFTFNNYGMHWVRQAPGMGLEWVAVIWHDGSDKYYADSVKGRFTISRDNSKNTLYLQMNSLRAEDTAVYYCARNWNLFDYWGQGTLVTVSS

SEQ ID NO: 395 - protein, 108 aa, synthetic construct
  REGION 1..108: note = Atoltivimab VL
DIQMTQSPSSLSASVGDRITITCRASQSISTYLHWYQQKPGKAPKLLIYAASTLQSGVPSRFSGSGSGTDFTLTISSLQPEDFATYYCQQSFSTPPINFGQGTKLEIK

SEQ ID NO: 396 - protein, 118 aa, synthetic construct
  REGION 1..118: note = Maftivimab VH
EVQLVESGGGLVQPGGSLRLSCAASGFTSSSYAMNWVRQAPGKGLEWVSTISGMGGSTYYADSVKGRFTISRDNSKNTLYLQMNSLRAEDTAVYYCAKRGYPHSFDIWGQGTMVTVSS

SEQ ID NO: 397 - protein, 106 aa, synthetic construct
  REGION 1..106: note = Maftivimab VL
DIQMTQSPSSLSASVGDRVTITCRASQSISSFLNWYQQKPGKAPKLLIYAASSLQSGVPSRFSGSGSGTDFTLTISSLQPEDFATYYCQQSYSTLTFGQGTRLEIK

SEQ ID NO: 398 - protein, 118 aa, synthetic construct
  REGION 1..118: note = Odesivimab VH
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARTWFGELYFDYWGQGTLVTVSS

SEQ ID NO: 399 - protein, 113 aa, synthetic construct
  REGION 1..113: note = Odesivimab VL
DIVMTQSPDSLAVSLGERATINCKSSQSVLYSSNNKNYLAWYQQKPGQPPKLLIYWASTRESGVPDRFSGSGSGTEFTLTITSLQAEDVAVYYCQQYYSSPLTFGGGTKVEIK

SEQ ID NO: 400 - protein, 120 aa, synthetic construct
  REGION 1..120: note = Casirivimab VH
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSS

SEQ ID NO: 401 - protein, 107 aa, synthetic construct
  REGION 1..107: note = Casirivimab VL
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIK

SEQ ID NO: 402 - protein, 120 aa, synthetic construct
  REGION 1..120: note = Imdevimab VH
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 403 - protein, 110 aa, synthetic construct
  REGION 1..110: note = Imdevimab VL
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 404 - protein, 120 aa, synthetic construct
  REGION 1..120: note = Brolucizumab VH
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 405 - protein, 110 aa, synthetic construct
  REGION 1..110: note = Brolucizumab VL
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVL

SEQ ID NO: 406 - protein, 497 aa, synthetic construct; identical to SEQ ID NO: 364
  REGION 1..497: note = Glucosylceramidase (GBA)

SEQ ID NO: 407 - protein, 415 aa, synthetic construct
  REGION 1..415: note = rFIX-Fc Coagulation Factor IX
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 408 - protein, 506 aa, synthetic construct
  REGION 1..506: note = Taliglucerase
EFARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQDLLVDTM

SEQ ID NO: 409 - protein, 398 aa, synthetic construct
  REGION 1..398: note = Agalsidase beta
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 410 - protein, 626 aa, synthetic construct
  REGION 1..626: note = Laronidase
APHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 411 - protein, 525 aa, synthetic construct
  REGION 1..525: note = Idursulfase
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 412 - protein, 339 aa, synthetic construct
  REGION 1..339: note = HLA Class I alpha chain (mouse K2-D1)
GPHSMRYFETAVSRPGLEEPRYISVGYVDNKEFVRFDSDAENPRYEPRAPWMEQEGPEYWERETQKAKGQEQWFRVSLRNLLGYYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKAR

SEQ ID NO: 413 - protein, 100 aa, Mus musculus
IQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMR

SEQ ID NO: 414 - protein, 1916 aa, Mus musculus
MDAESIRLNNENLWAWLVRLLSKNPEWLSAKLRSFLPTMDLDCSYEPSNPEVIHRQLNRLFAQGMATWKSFINDLCFELDVPLDMEIPLVSIWGPRDEFSKQLGAGEESCPGPQLYHGAKRPFQSYGSSPRRKNSKKQQLELAKKYLKLLKTSAQQWHGGVCPGAWLTPHSPQTYIPPVLQWSRATAPLDAQEGATLGDPEAADNIDVSIQDLFSFKAHKGPRVTVLLGKAGMGKTTLAYRLRWRWAQGQLDRFQALFLFEFRQLNMITQLPTLPQLLFDLYLMPESEPDAVFQYLKENAQEVLLIFDGLDEALHADSVGTDNAGSALTLFSELCHGNLLPGCWVMTTSRPGKLPSCVPTEAATVHMWGFDGLRVEKYVTCFFSDLLSQELALKEMRTNARLRGMCAIPALCTVTCFCLRRLLPGSSPGQSAALLPTITQLYLQMVETFSPSETLLDTSILGFGKVALRGLDTGKVVFSVEDISPQLMSFGAVHSLLTSFCIHTRPGHEEIGYAFVHLSLQEFFAALYLMASHTVDKDTLVEYVTLNSHWVLRTKGRLGLSDHLPAFLAGLASHTCHMFLCQLAQQDRAWVGSRQAAVIQVLRKLASRKLTGPKMIELYHCVAETQDLELARFTAQSLPSRLSFHNFPLTHADLAALANILEHRDDPIHLDFDGCPLEPHCPEALVGCGQVENLSFKSRKCGDAFAEALCRSLPTMGSLKTLGLTGSRITAQGISHLIQTLPLCSQLEEVSLHDNQLKDPEVLSLVELLPSLPKLQKLDLSRNSFSRSILLSLVKVAITCPTVRKLQVRELDLIFYLSPVTETATQQSGASDVQGKDSLKEGQSRSLQLRLQKCQLRIRDAEALVELFQKSPQLEEVNLSGNHLEDDGCRLVAEAASQLHIAQKLDLSDNGLSQTGVTYVLKAMSTCGTLEDLHISLLNNTVVLTFAQEPREQEGSCKGRAPLISFVSPVTSELSQRSRRIRLTHCGFLAKHTETLCEALRASCQTHNLDHLDLSDNSLGGKGVILLTELLPGLGPLKSLNLSRNGLSMDAVFSLVQCLSSLQWVFHLDVSLESDCIFLRGAGTSRDALEPKFQTGVQVLELSQRYTSRSFCLQECQLEPTSLTFLCATLEKSPGPLEVQLSCKSLSDDSLKILLQCLPQLPQLSLLQLRHTVLSSRSPFLLADIFNLCPRVRKVTLRSLCHAVLHFDSNEEQEGVCCGFPGCSLSQEHMETLCCALSKCNALSQLDLTDNLLGDIGLRCLLECLPQLPISGWLDLSHNNISQEGILYLLETLPSYPNIQEVSVSLSSEQIFRMCFSKKEGAGTSLRLCECSFSPEQVSKLASSLSQAQQLTELWLTKCHLDLPQLTMLLNLVNRPTGLLGLRLEEPWVDSVSLPALMEVCAQASGCLTELSISEIQRKLWLQLEFPHQEGNSDSMALRLAHCDLETEHSHLMIQLVETYARLQQLSLSQVSFNDNDGTSSKLLQNILLSSCELKSFRLTFSQVSTKSLTHLAFGLGHCHHLEELDFSNNSLREEDTELLMGALQGTCRLKKLHLSFLPLGASSLALLIQGLSRMTLLQDLCLSHNQIGDVGTQCLAAILPKLPELRKFDLSHNQIGDVGTQCLAAILPKLPELRKFNLSHNQIGHVGTQCLAAILPKLPELRKFDLSRNQIGDVGTQCLAAILPKLPELRKFDLSGNRIGPAGGVQLVKSLTHFEHLEEIKLGNNALGEPTALELAQRLPPQLRVLCLPSSHLGPEGALGLAQALEQCPHIEEVSLAENNLAGGVPRFSKRLPLLRQIDLEFCKIEDQAARHLAANLTLFPALEKLLLSGNLLGDEVAAELAQVLPQMGQLKKVNLEWNRITARGAQLLAQGLVQGSCVPVIRLWNNPILNDVAQSLQSQEPRLDFSITDQQTLR

SEQ ID NO: 415 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 416 - protein, 99 aa, Homo sapiens [sequence omitted]

SEQ ID NO: 417 - protein, 1866 aa, Homo sapiens
MDPVGLQLGNKNLWSCLVRLLTKDPEWLNAKMKFFLPNTDLDSRNETLDPEQRVILQLNKLHVQGSDTWQSFIHCVCMQLEVPLDLEVLLLSTFGYDDGFTSQLGAEGKSQPESQLHHGLKRPHQSCGSSPRRKQCKKQQLELAKKYLQLLRTSAQQRYRSQIPGSGQPHAFHQVYVPPILRRATASLDTPEGAIMGDVKVEDGADVSISDLFNTRVNKGPRVTVLLGKAGMGKTTLAHRLCQKWAEGHLNCFQALFLFEFRQLNLITRFLTPSELLFDLYLSPESDHDTVFQYLEKNADQVLLIFDGLDEALQPMGPDGPGPVLTLFSHLCNGTLLPGCRVMATSRPGKLPACLPAEAAMVHMLGFDGPRVEEYVNHFFSAQPSREGALVELQTNGRLRSLCAVPALCQVACLCLHHLLPDHAPGQSVALLPNMTQLYMQMVLALSPPGHLPTSSLLDLGEVALRGLETGKVIFYAKDIAPPLIAFGATHSLLTSFCVCTGPGHQQTGYAFTHLSLQEFLAALHLMASPKVNKDTLTQYVTLHSRWVQRTKARLGLSDHLPTFLAGLASCTCRPFLSHLAQGNEDCVGAKQAAVVQVLKKLATRKLTGPKVVELCHCVDETQEPELASLTAQSLPYQLPFHNFPLTCTDLATLTNILEHREAPIHLDFDGCPLEPHCPEALVGCGQIENLSFKSRKCGDAFAEALSRSLPTMGRLQMLGLAGSKITARGISHLVKALPLCPQLKEVSFRDNQLSDQVVLNIVEVLPHLPRLRKLDLSSNSICVSTLLCLARVAVTCPTVRMLQAREADLIFLLSPPTETTAELQRAPDLQESDGQRKGAQSRSLTLRLQKCQLQVHDAEALIALLQEGPHLEEVDLSGNQLEDEGCRLMAEAASQLHIARKLDLSNNGLSVAGVHCVLRAVSACWTLAELHISLQHKTVIFMFAQEPEEQKGPQERAAFLDSLMLQMPSELPLSSRRMRLTHCGLQEKHLEQLCKALGGSCHLGHLHLDFSGNALGDEGAARLAQLLPGLGALQSLNLSENGLSLDAVLGLVRCFSTLQWLFRLDISFESQHILLRGDKTSRDMWATGSLPDFPAAAKFLGFRQRCIPRSLCLSECPLEPPSLTRLCATLKDCPGPLELQLSCEFLSDQSLETLLDCLPQLPQLSLLQLSQTGLSPKSPFLLANTLSLCPRVKKVDLRSLHHATLHFRSNEEEEGVCCGRFTGCSLSQEHVESLCWLLSKCKDLSQVDLSANLLGDSGLRCLLECLPQVPISGLLDLSHNSISQESALYLLETLPSCPRVREASVNLGSEQSFRIHFSREDQAGKTLRLSECSFRPEHVSRLATGLSKSLQLTELTLTQCCLGQKQLAILLSLVGRPAGLFSLRVQEPWADRARVLSLLEVCAQASGSVTEISISETQQQLCVQLEFPRQEENPEAVALRLAHCDLGAHHSLLVGQLMETCARLQQLSLSQVNLCEDDDASSLLLQSLLLSLSELKTFRLTSSCVSTEGLAHLASGLGHCHHLEELDLSNNQFDEEGTKALMRALEGKWMLKRLDLSHLLLNSSTLALLTHRLSQMTCLQSLRLNRNSIGDVGCCHLSEALRAATSLEELDLSHNQIGDAGVQHLATILPGLPELRKIDLSGNSISSAGGVQLAESLVLCRRLEELMLGCNALGDPTALGLAQELPQHLRVLHLPFSHLGPGGALSLAQALDGSPHLEEISLAENNLAGGVLRFCMELPLLRQIDLVSCKIDNQTAKLLTSSFTSCPALEVILLSWNLLGDEAAAELAQVLPQMGRLKRVDLEKNQITALGAWLLAEGLAQGSSIQVIRLWNNPIPCDMAQHLKSQEPRLDFAFFDNQPQAPWGT

SEQ ID NO: 418 - protein, 313 aa, Mus musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 419 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 420 - protein, 883 aa, synthetic construct
  REGION 1..883: note = Alglucosidase alfa
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 421 - protein, 1438 aa, synthetic construct
  REGION 1..1438: note = B-domain deleted human Factor VIII (BDD FVIII)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 422 - protein, 2791 aa, synthetic construct
  REGION 1..2791: note = Von Willebrand Factor, recombinant
AEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

(19) United States
(12) Patent Application Publication
(10) Pub. No.: US 2024/0141373 A1
(43) Pub. Date: May 2, 2024

(54) SYSTEMS AND METHODS FOR PROTEIN EXPRESSION

(71) Applicant: Excepgen Inc., San Francisco, CA (US)

(72) Inventors: Barbara MERTINS, San Francisco, CA (US); Thomas FOLLIARD, San Francisco, CA (US); Imre MÄGER, San Francisco, CA (US)

(21) Appl. No.: 18/465,335

(22) Filed: Sep. 12, 2023

Related U.S. Application Data
(63) Continuation of application No. PCT/US2022/019984, filed on Mar. 11, 2022.
(60) Provisional application No. 63/160,672, filed on Mar. 12, 2021.

(51) Int. Cl. C12N 15/85; C07K 14/005; C07K 14/705; C07K 14/81; C07K 16/24; C12N 9/24

(52) U.S. Cl. CPC C12N 15/85; C07K 14/005; C07K 14/70503; C07K 14/70571; C07K 14/8121; C07K 16/241; C12N 9/2402; C12Y 302/01045; C12N 2770/32222; C12N 2800/40

(57) ABSTRACT

The present disclosure provides methods and compositions for the improved expression of target proteins via the co-expression of an enhancer protein in a subject. Provided herein are methods for expressing a target protein in a subject comprising administering a vector system of one or more polynucleotides encoding a target protein and an enhancer protein, wherein the polynucleotides are operatively linked, and wherein the enhancer protein is an inhibitor of nucleocytoplasmic transport (NCT) and/or the enhancer protein is selected from the group consisting of a picornavirus leader (L) protein, a picornavirus 2A protease, a rhinovirus 3C protease, a herpes simplex virus (HSV) ICP27 protein, and a rhabdovirus matrix (M) protein.

### CROSS REFERENCE TO RELATED APPLICATIONS

This application is a continuation of International Patent Application No. PCT/US2022/019984, filed Mar. 11, 2022, which claims priority to U.S. Provisional Patent Application No. 63/160,672, filed Mar. 12, 2021, the disclosures of each of which are incorporated by reference herein in their entirety.

### INCORPORATION OF THE SEQUENCE LISTING

The contents of the electronic sequence listing (EXCI_002_02US_SeqList_ST26.xml; Size: 585,364 bytes; and Date of Creation: Sep. 11, 2023) are herein incorporated by reference in their entirety.

### BACKGROUND

Recombinant expression of proteins in eukaryotic cells grown in culture has applications in scientific research and medicine. Recombinantly produced proteins (such as antibodies, enzymes, G-protein coupled receptors (GPCRs), secreted proteins, ion channels, viral proteins, and growth factors) are used within the pharmaceutical industry to develop new drugs (e.g., small molecule discovery), as therapeutics (e.g., antibodies and other biologic drugs), and as critical assets for analytical methods. In addition to their uses within the pharmaceutical industry, recombinantly produced mammalian proteins are increasingly used in the food industry (e.g., for so-called clean meat production). For many recombinant proteins, achieving expression of recombinant protein in a functional form remains challenging.

Transgene expression in living organisms like yeast, plants, animals and humans has applications in scientific research and medicine. The modern area of therapeutics is focused around the production of biologics like enzymes or antibodies. Currently, many therapeutics in this area are produced outside of the targeted subject and later injected. This process comes with its own challenges like finding the right expression system and producing the drug product stably and in high yields. Another emerging field in novel therapeutics is the expression of a gene within a living system by administering polynucleotides and using the body's own cells to produce the final drug product. The advantages around the production of a biologic in the body has many advantages including, e.g, native post-translational modifications. Additionally, by just using a polynucleotide as therapeutic, many shortfalls in the production and purification of the biologic is removed leaving a low cost, scalable, safe and stable drug product. This form of drug delivery has been researched since the beginning of the 1990s with little therapeutic success. There are many hypotheses around the unsuccessful production of these drugs within a living animal or human but still no solution.

There remains an unmet need for compositions and methods useful in the production of recombinant proteins and biologics in animals or humans in vivo.

### BRIEF DESCRIPTION OF FIGURES

FIG. 1 depicts six illustrative ways of regulating gene expression in eukaryotic cells.

FIGS. 2A-2X are schematic drawings of non-limiting, illustrative constructs: EG1, FIG. 2A; EG2, FIG. 2B; EG3 and EG4, FIG. 2C; EG5, FIG. 2D; EG6, FIG. 2E; EG7, FIG. 2F; EG8, FIG. 2G; EG9, FIG. 2H; EG10 and EG11, FIG. 2I; EG12 and EG4, FIG. 2J; EG10, FIG. 2K; EG13, FIG. 2L; EG14, FIG. 2M; EG15, FIG. 2N; EG16, FIG. 2O; EG17, FIG. 2P; EG18, FIG. 2Q; EG19, FIG. 2R; EG20, FIG. 2S; EG21, FIG. 2T; EG22, FIG. 2U; EG23, FIG. 2V; EG24, FIG. 2W; and EG25, FIG. 2X.

FIGS. 3A-3D show light and fluorescent microscopy of GFP expressed using construct EG2 (CMV-GFP-IRES-L) compared to a control vector EG1. FIG. 3A: light microscopy of cells comprising EG1. FIG. 3B: fluorescence microscopy of cells comprising EG1. FIG. 3C: light microscopy of cells comprising EG2. FIG. 3D: fluorescence microscopy of cells comprising EG2. Expression of the fluorescent GFP protein from the EG2 construct demonstrates the viability of the system. Reduction of deleterious over-expression in cells comprising EG2 (FIG. 3D) compared to cells comprising EG1 (FIG. 3B) demonstrates the improved regulation of GFP expression by introduction of the L-protein. The bar in FIGS. 3A-3D represents 400 microns.

FIGS. 4A-4D show light and fluorescent microscopy of GFP expressed using constructs EG3 and EG4 (T7-IRES-L-GFP and CMV-T7, respectively) compared to a control vector EG1. FIG. 4A: light microscopy of cells comprising EG1. FIG. 4B: fluorescence microscopy of cells comprising EG1. FIG. 4C: light microscopy of cells comprising EG3+EG4. FIG. 4D: fluorescence microscopy of cells comprising EG3+EG4. Expression of the fluorescent GFP protein from the EG3+EG4 constructs demonstrates the viability of the system. Reduction of expression in cells comprising EG3+EG4 (FIG. 4D) compared to cells comprising EG1 (FIG. 4B) demonstrates the improved regulation of GFP expression by introduction of the L-protein. The bar in FIGS. 4A-4D represents 400 microns.

FIGS. 5A-5D show fluorescent microscopy of a DRD1-GFP fusion from construct EG10 (CMV-[DRD1-GFP]) (FIG. 5A) or EG8 (CMV-[DRD1-GFP]IRES-L) (FIG. 5C). DRD1-GFP using construct EG10 is expressed but fails to transport the receptor into the outer membrane, leading to the formation of inclusion bodies (FIG. 5B, arrow). DRD1-GFP using construct EG8 is expressed and reliably transported into the membrane resulting in a high yield of the GPCR on the outer membrane with a high quality (FIG. 5D).

FIGS. 6A-6B show fluorescent microscopy of a DRD1-GFP fusion from construct EG10 (CMV-[DRD1-GFP]) (FIG. 6A) or EG12 and EG4 (T7-IRES-L-DRD1-GFP and CMV-T7, respectively) (FIG. 6B). DRD1-GFP using EG10 is expressed but fails to correctly transport the receptor into the outer membrane, leading to the formation of inclusion bodies (FIG. 6A, arrow). DRD1-GFP using EG12 in combination with EG4 is expressed and reliably transported into the membrane resulting in a high yield of the GPCR on the outer membrane with a high quality (FIG. 6B)

FIG. 7 shows an anti-CFTR Western blot. Co-expression of the L-protein and CFTR delivered as PCR product or as vector (left of a dashed line) leads to a decrease of yield but to a more homogenous sample compared to control expression of CFTR without co-expression of L-protein (right of dashed line).

FIG. 8A-8B show the results of His-tag purification of ITK. FIG. 8A shows SDS-PAGE of ITK affinity purified using a His tag. Lanes: lane 1, SeeBlue2 plus prestained; lane 2, 500 ng GFP; lane 3, 2 μg ITK; lane 4, 5 μg ITK; lane 5, 10 μg ITK. FIG. 8B shows Western Blot analysis after SDS-PAGE of FIG. 8A, with arrows pointing to the monomer and dimer of ITK.

FIG. 9A shows a schematic drawing of the luciferase gene construct under a CMV promoter. FIG. 9B shows the map of a plasmid having the construct depicted in FIG. 9A. FIG. 9C shows a schematic drawing of the luciferase reporter gene and the EMCV L1 protein linked by an IRES sequence under a shared CMV promoter. FIG. 9D shows the map of a plasmid having the construct depicted in FIG. 9C.

FIG. 10 shows luciferase expression as measured by bioluminescence readout. The use of plasmid in FIG. 9B results in higher luciferase expression initially, but expression decreases past day 18. The use of the plasmid disclosed herein (FIG. 9D) enables stable expression of the reporter gene over a prolonged period of time.

FIG. 11 shows bioluminescence images over time. Note: the injection of animal 4 in the test group was missed during the experiment, and therefore this animal was excluded from data analysis. The use of plasmid in FIG. 9B results in highly variable luciferase expression including loss of expression in two individual animals (animal 1 and 2) past day 18. The use of the plasmid disclosed herein (FIG. 9D) enables stable expression of the reporter gene over a prolonged period of time with low variability between animals.

FIG. 12 shows bioluminescence images over time for a representative mouse expressing just luciferase, and a representative mouse expressing luciferase in the presence of the enhancer protein. The use of the plasmid disclosed herein (FIG. 9D) enables stable expression of the reporter gene over a prolonged period of time.

FIG. 13A shows the map of a plasmid having a nucleic acid sequence encoding the adalimumab antibody under a CMV promoter. FIG. 13B shows the map of a plasmid having a nucleic acid sequence encoding the adalimumab antibody and a gene encoding the EMCV L1 protein linked by a nucleic acid sequence encoding an IRES under a shared CMV promoter.

FIG. 14A shows images from light microscopy (top) and images from immunofluorescence experiments (bottom) of HEK293T cells that express adalimumab from the EG140 control plasmid. FIG. 14B shows images from light microscopy (top) and images from immunofluorescence experiments of HEK293T cells that express adalimumab in combination with the L1 enhancer protein from the EG141 plasmid.

FIG. 15 shows the results from an ELISA experiment performed to detect the presence of adalimumab in the supernatant of HEK293T cells transiently transfected with either EG140 or EG141. A purified recombinant human anti-TNFa antibody (NBP2-62567 Novus Biologicals) was used as a positive control in this experiment.

FIG. 16 shows the results from an ELISA experiment performed to detect binding of human TNF-alpha to adalimumab secreted by HEK293T cells transiently transfected with either EG140 or EG141.

FIG. 17 shows the results from a luciferase reporter assay. The adalimumab in the supernatant of HEK293T cells transiently transfected with either EG140 or EG141 is able to suppress the TNF-alpha mediated activation of Luciferase expression in reporter cells.

FIG. 18A shows results from SDS PAGE and FIG. 18B shows results from western blot experiments depicting the heavy and light chains of adalimumab expressed from EG140-transfected cells or EG-141 transfected cells.

FIG. 19 shows a log quantification of bioluminescence imaging after 30 μg subcutaneous injection of plasmids expressing Firefly luciferase, alone (Fluc Std), and Fluc in combination with the L enhancer protein (Fluc EG).

FIG. 20 shows the schematic design of the pAAVtransfer_Adalimumab (Std) plasmid and enhancer protein plasmid pAAVtransfer_Adalimumab+L, demonstrating the position of the adalimumab expression cassette and enhancer protein L, relative to the 5′ and 3′ inverted terminal repeat (ITR) regions.

FIG. 21 shows a map of the pAAVtransfer_Adalimumab (Std) plasmid. The adalimumab expression cassette is positioned between the 5′ and 3′ inverted terminal repeat (ITR) regions of the AAV transfer vector.

FIG. 22 shows a map of the enhancer protein (EMCVgp1) pAAVtransfer_Adalimumab (EG) plasmid. The adalimumab expression cassette is positioned between the 5′ and 3′ inverted terminal repeat (ITR) regions of the AAV transfer vector.

FIG. 23 shows protein concentration of adalimumab (ng/ml) in cell culture supernatants of HEK293T cells transfected with pAdalimumab, pAdalimumab+enhancer L, pAAVtransfer adalimumab and pAAVtransfer adalimumab+enhancer L plasmids. Adalimumab protein concentration in cell culture supernatants was measured using quantitative ELISA.

FIG. 24 shows secreted adalimumab protein EC₅₀ values as measured with an HEK dual TNF-alpha reporter cells assay. Top, adalimumab EC₅₀ in cells transfected with the pAdalimumab_STD plasmid and the enhancer protein pAdalimumab_EG plasmid. Bottom, adalimumab EC₅₀ in cells transfected with pAAVtransfer_Adalimumab_STD and the enhancer L protein pAAVtransfer Adalimumab_EG plasmid. Tables 4 and 5 summarize these results.

FIG. 25 shows relative adalimumab activity, normalized to the amount of secreted adalimumab concentration and the activity of the respective control vector lacking the enhancer protein L.

FIGS. 26A and 26B show the concentration of adalimumab in mouse sera after treating mice with recombinant AAV vectors encoding adalimumab, alone, AAV_Adalimumab_STD, and with the enhancer protein L, AAV_Adalimumab_EG. FIG. 26A shows the results of AAV vectors administered via intramuscular injections. FIG. 26B shows the results of AAV vectors administered via subcutaneous injections.

FIG. 27 shows the schematic design of pGBA-NanoLuc_STD and enhancer protein pGBA-NanoLuc_EG plasmids.

FIGS. 28A and 28B show maps of the pGBA-NanoLuc_STD plasmid and the enhancer protein (EMCVgp1) pGBA-NanoLuc_EG plasmid, respectively.

FIG. 29 shows western blot results of pGBA-NanoLuc_STD and pGBA-NanoLuc_EG constructs expressed in HEK 293T cells. The predicted size of the pGBA-NanoLuc protein chimera is approximately 75 kDa.

FIGS. 30A-30D show the expression of the reporter protein and the enzymatic activity of GBA in HEK293T cell lysates (FIG. 30A and FIG. 30C, respectively) and supernatant (FIG. 30B and FIG. 30D, respectively), upon transfection with the pGBA-NanoLuc_STD plasmid and the enhancer protein pGBA-NanoLuc_EG plasmid. The total expression of both NanoLuc and GBA activity is higher in the absence of the enhancer protein L.

FIG. 31 shows the relative GBA activity, in a GBA-NanoLuc chimera protein, normalized to NanoLuc activity. Activity in HEK293T cell lysates and supernatant is shown upon transfection with the pGBA-NanoLuc_STD plasmid and the enhancer protein pGBA-NanoLuc_EG plasmid. In the cell culture supernatant, the relative GBA activity is significantly higher with enhancer protein co-expression, demonstrating that the enhancer protein increases the quality of the expressed GBA protein.

FIGS. 32A-32C show bioluminescence imaging results of Balb/c mice treated with pGBA-NanoLuc_STD plasmid and the enhancer protein GBA-NanoLuc_EG plasmid, formulated into lipid nanoparticles (LNPs). In FIG. 32A and FIG. 32B, the images were taken from the prone position and supine position, respectively. FIG. 32C and Table 6 demonstrate that the average coefficient of variation (CV %) of luciferase activity was higher without co-expression of the L enhancer protein.

### SUMMARY

The present inventors have recognized that co-expression of certain enhancer proteins with a target protein improves the expression quality and/or quantity, and/or prolongs the duration of expression, of recombinantly produced proteins, and the expression of a gene of interest in vitro, ex vivo and in vivo. In various embodiments, the disclosed compositions and methods exhibit one or more of the following advantages over the prior art: (1) they increase protein expression (yield) of a target protein within a eukaryotic cell line or a living subject; (2) they control the regulation of the expression of a target protein; (3) they express target protein that exhibits decreased undesirable properties (e.g., misfolding, altered activity, incorrect posttranslational modifications, and/or toxicity); (4) they increase correct folding and/or high yield of recombinant proteins; (5) they improve performance of the downstream activation pathways (e.g. GPCR signaling in a cell, or in the case of in vivo expression, immune system response to an expressed antigen); and/or (6) co-expression of the enhancer protein does not impact functionality of the target protein and/or downstream metabolism of the cell. The invention is not limited by these enumerated advantages, as some embodiments exhibit none, some, or all of these advantages.

In one aspect, the disclosure provides systems for recombinant expression of a target protein in eukaryotic cells, and methods for the expression of a target protein in vivo, that includes one or more vectors. The vectors (or a vector) have a first polynucleotide encoding the target protein and a second polynucleotide encoding an enhancer protein. The enhancer protein is an inhibitor of nucleocytoplasmic transport (NCT) and/or the enhancer protein is selected from the group consisting of a picomavirus leader (L) protein, a picomavirus 2A protease, a rhinovirus 3C protease, a herpes simplex virus (HSV) ICP27 protein, and a rhabdovirus matrix (M) protein. The first polynucleotide and the second polynucleotide are operatively linked to one or more promoters.

In another aspect, the disclosure provides a eukaryotic cell for expression of a target protein, where the cell includes an exogenous polynucleotide encoding an enhancer protein. The enhancer protein is an inhibitor of nucleocytoplasmic transport (NCT) and/or the enhancer protein is selected from the group consisting of a picomavirus leader (L) protein, a picomavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein. The exogenous polynucleotide is operatively linked to a promoter (optionally a native promoter or an exogenous promoter). In yet another aspect, the disclosure provides a method for recombinant expression of a target protein that includes introducing a polynucleotide encoding the target protein, operatively linked to a promoter, into this eukaryotic cell. In yet another aspect, the disclosure provides a method for recombinant expression of a target protein that includes introducing a vector system of the disclosure into a eukaryotic cell. In yet another aspect, the disclosure provides a cell produced by introducing of a vector system (or vector) of the disclosure into a eukaryotic cell. In yet another aspect, the disclosure provides a protein expressed by introduction of a vector system (or vector) of the disclosure into a eukaryotic cell. In yet another aspect, the disclosure provides a method for expressing a target protein in eukaryotic cells that includes introducing a polynucleotide encoding the target protein (the polynucleotide operatively linked to a promoter) into the eukaryotic cells. This method utilizes co-expression of an enhancer protein to enhance the expression level, solubility and/or activity of the target protein. The enhancer protein is an inhibitor of nucleocytoplasmic transport (NCT) and/or the enhancer protein is selected from the group consisting of a picomavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

In another aspect, the disclosure provides a method for generating an antibody against a target protein, comprising immunizing a subject with a cell or target protein produced using the systems or methods of the disclosure. In yet another aspect, the disclosure provides a method for antibody discovery by cell sorting, comprising providing a solution comprising a labeled cell or labeled target protein produced using the systems or methods of the disclosure, and a population of recombinant cells, wherein the recombinant cells express a library of polypeptides each comprising an antibody or antigen-binding fragment thereof; and sorting one or more recombinant cells from the solution by detecting recombinant cells bound to the labeled cell or the labeled target protein. In a further aspect, the disclosure provides, a method for panning a phage-display library, comprising mixing a phage-display library with a cell or target protein produced using the systems or methods of the disclosure; and purifying and/or enriching the members of the phage-display library that bind the cell or target protein.

Further aspects and embodiments are provided by the detailed disclosure that follows. The invention is not limited by this summary.

### DETAILED DESCRIPTION

In some embodiments, provided is a system for recombinant expression of a target protein that includes one or more vectors. In some embodiments, the expression is in eukaryotic cells. In some embodiments, the expression is in situ, in vivo, or ex vivo. In some embodiments, the vectors (or a vector) have a first polynucleotide encoding the target protein and a second polynucleotide encoding an enhancer protein. The enhancer protein is an inhibitor of nucleocytoplasmic transport (NCT) and/or the enhancer protein is selected from the group consisting of a picornavirus leader (L) protein, a picornavirus 2A protease, a rhinovirus 3C protease, a herpes simplex virus (HSV) ICP27 protein, and a rhabdovirus matrix (M) protein. The first polynucleotide and the second polynucleotide are operatively linked to one or more promoters.

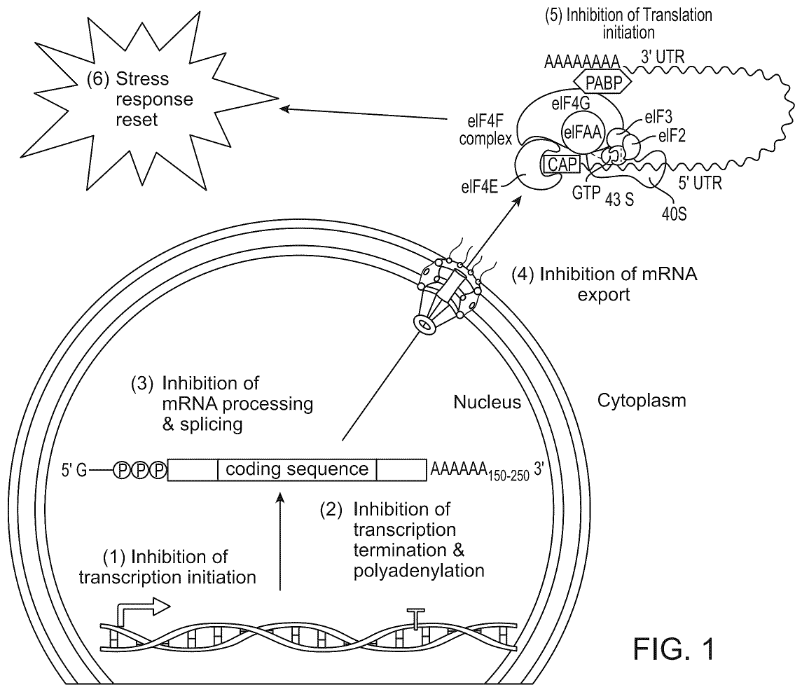

Without being bound by theory, it is believed that the compositions and methods of the disclosure prevent regulatory mechanisms of the cell from activating in response to expression of the recombinant target protein, and that this improves yields and/or functionality of the target protein. The methods and systems of the disclosure may inhibit or interfere with one or more cellular mechanisms, including but not limited to: (1) inhibition of transcription initiation, (2) inhibition of transcription termination and polyadenylation; (3) inhibition of mRNA processing and splicing, (4) inhibition of mRNA export; (5) inhibition of translation initiations; and (6) stress response (FIG. 1).

In various embodiments, the compositions and methods of the disclosure may improve target protein expression via co-expression of an enhancer protein, e.g. an L protein. The improved target protein expression associated with the compositions and methods of the disclosure may, for example, increase the activity of the target protein, lower expression levels, increase expression duration, increase stability, increase duration in a cell or subject, increase uniformity of delivery, reduce degradation, and/or reduce EC₅₀.

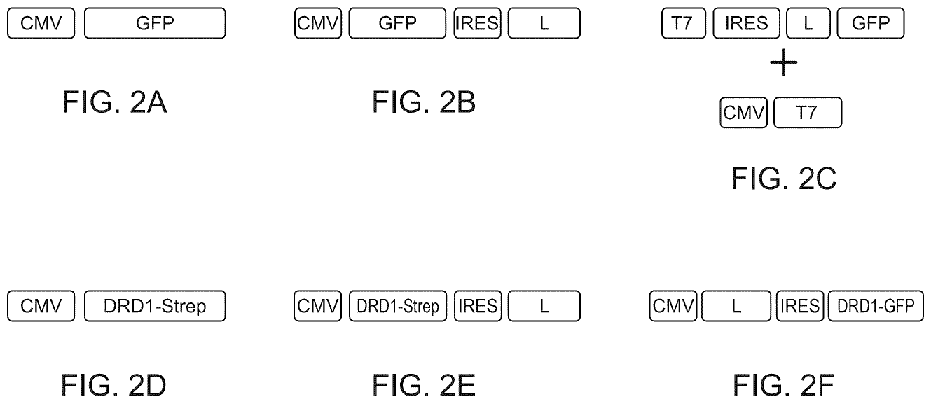

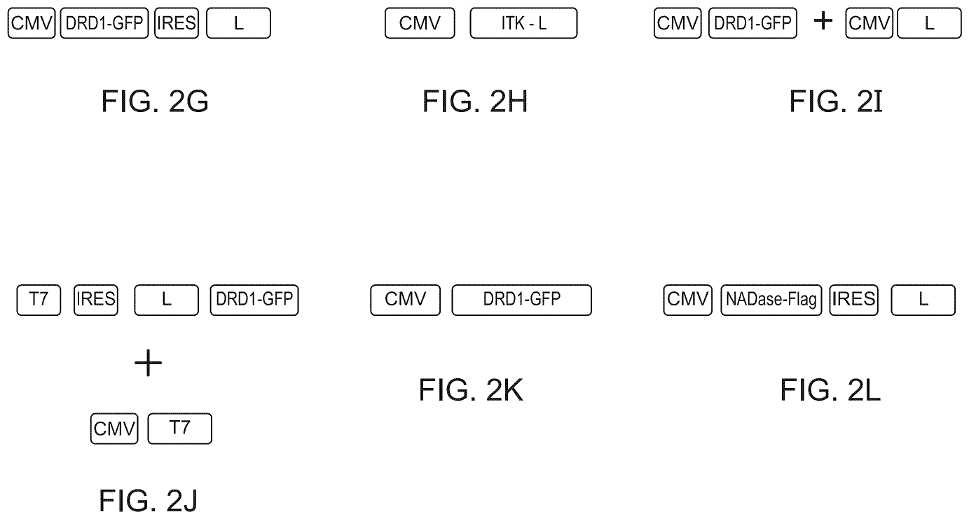

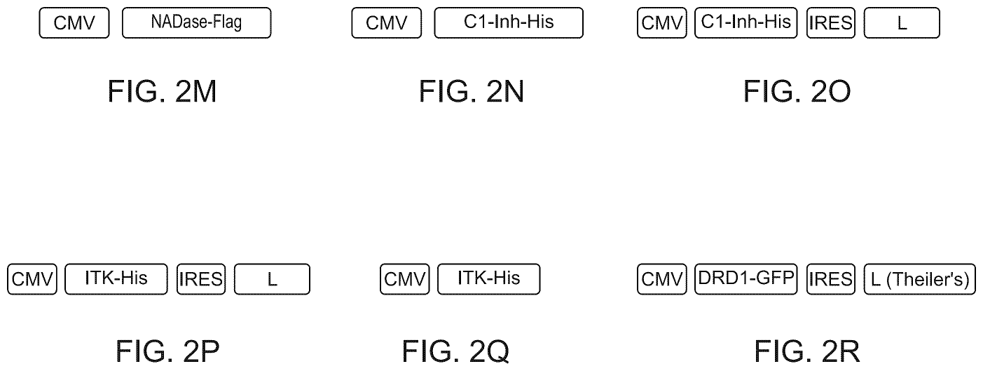

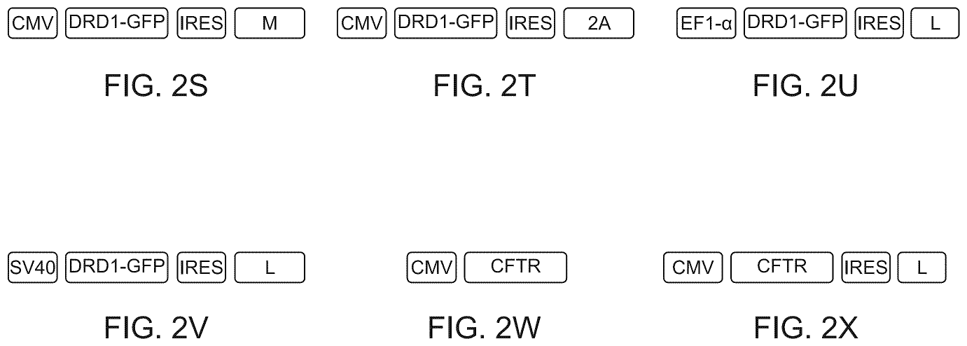

Various embodiments are depicted in FIGS. 2A-2Y and Table 1. In some embodiments, a first vector includes a polynucleotide encoding the target protein and a second vector includes a polynucleotide encoding the enhancer protein. In other embodiments, a single vector includes one or more polynucleotides encoding the target protein and the enhancer protein. The vector may comprise a single polynucleotide encoding both the target protein and the enhancer protein. In the alternative, more than one enhancer protein and/or more than one target protein are encoded by the vector or vectors.

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110.

Also as used herein, “and/or” refers to and encompasses any and all possible combinations of one or more of the associated listed items, as well as the lack of combinations when interpreted in the alternative (“or”).

As used herein, nucleotide sequences are listed in the 5′ to 3′ direction, and amino acid sequences are listed in the N-terminal to C-terminal direction, unless indicated otherwise.

The terms “nucleic acid sequence,” “nucleic acid,” “nucleotide,” “nucleotide sequence,” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Regulatory elements” include promoters, enhancers, internal ribosomal entry sites (IRES), and other expression control elements (e.g. transcription termination signals, such as polyadenylation signals and poly-U sequences). Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a regulatory element may be a pol I promoter, a pol II promoter, one a pol III promoter, or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I; SV40 enhancer; and the intron sequence between exons 2 and 3 of rabbit β-globin.

A “vector” is used to transfer genetic material into a target cell. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, adenoviruses, lentiviruses, and adeno-associated viruses). In embodiments, a viral vector may be replication incompetent. Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

The terms “polypeptide,” “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “subject” includes humans and other animals. Typically, the subject is a human. For example, the subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant. In other embodiments, subject is not a human; for example a non-human primate; for example, a baboon, a chimpanzee, a gorilla, or a macaque. In certain embodiments, the subject may be a pet, such as a dog or cat.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, “adalimumab” refers to the active pharmaceutical ingredient (API) in HUMIRA™, MABURA™, or EXEMPTIA™, or to functional variant thereof. Accordingly, the adalimumab may refer to adalimumab-adaz, adalimumab-adbm, adalimumab-afzb, adalimumab-atto, adalimumab-bwwd, or adalimumab-fkjp. In some embodiments, adalimumab comprises any of the CDRs of SEQ ID NOS: 137-142, according to WO2011153477, incorporated herein in its entirety.

As used herein, the terms “immunogen,” “antigen,” and “epitope” refer to substances such as proteins, including glycoproteins, and peptides that are capable of eliciting an immune response.

As used herein, an “immunogenic response” in a subject results in the development in the subject of a humoral and/or a cellular immune response to an antigen.

### Polynucleotides

The present disclosure relates to recombinant polynucleotides for the expression of one or more target proteins and one or more enhancer proteins. In some embodiments, the expression is in eukaryotic cells. In some embodiments, the expression is in situ, in vivo, or ex vivo. In some embodiments, polynucleotides (or nucleic acids or nucleic acid molecules) may comprise one or more genes of interest and is delivered to cells (e.g., eukaryotic cells) using the compositions and methods of the present disclosure. Polynucleotides of the present disclosure may include DNA, RNA, and DNA-RNA hybrid molecules. In some embodiments, polynucleotides are isolated from a natural source; prepared in vitro, using techniques such as PCR amplification, in vitro transcription, or chemical synthesis; prepared in vivo, e.g., via recombinant DNA technology; or prepared or obtained by any appropriate method. In some embodiments, polynucleotides are of any shape (linear, circular, etc.) or topology (single-stranded, double-stranded, linear, circular, supercoiled, torsional, nicked, etc.). Polynucleotides may also comprise nucleic acid derivatives such as peptide nucleic acids (PNAS) and polypeptide-nucleic acid conjugates; nucleic acids having at least one chemically modified sugar residue, backbone, internucleotide linkage, base, nucleotide, nucleoside, or nucleotide analog or derivative, or a basic site; as well as nucleic acids having chemically modified 5′ or 3′ ends; and nucleic acids having two or more of such modifications. Not all linkages in a polynucleotide need to be identical.

Examples of polynucleotides include without limitation oligonucleotides (including but not limited to antisense oligonucleotides, ribozymes and oligonucleotides useful in RNA interference (RNAi)), aptamers, nucleic acids, artificial chromosomes, cloning vectors and constructs, expression vectors and constructs, gene therapy vectors and constructs, rRNA, tRNA, mRNA, mtRNA, and tmRNA, and the like. In some embodiments, the polynucleotide is an in vitro transcribed (IVT) mRNA. In some embodiments, the polynucleotide is a plasmid.

A polynucleotide is said to “encode” a protein when it comprises a nucleic acid sequence that is capable of being transcribed and translated (e.g., DNA→RNA→protein) or translated (RNA→protein) in order to produce an amino acid sequence corresponding to the amino acid sequence of said protein. In vivo (e.g., within a eukaryotic cell) transcription and/or translation is performed by endogenous or exogenous enzymes. In some embodiments, transcription of the polynucleotides of the disclosure is performed by the endogenous polymerase II (polII) of the eukaryotic cell. In some embodiments, an exogenous RNA polymerase is provided on the same or a different vector. In some embodiments, the RNA polymerase is selected from a T3 RNA polymerase, a T5 RNA polymerase, a T7 RNA polymerase, and an H8 RNA polymerase.

Illustrative polynucleotides according to the present disclosure include a “first polynucleotide” encoding a target protein; a “second polynucleotide” encoding an enhancer protein; and a “coding polynucleotide” encoding one or more target proteins, one or more enhancer proteins, and/or one or more separating elements.

### Target Proteins

Polynucleotides according to the present disclosure may comprise a nucleic acid sequence encoding for one or more target proteins. The nucleic acid sequence encoding the target protein is referred to as the gene of interest (“GOP”).

In some embodiments, the expression of the protein may cause cell toxicity when expressed in a traditional expression system. In some embodiments, the protein is a protein with low yield expression in traditional expression systems. In some embodiments, the expression or quality of the protein is significantly improved by expression according to the disclosed methods, as compared to traditional expression systems. In some embodiments, expression of the target protein according to the disclosed methods causes less toxicity to the host cell, as compared to traditional expression systems. In some embodiments, expression of the target protein according to the disclosed methods does not cause toxicity to the host cell.

The target protein is not limited, and may be any protein for which expression is desired. In some embodiments, the target protein is a viral protein. In some embodiments, the target protein is a soluble protein, a secreted protein (such as, for example, C-Inh), or a membrane protein. The target protein may be derived from any protein or polypeptide. In some embodiments, the target protein is derived from one or more animal proteins, one or more human proteins, one or more microbial proteins, one or more viral proteins, one or more fungal proteins or a combination thereof. In some embodiments, the target protein can elicit an immunogenic response in a subject. In some embodiments, the target protein has one or more antigens.

In some embodiments, the target protein is comprised of one or more proteins, one or more protein domains, one or more isoforms, or chimeric proteins. In some embodiments, the protein domain is a structural domain, a functional domain, an extracellular domain, or an intracellular domain. In some embodiments, the target protein has an altered activity and/or altered circulation half-time, as compared to its naturally occurring counterpart. For instance, in some embodiments, the target protein is a chimeric protein comprised of a functional domain of protein A and a structural domain of protein B, wherein the chimeric protein has a functional activity, circulation half time, and/or other properties that are superior as compared to that of either protein A or protein B.

In some embodiments, the target protein is an antibody; an antibody-like molecule; a receptor; a monoclonal antibody; antibody parts or fragments; a nanobody; a bi-specific or multi-specific antibody; or a bi-specific or multi-specific antibody-like molecule. In some embodiments, the antibody is adalimumab. In some embodiments, the antibody is Abciximab, Alemtuzumab, Alirocumab, Amivantamab, Atezolizumab, Avelumab, Basiliximab, Belimumab, Benralizumab, Bevacizumab, Bezlotoxumab, Blinatumomab, Brentuximab vedotin, Brodalumab, Brolucizumab, Burosumab, Canakinumab, Caplacizumab, Capromab, Catumaxomab, Cemiplimab, Certolizumab pegol, Cetuximab, Crizanlizumab, Daclizumab, Daratumumab, Denosumab, Dinutuximab, Dupilumab, Durvalumab, Eculizumab, Elotuzumab, Emapalumab, Emicizumab, Enfortumab vedotin, Eptinezumab, Erenumab, Ertumaxomab, Etaracizumab, Evolocumab, Fremanezumab, Galcanezumab, Gemtuzumab ozogamicin, Golimumab, Guselkumab, Ibalizumab, Ibritumomab tiuxetan, Idarucizuma, Imciromab, Infliximab, Inotuzumab ozogamicin, Ipilimumab, Isatuximab, Itolizumab, Ixekizumab, Lanadelumab, Lokivetmab, Mepolizumab, Mogamulizumab, Moxetumomab Pasudotox, Natalizumab, Necitumumab, Nimotuzumab, Nivolumab, Obiltoxaximab, Obinutuzumab, Ocrelizumab, Ofatumumab, Olaratumab, Omalizumab, Palivizumab, Panitumumab, Pembrolizumab, Pertuzumab, Polatuzumab vedotin, Racotumomab, Ramucirumab, Ranibizumab, Raxibacumab, Ravulizumab, Reslizumab, Risankizumab, Rituximab, Rmab, Romosozumab, Rovelizumab, Ruplizumab, Sacituzumab govitecan, Sarilumab, Secukinumab, Sltuximab, Talquetamab, Teclistamab, Teprotumumab, Tildrakizumab, Tocilizumab, Tositumomab, Trastuzumab, Trastuzumab duocarmazine, Trastuzumab emtansine, Ustekinumab, and Vedolizumab. Polypeptide sequences for such antibodies are publicly available—for example, in the Thera-SAbDab database (at opig.stats.ox.ac.uk), described in Raybould et al. (2020) Thera-SAbDab: the Therapeutic Structural Antibody Database. Nucleic Acids Res. 48(D1):gkz827.

In some embodiments, the heavy chain of adalimumab has an amino acid sequence of SEQ ID NO: 132. In some embodiments, the light chain of adalimumab has an amino acid sequence of SEQ ID NO: 133. In some embodiments, the heavy chain of adalimumab is encoded by a nucleic acid sequence of SEQ ID NO: 134. In some embodiments, the light chain of adalimumab is encoded by a nucleic acid sequence of SEQ ID NO: 135.

In some embodiments, the target protein is a bi-specific or multi-specific antibody; or a bi-specific or multi-specific antibody-like molecule. In some embodiments, the bispecific antibody is Blinatumomab and Emicizumab. In some embodiments, the target protein is a bi-specific T-cell engager (BiTE), such as, for example, Blinatumomab (MT103) and Solitomab. In some embodiments, the target protein is a binding ligand or binder based on protein scaffold (such as, adnectin, anticalin, avimer, fynomer, Kunitz domain, Knottin, Affibody or DARPin).

In some embodiments, the target protein is a blood protein. Non-limiting examples of a blood protein include transferrin, t-PA, hirudin, C1 esterase inhibitor, anti-thrombin, plasma kallikrein inhibitor, plasmin, pro-thrombin complex, complement components, Prealbumin (transthyretin), Alpha 1 antitrypsin, Alpha-1-acid glycoprotein, Alpha-1-fetoprotein, alpha2-macroglobulin, Gamma globulins, Beta-2 microglobulin, Haptoglobin, Ceruloplasmin, Complement component 3, Complement component 4, C-reactive protein (CRP), Lipoproteins (chylomicrons, very low density lipoprotein (VLDL), low density lipoprotein (LDL), high density lipoprotein (HDL)), Transferrin, Prothrombin, mannose binding lectin (MBL), albumins, globulins, fibrinogen, regulatory factors, and coagulation factors, such as, Factor I, Factor II, Factor III, Factor IV, Factor V, Factor VI, Factor VII, Factor IX, Factor X, Factor XI, Factor XII, Factor XIII, von Willeband factor, prekallikrein, Fitzgerald factor, fibronectin, anti-thrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, and cancer procoagulant. In some embodiments, the target protein is a thrombolytic. Non-limiting examples of thrombolytics include Eminase (anistreplase), Retavase (reteplase), Streptase (streptokinase, kabikinase), alteplase, t-PA (class of drugs that includes Activase), TNKase (tenecteplase), Abbokinase, and Kinlytic (rokinase).

In some embodiments, the target protein is a growth factor. Non-limiting examples of growth factors include erythropoietin (EPO), Insulin like growth factor-1 (IGF-1), Granulocyte colony-stimulating factor (G-CSF), Granulocyte-macrophage colony-stimulating factor (GM-GCF), Bone morphogenetic protein-2 (BMP-2), Bone morphogenetic protein-7 (BMP-7), keratinocyte growth factor (KGF), Platelet-derived growth factor (PDGF), Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Colony-stimulating factors, Macrophage colony-stimulating factor (M-CSF), Epidermal growth factor (EGF), Ephrins—Ephrin A1, Ephrin A2, Ephrin A3, Ephrin A4, Ephrin A5, Ephrin B1, Ephrin B2, Ephrin B3, each of Fibroblast growth factor (FGF) 1, FGF2, FGF3, FGF4, FGF5, FGF6, FGF7, FGF8, FGF9, FGF10, FGF 11, FGF12, FGF13, FGF14, FGF15, FGF16, FGF17, FGF18, FGF19, FGF20, FGF21, FGF22, FGF23, Foetal Bovine Somatotrophin (FBS), GDNF family of ligands, Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factors, Insulin-like growth factor-1 (IGF-1), Insulin-like growth factor-2 (IGF-2), Interleukin-1 (IL-1), IL-2, IL-3, IL-4, IL-5, IL-6, IL-7, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1) Neuregulin 2 (NRG2), Neuregulin 3 (NRG3), Neuregulin 4 (NRG4), Neurotrophins, Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Vascular endothelial growth factor (VEGF), and Wnt Signaling Pathway. In some embodiments, the target protein is a hormone. Non-limiting examples of hormones include glucagon like peptide-1, insulin, human growth hormone, follicle stimulating hormone, calcitonin, lutropin, glucagon like peptide-2, leptin, parathyroid hormone, chorionic gonadotropin, thyroid stimulating hormone, and glucagon.

In some embodiments, the target protein is an enzyme. Non-limiting examples of an enzyme include Alpha-glycosidase, glucocerebrosidase, iduronate-2-sulfate, alpha-galactosidase, urate oxidase, N-acetyl-galactosidase, carboxypeptidase, hyaluronidase, DNAse, asparaginase, uricase, adenosine deaminase and other enterokinases, cyclases, caspases, cathepsins, oxidoreductases, transferases, hydrolases, lyases, isomerases, and ligases. A target protein for expression through the use of the present compositions and methods may include proteins related to enzyme replacement, such as Agalsidase beta, Agalsidase alfa, Imiglucerase, Taligulcerase alfa, Velaglucerase alfa, Alglucerase, Sebelipase alpha, Laronidase, Idursulfase, Elosulfase alpha, Galsulfase, Alglucosidase alpha, C3 inhibitor, Hurler and Hunter corrective factors. In some embodiments, the present compositions and methods are used for enzyme production. Such enzymes may be useful in the production of clinical testing kits or other diagnostic assays.

In some embodiments, a target protein is a membrane protein. Illustrative membrane proteins include ion channels, gap junctions, ionotropic receptors, transporters, integral membrane proteins such as cell surface receptors, proteins that shuttle between the membrane and cytosol in response to signaling, and the like. In some embodiments, the cell surface receptor is G-protein coupled receptors (GPCRs), tyrosine kinase receptors, integrins and the like. In some embodiments, the cell surface receptor is a G protein-coupled receptor. In some embodiments, the target protein is a seven-(pass)-transmembrane domain receptor, 7-transmembrane (7-TM) receptor, heptahelical receptor, serpentine receptor, or G protein— linked receptor (GPLR). In some embodiments, the target protein is a Class A GPCR, Class B GPCR, Class C GPCR, Class D GPCR, Class E GPCR, or Class F GPCR. In some embodiments, the target protein is a Class 1 GPCR, Class 2 GPCR, Class 3 GPCR, Class 4 GPCR, Class 5 GPCR, or Class 6 GPCR. In some embodiments, the target protein is a Rhodopsin-like GPCR, a Secretin receptor family GPCR, a Metabotropic glutamate/pheromone GPCR, a Fungal mating pheromone receptor, a Cyclic AMP receptor, or a Frizzled/Smoothened GPCR. In some embodiments, the cell surface receptor is IL-1 receptor, IL-1Ra, tumor necrosis factor receptor (TNFR), or vascular endothelial growth factor receptor (VEGFR). In some embodiments, the target protein is a receptor mimic. In some embodiments, the target protein is a protein that shuttles between the membrane and cytosol in response to signaling, such as, Ras protein, Rac protein, Raf protein, Ga subunits, arrestin, Src protein and other effector proteins.

In some embodiments, a target protein is a nucleosidase, an NAD+ nucleosidase, a hydrolase, a glycosylase, a glycosylase that hydrolyzes N-glycosyl compounds, an NAD+ glycohydrolase, an NADase, a DPNase, a DPN hydrolase, an NAD hydrolase, a diphosphopyridine nucleosidase, a nicotinamide adenine dinucleotide nucleosidase, an NAD glycohydrolase, an NAD nucleosidase, or a nicotinamide adenine dinucleotide glycohydrolase. In some embodiments, the target protein is an enzyme that participates in nicotinate and nicotinamide metabolism and calcium signaling pathway.

In some embodiments, the target protein is selected from the group consisting of Abatacept, Aflibercept, Agalsidase beta, Albiglutide, Aldesleukin, Alefacept, Alglucerase, Alglucosidase alfa, Aliskiren, Alpha-1-proteinase inhibitor, Alteplase, Anakinra, Ancestim, Anistreplase, Anthrax immune globulin human, Antihemophilic Factor, Antithrombin Alfa, Antithrombin III human, Antithymocyte globulin, Anti-thymocyte Globulin (Equine), Anti-thymocyte Globulin (Rabbit), Aprotinin, Arcitumomab, Asfotase Alfa, Asparaginase, Asparaginase Erwinia chrysanthemi, Becaplermin, Belatacept, Beractant, Bivalirudin, Botulinum Toxin Type A, Botulinum Toxin Type B, Buserelin, C1 Esterase Inhibitor (Human), C1 Esterase Inhibitor, Choriogonadotropin alfa, Chorionic Gonadotropin (Human), Chorionic Gonadotropin, Coagulation factor IX, Coagulation factor VIIa, Coagulation factor X human, Coagulation Factor XIII A-Subunit, Collagenase, Conestat alfa, Corticotropin, Cosyntropin, Daptomycin, Darbepoetin alfa, Defibrotide, Denileukin diftitox, Desirudin, Dornase alfa, Drotrecogin alfa, Dulaglutide, Efalizumab, Efmoroctocog alfa, Elosulfase alfa, Enfuvirtide, Epoetin alfa, Epoetin zeta, Eptifibatide, Etanercept, Exenatide, Factor IX Complex (Human), Fibrinogen Concentrate (Human), Fibrinolysin aka plasmin, Filgrastim, Filgrastim-sndz, Follitropin alpha, Follitropin beta, Galsulfase, Gastric intrinsic factor, Glatiramer acetate, Glucagon recombinant, Glucarpidase, Gramicidin D, Hepatitis A Vaccine, Hepatitis B immune globulin, Human calcitonin, Human Clostridium tetani toxoid immune globulin, Human rabies virus immune globulin, Human Rho(D) immune globulin, Human Serum Albumin, Human Varicella-Zoster Immune Globulin, Hyaluronidase, Hyaluronidase, Ibritumomab, Idursulfase, Imiglucerase, Immune Globulin Human, Infliximab, Insulin aspart, Insulin Beef, Insulin Degludec, Insulin detemir, Insulin Glargine, Insulin glulisine, Insulin Lispro, Insulin Pork, Insulin Regular, Insulin Regular, Insulin, porcine, Insulin, isophane, Interferon Alfa-2a, Recombinant, Interferon alfa-2b, Interferon alfacon-1, Interferon alfa-n1, Interferon alfa-n9, Interferon beta-1a, Interferon beta-1b, Interferon gamma-1b, Intravenous Immunoglobulin, Ipilimumab, Ixekizumab, Laronidase, Lenograstim, Lepirudin, Leuprolide, Liraglutide, Lucinactant, Lutropin alfa, Lutropin alfa, Mecasermin, Menotropins, Epoetin beta, Metreleptin, Muromonab, alpha interferon, Nesiritide, Ocriplasmin, Omalizumab, Oprelvekin, OspA lipoprotein, Oxytocin, Palifermin, Pancrelipase, Poractant alfa, Pramlintide, Preotact, Protein S human, Rasburicase, Reteplase, Rilonacept, Rituximab, Romiplostim, Sacrosidase, Salmon Calcitonin, Sargramostim, Satumomab Pendetide, Sebelipase alfa, Secretin, Secukinumab, Sermorelin, Serum albumin, Serum albumin iodonated, Simoctocog Alfa, Sipuleucel-T, Somatotropin Recombinant, Somatropin recombinant, Streptokinase, Sulodexide, Susoctocog alfa, Taliglucerase alfa, Teduglutide, Teicoplanin, Tenecteplase, Teriparatide, Tesamorelin, Thrombomodulin alfa, Thymalfasin, Thyroglobulin, Thyrotropin Alfa, Thyrotropin Alfa, Tocilizumab, Tositumomab, Tuberculin Purified Protein Derivative, Turoctocog alfa, Urofollitropin, Urokinase, Vasopressin, and Velaglucerase alfa.

In some embodiments, a target protein is a biosimilar. In some embodiments, the target protein is a therapeutic polypeptide, such as, a biopharmaceutical drug also known as biologics; a biomarker-enabling polypeptides, such as, a diagnostic, prognostic, or predictive biomarkers; a prophylactic polypeptide, such as, adjuvants, soluble antigens, sub viral particles, virus like particles; an auxiliary polypeptides, such as polypeptides supporting an activity or binding of another molecule or inhibiting another protein-protein interaction; a polypeptide used in research, such as antigens for generating novel monoclonal and polyclonal antibodies in animals, reporter proteins, or tool polypeptides for studying physiological or pathological processes and the effect of drugs on these processes in animal models. In some embodiments, the target protein is a protein that has applications in microscopy and imaging, such as, a fluorescent protein. In some embodiments, the target protein is not a reporter protein, such as, for example, luciferase. In some embodiments, the target protein is a human protein.

In some embodiments, the target protein is an immunomodulator. Non-limiting examples of immunomodulators include cytokines, chemokines, interleukins, interferons. In some embodiments, the target protein is an antigen for use as a vaccine or for research. In some embodiments, the target protein is a structural protein, such as a structural protein that functions in protein complex assembly. In some embodiments, the target protein is an anti-microbial polypeptide; or an anti-viral polypeptide. In some embodiments, the target protein is a tumor suppressor. In some embodiments, the target protein is a transcription factor or a translation factor. In some embodiments, the target protein is a pharmacokinetics modulating protein, a small molecule binding protein, an RNA binding protein, or a protein binding protein.

In some embodiments, the target protein is Dopamine receptor 1 (DRD1), Cystic fibrosis transmembrane conductance regulator (CFTR), C1 esterase inhibitor (C1-Inh), IL2 inducible T cell kinase (ITK), or an NADase. In some embodiments, the target protein is a firefly luciferase.

### Enhancer Proteins

The present disclosure relates to the co-expression of target proteins and enhancer proteins. In some embodiments, the enhancer proteins may improve one or more aspects of target protein expression, including but not limited to yield, quality, folding, posttranslational modification, activity, localization, and downstream activity, or may reduce one or more of misfolding, altered activity, incorrect posttranslational modifications, and/or toxicity.

In some embodiments, an enhancer protein is a nuclear pore blocking viral protein. In some embodiments, the enhancer protein is a native or synthetic peptide that is capable of blocking the nuclear pore, thereby inhibiting nucleocytoplasmic transport (“NCT”). In some embodiments, the enhancer protein is a viral protein. In some aspects, the viral protein is an NCT inhibitor.

In some embodiments, the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

The enhancer protein is a functional variant of any of the proteins disclosed herein. As used herein, the term “functional variant” refers to a protein that is homologous to an original protein and/or shares substantial sequence similarity to that original protein (e.g., more than 30%, 40%, 50%, 60%, 70%, 80%, 85% 90%, 95%, or 99% sequence identity) and shares one or more functional characteristics of the original protein. For example, a functional variant of an enhancer protein that is an NCT inhibitor retains the ability to inhibit NCT.

In some embodiments, the enhancer protein is a leader (L) protein from a picomavirus or a functional variant thereof. In some embodiments, the enhancer protein is a leader protein from the Cardiovirus, Hepatovirus, or Aphthovirus genera. For example, the enhancer protein may be from Bovine rhinitis A virus, Bovine rhinitis B virus, Equine rhinitis A virus, Foot-and-mouth disease virus, Hepatovirus A, Hepatovirus B, Marmota himalayana hepatovirus, Phopivirus, Cardiovirus A, Cardiovirus B, Theiler's Murine encephalomyelitis virus (TMEV), Vilyuisk human encephalomyelitis virus (VHEV), Theiler-like rat virus (TRV), or Saffold virus (SAF-V).

In some embodiments, the enhancer protein is the L protein of Theiler's virus or a functional variant thereof. In some embodiments, the L protein shares at least 90% identity to SEQ ID NO: 1. In some embodiments, the enhancer protein may comprise, consist of, or consist essentially of SEQ ID NO: 1. The enhancer protein may share at least 70%, 75%, 80%, 85%, 90%, 95%, 98%, 99% or 100% identity to SEQ ID NO: 1.

In some embodiments, the L protein is the L protein of Encephalomyocarditis virus (EMCV) or a functional variant thereof. In some embodiments, the L protein may share at least 90% identity to SEQ ID NO: 2. In some embodiments, the enhancer protein may comprise, consist of, or consist essentially of SEQ ID NO: 2. The enhancer protein may share at least 70%, 75%, 80%, 85%, 90%, 95%, 98%, 99% or 100% identity to SEQ ID NO: 2.

In some embodiments, the L protein is selected from the group consisting of the L protein of poliovirus, the L protein of HRV16, the L protein of mengo virus, and the L protein of Saffold virus 2 or a functional variant thereof.

In some embodiments, the enhancer protein is a picornavirus 2A protease or a functional variant thereof. In some embodiments, the enhancer protein is a 2A protease from Enterovirus, Rhinovirus, Aphtovirus, or Cardiovirus.

In some embodiments, the enhancer protein is a rhinovirus 3C protease or a functional variant thereof. In some embodiments, the enhancer protein is a Picomain 3C protease. In some embodiments, the enhancer protein is a 3C protease from enterovirus, rhinovirus, aphtovirus, or cardiovirus. For example, in some non-limiting embodiments, the enhancer protein is a 3C protease from Poliovirus, Coxsackievirus, Rhinovirus, Foot-and-mouth disease virus, or Hepatovirus A.

In some embodiments, the enhancer protein is a coronavirus ORF6 protein or a functional variant thereof. In some embodiments, the enhancer protein is a viral protein that disrupts nuclear import complex formation and/or disrupts STAT1 transport into the nucleus.

In some embodiments, the enhancer protein is an ebolavirus VP24 protein or a functional variant thereof. In some embodiments, the enhancer protein is an ebolavirus VP40 protein or VP35 protein. In some embodiments, the enhancer protein is a viral protein that binds to the importin protein karyopherin-α (KPNA). In some embodiments, the enhancer protein is a viral protein that inhibits the binding of STAT1 to KPNA.

In some embodiments, the enhancer protein is a Venezuelan equine encephalitis virus (VEEV) capsid protein or a functional variant thereof. In some embodiments, the enhancer protein is a viral capsid protein that interacts with the nuclear pore complex.

In some embodiments, the enhancer protein is a herpes simplex virus (HSV) ICP27 protein or a functional variant thereof. In some embodiments, the enhancer protein is an HSV ORF57 protein.

In some embodiments, the enhancer protein is a rhabdovirus matrix (M) protein or a functional variant thereof. In some embodiments, the enhancer protein is an M protein from Cytorhabdovirus, Dichorhavirus, Ephemerovirus, Lyssavirus, Novirhabdovirus, Nucleorhabdovirus, Perhabdovirus, Sigmavirus, Sprivivirus, Tibrovirus, Tupavirus, Varicosavirus, or Vesiculovirus.

In some embodiments, an enhancer protein is selected from the proteins listed in Table 1 or functional variants thereof. The polynucleotide encoding the enhancer protein may encode an amino acid sequence at least 70%, 75%, 80%, 85%, 90%, 95%, 98%, 99% or 100% identical to an amino acid sequence listed in Table 1. The amino acid sequence of the enhancer protein may be at least 70%, 75%, 80%, 85%, 90%, 95%, 98%, 99% or 100% identical to an amino acid sequence listed in Table 1. The amino acid sequence of the enhancer protein may be at least 70%, 75%, 80%, 85%, 90%, 95%, 98%, 99% or 100% identical to the amino acid sequence of SEQ ID NO: 1, 2, 3, 4, 5, 6, 7, 8, 9, 10, or 11. In some embodiments, an enhancer protein may have an amino acid sequence comprising, consisting of, or consisting essentially of one of the amino acid sequences listed in Table 1. In some embodiments, an enhancer protein may have an amino acid sequence comprising, consisting of, or consisting essentially of the amino acid sequence of SEQ ID NO: 1, 2, 3, 4, 5, 6, 7, 8, 9, 10, or 11.

TABLE 1
Illustrative enhancer proteins

| Nuclear pore | | | |
|---|---|---|---|
| blocking viral | | | |
| protein | Origin | Family | Amino acid sequence |
| Leader protein | Theiler's virus | Picornaviridae | MACKHGYPDVCPICTAVDAT |
| | | | PGFEYLLMADGEWYPTDLLC |
| | | | VDLDDDVFWPSDTSNQSQTM |
| | | | DWTDVPLIRDIVMEPQ |
| | | | (SEQ ID NO: 24) |
| Leader protein | Theiler's-like | Picornaviridae | MACKHGYPLMCPLCTALDK |
| | cardiovirus | | TSDGLFTLLFDNEWYPTDLLT |
| | | | VDLEDEVFYPDDPHMEWTDL |
| | | | PLIQDIEMEPQ |
| | | | (SEQ ID NO: 1) |
| Leader protein | EMCV | Picornaviridae | MATTMEQETCAHSLTFEECP |
| | | | KCSALQYRNGFYLLKYDEEW |
| | | | YPEELLTDGEDDVFDPELDM |
| | | | EVVFELQ |
| | | | (SEQ ID NO: 2) |
| Leader protein | Poliovirus | Picornaviridae | NYHLATQDDLQNAVNVMWS |
| | (Enterovirus C) | | RDLLVTESRAQGTDSIARCNC |
| | | | NAGVYYCESRRKYYPVSFVG |
| | | | PTFQYMEANNYYPARYQSH |
| | | | MLIGHGFASPGDCGGILRCHH |
| | | | GVIGIITAGGEGLVAFSDIRDL |
| | | | YAYEE |
| | | | (SEQ ID NO: 3) |
| Leader protein | Equine rhinitis | Picornaviridae | MVTMAGNMICNVFAGLATEI |
| | B virus 1 | | CSPKQGPLLDNELPLPLELAE |
| | | | FPNKDNNCWVAALSHYYTL |
| | | | CDVTNHVTKVTPTTSGIRYYL |
| | | | TAWQSILQTDLENGYYPAAF |
| | | | AVETGLCHGPFPMQQHGYVR |
| | | | NATSHPYNFCLCSEPVPGEDY |
| | | | WHAVVKVDLSRTEARVDKW |
| | | | LCIDDDRMYLSGPPTRVKLAS |
| | | | SYKIPTWIESLAQFCLQLHPV |
| | | | QHRRTLANSLRNEQCR |
| | | | (SEQ ID NO: 4) |
| Leader protein | Mengo virus | Picornaviridae | MATTMEQEICAHSMTFEECP |
| | (Cardiovirus) | | KCSALQYRNGFYLLKYDEEW |
| | | | YPEESLTDGEDDVFDPDLDM |
| | | | EVVFETQ |
| | | | (SEQ ID NO: 5) |
| Leader protein | Saffold virus 2 | Picornaviridae | MACKHGYPFLCPLCTAIDTT |
| | (Cardiovirus) | | HDGSFTLLIDNEWYPTDLLTV |
| | | | DLDDDVFHPDDSVMEWTDL |
| | | | PLIQDVVMEPQ |
| | | | (SEQ ID NO: 6) |
| 2A protease | Poliovirus | Picornaviridae | GFGHQNKAVYTAGYKICNY |
| | (Enterovirus C) | | HLATQDDLQNAVNVMWSRD |
| | | | LLVTESRAQGTDSIARCNCNA |
| | | | GVYYCESRRKYYPVSFVGPT |
| | | | FQYMEANNYYPARYQSHMLI |
| | | | GHGFASPGDCGGILRCHHGVI |
| | | | GIITAGGEGLVAFSDIRDLYA |
| | | | YEEEAMEQ |
| | | | (SEQ ID NO: 7) |
| 3C protease | HRV16 | Picornaviridae | GPEEEFGMSIIKNNTCVVTTT |
| | | | NGKFTGLGIYDRILILPTHADP |
| | | | GSEIQVNGIHTKVLDSYDLFN |
| | | | KEGVKLEITVLKLDRNEKFR |
| | | | DIRKYIPESEDDYPECNLALV |
| | | | ANQTEPTIIKVGDVVSYGNIL |
| | | | LSGTQTARMLKYNYPTKSGY |
| | | | CGGVLYKIGQILGIHVGGNGR |
| | | | DGFSSMLLRSYFTEQ |
| | | | (SEQ ID NO: 8) |
| M protein | Vesicular | Rhabdoviridae | MSSLKKILGLKGKGKKSKKL |
| | stomatitis virus | | GIAPPPYEEDTSMEYAPSAPID |
| | | | KSYFGVDEMDTYDPNQLRYE |
| | | | KFFFTVKMTVRSNRPFRTYSD |
| | | | VAAAVSHWDHMYIGMAGKR |
| | | | PFYKILAFLGSSNLKATPAVL |
| | | | ADQGQPEYHTHCEGRAYLPH |
| | | | RMGKTPPMLNVPEHFRRPFNI |
| | | | GLYKGTIELTMTIYDDESLEA |
| | | | APMIWDHFNSSKFSDFREKA |
| | | | LMFGLIVEKKASGAWVLDSIS |
| | | | HFK |
| | | | (SEQ ID NO: 9) |
| Non-structural | Influenza A | Orthomyxoviridae | MDPNTVSSFQVDCFLWHVRK |
| Protein 1 | virus | | RVADQELGDAPFLDRLRRDQ |
| | | | KSLRGRGSTLGLDIETATRAG |
| | | | KQIVERILKEESDEALKMTM |
| | | | ASVPASRYLTDMTLEEMSRD |
| | | | WSMLIPKQKVAGPLCIRMDQ |
| | | | AIMDKNIILKANFSVIFDRLET |
| | | | LILLRAFTEEGAIVGEISPLPSL |
| | | | PGHTAEDVKNAVGVLIGGLE |
| | | | WNDNTVRVSETLQRFAWRSS |
| | | | NENGRPPLTPKQKREMAGTI |
| | | | RSEV |
| | | | (SEQ ID NO: 10) |
| Immediate- | Simplexvirus | Herpesviridae | MATDIDMLIDLGLDLSDSDL |
| early protein | | | DEDPPEPAESRRDDLESDSSG |
| IE63 | | | ECSSSDEDMEDPHGEDGPEPI |
| | | | LDAARPAVRPSRPEDPGVPST |
| | | | QTPRPTERQGPNDPQPAPHSV |
| | | | WSRLGARRPSCSPEQHGGKV |
| | | | ARLQPPPTKAQPARGGRRGR |
| | | | RRGRGRGGPGAADGLSDPRR |
| | | | RAPRTNRNPGGPRPGAGWTD |
| | | | GPGAPHGEAWRGSEQPDPPG |
| | | | GQRTRGVRQAPPPLMTLAIAP |
| | | | PPADPRAPAPERKAPAADTID |
| | | | ATTRLVLRSISERAAVDRISES |
| | | | FGRSAQVMHDPFGGQPFPAA |
| | | | NSPWAPVLAGQGGPFDAETR |
| | | | RVSWETLVAHGPSLYRTFAG |
| | | | NPRAASTAKAMRDCVLRQE |
| | | | NFIEALASADETLAWCKMCI |
| | | | HHNLPLRPQDPIIGTTAAVLD |
| | | | NLATRLRPFLQCYLKARGLC |
| | | | GLDELCSRRRLADIKDIASFV |
| | | | FVILARLANRVERGVAEIDYA |
| | | | TLGVGVGEKMHFYLPGACM |
| | | | AGLIEILDTHRQECSSRVCELT |
| | | | ASHIVAPPYVHGKYFYCNSLF |
| | | | (SEQ ID NO: 11) |

### Fusion Proteins

In some embodiments, the target protein and the enhancer protein are comprised in a single fusion protein. In some embodiments, the fusion protein may comprise a linking element. In some embodiments, the linking element may comprise a cleavage site for enzymatic cleavage. In other embodiments, the fusion protein or the linking element does not comprise a cleavage site and the expressed fusion protein comprises both the target protein and the enhancer protein.

### Protein Modifications

The target proteins, enhancer proteins, and/or fusion proteins, or the polynucleotides encoding such, may be modified to comprise one or more markers, labels, or tags. For example, in some embodiments, a protein of the present disclosure may be labeled with any label that will allow its detection, e.g., a radiolabel, a fluorescent agent, biotin, a peptide tag, an enzyme fragment, or the like. The proteins may comprise an affinity tag, e.g., a His-tag, a GST-tag, a Strep-tag, a biotin-tag, an immunoglobulin binding domain, e.g., an IgG binding domain, a calmodulin binding peptide, and the like. In some embodiments, polynucleotides of the present disclosure comprise a selectable marker, e.g., an antibiotic resistance marker.

In some embodiments, the target protein bears one or more post-translational modifications. The type of post-translational modification is not limited, and may be any post translation known in the art. Non limiting examples of post translational modifications include glycosylation, acetylation, alkylation, methylation, biotinylation, glutamylation, glycylation, isoprenylation, lipoylation, phosphopantetheinylation, phosphorylation, sufation, selenation, C-terminal amidation, sumoylation, and any combination thereof.

### Polymerases

For the transcription of the polynucleotides encoding the target protein(s) and enhancer protein(s), an endogenous or exogenous polymerase may be used. In some embodiments, transcription of the polynucleotide(s) is performed by the natural polymerases comprised by the cell (e.g., eukaryotic cell). Viral polymerases may alternatively or additionally be used. In some embodiments, a viral promoter is used in combination with one or more viral polymerase. In some embodiments, eukaryotic promoters are used in combination with one or more eukaryotic polymerases. Illustrative viral polymerases include, but are not limited to, T7, T5, EMCV, HIV, Influenza, SP6, CMV, T3, T1, SP01, SP2, Phi15, and the like. Viral polymerases are RNA priming or capping polymerases. In some embodiments, IRES elements are used in conjunction with viral polymerases.

A vector or vectors according to the present disclosure may comprise a polynucleotide sequence encoding a polymerase. In some embodiments, the polymerase is a viral polymerase. The polynucleotide sequence encoding the polymerase may be comprised by a vector that comprises a target protein-encoding polynucleotide and/or an enhancer protein-encoding polynucleotide. In some embodiments, the polymerase may be comprised by a vector that does not comprise target protein or enhancer protein-encoding polynucleotides.

In some embodiments, at least one of the one or more vectors comprised by the systems, methods, or cells disclosed herein may comprise a polynucleotide sequence encoding a T7 RNA polymerase.

### Vectors

In some aspects, the present disclosure relates to vectors comprising nucleic acid sequences for the expression of one or more target proteins and one or more enhancer proteins. In some embodiments, the vectors (or a vector) have a first polynucleotide encoding the target protein and a second polynucleotide encoding an enhancer protein.

A vector for use according to the present disclosure may comprise any vector known in the art. In certain embodiments, the vector is any recombinant vector capable of expression of a protein or polypeptide of interest or a fragment thereof, for example, an adeno-associated virus (AAV) vector, a lentivirus vector, a retrovirus vector, a replication competent adenovirus vector, a replication deficient adenovirus vector, a herpes simplex virus, a retrovirus, a lentivirus, an alphavirus, a flavivirus, a rhabdovirus, a measles virus, a Newcastle disease virus, a poxvirus, a picornavirus, a herpes virus vector, a baculovirus vector, an adenoviral (Ad) vector or a nonviral plasmid. In some embodiments, the vector is a viral gene delivery vector based on an adeno-associated virus (AAV) vector, a lentivirus vector, a retrovirus vector, a replication competent adenovirus vector, a replication deficient adenovirus vector, a herpes simplex virus, a retrovirus, a lentivirus, an alphavirus, a flavivirus, a rhabdovirus, a measles virus, a Newcastle disease virus, a poxvirus, a picornavirus, a herpes virus vector, a baculovirus vector, an adenoviral (Ad) vector.

In some embodiments, the vector is a viral vector, a plasmid, a phage, a phagemid, a cosmid, a fosmid, a bacteriophage or an artificial chromosome. In some embodiments, the vector is a bacterial artificial chromosome (BAC), a plasmid, a bacteriophage P1-derived vector (PAC), a yeast artificial chromosome (YAC), or a mammalian artificial chromosome (MAC). In some embodiments, the vector is a naked or formulated plasmid DNA or minicircle. The formulation is not limited and may be based on non-viral DNA carriers such as, for example, peptides, lipids, polymers, or cations.

In some embodiments, the vector comprises polynucleotides that are expressed constitutively, transiently, or in a regulated manner. In some embodiments, the regulation involves safety switches. Regulated expression of polynucleotides from the vector may involve the use of any technology known in the art, such as inducible gene switches (for instance, synthetic receptors, protein-based switches, genetic circuits, genome editing tools, ribozymes or aptazymes); or the use of apoptotic suicide genes and pro-drugs. Protein-based switches are known in the art and may involve the use of dimerizing proteins or antibodies, such as rimiducid induced dimerization of monomeric Caspase 9.

Cells, systems, and methods disclosed herein may comprise one vector. In some embodiments, the cells, systems, and methods may comprise a single vector comprising a first polynucleotide encoding a target protein and a second polynucleotide encoding an enhancer protein.

Cells, systems, and methods disclosed herein may comprise two vectors. In some embodiments, the cells, systems, and methods may comprise a first vector comprising the first polynucleotide, operatively linked to a first promoter; and a second vector comprising the second polynucleotide, operatively linked to a second promoter.

Cells, systems, and methods disclosed herein may comprise more than two vectors, wherein the vectors may encode target protein(s) and enhancer protein(s) in a variety of combinations or configurations.

In some embodiments, provided is a cell comprising a vector or vectors of the disclosure. In some embodiments, provided is a cell comprising polynucleotides of the disclosure. In some embodiments, provided is a cell expressing target protein(s) and enhancer protein(s) of the disclosure.

Promoters

Vectors according to the present disclosure may comprise one or more promoters. The term “promoter” refers to a region or sequence located upstream or downstream from the start of transcription which is involved in recognition and binding of RNA polymerase and other proteins to initiate transcription. The polynucleotide(s) or vector(s) according to the present disclosure may comprise one or more promoters. The promoters may be any promoter known in the art. The promoter may be a forward promoter or a reverse promoter. In some embodiments, the promoter is a mammalian promoter. In some embodiments, one or more promoters are native promoters. In some embodiments, one or more promoters are non-native promoters. In some embodiments, one or more promoters are non-mammalian promoters. Non-limiting examples of RNA promoters for use in the disclosed compositions and methods include Ul, human elongation factor-1 alpha (EF-1 alpha), cytomegalovirus (CMV), human ubiquitin, spleen focus-forming virus (SFFV), U6, H1, tRNA^{Lys}, tRNA^{Ser} and tRNA^{Arg}, CAG, PGK, TRE, UAS, UbC, SV40, T7, Sp6, lac, araBad, trp, and Ptac promoters.

The term “operatively linked” as used herein refers to elements or structures in a nucleic acid sequence that are linked by operative ability and not physical location. The elements or structures are capable of, or characterized by, accomplishing a desired operation. It is recognized by one of ordinary skill in the art that it is not necessary for elements or structures in a nucleic acid sequence to be in a tandem or adjacent order to be operatively linked.

In some embodiments, a promoter comprised by a vector according to the present disclosure is an inducible promoter.

A vector according to the present disclosure may comprise one or more viral promoters that enable transcription of one or more polynucleotides by one or more viral polymerases. In some embodiments, for example, a vector may comprise a T7 promoter configured for transcription of either or both of the first polynucleotide (i.e., the target protein-encoding polynucleotide) or the second polynucleotide (i.e., the enhancer protein-encoding polynucleotide) by a T7 RNA polymerase.

Expression Cassettes

A vector or vectors according to the present disclosure may comprise one or more expression cassettes. The phrase “expression cassette” as used herein refers to a defined segment of a nucleic acid molecule that comprises the minimum elements needed for production of another nucleic acid or protein encoded by that nucleic acid molecule. In some embodiments, a vector may comprise an expression cassette, the expression cassette comprising a first polynucleotide encoding a target protein and a second polynucleotide encoding an enhancer protein. In some embodiments, the expression cassette comprises a first promoter, operatively linked to the first polynucleotide; and a second promoter, operatively linked to the second polynucleotide. In some embodiments, the expression cassette comprises a shared promoter operatively linked to both the first polynucleotide and the second polynucleotide.

In some embodiments, the expression cassette comprises a coding polynucleotide comprising the first polynucleotide and the second polynucleotide linked by a polynucleotide encoding a separating element (e.g., a ribosome skipping site or 2A element), the coding polynucleotide operatively linked to the shared promoter.

In some embodiments, the expression cassette comprises a coding polynucleotide, the coding polynucleotide encoding the enhancer protein and the target protein linked to by a separating element (e.g., a ribosome skipping site or 2A element), the coding polynucleotide operatively linked to the shared promoter.

In some embodiments, the expression cassette is configured for transcription of a single messenger RNA encoding both the target protein and the enhancer protein, linked by a separating element (e.g., a ribosome skipping site or 2A element); wherein translation of the messenger RNA results in expression of the target protein and the enhancer protein (e.g., the L protein) as distinct polypeptides.

In some embodiments, the expression cassette comprises a coding polynucleotide, the coding polynucleotide encoding the enhancer protein and the target protein as a fusion protein with or without a polypeptide linker, optionally wherein the polypeptide linker is a cleavable linker or an intein-based cleavage system.

Separating Elements

In some embodiments, target protein(s) and enhancer protein(s) according to the present disclosure are encoded on the same vector or are encoded on separate vectors. In some embodiments, if nucleic acid sequences for one or more target proteins and one or more enhancer proteins are comprised by the same vector, the vector may comprise a separating element for separate expression of the proteins. In various embodiments, the vector is a bicistronic vector or a polycistronic vector. The separating element may be an internal ribosomal entry site (IRES) or 2A element. In some embodiments, a vector may comprise a nucleic acid encoding a 2A self-cleaving peptide. Illustrative 2A self-cleaving peptides include P2A, E2A, F2A, and T2A.

In some embodiments, the first polynucleotide or the second polynucleotide, or both, are operatively linked to an internal ribosome entry site (IRES).

In some embodiments, the first polynucleotide or the second polynucleotide, or both, are operatively linked to a 2A element.

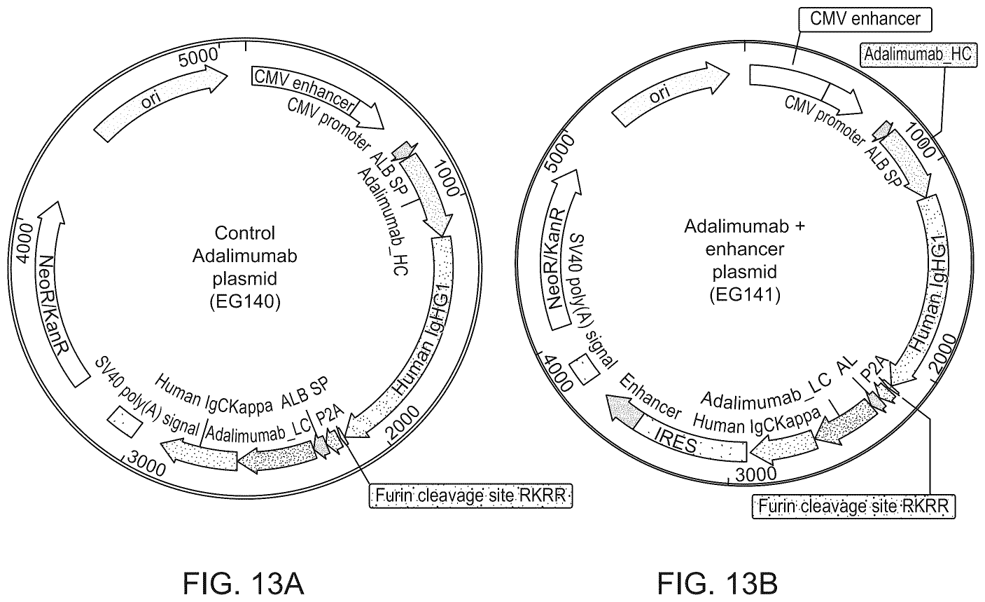

In some embodiments, the vector is as depicted in FIG. 13A or FIG. 13B. In some embodiments, the vector comprises a polynucleotide encoding SEQ ID NO: 132 and/or a polynucleotide encoding SEQ ID NO: 133. In some embodiments, the vector comprises a polynucleotide comprising the nucleic acid sequence of SEQ ID NO: 134 and/or a polynucleotide encoding SEQ ID NO: 135.

In some embodiments, the vector comprises the nucleic acid sequence of SEQ ID NO: 100, or a nucleic acid sequence with at least about 70%, at least about 75%, at least about 80%, at least about 85%, at least about 90%, at least about 95%, at least about 96%, at least about 97%, at least about 98%, or at least about 99% sequence identity to SEQ ID NO: 100.

SEQ ID NO: 100:
CGCGATGTACGGGCCAGATATACGCGTTGACATTGATTATTGACTAGTTATTAATAGTAATCAATTACGGGGTCATTAGTTC
ATAGCCCATATATGGAGTTCCGCGTTACATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGACGTCAATGGGTGGACTATTTACGGTAA
ACTGCCCACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTACCATGGT
GATGCGGTTTTGGCAGTACATCAATGGGCGTGGATAGCGGTTTGACTCACGGGGATTTCCAAGTCTCCACCCCATTGACGTC
AATGGGAGTTTGTTTTGGCACCAAAATCAACGGGACTTTCCAAAATGTCGTAACAACTCCGCCCCATTGACGCAAATGGGCG
GTAGGCGTGTACGGTGGGAGGTCTATATAAGCAGAGCTGGTTTAGTGAACCGTCAGATCCGCTAGCGCTACCGGACTCAGAT
CTCGAGCTCAAGCTTCGAATTCTGCAGTCGACGGTACCGCGGGCCCGGGATCCACCGGTCGCCACGATGAAGTGGGTGACCT
TCATCAGCCTGCTGTTCCTGTTTTCTTCCGCCTACAGCGAAGTGCAGCTGGTTGAAAGCGGAGGCGGACTGGTCCAGCCAGG
CAGAAGCCTGAGACTGTCTTGTGCCGCCTCTGGCTTCACCTTTGACGACTACGCCATGCACTGGGTGCGGCAGGCCCCTGGC
AAGGGACTCGAGTGGGTCAGCGCCATCACCTGGAATAGCGGCCACATCGACTACGCAGATAGCGTTGAAGGCAGATTCACCA
TCTCCAGGGACAACGCCAAGAATTCTCTGTACCTGCAGATGAACAGCCTGCGGGCCGAGGATACCGCTGTGTACTACTGCGC
CAAAGTGTCCTACCTGAGCACCGCCAGCTCCCTGGACTACTGGGGCCAGGGCACCCTGGTGACAGTGAGCTCTGCTAGCACA
AAAGGACCTAGCGTGTTTCCCCTGGCCCCTAGCAGCAAAAGCACCAGCGGCGGAACCGCCGCTCTGGGTTGTCTGGTGAAGG
ACTATTTCCCTGAACCTGTGACCGTGTCCTGGAACTCTGGCGCCCTGACTAGCGGCGTGCATACCTTCCCTGCCGTGCTGCA
AAGCTCTGGCCTGTATAGCCTTTCTTCTGTGGTGACCGTGCCTAGCAGCTCTCTGGGCACACAGACATACATCTGCAATGTG
AACCACAAGCCCTCCAACACCAAGGTGGACAAAAAGGTGGAACCCAAGAGCTGCGACAAGACCCACACCTGTCCTCCGTGCC
CCGCTCCTGAGCTGCTGGGCGGCCCTTCTGTGTTCCTGTTCCCCCCCAAACCTAAAGACACACTGATGATCAGCCGGACCCC
TGAGGTGACCTGCGTGGTGGTGGACGTGAGCCACGAGGACCCCGAGGTGAAGTTCAACTGGTACGTGGACGGCGTGGAGGTC
CACAACGCCAAGACCAAACCTAGAGAGGAACAATACAACAGCACATATAGAGTGGTGTCTGTGCTGACAGTGCTCCACCAGG
ACTGGCTGAACGGAAAGGAATACAAGTGCAAGGTGTCCAACAAGGCCCTCCCTGCTCCAATCGAGAAGACCATTAGCAAGGC
CAAGGGCCAACCTAGAGAGCCCCAGGTCTACACCCTGCCACCAAGTAGAGATGAGCTGACCAAGAACCAGGTGAGCCTAACA
TGCCTGGTGAAGGGCTTTTACCCCAGCGACATCGCCGTGGAATGGGAGAGCAACGGCCAGCCTGAGAACAACTACAAGACAA
CACCTCCTGTTCTGGATTCTGATGGCAGCTTCTTCCTGTACAGCAAGCTGACAGTGGATAAGAGCCGGTGGCAGCAGGGCAA
CGTGTTCAGCTGCTCCGTTATGCACGAGGCCCTGCATAATCACTACACCCAGAAGAGCCTGTCTCTGAGCCCTGGCAAGGAA
GTGCAGCTGGTTGAAAGCGGAGGCGGACTGGTCCAGCCAGGCAGAAGCCTGAGACTGTCTTGTGCCGCCTCTGGCTTCACCT
TTGACGACTACGCCATGCACTGGGTGCGGCAGGCCCCTGGCAAGGGACTCGAGTGGGTCAGCGCCATCACCTGGAATAGCGG
CCACATCGACTACGCAGATAGCGTTGAAGGCAGATTCACCATCTCCAGGGACAACGCCAAGAATTCTCTGTACCTGCAGATG
AACAGCCTGCGGGCCGAGGATACCGCTGTGTACTACTGCGCCAAAGTGTCCTACCTGAGCACCGCCAGCTCCCTGGACTACT
GGGGCCAGGGCACCCTGGTGACAGTGAGCTCTGCTAGCACAAAAGGACCTAGCGTGTTTCCCCTGGCCCCTAGCAGCAAAAG
CACCAGCGGCGGAACCGCCGCTCTGGGTTGTCTGGTGAAGGACTATTTCCCTGAACCTGTGACCGTGTCCTGGAACTCTGGC
GCCCTGACTAGCGGCGTGCATACCTTCCCTGCCGTGCTGCAAAGCTCTGGCCTGTATAGCCTTTCTTCTGTGGTGACCGTGC
CTAGCAGCTCTCTGGGCACACAGACATACATCTGCAATGTGAACCACAAGCCCTCCAACACCAAGGTGGACAAAAAGGTGGA
ACCCAAGAGCTGCGACAAGACCCACACCTGTCCTCCGTGCCCCGCTCCTGAGCTGCTGGGCGGCCCTTCTGTGTTCCTGTTC
CCCCCCAAACCTAAAGACACACTGATGATCAGCCGGACCCCTGAGGTGACCTGCGTGGTGGTGGACGTGAGCCACGAGGACC
CCGAGGTGAAGTTCAACTGGTACGTGGACGGCGTGGAGGTCCACAACGCCAAGACCAAACCTAGAGAGGAACAATACAACAG
CACATATAGAGTGGTGTCTGTGCTGACAGTGCTCCACCAGGACTGGCTGAACGGAAAGGAATACAAGTGCAAGGTGTCCAAC
AAGGCCCTCCCTGCTCCAATCGAGAAGACCATTAGCAAGGCCAAGGGCCAACCTAGAGAGCCCCAGGTCTACACCCTGCCAC
CAAGTAGAGATGAGCTGACCAAGAACCAGGTGAGCCTAACATGCCTGGTGAAGGGCTTTTACCCCAGCGACATCGCCGTGGA
ATGGGAGAGCAACGGCCAGCCTGAGAACAACTACAAGACAACACCTCCTGTTCTGGATTCTGATGGCAGCTTCTTCCTGTAC
AGCAAGCTGACAGTGGATAAGAGCCGGTGGCAGCAGGGCAACGTGTTCAGCTGCTCCGTTATGCACGAGGCCCTGCATAATC
ACTACACCCAGAAGAGCCTGTCTCTGAGCCCTGGCAAGCAAGCGAAAACGGCGCGGAAGCGGAGCTACTAACTTCAGCCTGC
TGAAGCAGGCTGGAGATGTGGAGGAGAACCCTGGACCTGATATCCAGATGACCCAGTCTCCATCTAGCCTGAGCGCCAGCGT
GGGAGATAGAGTGACCATCACCTGTAGAGCCTCTCAAGGCATCCGGAACTACCTGGCCTGGTATCAGCAGAAACCTGGCAAG
GCTCCTAAGCTGCTGATCTACGCCGCTTCCACCCTGCAGAGCGGCGTTCCTTCTAGATTCAGCGGCAGCGGCTCCGGAACAG
ACTTCACCCTGACAATTAGCTCCCTGCAACCTGAAGATGTGGCTACATACTACTGCCAGAGATACAATCGGGCCCCTTACAC
CTTTGGACAGGGCACCAAGGTGGAAATCAAGCGGACCGTGGCCGCCCCATCTGTGTTCATCTTCCCCCCCAGCGACGAGCAG
CTGAAAAGCGGCACAGCCAGCGTGGTGTGCCTGCTGAACAACTTCTACCCCAGGGAAGCCAAGGTGCAGTGGAAGGTGGACA
ATGCCCTGCAGAGCGGCAACAGCCAGGAGAGCGTGACCGAGCAGGACAGCAAGGACAGCACCTACAGCCTGAGCAGCACCCT
CACACTGTCTAAAGCCGACTACGAGAAGCACAAGGTCTACGCCTGCGAGGTGACCCACCAGGGCCTGTCCTCCCCTGTGACA
AAGAGCTTTAACAGAGGCGAGTGCTAAGATATCCAGATGACCCAGTCTCCATCTAGCCTGAGCGCCAGCGTGGGAGATAGAG
TGACCATCACCTGTAGAGCCTCTCAAGGCATCCGGAACTACCTGGCCTGGTATCAGCAGAAACCTGGCAAGGCTCCTAAGCT
GCTGATCTACGCCGCTTCCACCCTGCAGAGCGGCGTTCCTTCTAGATTCAGCGGCAGCGGCTCCGGAACAGACTTCACCCTG
ACAATTAGCTCCCTGCAACCTGAAGATGTGGCTACATACTACTGCCAGAGATACAATCGGGCCCCTTACACCTTTGGACAGG
GCACCAAGGTGGAAATCAAGCGGACCGTGGCCGCCCCATCTGTGTTCATCTTCCCCCCCAGCGACGAGCAGCTGAAAAGCGG
CACAGCCAGCGTGGTGTGCCTGCTGAACAACTTCTACCCCAGGGAAGCCAAGGTGCAGTGGAAGGTGGACAATGCCCTGCAG
AGCGGCAACAGCCAGGAGAGCGTGACCGAGCAGGACAGCAAGGACAGCACCTACAGCCTGAGCAGCACCCTCACACTGTCTA
AAGCCGACTACGAGAAGCACAAGGTCTACGCCTGCGAGGTGACCCACCAGGGCCTGTCCTCCCCTGTGACAAAGAGCTTTAA
CAGAGGCGAGTGCTAACCCCCCCCCCTAACGTTACTGGCCGAAGCCGCTTGGAATAAGGCCGGTGTGCGTTTGTCTATATGT
TATTTTCCACCATATTGCCGTCTTTTGGCAATGTGAGGGCCCGGAAACCTGGCCCTGTCTTCTTGACGAGCATTCCTAGGGG
TCTTTCCCCTCTCGCCAAAGGAATGCAAGGTCTGTTGAATGTCGTGAAGGAAGCAGTTCCTCTGGAAGCTTCTTGAAGACAA
ACAACGTCTGTAGCGACCCTTTGCAGGCAGCGGAACCCCCCACCTGGCGACAGGTGCCTCTGCGGCCAAAAGCCACGTGTAT
AAGATACACCTGCAAAGGCGGCACAACCCCAGTGCCACGTTGTGAGTTGGATAGTTGTGGAAAGAGTCAAATGGCTCTCCTC
AAGCGTATTCAACAAGGGGCTGAAGGATGCCCAGAAGGTACCCCATTGTATGGGATCTGATCTGGGGCCTCGGTGCACATGC
TTTACATGTGTTTAGTCGAGGTTAAAAAAACGTCTAGGCCCCCCGAACCACGGGGACGTGGTTTTCCTTTGAAAAACACGAT
GATAATATGGCCACAACCATGGAACAAGAGACTTGCGCGCACTCTCTCACTTTTGAGGAATGCCCAAAATGCTCTGCTCTAC
AATACCGTAATGGATTTTACCTGCTAAAGTATGATGAAGAATGGTACCCAGAGGAGTTATTGACTGATGGAGAGGATGATGT
CTTTGATCCCGAATTAGACATGGAAGTCGTTTTCGAGTTACAGTAAATCATAATCAGCCATACCACATTTGTAGAGGTTTTA
CTTGCTTTAAAAAACCTCCCACACCTCCCCCTGAACCTGAAACATAAAATGAATGCAATTGTTGTTGTTAACTTGTTTATTG
CAGCTTATAATGGTTACAAATAAAGCAATAGCATCACAAATTTCACAAATAAAGCATTTTTTTCACTGCATTCTAGTTGTGG
TTTGTCCAAACTCATCAATGTATCTTAAGGCGTCTTCTACTGGGCGGTTTTATGGACAGCAAGCGAACCGGAATTGCCAGCT
GGGGCGCCCTCTGGTAAGGTTGGGAAGCCCTGCAAAGTAAACTGGATGGCTTTCTTGCCGCCAAGGATCTGATGGCGCAGGG
GATCAAGCTCTGATCAAGAGACAGGATGAGGATCGTTTCGCATGATTGAACAAGATGGATTGCACGCAGGTTCTCCGGCCGC
TTGGGTGGAGAGGCTATTCGGCTATGACTGGGCACAACAGACAATCGGCTGCTCTGATGCCGCCGTGTTCCGGCTGTCAGCG
CAGGGGCGCCCGGTTCTTTTTGTCAAGACCGACCTGTCCGGTGCCCTGAATGAACTGCAAGACGAGGCAGCGCGGCTATCGT
GGCTGGCCACGACGGGCGTTCCTTGCGCAGCTGTGCTCGACGTTGTCACTGAAGCGGGAAGGGACTGGCTGCTATTGGGCGA
AGTGCCGGGGCAGGATCTCCTGTCATCTCACCTTGCTCCTGCCGAGAAAGTATCCATCATGGCTGATGCAATGCGGCGGCTG
CATACGCTTGATCCGGCTACCTGCCCATTCGACCACCAAGCGAAACATCGCATCGAGCGAGCACGTACTCGGATGGAAGCCG
GTCTTGTCGATCAGGATGATCTGGACGAAGAGCATCAGGGGCTCGCGCCAGCCGAACTGTTCGCCAGGCTCAAGGCGAGCAT
GCCCGACGGCGAGGATCTCGTCGTGACCCATGGCGATGCCTGCTTGCCGAATATCATGGTGGAAAATGGCCGCTTTTCTGGA
TTCATCGACTGTGGCCGGCTGGGTGTGGCGGACCGCTATCAGGACATAGCGTTGGCTACCCGTGATATTGCTGAAGAGCTTG
GCGGCGAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTTCACAC
CGCATACAGGTGGCACTTTTCGGGGAAATGTGCGCGGAACCCCTATTTGTTTATTTTTCTAAATACATTCAAATATGTATCC
GCTCATGAGACAATAACCCTGATAAATGCTTCAATAATAGCACGTGCTAAAACTTCATTTTTAATTTAAAAGGATCTAGGTG
AAGATCCTTTTTGATAATCTCATGACCAAAATCCCTTAACGTGAGTTTTCGTTCCACTGAGCGTCAGACCCCGTAGAAAAGA
TCAAAGGATCTTCTTGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATACCAAATACTGTCCTT
CTAGTGTAGCCGTAGTTAGGCCACCACTTCAAGAACTCTGTAGCACCGCCTACATACCTCGCTCTGCTAATCCTGTTACCAG
TGGCTGCTGCCAGTGGCGATAAGTCGTGTCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGAACTGAGATACCTACAGCGTGAGCTATGA
GAAAGCGCCACGCTTCCCGAAGGGAGAAAGGCGGACAGGTATCCGGTAAGCGGCAGGGTCGGAACAGGAGAGCGCACGAGGG
AGCTTCCAGGGGGAAACGCCTGGTATCTTTATAGTCCTGTCGGGTTTCGCCACCTCTGACTTGAGCGTCGATTTTTGTGATG
CTCGTCAGGGGGGCGGAGCCTATGGAAAAACGCCAGCAACGCGGCCTTTTTACGGTTCCTGGGCTTTTGCTGGCCTTTTGCT
CACATGTTCTTGACTCTT

In some embodiments, the vector comprises one or more the genetic elements below.

### Genetic Elements of Vector Shown in FIG. 13B

Misc.:
SEQ ID NO: 101
CGCGATGTACGGGCCAGATATACGCGTT
CMV enhancer: SEQ ID NO: 102
GACATTGATTATTGACTAGTTATTAATAGTAATCAATTACGGGGTCATTAGTTCATAGCCCATATATGGAGTTCCGCGT
TACATAACTTACGGTAAATGGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGACTATTTACGGTAAACTGCCCACTTGGCAGTAC
ATCAAGTGTATCATATGCCAAGTACGCCCCCTATTGACGTCAATGACGGTAAATGGCCCGCCTGGCATTATGCCCAGTA
CATGACCTTATGGGACTTTCCTACTTGGCAGTACATCTACGTATTAGTCATCGCTATTACCATG
CMV promoter:
SEQ ID NO: 103
GTGATGCGGTTTTGGCAGTACATCAATGGGCGTGGATAGCGGTTTGACTCACGGGGATTTCCAAGTCTCCACCCCATTG
ACGTCAATGGGAGTTTGTTTTGGCACCAAAATCAACGGGACTTTCCAAAATGTCGTAACAACTCCGCCCCATTGACGCA
AATGGGCGGTAGGCGTGTACGGTGGGAGGTCTATATAAGCAGAGCT
Misc.:
SEQ ID NO: 104
GGTTTAGTGAACCGTCAGATCCGCTAGCGCTACCGGACTCAGATCTCGAGCTCAAGCTTCGAATTCTGCAGTCGACGGT
ACCGCGGGCCCGGGATCCACCGGTCGCCACG
Albumin signal peptide (codon optimized):
SEQ ID NO: 105
ATGAAGTGGGTGACCTTCATCAGCCTGCTGTTCCTGTTTTCTTCCGCCTACAGC
Adalimumab Heavy Chain (variable region):
SEQ ID NO: 106
GAAGTGCAGCTGGTTGAAAGCGGAGGCGGACTGGTCCAGCCAGGCAGAAGCCTGAGACTGTCTTGTGCCGCCTCTGGCT
TCACCTTTGACGACTACGCCATGCACTGGGTGCGGCAGGCCCCTGGCAAGGGACTCGAGTGGGTCAGCGCCATCACCTG
GAATAGCGGCCACATCGACTACGCAGATAGCGTTGAAGGCAGATTCACCATCTCCAGGGACAACGCCAAGAATTCTCTG
TACCTGCAGATGAACAGCCTGCGGGCCGAGGATACCGCTGTGTACTACTGCGCCAAAGTGTCCTACCTGAGCACCGCCA
GCTCCCTGGACTACTGGGGCCAGGGCACCCTGGTGACAGTGAGCTCT
Human IgG1 Heavy Chain (constant region):
SEQ ID NO: 107
GCTAGCACAAAAGGACCTAGCGTGTTTCCCCTGGCCCCTAGCAGCAAAAGCACCAGCGGCGGAACCGCCGCTCTGGGTT
GTCTGGTGAAGGACTATTTCCCTGAACCTGTGACCGTGTCCTGGAACTCTGGCGCCCTGACTAGCGGCGTGCATACCTT
CCCTGCCGTGCTGCAAAGCTCTGGCCTGTATAGCCTTTCTTCTGTGGTGACCGTGCCTAGCAGCTCTCTGGGCACACAG
ACATACATCTGCAATGTGAACCACAAGCCCTCCAACACCAAGGTGGACAAAAAGGTGGAACCCAAGAGCTGCGACAAGA
CCCACACCTGTCCTCCGTGCCCCGCTCCTGAGCTGCTGGGCGGCCCTTCTGTGTTCCTGTTCCCCCCCAAACCTAAAGA
CACACTGATGATCAGCCGGACCCCTGAGGTGACCTGCGTGGTGGTGGACGTGAGCCACGAGGACCCCGAGGTGAAGTTC
AACTGGTACGTGGACGGCGTGGAGGTCCACAACGCCAAGACCAAACCTAGAGAGGAACAATACAACAGCACATATAGAG
TGGTGTCTGTGCTGACAGTGCTCCACCAGGACTGGCTGAACGGAAAGGAATACAAGTGCAAGGTGTCCAACAAGGCCCT
CCCTGCTCCAATCGAGAAGACCATTAGCAAGGCCAAGGGCCAACCTAGAGAGCCCCAGGTCTACACCCTGCCACCAAGT
AGAGATGAGCTGACCAAGAACCAGGTGAGCCTAACATGCCTGGTGAAGGGCTTTTACCCCAGCGACATCGCCGTGGAAT
GGGAGAGCAACGGCCAGCCTGAGAACAACTACAAGACAACACCTCCTGTTCTGGATTCTGATGGCAGCTTCTTCCTGTA
CAGCAAGCTGACAGTGGATAAGAGCCGGTGGCAGCAGGGCAACGTGTTCAGCTGCTCCGTTATGCACGAGGCCCTGCAT
AATCACTACACCCAGAAGAGCCTGTCTCTGAGCCCTGGCAAG
Adalimumab Heavy Chain complete:
SEQ ID NO: 134
GAAGTGCAGCTGGTTGAAAGCGGAGGCGGACTGGTCCAGCCAGGCAGAAGCCTGAGACTGTCTTGTGCCGCCTCTGGCT
TCACCTTTGACGACTACGCCATGCACTGGGTGCGGCAGGCCCCTGGCAAGGGACTCGAGTGGGTCAGCGCCATCACCTG
GAATAGCGGCCACATCGACTACGCAGATAGCGTTGAAGGCAGATTCACCATCTCCAGGGACAACGCCAAGAATTCTCTG
TACCTGCAGATGAACAGCCTGCGGGCCGAGGATACCGCTGTGTACTACTGCGCCAAAGTGTCCTACCTGAGCACCGCCA
GCTCCCTGGACTACTGGGGCCAGGGCACCCTGGTGACAGTGAGCTCTGCTAGCACAAAAGGACCTAGCGTGTTTCCCCT
GGCCCCTAGCAGCAAAAGCACCAGCGGCGGAACCGCCGCTCTGGGTTGTCTGGTGAAGGACTATTTCCCTGAACCTGTG
ACCGTGTCCTGGAACTCTGGCGCCCTGACTAGCGGCGTGCATACCTTCCCTGCCGTGCTGCAAAGCTCTGGCCTGTATA
GCCTTTCTTCTGTGGTGACCGTGCCTAGCAGCTCTCTGGGCACACAGACATACATCTGCAATGTGAACCACAAGCCCTC
CAACACCAAGGTGGACAAAAAGGTGGAACCCAAGAGCTGCGACAAGACCCACACCTGTCCTCCGTGCCCCGCTCCTGAG
CTGCTGGGCGGCCCTTCTGTGTTCCTGTTCCCCCCCAAACCTAAAGACACACTGATGATCAGCCGGACCCCTGAGGTGA
CCTGCGTGGTGGTGGACGTGAGCCACGAGGACCCCGAGGTGAAGTTCAACTGGTACGTGGACGGCGTGGAGGTCCACAA
CGCCAAGACCAAACCTAGAGAGGAACAATACAACAGCACATATAGAGTGGTGTCTGTGCTGACAGTGCTCCACCAGGAC
TGGCTGAACGGAAAGGAATACAAGTGCAAGGTGTCCAACAAGGCCCTCCCTGCTCCAATCGAGAAGACCATTAGCAAGG
CCAAGGGCCAACCTAGAGAGCCCCAGGTCTACACCCTGCCACCAAGTAGAGATGAGCTGACCAAGAACCAGGTGAGCCT
AACATGCCTGGTGAAGGGCTTTTACCCCAGCGACATCGCCGTGGAATGGGAGAGCAACGGCCAGCCTGAGAACAACTAC
AAGACAACACCTCCTGTTCTGGATTCTGATGGCAGCTTCTTCCTGTACAGCAAGCTGACAGTGGATAAGAGCCGGTGGC
AGCAGGGCAACGTGTTCAGCTGCTCCGTTATGCACGAGGCCCTGCATAATCACTACACCCAGAAGAGCCTGTCTCTGAG
CCCTGGCAAG
Furin cleavage site:
SEQ ID NO: 108
CAAGCGAAAACGGCGC
GSGlinker:
SEQ ID NO: 109
GGAAGCGGA
P2A self-cleaving peptide:
SEQ ID NO: 110
GCTACTAACTTCAGCCTGCTGAAGCAGGCTGGAGATGTGGAGGAGAACCCTGGACCT
Adalimumab Light Chain (variable region):
SEQ ID NO: 111
GATATCCAGATGACCCAGTCTCCATCTAGCCTGAGCGCCAGCGTGGGAGATAGAGTGACCATCACCTGTAGAGCCTCTC
AAGGCATCCGGAACTACCTGGCCTGGTATCAGCAGAAACCTGGCAAGGCTCCTAAGCTGCTGATCTACGCCGCTTCCAC
CCTGCAGAGCGGCGTTCCTTCTAGATTCAGCGGCAGCGGCTCCGGAACAGACTTCACCCTGACAATTAGCTCCCTGCAA
CCTGAAGATGTGGCTACATACTACTGCCAGAGATACAATCGGGCCCCTTACACCTTTGGACAGGGCACCAAGGTGGAAA
TCAAG
Human Ig Kappa Constant (constant region of light chain):
SEQ ID NO: 112
CGGACCGTGGCCGCCCCATCTGTGTTCATCTTCCCCCCCAGCGACGAGCAGCTGAAAAGCGGCACAGCCAGCGTGGTGT
GCCTGCTGAACAACTTCTACCCCAGGGAAGCCAAGGTGCAGTGGAAGGTGGACAATGCCCTGCAGAGCGGCAACAGCCA
GGAGAGCGTGACCGAGCAGGACAGCAAGGACAGCACCTACAGCCTGAGCAGCACCCTCACACTGTCTAAAGCCGACTAC
GAGAAGCACAAGGTCTACGCCTGCGAGGTGACCCACCAGGGCCTGTCCTCCCCTGTGACAAAGAGCTTTAACAGAGGCG
AGTGCTAA
Adalimumab Light Chain complete:
SEQ ID NO: 135
GATATCCAGATGACCCAGTCTCCATCTAGCCTGAGCGCCAGCGTGGGAGATAGAGTGACCATCACCTGTAGAGCCTCTC
AAGGCATCCGGAACTACCTGGCCTGGTATCAGCAGAAACCTGGCAAGGCTCCTAAGCTGCTGATCTACGCCGCTTCCAC
CCTGCAGAGCGGCGTTCCTTCTAGATTCAGCGGCAGCGGCTCCGGAACAGACTTCACCCTGACAATTAGCTCCCTGCAA
CCTGAAGATGTGGCTACATACTACTGCCAGAGATACAATCGGGCCCCTTACACCTTTGGACAGGGCACCAAGGTGGAAA
TCAAGCGGACCGTGGCCGCCCCATCTGTGTTCATCTTCCCCCCCAGCGACGAGCAGCTGAAAAGCGGCACAGCCAGCGT
GGTGTGCCTGCTGAACAACTTCTACCCCAGGGAAGCCAAGGTGCAGTGGAAGGTGGACAATGCCCTGCAGAGCGGCAAC
AGCCAGGAGAGCGTGACCGAGCAGGACAGCAAGGACAGCACCTACAGCCTGAGCAGCACCCTCACACTGTCTAAAGCCG
ACTACGAGAAGCACAAGGTCTACGCCTGCGAGGTGACCCACCAGGGCCTGTCCTCCCCTGTGACAAAGAGCTTTAACAG
AGGCGAGTGCTAA
Misc.:
SEQ ID NO: 113
CCCCCCCCCCTA
IRES:
SEQ ID NO: 114
ACGTTACTGGCCGAAGCCGCTTGGAATAAGGCCGGTGTGCGTTTGTCTATATGTTATTTTCCACCATATTGCCGTCTTT
TGGCAATGTGAGGGCCCGGAAACCTGGCCCTGTCTTCTTGACGAGCATTCCTAGGGGTCTTTCCCCTCTCGCCAAAGGA
ATGCAAGGTCTGTTGAATGTCGTGAAGGAAGCAGTTCCTCTGGAAGCTTCTTGAAGACAAACAACGTCTGTAGCGACCC
TTTGCAGGCAGCGGAACCCCCCACCTGGCGACAGGTGCCTCTGCGGCCAAAAGCCACGTGTATAAGATACACCTGCAAA
GGCGGCACAACCCCAGTGCCACGTTGTGAGTTGGATAGTTGTGGAAAGAGTCAAATGGCTCTCCTCAAGCGTATTCAAC
AAGGGGCTGAAGGATGCCCAGAAGGTACCCCATTGTATGGGATCTGATCTGGGGCCTCGGTGCACATGCTTTACATGTG
TTTAGTCGAGGTTAAAAAAACGTCTAGGCCCCCCGAACCACGGGGACGTGGTTTTCCTTTGAAAAACACGATGATAAT
Enhancer peptide:
SEQ ID NO: 115
ATGGCCACAACCATGGAACAAGAGACTTGCGCGCACTCTCTCACTTTTGAGGAATGCCCAAAATGCTCTGCTCTACAAT
ACCGTAATGGATTTTACCTGCTAAAGTATGATGAAGAATGGTACCCAGAGGAGTTATTGACTGATGGAGAGGATGATGT
CTTTGATCCCGAATTAGACATGGAAGTCGTTTTCGAGTTACAGTAA
Misc.:
SEQ ID NO: 116
ATCATAATCAGCCATACCACATTTGTAGAGGTTTTACTTGCTTTAAAAAACCTCCCACACCTCCCCCTGAACCTGAAAC
ATAAAATGAATGCAATTGTTGTTGTT
SV40 poly(A) signal:
SEQ ID NO: 117
AACTTGTTTATTGCAGCTTATAATGGTTACAAATAAAGCAATAGCATCACAAATTTCACAAATAAAGCATTTTTTTCAC
TGCATTCTAGTTGTGGTTTGTCCAAACTCATCAATGTATCTTA
Linker:
SEQ ID NO: 118
AGGCGTCTTCTACTGGGCGGTTTTATGGACAGCAAGCGAACCGGAATTGCCAGCTGGGGCGCCCTCTGGTAAGGTTGGG
AAGCCCTGCAAAGTAAACTGGATGGCTTTCTTGCCGCCAAGGATCTGATGGCGCAGGGGATCAAGCTCTGATCAAGAGA
CAGGATGAGGATCGTTTCGC
Neomycin/Kanamycin resistance gene:
SEQ ID NO: 119
ATGATTGAACAAGATGGATTGCACGCAGGTTCTCCGGCCGCTTGGGTGGAGAGGCTATTCGGCTATGACTGGGCACAAC
AGACAATCGGCTGCTCTGATGCCGCCGTGTTCCGGCTGTCAGCGCAGGGGCGCCCGGTTCTTTTTGTCAAGACCGACCT
GTCCGGTGCCCTGAATGAACTGCAAGACGAGGCAGCGCGGCTATCGTGGCTGGCCACGACGGGCGTTCCTTGCGCAGCT
GTGCTCGACGTTGTCACTGAAGCGGGAAGGGACTGGCTGCTATTGGGCGAAGTGCCGGGGCAGGATCTCCTGTCATCTC
ACCTTGCTCCTGCCGAGAAAGTATCCATCATGGCTGATGCAATGCGGCGGCTGCATACGCTTGATCCGGCTACCTGCCC
ATTCGACCACCAAGCGAAACATCGCATCGAGCGAGCACGTACTCGGATGGAAGCCGGTCTTGTCGATCAGGATGATCTG
GACGAAGAGCATCAGGGGCTCGCGCCAGCCGAACTGTTCGCCAGGCTCAAGGCGAGCATGCCCGACGGCGAGGATCTCG
TCGTGACCCATGGCGATGCCTGCTTGCCGAATATCATGGTGGAAAATGGCCGCTTTTCTGGATTCATCGACTGTGGCCG
GCTGGGTGTGGCGGACCGCTATCAGGACATAGCGTTGGCTACCCGTGATATTGCTGAAGAGCTTGGCGGCGAATGGGCT
GACCGCTTCCTCGTGCTTTACGGTATCGCCGCTCCCGATTCGCAGCGCATCGCCTTCTATCGCCTTCTTGACGAGTTCT
TCTGA
Misc.:
SEQ ID NO: 120
ATTATTAACGCTTACAATTTCCTGATGCGGTATTTTCTCCTTACGCATCTGTGCGGTATTTCACACCGCATACAGGTGG
CACTTTTCGGGGAAATGTGCGCGGAACCCCTATTTGTTTATTTTTCTAAATACATTCAAATATGTATCCGCTCATGAGA
CAATAACCCTGATAAATGCTTCAATAATAGCACGTGCTAAAACTTCATTTTTAATTTAAAAGGATCTAGGTGAAGATCC
TTTTTGATAATCTCATGACCAAAATCCCTTAACGTGAGTTTTCGTTCCACTGAGCGTCAGACCCCGTAGAAAAGATCAA
AGGATCTTC
Origin of replication:
SEQ ID NO: 121
TTGAGATCCTTTTTTTCTGCGCGTAATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAATCCTGTTACCAGTGGC
TGCTGCCAGTGGCGATAAGTCGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAGATACCTACAGCGTGAGCTAT
GAGAAAGCGCCACGCTTCCCGAAGGGAGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGCCACCTCTGACTTGAGCGTCGATTT
TTGTGATGCTCGTCAGGGGGGCGGAGCCTATGGAAA
Misc.:
SEQ ID NO: 122
AACGCCAGCAACGCGGCCTTTTTACGGTTCCTGGGCTTTTGCTGGCCTTTTGCTCACATGTTCTTGACTCTT

Amino Acid Sequences of Proteins Expressed from Vector Depicted in FIG. 13B

In some embodiments, adalimumab is expressed as a single precursor polypeptide (i.e. a single open reading frame), which is processed to mature antibody heavy and light chains co-translationally. The components of the protein are as follows:

Albumin signal peptide:
SEQ ID NO: 123
MKWVTFISLLFLFSSAYS
Adalimumab heavy chain (variable region):
SEQ ID NO: 124
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSS
Human IgG1 Heavy Chain (constant):
SEQ ID NO: 125
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
Furin cleavage site (\hat{ } denotes the cleavage
site):
SEQ ID NO: 126
RKRR^
Linker:
SEQ ID NO: 127
GSG
P2A self-cleaving peptide (\hat{ } denotes the
cleavage site):
SEQ ID NO: 128
ATNFSLLKQAGDVEENPG\hat{ }P
Adalimumab Light Chain (variable region):
SEQ ID NO: 129
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIK
Human Ig Kappa Constant (* denotes stop codon of
the precursor polypeptide):
SEQ ID NO: 130
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC*

The Enhancer peptide is expressed from an internal ribosomal entry site, as follows.

Enhancer peptide (* denotes stop codon)
SEQ ID NO: 131
MATTMEQETCAHSLTFEECPKCSALQYRNGFYLLKYDEEWYPEELLTDG
EDDVFDPELDMEVVFELQ
Adalimumab complete heavy chain:
SEQ ID NO: 132
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK
Adalimumab complete light chain:
SEQ ID NO: 133
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC*

In some embodiments, the vector comprises any of the complementary determining regions (CDR) of adalimumab, e.g., SEQ ID NOS: 137, 139, 141 of light chain CDRS:

SEQ ID NO: 137
QRYNRAPYX
SEQ ID NO: 139
AASTLQS
SEQ ID NO: 141
RASQGIRNYLA

In some embodiments, the vector comprises any of the complementary determining regions (CDR) of adalimumab, e.g., SEQ ID NOS: 138, 140, 142 of heavy chain CDRS:

SEQ ID NO: 138
VSYLSTASSLD
SEQ ID NO: 140
AITWNSGHIDYADSVEG
SEQ ID NO: 142
DYAMH

In some embodiments, the vector comprises the nucleic acid sequence with at least about 70% (for example, about 75%, about 80%, about 85%, about 90%, about 95%, about 98%, about 99%, or about 100%) identity to SEQ ID NO: 136.

### Nucleic Acid Sequence of Vector Shown in FIG. 13A

SEQ ID NO: 136
CGCGATGTACGGGCCAGATATACGCGTTGACATTGATTATTGACTAGTTATTAATAGTAATCAATTACGGGGTCAT
TAGTTCATAGCCCATATATGGAGTTCCGCGTTACATAACTTACGGTAAATGGCCCGCCTGGCTGACCGCCCAACGA
CCCCCGCCCATTGACGTCAATAATGACGTATGTTCCCATAGTAACGCCAATAGGGACTTTCCATTGACGTCAATGG
GTGGACTATTTACGGTAAACTGCCCACTTGGCAGTACATCAAGTGTATCATATGCCAAGTACGCCCCCTATTGACG
TCAATGACGGTAAATGGCCCGCCTGGCATTATGCCCAGTACATGACCTTATGGGACTTTCCTACTTGGCAGTACAT
CTACGTATTAGTCATCGCTATTACCATGGTGATGCGGTTTTGGCAGTACATCAATGGGCGTGGATAGCGGTTTGAC
TCACGGGGATTTCCAAGTCTCCACCCCATTGACGTCAATGGGAGTTTGTTTTGGCACCAAAATCAACGGGACTTTC
CAAAATGTCGTAACAACTCCGCCCCATTGACGCAAATGGGCGGTAGGCGTGTACGGTGGGAGGTCTATATAAGCAG
AGCTGGTTTAGTGAACCGTCAGATCCGCTAGCGCTACCGGACTCAGATCTCGAGCTCAAGCTTCGAATTCTGCAGT
CGACGGTACCGCGGGCCCGGGATCCACCGGTCGCCACGATGAAGTGGGTGACCTTCATCAGCCTGCTGTTCCTGTT
TTCTTCCGCCTACAGCGAAGTGCAGCTGGTTGAAAGCGGAGGCGGACTGGTCCAGCCAGGCAGAAGCCTGAGACTG
TCTTGTGCCGCCTCTGGCTTCACCTTTGACGACTACGCCATGCACTGGGTGCGGCAGGCCCCTGGCAAGGGACTCG
AGTGGGTCAGCGCCATCACCTGGAATAGCGGCCACATCGACTACGCAGATAGCGTTGAAGGCAGATTCACCATCTC
CAGGGACAACGCCAAGAATTCTCTGTACCTGCAGATGAACAGCCTGCGGGCCGAGGATACCGCTGTGTACTACTGC
GCCAAAGTGTCCTACCTGAGCACCGCCAGCTCCCTGGACTACTGGGGCCAGGGCACCCTGGTGACAGTGAGCTCTG
CTAGCACAAAAGGACCTAGCGTGTTTCCCCTGGCCCCTAGCAGCAAAAGCACCAGCGGCGGAACCGCCGCTCTGGG
TTGTCTGGTGAAGGACTATTTCCCTGAACCTGTGACCGTGTCCTGGAACTCTGGCGCCCTGACTAGCGGCGTGCAT
ACCTTCCCTGCCGTGCTGCAAAGCTCTGGCCTGTATAGCCTTTCTTCTGTGGTGACCGTGCCTAGCAGCTCTCTGG
GCACACAGACATACATCTGCAATGTGAACCACAAGCCCTCCAACACCAAGGTGGACAAAAAGGTGGAACCCAAGAG
CTGCGACAAGACCCACACCTGTCCTCCGTGCCCCGCTCCTGAGCTGCTGGGCGGCCCTTCTGTGTTCCTGTTCCCC
CCCAAACCTAAAGACACACTGATGATCAGCCGGACCCCTGAGGTGACCTGCGTGGTGGTGGACGTGAGCCACGAGG
ACCCCGAGGTGAAGTTCAACTGGTACGTGGACGGCGTGGAGGTCCACAACGCCAAGACCAAACCTAGAGAGGAACA
ATACAACAGCACATATAGAGTGGTGTCTGTGCTGACAGTGCTCCACCAGGACTGGCTGAACGGAAAGGAATACAAG
TGCAAGGTGTCCAACAAGGCCCTCCCTGCTCCAATCGAGAAGACCATTAGCAAGGCCAAGGGCCAACCTAGAGAGC
CCCAGGTCTACACCCTGCCACCAAGTAGAGATGAGCTGACCAAGAACCAGGTGAGCCTAACATGCCTGGTGAAGGG
CTTTTACCCCAGCGACATCGCCGTGGAATGGGAGAGCAACGGCCAGCCTGAGAACAACTACAAGACAACACCTCCT
GTTCTGGATTCTGATGGCAGCTTCTTCCTGTACAGCAAGCTGACAGTGGATAAGAGCCGGTGGCAGCAGGGCAACG
TGTTCAGCTGCTCCGTTATGCACGAGGCCCTGCATAATCACTACACCCAGAAGAGCCTGTCTCTGAGCCCTGGCAA
GCAAGCGAAAACGGCGCGGAAGCGGAGCTACTAACTTCAGCCTGCTGAAGCAGGCTGGAGATGTGGAGGAGAACCC
TGGACCTATGAAGTGGGTGACCTTCATCAGCCTGCTGTTCCTGTTTTCTTCCGCCTACAGCGATATCCAGATGACC
CAGTCTCCATCTAGCCTGAGCGCCAGCGTGGGAGATAGAGTGACCATCACCTGTAGAGCCTCTCAAGGCATCCGGA
ACTACCTGGCCTGGTATCAGCAGAAACCTGGCAAGGCTCCTAAGCTGCTGATCTACGCCGCTTCCACCCTGCAGAG
CGGCGTTCCTTCTAGATTCAGCGGCAGCGGCTCCGGAACAGACTTCACCCTGACAATTAGCTCCCTGCAACCTGAA
GATGTGGCTACATACTACTGCCAGAGATACAATCGGGCCCCTTACACCTTTGGACAGGGCACCAAGGTGGAAATCA
AGCGGACCGTGGCCGCCCCATCTGTGTTCATCTTCCCCCCCAGCGACGAGCAGCTGAAAAGCGGCACAGCCAGCGT
GGTGTGCCTGCTGAACAACTTCTACCCCAGGGAAGCCAAGGTGCAGTGGAAGGTGGACAATGCCCTGCAGAGCGGC
AACAGCCAGGAGAGCGTGACCGAGCAGGACAGCAAGGACAGCACCTACAGCCTGAGCAGCACCCTCACACTGTCTA
AAGCCGACTACGAGAAGCACAAGGTCTACGCCTGCGAGGTGACCCACCAGGGCCTGTCCTCCCCTGTGACAAAGAG
CTTTAACAGAGGCGAGTGCTAAATCATAATCAGCCATACCACATTTGTAGAGGTTTTACTTGCTTTAAAAAACCTC
CCACACCTCCCCCTGAACCTGAAACATAAAATGAATGCAATTGTTGTTGTTAACTTGTTTATTGCAGCTTATAATG
GTTACAAATAAAGCAATAGCATCACAAATTTCACAAATAAAGCATTTTTTTCACTGCATTCTAGTTGTGGTTTGTC
CAAACTCATCAATGTATCTTAAGGCGTCTTCTACTGGGCGGTTTTATGGACAGCAAGCGAACCGGAATTGCCAGCT
GGGGCGCCCTCTGGTAAGGTTGGGAAGCCCTGCAAAGTAAACTGGATGGCTTTCTTGCCGCCAAGGATCTGATGGC
GCAGGGGATCAAGCTCTGATCAAGAGACAGGATGAGGATCGTTTCGCATGATTGAACAAGATGGATTGCACGCAGG
TTCTCCGGCCGCTTGGGTGGAGAGGCTATTCGGCTATGACTGGGCACAACAGACAATCGGCTGCTCTGATGCCGCC
GTGTTCCGGCTGTCAGCGCAGGGGCGCCCGGTTCTTTTTGTCAAGACCGACCTGTCCGGTGCCCTGAATGAACTGC
AAGACGAGGCAGCGCGGCTATCGTGGCTGGCCACGACGGGCGTTCCTTGCGCAGCTGTGCTCGACGTTGTCACTGA
AGCGGGAAGGGACTGGCTGCTATTGGGCGAAGTGCCGGGGCAGGATCTCCTGTCATCTCACCTTGCTCCTGCCGAG
AAAGTATCCATCATGGCTGATGCAATGCGGCGGCTGCATACGCTTGATCCGGCTACCTGCCCATTCGACCACCAAG
CGAAACATCGCATCGAGCGAGCACGTACTCGGATGGAAGCCGGTCTTGTCGATCAGGATGATCTGGACGAAGAGCA
TCAGGGGCTCGCGCCAGCCGAACTGTTCGCCAGGCTCAAGGCGAGCATGCCCGACGGCGAGGATCTCGTCGTGACC
CATGGCGATGCCTGCTTGCCGAATATCATGGTGGAAAATGGCCGCTTTTCTGGATTCATCGACTGTGGCCGGCTGG
GTGTGGCGGACCGCTATCAGGACATAGCGTTGGCTACCCGTGATATTGCTGAAGAGCTTGGCGGCGAATGGGCTGA
CCGCTTCCTCGTGCTTTACGGTATCGCCGCTCCCGATTCGCAGCGCATCGCCTTCTATCGCCTTCTTGACGAGTTC
TTCTGAATTATTAACGCTTACAATTTCCTGATGCGGTATTTTCTCCTTACGCATCTGTGCGGTATTTCACACCGCA
TACAGGTGGCACTTTTCGGGGAAATGTGCGCGGAACCCCTATTTGTTTATTTTTCTAAATACATTCAAATATGTAT
CCGCTCATGAGACAATAACCCTGATAAATGCTTCAATAATAGCACGTGCTAAAACTTCATTTTTAATTTAAAAGGA
TCTAGGTGAAGATCCTTTTTGATAATCTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCTTTTTCCGAAGGTAACTGGCTTCAGCA
GAGCGCAGATACCAAATACTGTCCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTTGTGATGCTCGTCAGGGGGGCGGAGCC
TATGGAAAAACGCCAGCAACGCGGCCTTTTTACGGTTCCTGGGCTTTTGCTGGCCTTTTGCTCACATGTTCTTGAC
TCTT

### Transfection, Transduction, Transformation

The terms “transfection,” “transduction,” and “transformation” refer to the process of introducing nucleic acids into cells (e.g., eukaryotic cells). In some embodiments, a polynucleotide or vector described herein can be introduced into a cell (e.g., a eukaryotic cell) using any method known in the art. A polynucleotide or vector may be introduced into a cell by a variety of methods, which are well known in the art and selected, in part, based on the particular host cell. For example, the polynucleotide can be introduced into a cell using chemical, physical, biological, or viral means. Methods of introducing a polynucleotide or a vector into a cell include, but are not limited to, the use of calcium phosphate, dendrimers, cationic polymers, lipofection, fugene, cell-penetrating peptides, peptide dendrimers, electroporation, cell squeezing, sonoporation, optical transfection, protoplast fusion, impalefection, hydrodynamic delivery, gene gun, magnetofection, particle bombardment, nucleofection, and viral transduction.

Vectors comprising targeting DNA and/or nucleic acid encoding a target protein and an enhancer protein can be introduced into a cell by a variety of methods (e.g., injection, transformation, transfection, direct uptake, projectile bombardment, liposomes). Target proteins and enhancer proteins can be stably or transiently expressed in cells using expression vectors. Techniques of expression in eukaryotic cells are well known to those in the art. (See Current Protocols in Human Genetics: Chapter 12 “Vector Therapy” & Chapter 13 “Delivery Systems for Gene Therapy”).

In some embodiments, polynucleotides or vectors can be introduced into a host cell by insertion into the genome using standard methods to produce stable cell lines, optionally through the use of lentiviral transfection, baculovirus gene transfer into mammalian cells (BacMam), retroviral transfection, CRISPR/Cas9, and/or transposons. In some embodiments, polynucleotides or vectors can be introduced into a host cell for transient transfection. In some embodiments, transient transfection may be effected through the use of viral vectors, helper lipids, e.g., PEI, Lipofectamine, and/or Fectamine 293. The genetic elements can be encoded as DNA on e.g. a vector or as RNA from e.g. PCR. The genetic elements can be separated in different or combined on the same vector.

A polynucleotide or vector may be introduced into a cell by a variety of methods, which are well known in the art. For example, the polynucleotide can be introduced into a cell using chemical, physical, biological, or viral means.

### In Vivo Delivery of the Target Protein

In some embodiments, a polynucleotide or vector described herein can be introduced into a subject using any method known in the art. A polynucleotide or vector may be introduced into a subject by a variety of methods, which are well known in the art. Vectors comprising targeting DNA and/or nucleic acid encoding a target protein and an enhancer protein can be administered to a subject by a variety of methods (e.g., injection, viral transfection, direct uptake, projectile bombardment).

Administration by injection may comprise, e.g., intramuscular, intravenous, intracardiac, intraperitoneal, intravenous, intraarterial, intradermal, subcutaneous, intracranial, lumbar, intravitreal, intranasal, or other injection. Vectors or polynucleotide can be introduced into the cells of a subject using chemical, physical, biological, or viral means. Methods of administering a polynucleotide or a vector into a subject and/or introducing a polynucleotide or a vector into a cell of a subject include, but are not limited to, direct injection with or without electroporation/sonoporation while using or not using cationic or other polymers, lipids, lipid formulations, cell-penetrating peptides, nanoparticle-based delivery vehicles, nanogels, gene gun, jet-gene devices, particle bombardment and viral transduction. In some embodiments, the administration is by injection under the skin. As also described elsewhere in the application, in some embodiments, the vectors or polynucleotides disclosed herein may be introduced into the cells of the subject using any viral gene delivery vectors, such as, adenoviruses, adeno-associated viruses, herpes simplex viruses, retroviruses, lentiviruses, alphaviruses, flaviviruses, rhabdoviruses, measles virus, Newcastle disease virus, poxviruses, picornaviruses, or any other viral delivery system.

In some embodiments, the polynucleotide or vector encoding a target protein and an enhancer protein described herein may be administered to the subject to treat, prevent or manage at least one symptom of a disease. In some embodiments, the target protein is an antibody, such as a monoclonal antibody (e.g. adalimumab). In some embodiments, the subject is a subject having any condition that is known or is discovered in the future to be treated, prevented or managed by the expression of the target protein (e.g. adalimumab). For instance, non-limiting examples of conditions that may be treated by administration of polynucleotides or vectors encoding adalimumab include rheumatoid arthritis, psoriatic arthritis, ankylosing spondylitis, Crohn's disease, ulcerative colitis, psoriasis, hidradenitis suppurativa, uveitis, and juvenile idiopathic arthritis.

Thus, the disclosure provides methods of treating or preventing a disease in a subject, comprising: administering to the subject, a therapeutically effective amount of any one of the vectors or polynucleotides encoding a target protein and an enhancer protein disclosed herein. The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the tissue to be imaged, and the physical delivery system in which it is carried.

The disclosed methods of expressing a target protein in the presence of an enhancer have several advantages, as described below. In some embodiments, the target protein expressed in the presence of an enhancer protein using the compositions or methods disclosed herein is more functionally active than a target protein that is expressed in the absence of the enhancer protein. In some embodiments, the target protein expressed in the presence of an enhancer protein using the compositions or methods disclosed herein is at least about 1.2 times (for example, about 1.5 times, about 1.7 times, about 2 times, about 2.5 times, about 3 times, about 3.5 times, about 4 times, about 4.5 times, about 5 times, about 5.5 times, about 6 times, about 7 times, about 8 times, about 9 times, about 10 times, about 20 times, or about 50 times, including all values and subranges that lie therebetween) more active than a target protein that is expressed in the absence of the enhancer protein.

In some embodiments, the target protein expressed in the presence of an enhancer protein using the compositions or methods disclosed herein is expressed for a longer duration as compared to the target protein expressed in the absence of the enhancer protein. In some embodiments, the target protein expressed in the presence of an enhancer protein using the compositions or methods disclosed herein is expressed for at least about 1 hour (for example, about 12 hours, about 1 day, about 2 days, about 3 days, about 4 days, about 5 days, about 6 days, about 1 week, about 2 weeks, about 3 weeks, about 1 months, about 2 months, about 6 months, or about 1 year) longer as compared to the target protein expressed in the absence of the enhancer protein.

In some embodiments, a lesser proportion of the target protein expressed in the presence of an enhancer protein using the compositions or methods disclosed herein exhibits undesirable properties (e.g., misfolding, altered activity, incorrect posttranslational modifications, and/or toxicity), as compared to the target protein expressed in the absence of the enhancer protein. For instance, in some embodiments, less than about 30% (for example, less than about 25%, less than about 20%, less than about 15%, less than about 10%, less than about 5%, less than about 2%, or less than about 1%, including all values and subranges that lie therebetween) of the target protein expressed in the presence of an enhancer protein using the compositions or methods disclosed herein exhibits undesirable properties. In some embodiments, a higher proportion of the target protein expressed in the presence of an enhancer protein using the compositions or methods disclosed herein exhibits correct folding, as compared to the target protein expressed in the absence of the enhancer protein.

In some embodiments, the therapeutically effective amount of a vector or a polynucleotide encoding a target protein and an enhancer protein administered to the subject is lower than the therapeutically effective amount of a control vector or control polynucleotide encoding just the target protein. Without being bound to a theory, it is thought that due to the improved expression quality and/or quantity, and/or longer duration of expression of the target protein when expressed in the presence of the enhancer protein, lower doses of a vector or a polynucleotide encoding a target protein and an enhancer protein (as compared to a control vector or control polynucleotide encoding just the target protein), are sufficient to elicit a similar biological effect.

In some embodiments, a subject who is administered vectors or polynucleotides encoding a target protein and an enhancer protein exhibits reduced generation of anti-target protein antibodies, as compared to a control subject who is administered vectors or polynucleotides encoding just the target protein. Without being bound by a theory, it is also thought that the formation of poorly folded or unfolded target proteins expressed in the absence of an enhancer promotes the generation of anti-target protein antibodies. On the other hand, the improved expression quality and/or quantity of the target protein when expressed in the presence of the enhancer protein reduces the generation of anti-target protein antibodies.

### Cells, Cell Lines, Host Cells

Another aspect of the present disclosure relates to cells comprising polynucleotides and/or vectors encoding one or more target proteins and one or more enhancer proteins. The polynucleotides, vectors, target protein, and enhancer proteins may be any of those described herein.

In some embodiments, the cell is any eukaryotic cell or cell line. The disclosed polynucleotides, vectors, systems, and methods may be used in any eukaryotic primary cells and cell lines. Eukaryotic cell lines may include mammalian cell lines, such as human and animal cell lines. Eukaryotic cell lines may also include insect, plant, or fungal cell lines. Non-limiting examples of such cells or cell lines generated from such cells include Bc HROC277, COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, 5P2/0-Ag14, HeLa, HEK293 (e.g., HEK293-F, HEK293-H, HEK293-T), and perC6 cells as well as insect cells such as Spodoptera fugiperda (Sf, e.g., Sf9), or fungal cells such as Saccharomyces, Pichia and Schizosaccharomyces.

In some embodiments, a cell or cell line for expressing target protein(s) and enhancer protein(s) is a human cell or cell line. In certain aspects, the choice of a human cell line is beneficial, e.g., for post-translational modifications (“PTMs”), such as glycosylation, phosphorylation, disulfide bonds, in target proteins. In some embodiments, a human cell or cell line is used for expression of a human target protein.

In some embodiments, the present disclosure provides a eukaryotic cell for expression of a target protein, wherein the cell comprises an exogenous polynucleotide encoding an enhancer protein. In some embodiments, the exogenous polynucleotide encoding an enhancer protein is transiently transduced and/or not integrated into the genome of the cell. In some embodiments, the exogenous polynucleotide encoding an enhancer protein is stably integrated. In some embodiments, the enhancer protein is an inhibitor of nucleocytoplasmic transport (NCT). In some embodiments, the enhancer protein is selected from the group consisting of a picomavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein. The exogenous polynucleotide is operatively linked to a promoter (optionally a native promoter or an exogenous promoter). In some embodiments, the polynucleotide is operatively linked to an internal ribosome entry site (IRES). In some embodiments, the promoter is an inducible promoter.

### In Vitro and Ex Vivo Methods

The present disclosure provides a method for expressing a target protein in eukaryotic cells. The method may comprise introducing a polynucleotide encoding the target protein (the polynucleotide operatively linked to a promoter) into the eukaryotic cells. This method utilizes co-expression of an enhancer protein to enhance the expression level, solubility and/or activity of the target protein. In addition, the method utilizes co-expression of an enhancer protein to prolong the expression of the target protein over a longer period of time. In some embodiments, the enhancer protein is an inhibitor of nucleocytoplasmic transport (NCT). In some embodiments, the enhancer protein is selected from the group consisting of a picomavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

In some aspects, the present disclosure relates to methods of producing target proteins through the use of cells comprising polynucleotides encoding one or more target proteins and one or more enhancer proteins. In some embodiments, the method is carried out in eukaryotic cells comprising one or more vectors. In some embodiments, the method is carried out using the polynucleotides, vectors, and cells described in the foregoing sections. In some embodiments, the vectors (or a vector) may have a first polynucleotide encoding the target protein and a second polynucleotide encoding an enhancer protein. In some embodiments, the first polynucleotide and the second polynucleotide are operatively linked to one or more promoters.

In some embodiments, the method may comprise introducing into a eukaryotic cell a polynucleotide encoding an enhancer protein, operatively linked to a promoter. In some embodiments, the method may comprise transfection of the eukaryotic cells with one or more DNA molecules, transduction of the eukaryotic cells with a single viral vector, and/or transduction of the eukaryotic cells with two or more viral vectors.

Further provided is a method for recombinant expression of a target protein that includes introducing a polynucleotide encoding the target protein, operatively linked to a promoter, into a eukaryotic cell. In some embodiments, the method of target protein expression comprises introducing a vector system of the disclosure into a eukaryotic cell. In some embodiments, the target protein is a membrane protein. In some embodiments, localization of the membrane protein to the cellular membrane is increased compared to the localization observed when the membrane protein is expressed without the enhancer protein.

### In Vivo Methods

The present disclosure provides methods for expressing a target protein in vivo. In some embodiments, the methods comprise introducing a polynucleotide encoding the target protein (the polynucleotide operatively linked to a promoter) into cells of a subject. This method utilizes co-expression of an enhancer protein to enhance the expression level, solubility and/or activity of the target protein. In some embodiments, the enhancer protein is an inhibitor of nucleocytoplasmic transport (NCT). In some embodiments, the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

In some embodiments, the method elicits an immune response in the subject. The immune response can be both immunogenic as well as immunosuppressive or immunomodulatory in nature. In some embodiments, the method treats a disease in the subject, wherein the disease is caused by, correlated with, or associated with the target protein. In some embodiments, the method treats a disease in the subject, wherein the expression levels of the target protein in the subject is lower than the expression levels of the target protein in a control subject, wherein the control subject does not have the disease.

In some embodiments, the present disclosure relates to methods of producing target proteins through the use of polynucleotides encoding one or more target proteins and one or more enhancer proteins. In some embodiments, the method is carried out in vivo comprising one or more vectors. In some embodiments, the method is carried out using the polynucleotides, vectors, and cells described in the foregoing sections. In some embodiments, the vectors (or a vector) may have a first polynucleotide encoding the target protein and a second polynucleotide encoding an enhancer protein. In some embodiments, the first polynucleotide and the second polynucleotide are operatively linked to one or more promoters.

In some embodiments, the method may comprise introducing into a subject a polynucleotide encoding an enhancer protein, operatively linked to a promoter. In some embodiments, the method may comprise injections with one or more DNA molecules, with a single viral vector, and/or with two or more viral vectors.

Further provided is a method for in vivo expression of a target protein that includes introducing a polynucleotide encoding the target protein, operatively linked to a promoter, into a subject. In some embodiments, the method of target protein expression comprises introducing a vector system of the disclosure into a subject.

In some embodiments, a target protein and enhancer protein DNA construct are delivered via a lipid nanoparticle (LNP). In some embodiments, the LNP comprises a PEGylated lipid, a cholesterol, and one or more ionizable lipids. In some embodiments, the LNP comprises about 0.5% to about 2% PEGylated lipid, about 35% to about 45% cholesterol, and about 5% to about 65% one or more ionizable lipids. In some embodiments, the LNP comprises DMG-PEG(2000), cholesterol, DOPC and DLin-KC2-DMA in a ratio of about 1% DMG-PEG(2000), to about 40% cholesterol, to about 10% DOPC and about 50% DLin-KC2-DMA.

### Downstream Applications

In some embodiments, target proteins, produced through the use of the present compositions, systems, and methods are used as therapeutics, diagnostics or for research and development. Illustrative applications include, but are not limited to, vaccines, enzyme replacement therapies, hormone replacement therapies, antibody therapies, antiviral treatments, antimicrobial treatments, immunomodulators, therapeutic cancer vaccines, immuno-oncology applications, bispecific T-cell engagers, screening assays, diagnostic assays, clinical testing kits, drug discovery, antibody discovery, and the like.

In some embodiments, target proteins, and cells expressing such proteins, produced through the use of the present compositions, systems, and methods are isolated, purified, and/or used for downstream applications. Illustrative applications include, but are not limited to, small molecule screening, structural determination (e.g., X-ray crystallography, cryo-electron microscopy, and the like), activity assays, therapeutics, enzyme replacement therapy, screening assays, diagnostic assays, clinical testing kits, drug discovery, antibody discovery, and the like. In some embodiments, the present compositions and methods are used to produce antibodies or to produce antigens for antibody screening assays. In some embodiments, the cells expressing the target proteins can be used as an assay system to screen, e.g., cell interactions, antibody binding, or small molecule influences in a whole cell system.

In some embodiments, the disclosure provides systems and methods for antibody discovery. In some embodiments, the disclosure provides methods for generating an antibody against a target protein, comprising immunizing a subject with a cell or target protein produced using the systems or methods of the disclosure. In various embodiments, the immunized subject is a mouse, rat, rabbit, non-human primate, lama, camel, or human. Cells isolated from the subject can be subjected to further rounds of the selection as isolated cells, or optionally after generation of hybridomas from the isolated cells. Gene cloning and/or sequencing can be used to isolate polynucleotide sequence(s) encoding heavy and light chains form the isolated cells or hybridomas. Gene cloning and/or sequencing can be applied to single cells or populations of cells. In some embodiments, the compositions and methods of the disclosure are used for generating a polyclonal antibody through immunization of a subject followed by harvesting of serum from the subject.

The disclosure further provides methods for antibody discovery by cell sorting, comprising providing a solution comprising a labeled cell or target protein produced using the systems or methods of the disclosure, and a population of recombinant cells, wherein the recombinant cells express a library of polypeptides each comprising an antibody or antigen-binding fragment thereof; and sorting one or more recombinant cells from the solution by detecting recombinant cells bound to the labeled cell or the labeled target protein. In other variations, cell sorting is performed on cells derived from an immunized subject. The subject may be immunized with a cell or target protein produced according the methods of the disclosure, or using another suitable immunogen. In some embodiments, the recombinant cells comprise a naïve antibody library, optionally a human naïve antibody library. Various antibody library generation methods are known in the art and can be combined with the methods of the present disclosure. As used herein, the terms “sorting” or “cell sorting” refer to fluorescence-activated cell sorting, magnetic assisted cell sorting, and other means of selecting labeled cells in a population of labeled and unlabeled cells.

The disclosure further provides, a method for panning a phage-display library, comprising mixing a phage-display library with a cell or target protein produced using the systems or methods of the disclosure; and purifying and/or enriching the members of the phage-display library that bind the cell or target protein. In some embodiments, the phage-display library expresses a population of single-chain variable fragments (scFvs) or other types of antibody/antibody fragments (Fabs etc.).

In further embodiments, the disclosure provides methods for screening for protein binders of any type. The cells and target proteins of the disclosure can be used to screen libraries of various types of molecule, including drugs and macromolecules (proteins, nucleic acids, and protein:nucleic acid complexes) to identify binding partners for the target protein. In other embodiments, the systems and methods of the disclosure are used to express libraries of target proteins in single wells, in pools of several sequences, or in libraries of gene sequences.

The ability to express an antigen in its native or disease-relevant form in high yields and/or present on the surface of cells enables more reliable discovery and/or generation of antibodies, antibody fragments, and other molecules than prior art methods. Such antibody, antibody fragments, and other molecules may be useful as therapeutics and/or research tools, or for other applications.

In some embodiments, the systems and methods of the disclosure are suitable for use in discovery of antibodies that bind to and/or are specific to particular glycosylation patterns on target molecules (e.g. glycoproteins). In some embodiments, the antibody library is sorted against the natively glycosylated protein and counter-sorted against an improperly glycosylated or de-glycosylated cognate protein. Similarly stated, by using a deglycosylation enzyme, antibodies can be sorted specifically against the glycosylation pattern. In further embodiments, the cells and/or target proteins of the disclosure are used to confirm the binding and/or functional activity of novel antibodies or other macromolecules.

In some embodiments, target proteins, produced through the use of the present compositions, systems, and methods are used as therapeutics, diagnostics or for research and development. Illustrative applications include, but are not limited to, vaccines, enzyme replacement therapies, hormone replacement therapies, antibody therapies, antiviral treatments, antimicrobial treatments, immunomodulators, therapeutic cancer vaccines, bispecific T-cell engagers screening assays, diagnostic assays, clinical testing kits, drug discovery, antibody discovery, and the like.

### Illustrative Advantages

The present compositions, systems, and methods may have numerous advantages. For example, as demonstrated in Example 11, a human NADase that usually results in apoptosis and therefore produces non-detectable yields when overexpressed in human cell lines, can be reliably expressed to produce yields of greater than 20 mg/L when an enhancer protein is co-expressed with this target protein. Additionally, the NADase expressed through this illustrative method is functional (as demonstrated by a phosphate release assay) and shows a low batch to batch variation.

Similarly, in some embodiments, the present methods, systems, and cells are used for the reliable expression of difficult to express proteins. In some embodiments, the present disclosure relates to the production of proteins with low batch-to-batch variation. The proteins produced according to the present disclosure may exhibit one or more of the following improvements: purification without purification tag fusions; improved functional activity; reliable production; consistent activity; and suitability for therapeutic applications.

Cells of the present disclosure may have one or more of the following advantages in terms of target protein expression: higher concentration of target membrane proteins in the membrane; slower/decreased target protein degradation; improved signal to noise ratio in whole cell assays; target protein and/or enhancer protein expression without affecting downstream cell metabolism; increased stability against desensitization of membrane-bound membrane proteins; and higher target protein yield. Example 1 provides an illustrative example of expression of enhancer protein without affecting downstream metabolism of cells. The GPCR exemplified in Example 1 was able to interact with its natural substrate and produce activation that could be measured in vitro.

The present systems and methods may, in some embodiments, have one or more of the following advantages: suitability for any eukaryotic cell type; decreased need for target protein expression optimization; and reliable expression of difficult-to-express proteins.

The methods of expressing a target protein in vivo disclosed herein have superior properties as compared to the standard methods used for this purpose in the art. For instance, as demonstrated in the Examples, the methods disclosed herein ensure stable expression of the target protein over longer period of time, and reduce variability in expression levels among animals. These properties enable the application of the methods disclosed herein in the prevention and treatment of diseases.

### Systems

The present disclosure provides a system for recombinant expression of a target protein in eukaryotic cells that includes one or more vectors. The present disclosure further provides methods of expressing a target protein in a subject in need thereof, comprising administering to the subject a vector system comprising one or more vectors, the one or more vectors, comprising: a) a first polynucleotide encoding the target protein; and b) a second polynucleotide encoding an enhancer protein wherein: i) the enhancer protein is an inhibitor of nucleocytoplasmic transport (NCT) and/or ii) the enhancer protein is selected from the group consisting of a picornavirus leader (L) protein, a picornavirus 2A protease, a rhinovirus 3C protease, a herpes simplex virus (HSV) ICP27 protein, and a rhabdovirus matrix (M) protein, wherein the first polynucleotide and the second polynucleotide are operatively linked to one or more promoters. The vectors (or a vector) may have a first polynucleotide encoding a target protein and a second polynucleotide encoding an enhancer protein. The enhancer protein may be an inhibitor of nucleocytoplasmic transport (NCT). In some embodiments, the enhancer protein may be selected from the group consisting of a picomavirus leader (L) protein, a picomavirus 2A protease, a rhinovirus 3C protease, a herpes simplex virus (HSV) ICP27 protein, and a rhabdovirus matrix (M) protein. The first polynucleotide and the second polynucleotide may be operatively linked to one or more promoters.

In some embodiments, the enhancer protein is an inhibitor of nucleocytoplasmic transport (NCT). In some embodiments, the NCT inhibitor is a viral protein.

In some embodiments, the enhancer protein is an NCT inhibitor selected from the group consisting of a picornavirus leader (L) protein, a picomavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

The NCT inhibitor may be a picornavirus leader (L) protein or a functional variant thereof. In some embodiments, the NCT inhibitor may be a picomavirus 2A protease or a functional variant thereof. In some embodiments, the NCT inhibitor may be a rhinovirus 3C protease or a functional variant thereof. In some embodiments, the NCT inhibitor may be a coronavirus ORF6 protein or a functional variant thereof. In some embodiments, the NCT inhibitor may be an ebolavirus VP24 protein or a functional variant thereof. In some embodiments, the NCT inhibitor may be a Venezuelan equine encephalitis virus (VEEV) capsid protein or a functional variant thereof. In some embodiments, the NCT inhibitor is a herpes simplex virus (HSV) ICP27 protein or a functional variant thereof. In some embodiments, the NCT inhibitor is a rhabdovirus matrix (M) protein or a functional variant thereof.

In some embodiments, the enhancer protein is an L protein, which is the L protein of Theiler's virus or a functional variant thereof. In some embodiments, the L protein may share at least 90% identity to SEQ ID NO: 1.

In some embodiments, the L protein is the L protein of Encephalomyocarditis virus (EMCV) or a functional variant thereof. In some embodiments, the L protein may share at least 90% identity to SEQ ID NO: 2.

In some embodiments, the L protein is selected from the group consisting of the L protein of poliovirus, the L protein of HRV16, the L protein of mengo virus, and the L protein of Saffold virus 2 or a functional variant thereof.

The system may comprise a single vector comprising an expression cassette, the expression cassette comprising the first polynucleotide and the second polynucleotide. In some embodiments, the expression cassette comprises a first promoter, operatively linked to the first polynucleotide; and a second promoter, operatively linked to the second polynucleotide. In some embodiments, the expression cassette comprises a shared promoter operatively linked to both the first polynucleotide and the second polynucleotide.

In some embodiments, the expression cassette comprises a coding polynucleotide comprising the first polynucleotide and the second polynucleotide linked by a polynucleotide encoding a ribosome skipping site, the coding polynucleotide operatively linked to the shared promoter.

In some embodiments, the expression cassette comprises a coding polynucleotide, the coding polynucleotide encoding the enhancer protein and the target protein linked to by a ribosome skipping site, the coding polynucleotide operatively linked to the shared promoter.

In some embodiments, the expression cassette is configured for transcription of a single messenger RNA encoding both the target protein and the enhancer protein, linked by a ribosome skipping site; wherein translation of the messenger RNA results in expression of the target protein and the enhancer protein (e.g., an L protein) as distinct polypeptides.

The system may comprise one vector. In some embodiments, the system may comprise a single vector comprising a first polynucleotide encoding a target protein and a second polynucleotide encoding an enhancer protein.

The system may comprise two vectors. In some embodiments, the system may comprise a first vector comprising the first polynucleotide, operatively linked to a first promoter; and a second vector comprising the second polynucleotide, operatively linked to a second promoter.

In some embodiments, the first polynucleotide or the second polynucleotide, or both, are operatively linked to an internal ribosome entry site (IRES).

In some embodiments, at least one of the one or more vectors comprised by the system may comprise a T7 promoter configured for transcription of either or both of the first polynucleotide or the second polynucleotide by a T7 RNA polymerase.

In some embodiments, at least one of the one or more vectors comprised by the system may comprise a polynucleotide sequence encoding a T7 RNA polymerase.

The compositions and methods of the disclosure provide improved expression of a target protein when co-expressed with an enhancer protein, e.g. an L protein. As used herein, “improved expression of the target protein” includes, but is not limited to one or more of the following relative to the target protein: increased activity, lower expression levels, increased expression duration, increased stability, increased duration of detection in a cell or subject, increased uniformity of delivery, reduced degradation, and reduced EC₅₀.

In some embodiments, co-expression of the enhancer protein increases the activity of the target protein in a cell or subject by about 10-fold, about 20-fold, about 30-fold, about 40-fold, about 50-fold, about 60-fold, about 70-fold, about 80-fold, about 90-fold, about 100-fold, about 150-fold, about 200-fold, or about 300×.

In some embodiments, co-expression of the enhancer protein lowers the expression level of the target protein by about 10%, about 20%, about 30%, about 40%, about 50%, about 60%, about 70%, about 80%, or about 90%.

In some embodiments, co-expression of the enhancer protein increases the duration of time in which active target protein is found in the cell or subject by about 2-fold, about 3-fold, about 4-fold, about 5-fold, about 6-fold, about 7-fold, about 8-fold, about 9-fold, about 10, about 11-fold, about 12-fold, about 13-fold, about 14-fold, about 15-fold, about 16-fold, about 17-fold, about 18-fold, about 19-fold, or about 20×.

The coefficient of variation (CV %) is provided as a measure of uniformity of target protein expression, and is defined as the standard deviation of a diagnostic moiety (e.g., a fluorophore or radiolabel) signal divided by the signal average. In some embodiments, co-expression of the enhancer protein increases the uniformity of expression of the target protein in a tissue or subject by about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.7-fold, about 2.8-fold, about 2.9-fold, or about 3-fold.

In some embodiments, co-expression of the enhancer protein reduces the degradation of the target protein by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

In some embodiments, co-expression of the enhancer protein reduces the concentration of target protein effective in producing 50% of the maximal response (EC50). In some embodiments, the target protein is adalimumab and the response is neutralization of tumor necrosis factor-alpha (TNF-alpha) in a cell or subject. In some embodiments, the EC50 of adalimumab is reduced in a cell or subject by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

In some embodiments, co-expression of the enhancer protein, e.g. an L protein, with an adalimumab protein improves the treatment of a disease selected from the following: Rheumatoid Arthritis, Juvenile Idiopathic Arthritis (JIA), Psoriatic Arthritis (PsA), Ankylosing Spondylitis (AS), Crohn's Disease (CD), Ulcerative Colitis (UC), Plaque Psoriasis (Ps), Hidradenitis Suppurativa (HS), and Uveitis (UV). In some embodiments, co-expression of the enhancer protein with adalimumab as provided herein, improves the treatment of the aforementioned diseases by about 10%, about 20%, about 30%, about 40%, about 50%, about 60%, about 70%, about 80%, or about 90% relative to adalimumab treatment without co-expression of the enhancer protein.

In some embodiments, co-expression of the enhancer protein, e.g., an L protein, improves the expression of a target protein, wherein the target protein is an antibody selected from the group comprising (also see Table 8): Adalimumab, Pembrolizumab, Nivolumab, Trastuzumab, Bevacizumab, Ustekinumab, Ocrelizumab, Secukinumab, Vedolizumab, Ibalizumab, Nirsevimab, Atoltivimab, Maftivimab, Odesivimab, Casirivimab, Imdevimab, and Brolucizumab.

TABLE 8
Example antibody target proteins

| Antibody | | | | |
|---|---|---|---|---|
| target protein | VH | SEQ ID NO: | VL | SEQ ID NO: |
| Adalimumab | EVQLVESGGGLVQPGRSLRLSCAASG | 124 | DIQMTQSPSSLSASVGDRVTI | 129 |
| | FTFDDYAMHWVRQAPGKGLEWVSAI | | TCRASQGIRNYLAWYQQKP | |
| | TWNSGHIDYADSVEGRFTISRDNAKN | | GKAPKLLIYAASTLQSGVPS | |
| | SLYLQMNSLRAEDTAVYYCAKVSYL | | RFSGSGSGTDFTLTISSLQPE | |
| | STASSLDYWGQGTLVTVSS | | DVATYYCQRYNRAPYTFGQ | |
| | | | GTKVEIK | |
| Pembrolizumab | QVQLVQSGVEVKKPGASVKVSCKAS | 374 | EIVLTQSPATLSLSPGERATL | 375 |
| | GYTFTNYYMYWVRQAPGQGLEWMG | | SCRASKGVSTSGYSYLHWY | |
| | GINPSNGGTNFNEKFKNRVTLTTDSST | | QQKPGQAPRLLIYLASYLES | |
| | TTAYMELKSLQFDDTAVYYCARRDY | | GVPARFSGSGSGTDFTLTISS | |
| | RFDMGFDYWGQGTTVTVSS | | LEPEDFAVYYCQHSRDLPLT | |
| | | | FGGGTKVEIK | |
| Nivolumab | QVQLVESGGGVVQPGRSLRLDCKAS | 376 | EIVLTQSPATLSLSPGERATL | 377 |
| | GITFSNSGMHWVRQAPGKGLEWVAV | | SCRASQSVSSYLAWYQQKP | |
| | IWYDGSKRYYADSVKGRFTISRDNSK | | GQAPRLLIYDASNRATGIPA | |
| | NTLFLQMNSLRAEDTAVYYCATNDD | | RFSGSGSGTDFTLTISSLEPE | |
| | YWGQGTLVTVSS | | DFAVYYCQQSSNWPRTFGQ | |
| | | | GTKVEIK | |
| Trastuzumab | EVQLVESGGGLVQPGGSLRLSCAASG | 378 | DIQMTQSPSSLSASVGDRVTI | 379 |
| | FNIKDTYIHWVRQAPGKGLEWVARIY | | TCRASQDVNTAVAWYQQKP | |
| | PTNGYTRYADSVKGRFTISADTSKNT | | GKAPKLLIYSASFLYSGVPSR | |
| | AYLQMNSLRAEDTAVYYCSRWGGD | | FSGSRSGTDFTLTISSLQPEDF | |
| | GFYAMDYWGQGTLVTVSS | | ATYYCQQHYTTPPTFGQGT | |
| | | | KVEIK | |
| Bevacizumab | EVQLVESGGGLVQPGGSLRLSCAASG | 380 | DIQMTQSPSSLSASVGDRVTI | 381 |
| | YTFTNYGMNWVRQAPGKGLEWVGW | | TCSASQDISNYLNWYQQKP | |
| | INTYTGEPTYAADFKRRFTFSLDTSKS | | GKAPKVLIYFTSSLHSGVPSR | |
| | TAYLQMNSLRAEDTAVYYCAKYPHY | | FSGSGSGTDFTLTISSLQPED | |
| | YGSSHWYFDVWGQGTLVTVSS | | FATYYCQQYSTVPWTFGQG | |
| | | | TKVEIK | |
| Ustekinumab | EVQLVQSGAEVKKPGESLKISCKGSG | 382 | DIQMTQSPSSLSASVGDRVTI | 383 |
| | YSFTTYWLGWVRQMPGKGLDWIGIM | | TCRASQGISSWLAWYQQKP | |
| | SPVDSDIRYSPSFQGQVTMSVDKSITT | | EKAPKSLIYAASSLQSGVPSR | |
| | AYLQWNSLKASDTAMYYCARRRPG | | FSGSGSGTDFTLTISSLQPED | |
| | QGYFDFWGQGTLVTVSS | | FATYYCQQYNIYPYTFGQGT | |
| | | | KLEIK | |
| Ocrelizumab | EVQLVESGGGLVQPGGSLRLSCAASG | 384 | DIQMTQSPSSLSASVGDRVTI | 385 |
| | YTFTSYNMHWVRQAPGKGLEWVGAI | | TCRASSSVSYMHWYQQKPG | |
| | YPGNGDTSYNQKFKGRFTISVDKSKN | | KAPKPLIYAPSNLASGVPSRF | |
| | TLYLQMNSLRAEDTAVYYCARVVYY | | SGSGSGTDFTLTISSLQPEDF | |
| | SNSYWYFDVWGQGTLVTVSS | | ATYYCQQWSFNPPTFGQGT | |
| | | | KVEIK | |
| Secukinumab | EVQLVESGGGLVQPGGSLRLSCAASG | 386 | EIVLTQSPGTLSLSPGERATL | 387 |
| | FTFSNYWMNWVRQAPGKGLEWVAA | | SCRASQSVSSSYLAWYQQKP | |
| | INQDGSEKYYVGSVKGRFTISRDNAK | | GQAPRLLIYGASSRATGIPDR | |
| | NSLYLQMNSLRVEDTAVYYCVRDYY | | FSGSGSGTDFTLTISRLEPED | |
| | DILTDYYIHYWYFDLWGRGTLVTVSS | | FAVYYCQQYGSSPCTFGQGT | |
| | | | RLEIK | |
| Vedolizumab | QVQLVQSGAEVKKPGASVKVSCKGS | 388 | DVVMTQSPLSLPVTPGEPASI | 389 |
| | GYTFTSYWMHWVRQAPGQRLEWIGE | | SCRSSQSLAKSYGNTYLSWY | |
| | IDPSESNTNYNQKFKGRVTLTVDISAS | | LQKPGQSPQLLIYGISNRFSG | |
| | TAYMELSSLRSEDTAVYYCARGGYD | | VPDRFSGSGSGTDFTLKISRV | |
| | GWDYAIDYWGQGTLVTVSS | | EAEDVGVYYCLQGTHQPYT | |
| | | | FGQGTKVEIK | |
| Ibalizumab | QVQLQQSGPEVVKPGASVKMSCKAS | 390 | DIVMTQSPDSLAVSLGERVT | 391 |
| | GYTFTSYVIHWVRQKPGQGLDWIGYI | | MNCKSSQSLLYSTNQKNYL | |
| | NPYNDGTDYDEKFKGKATLTSDTSTS | | AWYQQKPGQSPKLLIYWAS | |
| | TAYMELSSLRSEDTAVYYCAREKDN | | TRESGVPDRFSGSGSGTDFT | |
| | YATGAWFAYWGQGTLVTVSS | | LTISSVQAEDVAVYYCQQY | |
| | | | YSYRTFGGGTKLEIK | |
| Nirsevimab | QVQLVQSGAEVKKPGSSVMVSCQAS | 392 | DIQMTQSPSSLSAAVGDRVT | 393 |
| | GGLLEDYIINWVRQAPGQGPEWMGG | | ITCQASQDIVNYLNWYQQKP | |
| | IIPVLGTVHYGPKFQGRVTITADESTD | | GKAPKLLIYVASNLETGVPS | |
| | TAYMELSSLRSEDTAMYYCATETAL | | RFSGSGSGTDFSLTISSLQPE | |
| | VVSETYLPHYFDNWGQGTLVTVSS | | DVATYYCQQYDNLPLTFGG | |
| | | | GTKVEIK | |
| Atoltivimab | QVQLVESGGGVVQPGRSLRLSCAASG | 394 | DIQMTQSPSSLSASVGDRITI | 395 |
| | FTFNNYGMHWVRQAPGMGLEWVAV | | TCRASQSISTYLHWYQQKPG | |
| | IWHDGSDKYYADSVKGRFTISRDNSK | | KAPKLLIYAASTLQSGVPSRF | |
| | NTLYLQMNSLRAEDTAVYYCARNW | | SGSGSGTDFTLTISSLQPEDF | |
| | NLFDYWGQGTLVTVSS | | ATYYCQQSFSTPPINFGQGT | |
| | | | KLEIK | |
| Maftivimab | EVQLVESGGGLVQPGGSLRLSCAASG | 396 | DIQMTQSPSSLSASVGDRVTI | 397 |
| | FTSSSYAMNWVRQAPGKGLEWVSTIS | | TCRASQSISSFLNWYQQKPG | |
| | GMGGSTYYADSVKGRFTISRDNSKNT | | KAPKLLIYAASSLQSGVPSRF | |
| | LYLQMNSLRAEDTAVYYCAKRGYPH | | SGSGSGTDFTLTISSLQPEDF | |
| | SFDIWGQGTMVTVSS | | ATYYCQQSYSTLTFGQGTRL | |
| | | | EIK | |
| Odesivimab | EVQLVESGGGLVQPGGSLRLSCAASG | 398 | DIVMTQSPDSLAVSLGERATI | 399 |
| | FTFSSYDMHWVRQATGKGLEWVSAI | | NCKSSQSVLYSSNNKNYLA | |
| | GTAGDTYYPGSVKGRFTISRENAKNS | | WYQQKPGQPPKLLIYWAST | |
| | LYLQMNSLRAGDTAVYYCARTWFGE | | RESGVPDRFSGSGSGTEFTLT | |
| | LYFDYWGQGTLVTVSS | | ITSLQAEDVAVYYCQQYYSS | |
| | | | PLTFGGGTKVEIK | |
| Casirivimab | QVQLVESGGGLVKPGGSLRLSCAASG | 400 | DIQMTQSPSSLSASVGDRVTI | 401 |
| | FTFSDYYMSWIRQAPGKGLEWVSYIT | | TCQASQDITNYLNWYQQKP | |
| | YSGSTIYYADSVKGRFTISRDNAKSSL | | GKAPKLLIYAASNLETGVPS | |
| | YLQMNSLRAEDTAVYYCARDRGTTM | | RFSGSGSGTDFTFTISGLQPE | |
| | VPFDYWGQGTLVTVSS | | DIATYYCQQYDNLPLTFGGG | |
| | | | TKVEIK | |
| Imdevimab | QVQLVESGGGVVQPGRSLRLSCAASG | 402 | QSALTQPASVSGSPGQSITIS | 403 |
| | FTFSNYAMYWVRQAPGKGLEWVAVI | | CTGTSSDVGGYNYVSWYQQ | |
| | SYDGSNKYYADSVKGRFTISRDNSKN | | HPGKAPKLMIYDVSKRPSGV | |
| | TLYLQMNSLRTEDTAVYYCASGSDY | | SNRFSGSKSGNTASLTISGLQ | |
| | GDYLLVYWGQGTLVTVSS | | SEDEADYYCNSLTSISTWVF | |
| | | | GGGTKLTVL | |
| Brolucizumab | EVQLVESGGGLVQPGGSLRLSCTASG | 404 | EIVMTQSPSTLSASVGDRVII | 405 |
| | FSLTDYYYMTWVRQAPGKGLEWVG | | TCQASEIIHSWLAWYQQKPG | |
| | FIDPDDDPYYATWAKGRFTISRDNSK | | KAPKLLIYLASTLASGVPSRF | |
| | NTLYLQMNSLRAEDTAVYYCAGGDH | | SGSGSGAEFTLTISSLQPDDF | |
| | NSGWGLDIWGQGTLVTVSS | | ATYYCQNVYLASTNGANFG | |
| | | | QGTKLTVL | |

In some embodiments, co-expression of the enhancer protein, e.g., an L protein, improves the expression of a target protein, wherein the target protein is a blood protein or immune-oncology protein selected from the group comprising (also see Table 9): rFIX-Fc Coagulation Factor IX, Taliglucerase, Agalsidase beta, Alglucosidase alfa, Laronidase, Idursulfase, HLA Class I alpha chain (mouse K2-D1) & B2m (mouse), Nlrc5 (mouse), NLRC5 (human), sclL-12 (mouse), sclL-12 (human), and HLA Class I alpha chain (human) and B2M (human).

TABLE 9
Example blood and immuno-oncology
target proteins

| | | SEQ |
|---|---|---|
| | | ID |
| Target protein | Amino acid sequence | NO: |
| Glucosylceramidase | ARPCIPKSFGYSSVVCVCNATYCDS | 406 |
| (GBA) | FDPPTFPALGTFSRYESTRSGRRME | |
| | LSMGPIQANHTGTGLLLTLQPEQKF | |
| | QKVKGFGGAMTDAAALNILALSPPA | |
| | QNLLLKSYFSEEGIGYNIIRVPMAS | |
| | CDFSIRTYTYADTPDDFQLHNFSLP | |
| | EEDTKLKIPLIHRALQLAQRPVSLL | |
| | ASPWTSPTWLKTNGAVNGKGSLKGQ | |
| | PGDIYHQTWARYFVKFLDAYAEHKL | |
| | QFWAVTAENEPSAGLLSGYPFQCLG | |
| | FTPEHQRDFIARDLGPTLANSTHHN | |
| | VRLLMLDDQRLLLPHWAKVVLTDPE | |
| | AAKYVHGIAVHWYLDFLAPAKATLG | |
| | ETHRLFPNTMLFASEACVGSKFWEQ | |
| | SVRLGSWDRGMQYSHSIITNLLYHV | |
| | VGWTDWNLALNPEGGPNWVRNFVDS | |
| | PIIVDITKDTFYKQPMFYHLGHFSK | |
| | FIPEGSQRVGLVASQKNDLDAVALM | |
| | HPDGSAVVVVLNRSSKDVPLTIKDP | |
| | AVGFLETISPGYSIHTYLWRRQ | |
| rFIX-Fc | YNSGKLEEFVQGNLERECMEEKCSF | 407 |
| Coagulation | EEAREVFENTERTTEFWKQYVDGDQ | |
| Factor IX | CESNPCLNGGSCKDDINSYECWCPF | |
| | GFEGKNCELDVTCNIKNGRCEQFCK | |
| | NSADNKVVCSCTEGYRLAENQKSCE | |
| | PAVPFPCGRVSVSQTSKLTRAETVF | |
| | PDVDYVNSTEAETILDNITQSTQSF | |
| | NDFTRVVGGEDAKPGQFPWQVVLNG | |
| | KVDAFCGGSIVNEKWIVTAAHCVET | |
| | GVKITVVAGEHNIEETEHTEQKRNV | |
| | IRIIPHHNYNAAINKYNHDIALLEL | |
| | DEPLVLNSYVTPICIADKEYTNIFL | |
| | KFGSGYVSGWGRVFHKGRSALVLQY | |
| | LRVPLVDRATCLRSTKFTIYNNMFC | |
| | AGFHEGGRDSCQGDSGGPHVTEVEG | |
| | TSFLTGIISWGEECAMKGKYGIYTK | |
| | VSRYVNWIKEKTKLT | |
| Taliglucerase | EFARPCIPKSFGYSSVVCVCNATYC | 408 |
| | DSFDPPTFPALGTFSRYESTRSGRR | |
| | MELSMGPIQANHTGTGLLLTLQPEQ | |
| | KFQKVKGFGGAMTDAAALNILALSP | |
| | PAQNLLLKSYFSEEGIGYNIIRVPM | |
| | ASCDFSIRTYTYADTPDDFQLHNFS | |
| | LPEEDTKLKIPLIHRALQLAQRPVS | |
| | LLASPWTSPTWLKTNGAVNGKGSLK | |
| | GQPGDIYHQTWARYFVKFLDAYAEH | |
| | KLQFWAVTAENEPSAGLLSGYPFQC | |
| | LGFTPEHQRDFIARDLGPTLANSTH | |
| | HNVRLLMLDDQRLLLPHWAKVVLTD | |
| | PEAAKYVHGIAVHWYLDFLAPAKAT | |
| | LGETHRLFPNTMLFASEACVGSKFW | |
| | EQSVRLGSWDRGMQYSHSIITNLLY | |
| | HVVGWTDWNLALNPEGGPNWVRNFV | |
| | DSPIIVDITKDTFYKQPMFYHLGHF | |
| | SKFIPEGSQRVGLVASQKNDLDAVA | |
| | LMHPDGSAVVVVLNRSSKDVPLTIK | |
| | DPAVGFLETISPGYSIHTYLWHRQD | |
| | LLVDTM | |
| Agalsidase beta | LDNGLARTPTMGWLHWERFMCNLDC | 409 |
| | QEEPDSCISEKLFMEMAELMVSEGW | |
| | KDAGYEYLCIDDCWMAPQRDSEGRL | |
| | QADPQRFPHGIRQLANYVHSKGLKL | |
| | GIYADVGNKTCAGFPGSFGYYDIDA | |
| | QTFADWGVDLLKFDGCYCDSLENLA | |
| | DGYKHMSLALNRTGRSIVYSCEWPL | |
| | YMWPFQKPNYTEIRQYCNHWRNFAD | |
| | IDDSWKSIKSILDWTSFNQERIVDV | |
| | AGPGGWNDPDMLVIGNFGLSWNQQV | |
| | TQMALWAIMAAPLFMSNDLRHISPQ | |
| | AKALLQDKDVIAINQDPLGKQGYQL | |
| | RQGDNFEVWERPLSGLAWAVAMINR | |
| | QEIGGPRSYTIAVASLGKGVACNPA | |
| | CFITQLLPVKRKLGFYEWTSRLRSH | |
| | INPTGTVLLQLENTMQMSLKDLL | |
| Laronidase | APHLVQVDAARALWPLRRFWRSTGF | 410 |
| | CPPLPHSQADQYVLSWDQQLNLAYV | |
| | GAVPHRGIKQVRTHWLLELVTTRGS | |
| | TGRGLSYNFTHLDGYLDLLRENQLL | |
| | PGFELMGSASGHFTDFEDKQQVFEW | |
| | KDLVSSLARRYIGRYGLAHVSKWNF | |
| | ETWNEPDHHDFDNVSMTMQGFLNYY | |
| | DACSEGLRAASPALRLGGPGDSFHT | |
| | PPRSPLSWGLLRHCHDGTNFFTGEA | |
| | GVRLDYISLHRKGARSSISILEQEK | |
| | VVAQQIRQLFPKFADTPIYNDEADP | |
| | LVGWSLPQPWRADVTYAAMVVKVIA | |
| | QHQNLLLANTTSAFPYALLSNDNAF | |
| | LSYHPHPFAQRTLTARFQVNNTRPP | |
| | HVQLLRKPVLTAMGLLALLDEEQLW | |
| | AEVSQAGTVLDSNHTVGVLASAHRP | |
| | QGPADAWRAAVLIYASDDTRAHPNR | |
| | SVAVTLRLRGVPPGPGLVYVTRYLD | |
| | NGLCSPDGEWRRLGRPVFPTAEQFR | |
| | RMRAAEDPVAAAPRPLPAGGRLTLR | |
| | PALRLPSLLLVHVCARPEKPPGQVT | |
| | RLRALPLTQGQLVLVWSDEHVGSKC | |
| | LWTYEIQFSQDGKAYTPVSRKPSTF | |
| | NLFVFSPDTGAVSGSYRVRALDYWA | |
| | RPGPFSDPVPYLEVPVPRGPPSPGN | |
| | P | |
| Idursulfase | SETQANSTTDALNVLLIIVDDLRPS | 411 |
| | LGCYGDKLVRSPNIDQLASHSLLFQ | |
| | NAFAQQAVCAPSRVSFLTGRRPDTT | |
| | RLYDFNSYWRVHAGNFSTIPQYFKE | |
| | NGYVTMSVGKVFHPGISSNHTDDSP | |
| | YSWSFPPYHPSSEKYENTKTCRGPD | |
| | GELHANLLCPVDVLDVPEGTLPDKQ | |
| | STEQAIQLLEKMKTSASPFFLAVGY | |
| | HKPHIPFRYPKEFQKLYPLENITLA | |
| | PDPEVPDGLPPVAYNPWMDIRQRED | |
| | VQALNISVPYGPIPVDFQRKIRQSY | |
| | FASVSYLDTQVGRLLSALDDLQLAN | |
| | STIIAFTSDHGWALGEHGEWAKYSN | |
| | FDVATHVPLIFYVPGRTASLPEAGE | |
| | KLFPYLDPFDSASQLMEPGRQSMDL | |
| | VELVSLFPTLAGLAGLQVPPRCPVP | |
| | SFHVELCREGKNLLKHFRFRDLEED | |
| | PYLPGNPRELIAYSQYPRPSDIPQW | |
| | NSDKPSLKDIKIMGYSIRTIDYRYT | |
| | VWVGFNPDEFLANFSDIHAGELYFV | |
| | DSDPLQDHNMYNDSQGGDLFQLLMP | |
| HLA Class I | GPHSMRYFETAVSRPGLEEPRYISV | 412 |
| alpha chain | GYVDNKEFVRFDSDAENPRYEPRAP | |
| (mouse K2-D1) | WMEQEGPEYWERETQKAKGQEQWFR | |
| | VSLRNLLGYYNQSAGGSHTLQQMSG | |
| | CDLGSDWRLLRGYLQFAYEGRDYIA | |
| | LNEDLKTWTAADMAAQITRRKWEQS | |
| | GAAEHYKAYLEGECVEWLHRYLKNG | |
| | NATLLRTDSPKAHVTHHPRSKGEVT | |
| | LRCWALGFYPADITLTWQLNGEELT | |
| | QDMELVETRPAGDGTFQKWASVVVP | |
| | LGKEQNYTCRVYHEGLPEPLTLRWE | |
| | PPPSTDSYMVIVAVLGVLGAMAIIG | |
| | AVVAFVMKRRRNTGGKGGDYALAPG | |
| | SQSSEMSLRDCKAR | |
| B2m (mouse) | IQKTPQIQVYSRHPPENGKPNILNC | 413 |
| | YVTQFHPPHIEIQMLKNGKKIPKVE | |
| | MSDMSFSKDWSFYILAHTEFTPTET | |
| | DTYACRVKHASMAEPKTVYWDRDMR | |
| Nlrc5 (mouse) | MDAESIRLNNENLWAWLVRLLSKNP | 414 |
| | EWLSAKLRSFLPTMDLDCSYEPSNP | |
| | EVIHRQLNRLFAQGMATWKSFINDL | |
| | CFELDVPLDMEIPLVSIWGPRDEFS | |
| | KQLGAGEESCPGPQLYHGAKRPFQS | |
| | YGSSPRRKNSKKQQLELAKKYLKLL | |
| | KTSAQQWHGGVCPGAWLTPHSPQTY | |
| | IPPVLQWSRATAPLDAQEGATLGDP | |
| | EAADNIDVSIQDLFSFKAHKGPRVT | |
| | VLLGKAGMGKTTLAYRLRWRWAQGQ | |
| | LDRFQALFLFEFRQLNMITQLPTLP | |
| | QLLFDLYLMPESEPDAVFQYLKENA | |
| | QEVLLIFDGLDEALHADSVGTDNAG | |
| | SALTLFSELCHGNLLPGCWVMTTSR | |
| | PGKLPSCVPTEAATVHMWGFDGLRV | |
| | EKYVTCFFSDLLSQELALKEMRTNA | |
| | RLRGMCAIPALCTVTCFCLRRLLPG | |
| | SSPGQSAALLPTITQLYLQMVETFS | |
| | PSETLLDTSILGFGKVALRGLDTGK | |
| | VVFSVEDISPQLMSFGAVHSLLTSF | |
| | CIHTRPGHEEIGYAFVHLSLQEFFA | |
| | ALYLMASHTVDKDTLVEYVTLNSHW | |
| | VLRTKGRLGLSDHLPAFLAGLASHT | |
| | CHMFLCQLAQQDRAWVGSRQAAVIQ | |
| | VLRKLASRKLTGPKMIELYHCVAET | |
| | QDLELARFTAQSLPSRLSFHNFPLT | |
| | HADLAALANILEHRDDPIHLDFDGC | |
| | PLEPHCPEALVGCGQVENLSFKSRK | |
| | CGDAFAEALCRSLPTMGSLKTLGLT | |
| | GSRITAQGISHLIQTLPLCSQLEEV | |
| | SLHDNQLKDPEVLSLVELLPSLPKL | |
| | QKLDLSRNSFSRSILLSLVKVAITC | |
| | PTVRKLQVRELDLIFYLSPVTETAT | |
| | QQSGASDVQGKDSLKEGQSRSLQLR | |
| | LQKCQLRIRDAEALVELFQKSPQLE | |
| | EVNLSGNHLEDDGCRLVAEAASQLH | |
| | IAQKLDLSDNGLSQTGVTYVLKAMS | |
| | TCGTLEDLHISLLNNTVVLTFAQEP | |
| | REQEGSCKGRAPLISFVSPVTSELS | |
| | QRSRRIRLTHCGFLAKHTETLCEAL | |
| | RASCQTHNLDHLDLSDNSLGGKGVI | |
| | LLTELLPGLGPLKSLNLSRNGLSMD | |
| | AVFSLVQCLSSLQWVFHLDVSLESD | |
| | CIFLRGAGTSRDALEPKFQTGVQVL | |
| | ELSQRYTSRSFCLQECQLEPTSLTF | |
| | LCATLEKSPGPLEVQLSCKSLSDDS | |
| | LKILLQCLPQLPQLSLLQLRHTVLS | |
| | SRSPFLLADIFNLCPRVRKVTLRSL | |
| | CHAVLHFDSNEEQEGVCCGFPGCSL | |
| | SQEHMETLCCALSKCNALSQLDLTD | |
| | NLLGDIGLRCLLECLPQLPISGWLD | |
| | LSHNNISQEGILYLLETLPSYPNIQ | |
| | EVSVSLSSEQIFRMCFSKKEGAGTS | |
| | LRLCECSFSPEQVSKLASSLSQAQQ | |
| | LTELWLTKCHLDLPQLTMLLNLVNR | |
| | PTGLLGLRLEEPWVDSVSLPALMEV | |
| | CAQASGCLTELSISEIQRKLWLQLE | |
| | FPHQEGNSDSMALRLAHCDLETEHS | |
| | HLMIQLVETYARLQQLSLSQVSFND | |
| | NDGTSSKLLQNILLSSCELKSFRLT | |
| | FSQVSTKSLTHLAFGLGHCHHLEEL | |
| | DFSNNSLREEDTELLMGALQGTCRL | |
| | KKLHLSFLPLGASSLALLIQGLSRM | |
| | TLLQDLCLSHNQIGDVGTQCLAAIL | |
| | PKLPELRKFDLSHNQIGDVGTQCLA | |
| | AILPKLPELRKFNLSHNQIGHVGTQ | |
| | CLAAILPKLPELRKFDLSRNQIGDV | |
| | GTQCLAAILPKLPELRKFDLSGNRI | |
| | GPAGGVQLVKSLTHFEHLEEIKLGN | |
| | NALGEPTALELAQRLPPQLRVLCLP | |
| | SSHLGPEGALGLAQALEQCPHIEEV | |
| | SLAENNLAGGVPRFSKRLPLLRQID | |
| | LEFCKIEDQAARHLAANLTLFPALE | |
| | KLLLSGNLLGDEVAAELAQVLPQMG | |
| | QLKKVNLEWNRITARGAQLLAQGLV | |
| | QGSCVPVIRLWNNPILNDVAQSLQS | |
| | QEPRLDFSITDQQTLR | |
| HLA Class I | MAVMAPRTLLLLLSGALALTQTWAG | 415 |
| alpha chain | SHSMRYFFTSVSRPGRGEPRFIAVG | |
| | YVDDTQFVRFDSDAASQKMEPRAPW | |
| | IEQEGPEYWDQETRNMKAHSQTDRA | |
| | NLGTLRGYYNQSEDGSHTIQIMYGC | |
| | DVGPDGRFLRGYRQDAYDGKDYIAL | |
| | NEDLRSWTAADMAAQITKRKWEAVH | |
| | AAEQRRVYLEGRCVDGLRRYLENGK | |
| | ETLQRTDPPKTHMTHHPISDHEATL | |
| | RCWALGFYPAEITLTWQRDGEDQTQ | |
| | DTELVETRPAGDGTFQKWAAVVVPS | |
| | GEEQRYTCHVQHEGLPKPLTLRWEL | |
| | SSQPTIPIVGIIAGLVLLGAVITGA | |
| | VVAAVMWRRKSSDRKGGSYTQAASS | |
| | DSAQGSDVSLTACKV | |
| B2M (Human) | IQRTPKIQVYSRHPAENGKSNFLNC | 416 |
| | YVSGFHPSDIEVDLLKNGERIEKVE | |
| | HSDLSFSKDWSFYLLYYTEFTPTEK | |
| | DEYACRVNHVTLSQPKIVKWDRDM | |
| Nlrc5 (human) | MDPVGLQLGNKNLWSCLVRLLTKDP | 417 |
| | EWLNAKMKFFLPNTDLDSRNETLDP | |
| | EQRVILQLNKLHVQGSDTWQSFIHC | |
| | VCMQLEVPLDLEVLLLSTFGYDDGF | |
| | TSQLGAEGKSQPESQLHHGLKRPHQ | |
| | SCGSSPRRKQCKKQQLELAKKYLQL | |
| | LRTSAQQRYRSQIPGSGQPHAFHQV | |
| | YVPPILRRATASLDTPEGAIMGDVK | |
| | VEDGADVSISDLFNTRVNKGPRVTV | |
| | LLGKAGMGKTTLAHRLCQKWAEGHL | |
| | NCFQALFLFEFRQLNLITRFLTPSE | |
| | LLFDLYLSPESDHDTVFQYLEKNAD | |
| | QVLLIFDGLDEALQPMGPDGPGPVL | |
| | TLFSHLCNGTLLPGCRVMATSRPGK | |
| | LPACLPAEAAMVHMLGFDGPRVEEY | |
| | VNHFFSAQPSREGALVELQTNGRLR | |
| | SLCAVPALCQVACLCLHHLLPDHAP | |
| | GQSVALLPNMTQLYMQMVLALSPPG | |
| | HLPTSSLLDLGEVALRGLETGKVIF | |
| | YAKDIAPPLIAFGATHSLLTSFCVC | |
| | TGPGHQQTGYAFTHLSLQEFLAALH | |
| | LMASPKVNKDTLTQYVTLHSRWVQR | |
| | TKARLGLSDHLPTFLAGLASCTCRP | |
| | FLSHLAQGNEDCVGAKQAAVVQVLK | |
| | KLATRKLTGPKVVELCHCVDETQEP | |
| | ELASLTAQSLPYQLPFHNFPLTCTD | |
| | LATLTNILEHREAPIHLDFDGCPLE | |
| | PHCPEALVGCGQIENLSFKSRKCGD | |
| | AFAEALSRSLPTMGRLQMLGLAGSK | |
| | ITARGISHLVKALPLCPQLKEVSFR | |
| | DNQLSDQVVLNIVEVLPHLPRLRKL | |
| | DLSSNSICVSTLLCLARVAVTCPTV | |
| | RMLQAREADLIFLLSPPTETTAELQ | |
| | RAPDLQESDGQRKGAQSRSLTLRLQ | |
| | KCQLQVHDAEALIALLQEGPHLEEV | |
| | DLSGNQLEDEGCRLMAEAASQLHIA | |
| | RKLDLSNNGLSVAGVHCVLRAVSAC | |
| | WTLAELHISLQHKTVIFMFAQEPEE | |
| | QKGPQERAAFLDSLMLQMPSELPLS | |
| | SRRMRLTHCGLQEKHLEQLCKALGG | |
| | SCHLGHLHLDFSGNALGDEGAARLA | |
| | QLLPGLGALQSLNLSENGLSLDAVL | |
| | GLVRCFSTLQWLFRLDISFESQHIL | |
| | LRGDKTSRDMWATGSLPDFPAAAKF | |
| | LGFRQRCIPRSLCLSECPLEPPSLT | |
| | RLCATLKDCPGPLELQLSCEFLSDQ | |
| | SLETLLDCLPQLPQLSLLQLSQTGL | |
| | SPKSPFLLANTLSLCPRVKKVDLRS | |
| | LHHATLHFRSNEEEEGVCCGRFTGC | |
| | SLSQEHVESLCWLLSKCKDLSQVDL | |
| | SANLLGDSGLRCLLECLPQVPISGL | |
| | LDLSHNSISQESALYLLETLPSCPR | |
| | VREASVNLGSEQSFRIHFSREDQAG | |
| | KTLRLSECSFRPEHVSRLATGLSKS | |
| | LQLTELTLTQCCLGQKQLAILLSLV | |
| | GRPAGLFSLRVQEPWADRARVLSLL | |
| | EVCAQASGSVTEISISETQQQLCVQ | |
| | LEFPRQEENPEAVALRLAHCDLGAH | |
| | HSLLVGQLMETCARLQQLSLSQVNL | |
| | CEDDDASSLLLQSLLLSLSELKTFR | |
| | LTSSCVSTEGLAHLASGLGHCHHLE | |
| | ELDLSNNQFDEEGTKALMRALEGKW | |
| | MLKRLDLSHLLLNSSTLALLTHRLS | |
| | QMTCLQSLRLNRNSIGDVGCCHLSE | |
| | ALRAATSLEELDLSHNQIGDAGVQH | |
| | LATILPGLPELRKIDLSGNSISSAG | |
| | GVQLAESLVLCRRLEELMLGCNALG | |
| | DPTALGLAQELPQHLRVLHLPFSHL | |
| | GPGGALSLAQALDGSPHLEEISLAE | |
| | NNLAGGVLRFCMELPLLRQIDLVSC | |
| | KIDNQTAKLLTSSFTSCPALEVILL | |
| | SWNLLGDEAAAELAQVLPQMGRLKR | |
| | VDLEKNQITALGAWLLAEGLAQGSS | |
| | IQVIRLWNNPIPCDMAQHLKSQEPR | |
| | LDFAFFDNQPQAPWGT | |
| scIL-12 (mouse) | MWELEKDVYVVEVDWTPDAPGETVN | 418 |
| | LTCDTPEEDDITWTSDQRHGVIGSG | |
| | KTLTITVKEFLDAGQYTCHKGGETL | |
| | SHSHLLLHKKENGIWSTEILKNFKN | |
| | KTFLKCEAPNYSGRFTCSWLVQRNM | |
| | DLKFNIKSSSSSPDSRAVTCGMASL | |
| | SAEKVTLDQRDYEKYSVSCQEDVTC | |
| | PTAEETLPIELALEARQQNKYENYS | |
| | TSFFIRDIIKPDPPKNLQMKPLKNS | |
| | QVEVSWEYPDSWSTPHSYFSLKFFV | |
| | RIQRKKEKMKETEEGCNQKGAFLVE | |
| | KTSTEVQCKGGNVCVQAQDRYYNSS | |
| | CSKWACVPCRVRS | |
| scIL-12 (human) | IWELKKDVYVVELDWYPDAPGEMVV | 419 |
| | LTCDTPEEDGITWTLDQSSEVLGSG | |
| | KTLTIQVKEFGDAGQYTCHKGGEVL | |
| | SHSLLLLHKKEDGIWSTDILKDQKE | |
| | PKNKTFLRCEAKNYSGRFTCWWLTT | |
| | ISTDLTFSVKSSRGSSDPQGVTCGA | |
| | ATLSAERVRGDNKEYEYSVECQEDS | |
| | ACPAAEESLPIEVMVDAVHKLKYEN | |
| | YTSSFFIRDIIKPDPPKNLQLKPLK | |
| | NSRQVEVSWEYPDTWSTPHSYFSLT | |
| | FCVQVQGKSKREKKDRVFTDKTSAT | |
| | VICRKNASISVRAQDRYYSSSWSEW | |
| | ASVPCS | |
| Alglucosidase | AHPGRPRAVPTQCDVPPNSRFDCAP | 420 |
| alfa | DKAITQEQCEARGCCYIPAKQGLQG | |
| | AQMGQPWCFFPPSYPSYKLENLSSS | |
| | EMGYTATLTRTTPTFFPKDILTLRL | |
| | DVMMETENRLHFTIKDPANRRYEVP | |
| | LETPHVHSRAPSPLYSVEFSEEPFG | |
| | VIVRRQLDGRVLLNTTVAPLFFADQ | |
| | FLQLSTSLPSQYITGLAEHLSPLML | |
| | STSWTRITLWNRDLAPTPGANLYGS | |
| | HPFYLALEDGGSAHGVFLLNSNAMD | |
| | VVLQPSPALSWRSTGGILDVYIFLG | |
| | PEPKSVVQQYLDVVGYPFMPPYWGL | |
| | GFHLCRWGYSSTAITRQVVENMTRA | |
| | HFPLDVQWNDLDYMDSRRDFTFNKD | |
| | GFRDFPAMVQELHQGGRRYMMIVDP | |
| | AISSSGPAGSYRPYDEGLRRGVFIT | |
| | NETGQPLIGKVWPGSTAFPDFTNPT | |
| | ALAWWEDMVAEFHDQVPFDGMWIDM | |
| | NEPSNFIRGSEDGCPNNELENPPYV | |
| | PGVVGGTLQAATICASSHQFLSTHY | |
| | NLHNLYGLTEAIASHRALVKARGTR | |
| | PFVISRSTFAGHGRYAGHWTGDVWS | |
| | SWEQLASSVPEILQFNLLGVPLVGA | |
| | DVCGFLGNTSEELCVRWTQLGAFYP | |
| | FMRNHNSLLSLPQEPYSFSEPAQQA | |
| | MRKALTLRYALLPHLYTLFHQAHVA | |
| | GETVARPLFLEFPKDSSTWTVDHQL | |
| | LWGEALLITPVLQAGKAEVTGYFPL | |
| | GTWYDLQTVPVEALGSLPPPPAAPR | |
| | EPAIHSEGQWVTLPAPLDTINVHLR | |
| | AGYIIPLQGPGLTTTESRQQPMALA | |
| | VALTKGGEARGELFWDDGESLEVLE | |
| | RGAYTQVIFLARNNTIVNELVRVTS | |
| | EGAGLQLQKVTVLGVATAPQQVLSN | |
| | GVPVSNFTYSPDTKVLDICVSLLMG | |
| | EQFLVSWC | |
| B-domain | ATRRYYLGAVELSWDYMQSDLGELP | 421 |
| deleted human | VDARFPPRVPKSFPFNTSVVYKKTL | |
| Factor VIII | FVEFTDHLFNIAKPRPPWMGLLGPT | |
| (BDD FVIII) | IQAEVYDTVVITLKNMASHPVSLHA | |
| | VGVSYWKASEGAEYDDQTSQREKED | |
| | DKVFPGGSHTYVWQVLKENGPMASD | |
| | PLCLTYSYLSHVDLVKDLNSGLIGA | |
| | LLVCREGSLAKEKTQTLHKFILLFA | |
| | VFDEGKSWHSETKNSLMQDRDAASA | |
| | RAWPKMHTVNGYVNRSLPGLIGCHR | |
| | KSVYWHVIGMGTTPEVHSIFLEGHT | |
| | FLVRNHRQASLEISPITFLTAQTLL | |
| | MDLGQFLLFCHISSHQHDGMEAYVK | |
| | VDSCPEEPQLRMKNNEEAEDYDDDL | |
| | TDSEMDVVRFDDDNSPSFIQIRSVA | |
| | KKHPKTWVHYIAAEEEDWDYAPLVL | |
| | APDDRSYKSQYLNNGPQRIGRKYKK | |
| | VRFMAYTDETFKTREAIQHESGILG | |
| | PLLYGEVGDTLLIIFKNQASRPYNI | |
| | YPHGITDVRPLYSRRLPKGVKHLKD | |
| | FPILPGEIFKYKWTVTVEDGPTKSD | |
| | PRCLTRYYSSFVNMERDLASGLIGP | |
| | LLICYKESVDQRGNQIMSDKRNVIL | |
| | FSVFDENRSWYLTENIQRFLPNPAG | |
| | VQLEDPEFQASNIMHSINGYVFDSL | |
| | QLSVCLHEVAYWYILSIGAQTDFLS | |
| | VFFSGYTFKHKMVYEDTLTLFPFSG | |
| | ETVFMSMENPGLWILGCHNSDFRNR | |
| | GMTALLKVSSCDKNTGDYYEDSYED | |
| | ISAYLLSKNNAIEPRSFSQNPPVLK | |
| | RHQREITRTTLQSDQEEIDYDDTIS | |
| | VEMKKEDFDIYDEDENQSPRSFQKK | |
| | TRHYFIAAVERLWDYGMSSSPHVLR | |
| | NRAQSGSVPQFKKVVFQEFTDGSFT | |
| | QPLYRGELNEHLGLLGPYIRAEVED | |
| | NIMVTFRNQASRPYSFYSSLISYEE | |
| | DQRQGAEPRKNFVKPNETKTYFWKV | |
| | QHHMAPTKDEFDCKAWAYFSDVDLE | |
| | KDVHSGLIGPLLVCHTNTLNPAHGR | |
| | QVTVQEFALFFTIFDETKSWYFTEN | |
| | MERNCRAPCNIQMEDPTFKENYRFH | |
| | AINGYIMDTLPGLVMAQDQRIRWYL | |
| | LSMGSNENIHSIHFSGHVFTVRKKE | |
| | EYKMALYNLYPGVFETVEMLPSKAG | |
| | IWRVECLIGEHLHAGMSTLFLVYSN | |
| | KCQTPLGMASGHIRDFQITASGQYG | |
| | QWAPKLARLHYSGSINAWSTKEPFS | |
| | WIKVDLLAPMIIHGIKTQGARQKFS | |
| | SLYISQFIIMYSLDGKKWQTYRGNS | |
| | TGTLMVFFGNVDSSGIKHNIFNPPI | |
| | IARYIRLHPTHYSIRSTLRMELMGC | |
| | DLNSCSMPLGMESKAISDAQITASS | |
| | YFTNMFATWSPSKARLHLQGRSNAW | |
| | RPQVNNPKEWLQVDFQKTMKVTGVT | |
| | TQGVKSLLTSMYVKEFLISSSQDGH | |
| | QWTLFFQNGKVKVFQGNQDSFTPVV | |
| | NSLDPPLLTRYLRIHPQSWVHQIAL | |
| | RMEVLGCEAQDLY | |
| Von Willebrand | AEGTRGRSSTARCSLFGSDFVNTFD | 422 |
| Factor, | GSMYSFAGYCSYLLAGGCQKRSFSI | |
| recombinant | IGDFQNGKRVSLSVYLGEFFDIHLF | |
| | VNGTVTQGDQRVSMPYASKGLYLET | |
| | EAGYYKLSGEAYGFVARIDGSGNFQ | |
| | VLLSDRYFNKTCGLCGNFNIFAEDD | |
| | FMTQEGTLTSDPYDFANSWALSSGE | |
| | QWCERASPPSSSCNISSGEMQKGLW | |
| | EQCQLLKSTSVFARCHPLVDPEPFV | |
| | ALCEKTLCECAGGLECACPALLEYA | |
| | RTCAQEGMVLYGWTDHSACSPVCPA | |
| | GMEYRQCVSPCARTCQSLHINEMCQ | |
| | ERCVDGCSCPEGQLLDEGLCVESTE | |
| | CPCVHSGKRYPPGTSLSRDCNTCIC | |
| | RNSQWICSNEECPGECLVTGQSHFK | |
| | SFDNRYFTFSGICQYLLARDCQDHS | |
| | FSIVIETVQCADDRDAVCTRSVTVR | |
| | LPGLHNSLVKLKHGAGVAMDGQDVQ | |
| | LPLLKGDLRIQHTVTASVRLSYGED | |
| | LQMDWDGRGRLLVKLSPVYAGKTCG | |
| | LCGNYNGNQGDDFLTPSGLAEPRVE | |
| | DFGNAWKLHGDCQDLQKQHSDPCAL | |
| | NPRMTRFSEEACAVLTSPTFEACHR | |
| | AVSPLPYLRNCRYDVCSCSDGRECL | |
| | CGALASYAAACAGRGVRVAWREPGR | |
| | CELNCPKGQVYLQCGTPCNLTCRSL | |
| | SYPDEECNEACLEGCFCPPGLYMDE | |
| | RGDCVPKAQCPCYYDGEIFQPEDIF | |
| | SDHHTMCYCEDGFMHCTMSGVPGSL | |
| | LPDAVLSSPLSHRSKRSLSCRPPMV | |
| | KLVCPADNLRAEGLECTKTCQNYDL | |
| | ECMSMGCVSGCLCPPGMVRHENRCV | |
| | ALERCPCFHQGKEYAPGETVKIGCN | |
| | TCVCQDRKWNCTDHVCDATCSTIGM | |
| | AHYLTFDGLKYLFPGECQYVLVQDY | |
| | CGSNPGTFRILVGNKGCSHPSVKCK | |
| | KRVTILVEGGEIELFDGEVNVKRPM | |
| | KDETHFEVVESGRYIILLLGKALSV | |
| | VWDRHLSISVVLKQTYQEKVCGLCG | |
| | NFDGIQNNDLTSSNLQVEEDPVDFG | |
| | NSWKVSSQCADTRKVPLDSSPATCH | |
| | NNIMKQTMVDSSCRILTSDVFQDCN | |
| | KLVDPEPYLDVCIYDTCSCESIGDC | |
| | ACFCDTIAAYAHVCAQHGKVVTWRT | |
| | ATLCPQSCEERNLRENGYECEWRYN | |
| | SCAPACQVTCQHPEPLACPVQCVEG | |
| | CHAHCPPGKILDELLQTCVDPEDCP | |
| | VCEVAGRRFASGKKVTLNPSDPEHC | |
| | QICHCDVVNLTCEACQEPGGLVVPP | |
| | TDAPVSPTTLYVEDISEPPLHDFYC | |
| | SRLLDLVFLLDGSSRLSEAEFEVLK | |
| | AFVVDMMERLRISQKWVRVAVVEYH | |
| | DGSHAYIGLKDRKRPSELRRIASQV | |
| | KYAGSQVASTSEVLKYTLFQIFSKI | |
| | DRPEASRITLLLMASQEPQRMSRNF | |
| | VRYVQGLKKKKVIVIPVGIGPHANL | |
| | KQIRLIEKQAPENKAFVLSSVDELE | |
| | QQRDEIVSYLCDLAPEAPPPTLPPD | |
| | MAQVTVGPGLLGVSTLGPKRNSMVL | |
| | DVAFVLEGSDKIGEADFNRSKEFME | |
| | EVIQRMDVGQDSIHVTVLQYSYMVT | |
| | VEYPFSEAQSKGDILQRVREIRYQG | |
| | GNRTNTGLALRYLSDHSFLVSQGDR | |
| | EQAPNLVYMVTGNPASDEIKRLPGD | |
| | IQVVPIGVGPNANVQELERIGWPNA | |
| | PILIQDFETLPREAPDLVLQRCCSG | |
| | EGLQIPTLSPAPDCSQPLDVILLLD | |
| | GSSSFPASYFDEMKSFAKAFISKAN | |
| | IGPRLTQVSVLQYGSITTIDVPWNV | |
| | VPEKAHLLSLVDVMQREGGPSQIGD | |
| | ALGFAVRYLTSEMHGARPGASKAVV | |
| | ILVTDVSVDSVDAAADAARSNRVTV | |
| | FPIGIGDRYDAAQLRILAGPAGDSN | |
| | VVKLQRIEDLPTMVTLGNSFLHKLC | |
| | SGFVRICMDEDGNEKRPGDVWTLPD | |
| | QCHTVTCQPDGQTLLKSHRVNCDRG | |
| | LRPSCPNSQSPVKVEETCGCRWTCP | |
| | CVCTGSSTRHIVTFDGQNFKLTGSC | |
| | SYVLFQNKEQDLEVILHNGACSPGA | |
| | RQGCMKSIEVKHSALSVELHSDMEV | |
| | TVNGRLVSVPYVGGNMEVNVYGAIM | |
| | HEVRFNHLGHIFTFTPQNNEFQLQL | |
| | SPKTFASKTYGLCGICDENGANDFM | |
| | LRDGTVTTDWKTLVQEWTVQRPGQT | |
| | CQPILEEQCLVPDSSHCQVLLLPLF | |
| | AECHKVLAPATFYAICQQDSCHQEQ | |
| | VCEVIASYAHLCRTNGVCVDWRTPD | |
| | FCAMSCPPSLVYNHCEHGCPRHCDG | |
| | NVSSCGDHPSEGCFCPPDKVMLEGS | |
| | CVPEEACTQCIGEDGVQHQFLEAWV | |
| | PDHQPCQICTCLSGRKVNCTTQPCP | |
| | TAKAPTCGLCEVARLRQNADQCCPE | |
| | YECVCDPVSCDLPPVPHCERGLQPT | |
| | LTNPGECRPNFTCACRKEECKRVSP | |
| | PSCPPHRLPTLRKTQCCDEYECACN | |
| | CVNSTVSCPLGYLASTATNDCGCTT | |
| | TTCLPDKVCVHRSTIYPVGQFWEEG | |
| | CDVCTCTDMEDAVMGLRVAQCSQKP | |
| | CEDSCRSGFTYVLHEGECCGRCLPS | |
| | ACEVVTGSPRGDSQSSWKSVGSQWA | |
| | SPENPCLINECVRVKEEVFIQQRNV | |
| | SCPQLEVPVCPSGFQLSCKTSACCP | |
| | SCRCERMEACMLNGTVIGPGKTVMI | |
| | DVCTTCRCMVQVGVISGFKLECRKT | |
| | TCNPCPLGYKEENNTGECCGRCLPT | |
| | ACTIQLRGGQIMTLKRDETLQDGCD | |
| | THFCKVNERGEYFWEKRVTGCPPFD | |
| | EHKCLAEGGKIMKIPGTCCDTCEEP | |
| | ECNDITARLQYVKVGSCKSEVEVDI | |
| | HYCQGKCASKAMYSIDINDVQDQCS | |
| | CCSPTRTEPMQVALHCTNGSVVYHE | |
| | VLNAMECKCSPRKCSK | |

In some embodiments, co-expression of an enhancer protein, e.g. an L protein, with the set of polynucleotides of SEQ ID NOS: 191-216 may be used to improve the expression of an antibody or target protein from either of Tables 8 or 9, wherein the target protein is expressed in place of adalimumab.

In some embodiments, co-expression of an enhancer protein, e.g. an L protein, with the set of polynucleotides of SEQ ID NOS: 243-272 (an AAV vector) may be used to improve the expression of an antibody or target protein from either of Tables 8 or 9, wherein the target protein is expressed in place of adalimumab.

### EXEMPLARY EMBODIMENTS

### Embodiments I

Embodiment I-1. A system for recombinant expression of a target protein in eukaryotic cells, comprising one or more vectors, the one or more vectors comprising:
a) a first polynucleotide encoding the target protein; and
b) a second polynucleotide encoding an enhancer protein wherein:
   i) the enhancer protein is an inhibitor of nucleocytoplasmic transport (NCT) and/or
   ii) the enhancer protein is selected from the group consisting of a picornavirus leader (L) protein, a picornavirus 2A protease, a rhinovirus 3C protease, a herpes simplex virus (HSV) ICP27 protein, and a rhabdovirus matrix (M) protein,
      wherein the first polynucleotide and the second polynucleotide are operatively linked to one or more promoters.

Embodiment I-2. The system of embodiment I-1, wherein the enhancer protein is an inhibitor of nucleocytoplasmic transport (NCT).

Embodiment I-3. The system of embodiment I-2, wherein the NCT inhibitor is a viral protein.

Embodiment I-4. The system of any one of embodiments I-1 to I-3, wherein the NCT inhibitor is selected from the group consisting of a picornavirus leader (L) protein, a picomavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

Embodiment I-5. The system of embodiment I-4, wherein the NCT inhibitor is a picomavirus leader (L) protein or a functional variant thereof.

Embodiment I-6. The system of embodiment I-4, wherein the NCT inhibitor is a picornavirus 2A protease or a functional variant thereof.

Embodiment I-7. The system of embodiment I-4, wherein the NCT inhibitor is a rhinovirus 3C protease or a functional variant thereof.

Embodiment I-8. The system of embodiment I-4, wherein the NCT inhibitor is a coronavirus ORF6 protein or a functional variant thereof.

Embodiment I-9. The system of embodiment I-4, wherein the NCT inhibitor is an ebolavirus VP24 protein or a functional variant thereof.

Embodiment I-10. The system of embodiment I-4, wherein the NCT inhibitor is a Venezuelan equine encephalitis virus (VEEV) capsid protein or a functional variant thereof.

Embodiment I-11. The system of embodiment I-4, wherein the NCT inhibitor is a herpes simplex virus (HSV) ICP27 protein or a functional variant thereof.

Embodiment I-12. The system of embodiment I-4, wherein the NCT inhibitor is a rhabdovirus matrix (M) protein or a functional variant thereof.

Embodiment I-13. The system of embodiment I-5, wherein the L protein is the L protein of Theiler's virus or a functional variant thereof.

Embodiment I-14. The system of embodiment I-5, wherein the L protein shares at least 90% identity to SEQ ID NO: 1.

Embodiment I-15. The system of embodiment I-5, wherein the L protein is the L protein of Encephalomyocarditis virus (EMCV) or a functional variant thereof.

Embodiment I-16. The system of embodiment I-5, wherein the L protein shares at least 90% identity to SEQ ID NO: 2.

Embodiment I-17. The system of embodiment I-5, wherein the L protein is selected from the group consisting of the L protein of poliovirus, the L protein of HRV16, the L protein of mengo virus, and the L protein of Saffold virus 2 or a functional variant thereof.

Embodiment I-18. The system of any one of embodiments I-1 to I-17, wherein the system comprises a single vector comprising an expression cassette, the expression cassette comprising the first polynucleotide and the second polynucleotide.

Embodiment I-19. The system of embodiment I-18, wherein the expression cassette comprises a first promoter, operatively linked to the first polynucleotide; and a second promoter, operatively linked to the second polynucleotide.

Embodiment I-20. The system of embodiment I-18, wherein the expression cassette comprises a shared promoter operatively linked to both the first polynucleotide and the second polynucleotide.

Embodiment I-21. The system of embodiment I-20, wherein the expression cassette comprises a coding polynucleotide comprising the first polynucleotide and the second polynucleotide linked by a polynucleotide encoding ribosome skipping site, the coding polynucleotide operatively linked to the shared promoter.

Embodiment I-22. The system of embodiment I-20, wherein the expression cassette comprises a coding polynucleotide, the coding polynucleotide encoding the enhancer protein and the target protein linked to by a ribosome skipping site, the coding polynucleotide operatively linked to the shared promoter.

Embodiment I-23. The system of any one of embodiments I-18 to I-22, wherein the expression cassette is configured for transcription of a single messenger RNA encoding both the target protein and the enhancer protein, linked by a ribosome skipping site; wherein translation of the messenger RNA results in expression of the target protein and the L protein as distinct polypeptides.

Embodiment I-24. The system of any one of embodiments I-1 to I-23, wherein the system comprises one vector.

Embodiment I-25. The system of any one of embodiments I-1 to I-17, wherein the system comprises:
a) a first vector comprising the first polynucleotide, operatively linked to a first promoter; and
b) a second vector comprising the second polynucleotide, operatively linked to a second promoter.

Embodiment I-26. The system of any one of embodiments I-1 to I-17 or embodiment I-25, wherein the system comprises two vectors.

Embodiment I-27. The system of any one of embodiments I-1 to I-26, wherein either the first polynucleotide or the second polynucleotide, or both, are operatively linked to an internal ribosome entry site (IRES).

Embodiment I-28. The system of any one of embodiments I-1 to I-27, wherein at least one of the one or more vectors comprises a T7 promoter configured for transcription of either or both of the first polynucleotide or the second polynucleotide by a T7 RNA polymerase.

Embodiment I-29. The system of any one of embodiments I-1 to I-28, wherein at least one of the one or more vectors comprises a polynucleotide sequence encoding a T7 RNA polymerase.

Embodiment I-30. A vector for recombinant expression of a target protein in eukaryotic cells, comprising:
a) a first polynucleotide encoding the target protein; and
b) a second polynucleotide encoding an enhancer protein wherein:
   i) the enhancer protein is an inhibitor of nucleocytoplasmic transport (NCT) and/or
   ii) the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.
      wherein the first polynucleotide and the second polynucleotide are operatively linked to at least one promoter.

Embodiment I-31. The vector of embodiment I-30, wherein the expression cassette comprises a first promoter, operatively linked to the first polynucleotide; and a second promoter, operatively linked to the second polynucleotide.

Embodiment I-32. The vector of embodiment I-30, wherein the expression cassette comprises a shared promoter operatively linked to both the first polynucleotide and the second polynucleotide.

Embodiment I-32.1 The vector of embodiment I-30, wherein the vector comprises a nucleic acid sequence having at least 80% identity to SEQ ID NO: 100.

Embodiment I-32.2 The vector of embodiment I-30, wherein the vector comprises a polynucleotide encoding SEQ ID NO: 132 and/or a polynucleotide encoding SEQ ID NO: 133.

Embodiment I-32.3 The vector of embodiment I-30, wherein the vector comprises a polynucleotide comprising the nucleic acid sequence of SEQ ID NO: 134 and/or a polynucleotide encoding SEQ ID NO: 135.

Embodiment I-33. A eukaryotic cell for expression of a target protein, comprising an exogenous polynucleotide encoding an enhancer protein wherein:
a) the enhancer protein is an inhibitor of nucleocytoplasmic transport (NCT) and/or
b) the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein, wherein the exogenous polynucleotide is operatively linked to a promoter

Embodiment I-34. The cell of embodiment I-33, wherein the polynucleotide is operatively linked to an internal ribosome entry site (IRES).

Embodiment I-35. The cell of embodiment I-33 or embodiment I-34, wherein the promoter is an inducible promoter.

Embodiment I-36. A method for recombinant expression of a target protein, comprising introducing a polynucleotide encoding the target protein, operatively linked to a promoter, into the cell of any one of embodiments I-33 to I-35.

Embodiment I-37. A method for recombinant expression of a target protein, comprising introducing the system of any one of embodiments I-1 to I-29 or the vector of any one of embodiments I-30 to I-32 into eukaryotic cell.

Embodiment I-38. The method of embodiment I-36 or embodiment I-37, wherein the target protein is a membrane protein

Embodiment I-39. The method of any embodiment I-38, wherein localization of the membrane protein to the cellular membrane is increased compared to the localization observed when the membrane protein is expressed without the enhancer protein.

Embodiment I-40. A cell produced by introduction of the system of any one of embodiments I-1 to I-29 or the vector of any one of embodiments I-30 to I-32 into a eukaryotic cell.

Embodiment I-41. A target protein expressed by introduction of the system of any one of embodiments I-1 to I-29 or the vector of any one of embodiments I-30 to I-32 into a eukaryotic cell.

Embodiment I-42. A method for expressing a target protein in eukaryotic cells, comprising introducing a polynucleotide encoding the target protein, the polynucleotide operatively linked to a promoter, into the eukaryotic cells,
wherein the method utilizes co-expression of an enhancer protein to enhance the expression level, solubility and/or activity of the target protein,
wherein:
a) the enhancer protein is an inhibitor of nucleocytoplasmic transport (NCT) and/or
b) the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

Embodiment I-43. The method of embodiment I-42, wherein the co-expression of enhancer protein comprises introducing into the eukaryotic cell a polynucleotide encoding the enhancer protein, operatively linked to a promoter.

Embodiment I-44. The method of embodiment I-42 or embodiment I-43, wherein the introducing step or steps comprise transfection of the eukaryotic cells with one or more DNA molecules, transduction of the eukaryotic cells with a single viral vector, and/or transduction of the eukaryotic cells with two viral vectors.

Embodiment I-45. The vector system of any one of embodiments I-1 to I-29, vector of any one of embodiments I-30 to I-32, the cell of any one of embodiments I-33 to I-35, or the method of any one of embodiments I-36 to I-44, wherein the target protein is a soluble protein.

Embodiment I-46. The vector system of any one of embodiments I-1 to I-29, the vector of any one of embodiments I-30 to I-32, the cell of any one of embodiments I-33 to I-35, or the method of any one of embodiments I-36 to I-44, wherein the target protein is a secreted protein.

Embodiment I-47. The vector system of any one of embodiments I-1 to I-29, the vector of any one of embodiments I-30 to I-32, the cell of any one of embodiments I-33 to I-35, or the method of any one of embodiments I-36 to I-44, wherein the target protein is a membrane protein.

Embodiment I-48. The vector system of any one of embodiments I-1 to I-29, the vector of any one of embodiments I-30 to I-32, the cell of any one of embodiments I-33 to I-35, or the method of any one of embodiments I-36 to I-44, wherein the target protein is Dopamine receptor 1 (DRD1).

Embodiment I-49. The vector system of any one of embodiments I-1 to I-29, the vector of any one of embodiments I-30 to I-32, the cell of any one of embodiments I-33 to I-35, or the method of any one of embodiments I-36 to I-44, wherein the target protein is Cystic fibrosis transmembrane conductance regulator (CFTR).

Embodiment I-50. The vector system of any one of embodiments I-1 to I-29, the vector of any one of embodiments I-30 to I-32, the cell of any one of embodiments I-33 to I-35, or the method of any one of embodiments I-36 to I-44, wherein the target protein is C1 esterase inhibitor (C1-Inh).

Embodiment I-51. The vector system of any one of embodiments I-1 to I-29, the vector of any one of embodiments I-30 to I-32, the cell of any one of embodiments I-33 to I-35, or the method of any one of embodiments I-36 to I-44, wherein the target protein is IL2 inducible T cell kinase (ITK).

Embodiment I-52. The vector system of any one of embodiments I-1 to I-29, the vector of any one of embodiments I-30 to I-32, the cell of any one of embodiments I-33 to I-35, or the method of any one of embodiments I-36 to I-44, wherein the target protein is an NADase.

Embodiment I-53. A method for generating an antibody against a target protein, comprising immunizing a subject with the cell of any one of embodiments I-33 to I-35, the cell of embodiment I-40, or the target protein of embodiment I-41.

Embodiment I-54. The method of embodiment I-53, further comprising isolating one or more immune cells expressing an immunoglobulin protein specific for the target protein.

Embodiment I-55. The method of embodiment I-53 or embodiment I-54, comprising generating one or more hybridomas from the one or more immune cells.

Embodiment I-56. The method of any one of embodiments I-53 to I-55, comprising cloning one or more immunoglobulin genes from the one or more immune cells.

Embodiment I-57. A method for antibody discovery by cell sorting, comprising providing a solution comprising:

a) the cell of any one of embodiments I-33 to I-35, the cell of embodiment I-40, or the target protein of embodiment I-41, wherein the cell or target protein is labeled, and
b) a population of recombinant cells, wherein the recombinant cells express a library of polypeptides each comprising an antibody or antigen-binding fragment thereof; and isolating one or more recombinant cells from the solution by sorting for recombinant cells bound to the labeled cell or the labeled target protein.

Embodiment I-58. A method for panning a phage-display library, comprising:
a) mixing a phage-display library with the cell of any one of embodiments I-33 to 1-35, the cell of embodiment I-40, or the target protein of embodiment I-41; and
b) purifying and/or enriching the members of the phage-display library that bind the cell or target protein.

Embodiment I-59. A method of expressing a target protein in a subject in need thereof, comprising administering to the subject a vector system comprising one or more vectors, the one or more vectors, comprising:
a) a first polynucleotide encoding the target protein; and
b) a second polynucleotide encoding an enhancer protein wherein:
   i) the enhancer protein is an inhibitor of nucleocytoplasmic transport (NCT) and/or
   ii) the enhancer protein is selected from the group consisting of a picornavirus leader (L) protein, a picornavirus 2A protease, a rhinovirus 3C protease, a herpes simplex virus (HSV) ICP27 protein, and a rhabdovirus matrix (M) protein,
      wherein the first polynucleotide and the second polynucleotide are operatively linked to one or more promoters.

Embodiment I-60. The method of embodiment I-59, wherein the enhancer protein is an inhibitor of nucleocytoplasmic transport (NCT).

Embodiment I-61. The method of embodiment I-60, wherein the NCT inhibitor is a viral protein.

Embodiment I-62. The method of any one of embodiments I-59-61, wherein the NCT inhibitor is selected from the group consisting of a picomavirus leader (L) protein, a picomavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

Embodiment I-63. The method of embodiment I-62, wherein the NCT inhibitor is a picomavirus leader (L) protein or a functional variant thereof.

Embodiment I-64. The method of embodiment I-62, wherein the NCT inhibitor is a picornavirus 2A protease or a functional variant thereof.

Embodiment I-65. The method of embodiment I-62, wherein the NCT inhibitor is a rhinovirus 3C protease or a functional variant thereof.

Embodiment I-66. The method of embodiment I-62, wherein the NCT inhibitor is a coronavirus ORF6 protein or a functional variant thereof.

Embodiment I-67. The method of embodiment I-62, wherein the NCT inhibitor is an ebolavirus VP24 protein or a functional variant thereof.

Embodiment I-68. The method of embodiment I-62, wherein the NCT inhibitor is a Venezuelan equine encephalitis virus (VEEV) capsid protein or a functional variant thereof.

Embodiment I-69. The method of embodiment I-62, wherein the NCT inhibitor is a herpes simplex virus (HSV) ICP27 protein or a functional variant thereof.

Embodiment I-70. The method of embodiment I-62, wherein the NCT inhibitor is a rhabdovirus matrix (M) protein or a functional variant thereof.

Embodiment I-71. The method of embodiment I-63, wherein the L protein is the L protein of Theiler's virus or a functional variant thereof.

Embodiment I-72. The method of embodiment I-63, wherein the L protein shares at least 90% identity to SEQ ID NO: 1.

Embodiment I-73. The method of embodiment I-63, wherein the L protein is the L protein of Encephalomyocarditis virus (EMCV) or a functional variant thereof.

Embodiment I-74. The method of embodiment I-63, wherein the L protein shares at least 90% identity to SEQ ID NO: 2.

Embodiment I-75. The method of embodiment I-63, wherein the L protein is selected from the group consisting of the L protein of poliovirus, the L protein of HRV16, the L protein of mengo virus, and the L protein of Saffold virus 2 or a functional variant thereof.

Embodiment I-76. The method of any one of embodiments I-59-75, wherein the system comprises a single vector comprising an expression cassette, the expression cassette comprising the first polynucleotide and the second polynucleotide.

Embodiment I-77. The method of embodiment I-76, wherein the expression cassette comprises a first promoter, operatively linked to the first polynucleotide; and a second promoter, operatively linked to the second polynucleotide.

Embodiment I-78. The method of embodiment I-76, wherein the expression cassette comprises a shared promoter operatively linked to both the first polynucleotide and the second polynucleotide.

Embodiment I-79. The method of embodiment I-78, wherein the expression cassette comprises a coding polynucleotide comprising the first polynucleotide and the second polynucleotide linked by a polynucleotide encoding ribosome skipping site, the coding polynucleotide operatively linked to the shared promoter.

Embodiment I-80. The method of embodiment I-78, wherein the expression cassette comprises a coding polynucleotide, the coding polynucleotide encoding the enhancer protein and the target protein linked to by a ribosome skipping site, the coding polynucleotide operatively linked to the shared promoter.

Embodiment I-81. The method of any one of embodiments I-76 to I-80, wherein the expression cassette is configured for transcription of a single messenger RNA encoding both the target protein and the enhancer protein, linked by a ribosome skipping site; wherein translation of the messenger RNA results in expression of the target protein and the L protein as distinct polypeptides.

Embodiment I-82. The method of any one of embodiments I-59 to I-75, wherein the system comprises one vector.

Embodiment I-83. The method of any one of embodiments I-59 to I-75, wherein the system comprises:
a) a first vector comprising the first polynucleotide, operatively linked to a first promoter; and
b) a second vector comprising the second polynucleotide, operatively linked to a second promoter.

Embodiment I-84. The method of any one of embodiments I-59 to I-75, wherein the system comprises two vectors.

Embodiment I-85. The method of any one of embodiments I-59 to I-84, wherein either the first polynucleotide or the second polynucleotide, or both, are operatively linked to an internal ribosome entry site (IRES).

Embodiment I-86. The method of any one of embodiments I-59 to I-85, wherein at least one of the one or more vectors comprises a T7 promoter configured for transcription of either or both of the first polynucleotide or the second polynucleotide by a T7 RNA polymerase.

Embodiment I-87. The method of any one of embodiments I-59 to I-86, wherein at least one of the one or more vectors comprises a polynucleotide sequence encoding a T7 RNA polymerase.

Embodiment I-88. A method of expressing a target protein in a subject in need thereof, comprising administering to the subject a vector, the vector comprising:
a) a first polynucleotide encoding the target protein; and
b) a second polynucleotide encoding an enhancer protein wherein:
   i) the enhancer protein is an inhibitor of nucleocytoplasmic transport (NCT) and/or
   ii) the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.
      wherein the first polynucleotide and the second polynucleotide are operatively linked to at least one promoter.

Embodiment I-89. The method of embodiment I-88, wherein the expression cassette comprises a first promoter, operatively linked to the first polynucleotide; and a second promoter, operatively linked to the second polynucleotide.

Embodiment I-90. The method of embodiment I-88, wherein the expression cassette comprises a shared promoter operatively linked to both the first polynucleotide and the second polynucleotide.

Embodiment I-91. The method of any one of embodiments I-59 to I-90, wherein the target protein is a therapeutic protein.

Embodiment I-92. The method of any one of embodiments I-59 to I-91, wherein the target protein is an immunogenic protein.

Embodiment I-93. The method of any one of embodiments I-59 to I-92, wherein the target protein is an antibody, a nanobody, a receptor, a bi-specific T-cell engager (BiTE), a growth factor, a hormone, an enzyme, an immunomodulatory protein, an antigen, a structural protein, a blood protein, an anti-microbial polypeptide, an anti-viral polypeptide, a tumor suppressor, a transcription factor, or a translation factor.

Embodiment I-94. The method of embodiment I-93, wherein the target protein is an antibody.

Embodiment I-95. The method of embodiment I-93, wherein the target protein is a blood protein.

Embodiment I-96. The method of any one of embodiments I-59-95, wherein the method elicits an immune response in the subject.

Embodiment I-97. The method of any one of embodiments I-59-96, wherein the method treats a disease in the subject, wherein the disease is caused by, correlated with, or associated with the target protein.

Embodiment I-98. The method of embodiment I-97, wherein the method treats a disease in the subject, wherein the expression levels of the target protein in the subject is lower than the expression levels of the target protein in a control subject, wherein the control subject does not have the disease.

Embodiment I-99. The method of any one of embodiments I-59 to I-98, wherein the target protein is selected from the group consisting of Abciximab, Alemtuzumab, Alirocumab, Amivantamab, Atezolizumab, Avelumab, Basiliximab, Belimumab, Benralizumab, Bevacizumab, Bezlotoxumab, Blinatumomab, Brentuximab vedotin, Brodalumab, Brolucizumab, Burosumab, Canakinumab, Caplacizumab, Capromab, Catumaxomab, Cemiplimab, Certolizumab pegol, Cetuximab, Crizanlizumab, Daclizumab, Daratumumab, Denosumab, Dinutuximab, Dupilumab, Durvalumab, Eculizumab, Elotuzumab, Emapalumab, Emicizumab, Enfortumab vedotin, Eptinezumab, Erenumab, Ertumaxomab, Etaracizumab, Evolocumab, Fremanezumab, Galcanezumab, Gemtuzumab ozogamicin, Golimumab, Guselkumab, Ibalizumab, Ibritumomab tiuxetan, Idarucizuma, Imciromab, Infliximab, Inotuzumab ozogamicin, Ipilimumab, Isatuximab, Itolizumab, Ixekizumab, Lanadelumab, Lokivetmab, Mepolizumab, Mogamulizumab, Moxetumomab Pasudotox, Natalizumab, Necitumumab, Nimotuzumab, Nivolumab, Obiltoxaximab, Obinutuzumab, Ocrelizumab, Ofatumumab, Olaratumab, Omalizumab, Palivizumab, Panitumumab, Pembrolizumab, Pertuzumab, Polatuzumab vedotin, Racotumomab, Ramucirumab, Ranibizumab, Raxibacumab, Ravulizumab, Reslizumab, Risankizumab, Rituximab, Rmab, Romosozumab, Rovelizumab, Ruplizumab, Sacituzumab govitecan, Sarilumab, Secukinumab, Siltuximab, Talquetamab, Teclistamab, Teprotumumab, Tildrakizumab, Tocilizumab, Tositumomab, Trastuzumab, Trastuzumab duocarmazine, Trastuzumab emtansine, Ustekinumab, and Vedolizumab, Blinatumomab, Emicizumab, Solitomab, adnectin, anticalin, avimer, fynomer, Kunitz domain, Knottin, Affibody, DARPin, a thrombolytic, transferrin, t-PA, hirudin, C1 esterase inhibitor, anti-thrombin, plasma kallikrein inhibitor, plasmin, pro-thrombin complex, complement components, Prealbumin (transthyretin), Alpha 1 antitrypsin, Alpha-1-acid glycoprotein, Alpha-1-fetoprotein, alpha2-macroglobulin, Gamma globulins, Beta-2 microglobulin, Haptoglobin, Ceruloplasmin, Complement component 3, Complement component 4, C-reactive protein (CRP), Lipoproteins (chylomicrons, VLDL, LDL, HDL), Transferrin, Prothrombin, mannose binding lectin (MBL), albumins, globulins, fibrinogen, regulatory factors, and coagulation factors, such as, Factor I, Factor II, Factor III, Factor IV, Factor V, Factor VI, Factor VII, Factor IX, Factor X, Factor XI, Factor XII, Factor XIII, von Willeband factor, prekallikrein, Fitzgerald factor, fibronectin, anti-thrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, cancer procoagulant, EPO, IGF-1, G-CSF, GM-GCF, BMP-2, BMP-7, KGF, PDGF-BB, TMP, Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Colony-stimulating factors, Macrophage colony-stimulating factor (M-CSF), Granulocyte colony-stimulating factor (G-CSF), Granulocyte macrophage colony-stimulating factor (GM-CSF), Epidermal growth factor (EGF), Ephrins-Ephrin A1, Ephrin A2, Ephrin A3, Ephrin A4, Ephrin A5, Ephrin B1, Ephrin B2, Ephrin B3, Erythropoietin (EPO), each of Fibroblast growth factor (FGF) 1, FGF2, FGF3, FGF4, FGF5, FGF6, FGF7, FGF8, FGF9, FGF10, FGF 11, FGF12, FGF13, FGF14, FGF15, FGF16, FGF17, FGF18, FGF19, FGF20, FGF21, FGF22, FGF23, Foetal Bovine Somatotrophin (FBS), GDNF family of ligands, Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factors, Insulin-like growth factor-1 (IGF-1), Insulin-like growth factor-2 (IGF-2), Interleukin-1 (IL-1), IL-2, IL-3, IL-4, IL-5, IL-6, IL-7, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1) Neuregulin 2 (NRG2), Neuregulin 3 (NRG3), Neuregulin 4 (NRG4), Neurotrophins, Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Vascular endothelial growth factor (VEGF), Wnt Signaling Pathway, glucagon like peptide-1, insulin, human growth hormone, follicle stimulating hormone, calcitonin, lutropin, glucagon like peptide-2, leptin, parathyroid hormone, chorionic gonadotropin, thyroid stimulating hormone, and glucagon, Alpha-glycosidase, glucocerebrosidase, iduronate-2-sulfate, alpha-galactosidase, urate oxidase, N-acetyl-galactosidase, carboxypeptidase, hyaluronidase, DNAse, asparaginase, uricase, adenosine deaminase and other enterokinases, cyclases, caspases, cathepsins, oxidoreductases, transferases, hydrolases, lyases, isomerases, and ligases, Agalsidase beta, Agalsidase alfa, Imiglucerase, Taligulcerase alfa, Velaglucerase alfa, Alglucerase, Sebelipase alpha, Laronidase, Idursulfase, Elosulfase alpha, Galsulfase, Alglucosidase alpha, C3 inhibitor, Hurler and Hunter corrective factors, ion channels, gap junctions, ionotropic receptors, transporters, cell surface receptors, signaling proteins, Dopamine receptor 1 (DRD1), Cystic fibrosis transmembrane conductance regulator (CFTR), C1 esterase inhibitor (C1-Inh), IL2 inducible T cell kinase (ITK), and NADase.

Embodiment I-100. The system of any one of embodiments I-1-29, the vector of any one of embodiments I-30-32, the eukaryotic cell of any one of embodiments I-33-35, the method of any one of embodiments I-36-39, the cell of embodiment I-40, the target protein of embodiments I-41, the method of any one of embodiments I-42-44, the vector system of any one of embodiments I-45-52, the method of any one of embodiments I-53-93 and 96-98, wherein the target protein is an antibody.

Embodiment I-101. The system of any one of embodiments I-1-29, the vector of any one of embodiments I-30-32, the eukaryotic cell of any one of embodiments I-33-35, the method of any one of embodiments I-36-39, the cell of embodiment I-40, the target protein of embodiments I-41, the method of any one of embodiments I-42-44, the vector system of any one of embodiments I-45-52, the method of any one of embodiments I-53-93 and 96-98, wherein the target protein is adalimumab.

Embodiment I-102. The system, vector, vector system, eukaryotic cell, method, cell, or target protein of embodiment I-101, wherein the heavy chain of adalimumab has an amino acid sequence of SEQ ID NO: 132.

Embodiment I-103. The system, vector, vector system, eukaryotic cell, method, cell, or target protein of embodiment I-101 or 102, wherein the light chain of adalimumab has an amino acid sequence of SEQ ID NO: 133.

Embodiment I-104. The system, vector, vector system, eukaryotic cell, method, cell, or target protein of any one of embodiments I-101-103, wherein the heavy chain of adalimumab is encoded by a nucleic acid sequence of SEQ ID NO: 134.

Embodiment I-105. The system, vector, vector system, eukaryotic cell, method, cell, or target protein of any one of embodiments I-101-104, wherein the light chain of adalimumab is encoded by a nucleic acid sequence of SEQ ID NO: 135.

Embodiment I-106. The method of any of embodiments I-88-99, wherein the enhancer protein increases the activity of the target protein.

Embodiment I-107. The method of any of embodiments I-88-99 and 106, wherein the enhancer protein lowers the expression level of the target protein.

Embodiment I-108. The method of any of embodiments I-88-99 and 106-107, wherein the enhancer protein increases the uniformity of expression of the target protein in vivo.

Embodiment I-109. The method of any of embodiments I-88-99 and 106-107, wherein the enhancer protein increases the duration of active target protein in the cell or organism.

Embodiment I-110. A lipid nanoparticle (LNP) comprising the vector of any one of embodiments I-30-32 and one or more lipids.

Embodiment I-111. A polynucleotide encoding a Leader protein and an adalimumab protein.

Embodiment I-112. The polynucleotide of embodiment I-111, wherein the polynucleotide encodes a Leader protein with an amino acid sequence selected from the group consisting of SEQ ID NOS: 1-6, and 24, or an amino acid sequence with at least 70%, at least 75%, at least 80%, at least 85%, at least 90%, at least 95%, at least 96%, at least 97%, at least 98%, or at least 99% sequence identity thereto.

Embodiment I-113. The polynucleotide sequence of embodiments I-111 or 112 wherein the polynucleotide encodes an adalimumab variable heavy chain sequence of SEQ ID NO: 124, or an amino acid sequence with at least 70%, at least 75%, at least 80%, at least 85%, at least 90%, at least 95%, at least 96%, at least 97%, at least 98%, or at least 99% sequence identity thereto; and an adalimumab variable light chain sequence of SEQ ID NO: 129 or an amino acid sequence with at least 70%, at least 75%, at least 80%, at least 85%, at least 90%, at least 95%, at least 96%, at least 97%, at least 98%, or at least 99% sequence identity thereto.

Embodiment I-114. The polynucleotide of any one of embodiments I-111-113, wherein the co-expression of the Leader protein and the adalimumab protein reduces expression level of the adalimumab protein in a cell or a subject by about 10%, about 20%, about 30%, about 40%, about 50%, about 60%, about 70%, about 80%, or about 90%.

Embodiment I-120. The polynucleotide of embodiment I-111, wherein the polynucleotide comprises the sequences of the set of SEQ ID NOS: 191-216 or the sequences of the set of SEQ ID NOS: 217-242.

Embodiment I-121. A vector comprising the polynucleotide of embodiment I-111.

Embodiment I-122. The vector of embodiment I-121 wherein the vector is an Adeno-associated virus (AAV) vector.

Embodiment I-123. A system comprising the transfer plasmid of embodiment I-121 and one or more polynucleotides encoding adenovirus genes E4, E2A, VA, and a Cap protein of AAV.

Embodiment I-124. A lipid nanoparticle (LNP) comprising the vector of embodiment I-120.

Embodiment I-125. The LNP of embodiment I-123, wherein the LNP comprises, the LNP comprises a PEGylated lipid, a cholesterol, and one or more ionizable lipids.

Embodiment I-126. The LNP of embodiment I-123, wherein the LNP comprises about 0.5% to about 2% PEGylated lipid, about 35% to about 45% cholesterol, and about 5% to about 65% one or more ionizable lipids.

Embodiment I-127. The LNP of embodiment I-123, wherein the LNP comprises DMG-PEG(2000), cholesterol, DOPC and DLin-KC2-DMA in a ratio of about 1% DMG-PEG(2000), to about 40% cholesterol, to about 10% DOPC and about 50% DLin-KC2-DMA.

Embodiment I-128. A method of treatment for a subject in need thereof, comprising delivering the system of embodiment I-122 and/or the LNP of any one of embodiments I-123-126.

Embodiment I-129. The method of embodiment I-127, wherein the system is delivered intramuscularly or subcutaneously.

Embodiment I-130. A polynucleotide encoding a Leader protein and a Glucosylceramidase (GBA) protein.

Embodiment I-131. The polynucleotide of embodiment I-130, wherein the polynucleotide encodes a Leader protein with an amino acid sequence selected from the group consisting of SEQ ID NOS: 1-6, and 24, or an amino acid sequence with at least 70%, at least 75%, at least 80%, at least 85%, at least 90%, at least 95%, at least 96%, at least 97%, at least 98%, or at least 99% sequence identity thereto.

Embodiment I-132. The polynucleotide of embodiments I-130 or 131 wherein the polynucleotide encodes a GBA amino acid sequence of SEQ ID NO: 406, or an amino acid sequence with at least 70%, at least 75%, at least 80%, at least 85%, at least 90%, at least 95%, at least 96%, at least 97%, at least 98%, or at least 99% sequence identity thereto.

Embodiment I-139. A vector comprising the polynucleotide of embodiment I-130.

Embodiment I-140. The vector of embodiment I-139 wherein the vector is an Adeno-associated virus (AAV) vector.

Embodiment I-141. A system comprising the transfer plasmid of embodiment I-140 and one or more polynucleotides encoding adenovirus genes E4, E2A, VA, and a Cap protein of AAV.

Embodiment I-142. A lipid nanoparticle (LNP) comprising the vector of embodiment I-139.

Embodiment I-143. The LNP of embodiment I-142, wherein the LNP comprises, the LNP comprises a PEGylated lipid, a cholesterol, and one or more ionizable lipids.

Embodiment I-144. The LNP of embodiment I-142, wherein the LNP comprises about 0.5% to about 2% PEGylated lipid, about 35% to about 45% cholesterol, and about 5% to about 65% one or more ionizable lipids.

Embodiment I-145. The LNP of embodiment I-142, wherein the LNP comprises DMG-PEG(2000), cholesterol, DOPC and DLin-KC2-DMA in a ratio of about 1% DMG-PEG(2000), to about 40% cholesterol, to about 10% DOPC and about 50% DLin-KC2-DMA.

Embodiment I-146. A method of treatment for a subject in need thereof, comprising delivering the system of embodiment I-141 and/or the LNP of any one of embodiments I-142-145.

Embodiment I-147. The method of embodiment I-146, wherein the system is delivered intramuscularly or subcutaneously.

Embodiment I-148. A polynucleotide encoding a Leader protein and a target protein.

Embodiment I-149. The polynucleotide of embodiment I-130, wherein the polynucleotide encodes a Leader protein with an amino acid sequence selected from the group consisting of SEQ ID NOS: 1-6, and 24, or an amino acid sequence with at least 70%, at least 75%, at least 80%, at least 85%, at least 90%, at least 95%, at least 96%, at least 97%, at least 98%, or at least 99% sequence identity thereto.

Embodiment I-150. The polynucleotide of embodiments I-130 or 131 wherein the polynucleotide encodes a target protein amino acid sequence any of SEQ ID NOS: 124, 129, 374-405, and/or any of SEQ ID NOS: 406-422, or an amino acid sequence with at least 70%, at least 75%, at least 80%, at least 85%, at least 90%, at least 95%, at least 96%, at least 97%, at least 98%, or at least 99% sequence identity thereto.

Embodiment I-157. A vector comprising the polynucleotide of embodiment I-130.

Embodiment I-158. The vector of embodiment I-139 wherein the vector is an Adeno-associated virus (AAV) vector.

Embodiment I-159. A system comprising the transfer plasmid of embodiment I-140 and one or more polynucleotides encoding adenovirus genes E4, E2A, VA, and a Cap protein of AAV.

Embodiment I-160. A lipid nanoparticle (LNP) comprising the vector of embodiment I-139.

Embodiment I-161. The LNP of embodiment I-142, wherein the LNP comprises, the LNP comprises a PEGylated lipid, a cholesterol, and one or more ionizable lipids.

Embodiment I-162. The LNP of embodiment I-142, wherein the LNP comprises about 0.5% to about 2% PEGylated lipid, about 35% to about 45% cholesterol, and about 5% to about 65% one or more ionizable lipids.

Embodiment I-163. The LNP of embodiment I-142, wherein the LNP comprises DMG-PEG(2000), cholesterol, DOPC and DLin-KC2-DMA in a ratio of about 1% DMG-PEG(2000), to about 40% cholesterol, to about 10% DOPC and about 50% DLin-KC2-DMA.

Embodiment I-164. A method of treatment for a subject in need thereof, comprising delivering the system of embodiment I-141 and/or the LNP of any one of embodiments I-142-145.

Embodiment I-165. The method of embodiment I-146, wherein the system is delivered intramuscularly or subcutaneously.

### Embodiments II

Embodiment II-52. A method of expressing an adalimumab protein in a subject in need thereof, comprising administering to the subject a vector system comprising one or more vectors, the one or more vectors, comprising:
a) a first polynucleotide encoding an adalimumab protein; and
b) a second polynucleotide encoding a picornavirus leader (L) protein with an amino acid sequence selected from the group consisting of SEQ ID NOS: 1-6, and 24, or an amino acid sequence with at least 70%, at least 75%, at least 80%, at least 85%, at least 90%, at least 95%, at least 96%, at least 97%, at least 98%, or at least 99% sequence identity thereto; wherein the first polynucleotide encoding the adalimumab protein and the second polynucleotide encoding the L protein are operatively linked to one or more promoters; and wherein the adalimumab protein and the L protein are co-expressed.

Embodiment II-53. The method of any 52, wherein the first polynucleotide encodes an adalimumab variable heavy chain sequence of SEQ ID NO: 124, or an amino acid sequence with at least 70%, at least 75%, at least 80%, at least 85%, at least 90%, at least 95%, at least 96%, at least 97%, at least 98%, or at least 99% sequence identity thereto; and an adalimumab variable light chain sequence of SEQ ID NO: 129 or an amino acid sequence with at least 70%, at least 75%, at least 80%, at least 85%, at least 90%, at least 95%, at least 96%, at least 97%, at least 98%, or at least 99% sequence identity thereto.

Embodiment II-54. The method of embodiments II-52 to II-53, wherein the co-expression of the leader protein and the adalimumab protein reduces the expression level of the adalimumab protein in a cell or a subject by about 10%, about 20%, about 30%, about 40%, about 50%, about 60%, about 70%, about 80%, or about 90%.

Embodiment II-55. The method of embodiments II-52 to II-54, wherein the co-expression of the leader protein and the adalimumab protein increases the activity of the adalimumab protein in a cell of the subject or the subject by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment II-56. The method of embodiments II-52 to II-55, wherein the co-expression of the leader protein and the adalimumab protein increases the duration of time in which the adalimumab protein is found in a cell of the subject or the subject by about 2-fold, about 3-fold, about 4-fold, about 5-fold, about 6-fold, about 7-fold, about 8-fold, about 9-fold, about 10, about 11-fold, about 12-fold, about 13-fold, about 14-fold, about 15-fold, about 16-fold, about 17-fold, about 18-fold, about 19-fold, or about 20-fold.

Embodiment II-57. The method of any one of embodiments II-52 to II-56, wherein the co-expression of the leader protein and the adalimumab protein increases the coefficient of variation (CV %) of the target protein in the tissue of the subject or the subject by about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.7-fold, about 2.8-fold, about 2.9-fold, or about 3-fold.

Embodiment II-58. The method of any one of embodiments II-52 to II-57, wherein the co-expression of the leader protein and the adalimumab protein reduces the degradation of the target protein by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment II-59. The method of any one of embodiments II-52 to II-58, wherein the co-expression of the leader protein and the adalimumab protein reduces the EC₅₀ of adalimumab by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment II-60. The method of any one of embodiments II-52 to II-59, wherein the vector system comprises the polynucleotide sequences of the set of SEQ ID NOS: 191-216 or the sequences of the set of SEQ ID NOS: 217-242.

Embodiment II-61. The method of any one of embodiments II-52 to II-60, wherein the vector system comprises one or more polynucleotides encoding adenovirus genes E4, E2A, VA, and a Cap protein of AAV.

Embodiment II-62. The method of any one of embodiments II-52 to II-61, wherein the vector system is administered via a lipid nanoparticle (LNP).

Embodiment II-63. The method of embodiment II-62, wherein the LNP comprises a PEGylated lipid, a cholesterol, and one or more ionizable lipids.

Embodiment II-64. The method of embodiment II-62, wherein the LNP comprises about 0.5% to about 2% PEGylated lipid, about 35% to about 45% cholesterol, and about 5% to about 65% one or more ionizable lipids.

Embodiment II-65. The method of embodiment II-62, wherein the LNP comprises DMG-PEG(2000), cholesterol, DOPC and DLin-KC2-DMA in a ratio of about 1% DMG-PEG(2000), to about 40% cholesterol, to about 10% DOPC and about 50% DLin-KC2-DMA.

Embodiment II-66. The method of any one of embodiments II-52 to II-65, wherein the system is delivered intramuscularly or subcutaneously.

Embodiment II-67. A method of expressing a Glucosylceramidase (GBA) protein in a subject in need thereof, comprising administering to the subject a vector system comprising one or more vectors, the one or more vectors, comprising:
a) a first polynucleotide encoding a Glucosylceramidase (GBA) protein; and
b) a second polynucleotide encoding a picornavirus leader (L) protein with an amino acid sequence selected from the group consisting of SEQ ID NOS: 1-6, and 24, or an amino acid sequence with at least 70%, at least 75%, at least 80%, at least 85%, at least 90%, at least 95%, at least 96%, at least 97%, at least 98%, or at least 99% sequence identity thereto; wherein the first polynucleotide encoding the Glucosylceramidase (GBA) protein and the second polynucleotide encoding the L protein are operatively linked to one or more promoters; and wherein the GBA protein and the L protein are co-expressed.

Embodiment II-68. The method of embodiment II-67, wherein the first polynucleotide encodes a GBA amino acid sequence of SEQ ID NO: 406, or an amino acid sequence with at least 70%, at least 75%, at least 80%, at least 85%, at least 90%, at least 95%, at least 96%, at least 97%, at least 98%, or at least 99% sequence identity thereto.

Embodiment II-69. The method of embodiments II-67 to II-68, wherein the co-expression of the leader protein and the GBA protein reduces expression level of the GBA protein in a cell of the subject or the subject by about 10%, about 20%, about 30%, about 40%, about 50%, about 60%, about 70%, about 80%, or about 90%.

Embodiment II-70. The method of any one of embodiments II-67 to II-69, wherein the co-expression of the leader protein and the GBA protein increases the activity of GBA in a cell of the subject or the subject by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment II-71. The method of any one of embodiments II-67 to II-70, wherein the co-expression of the leader protein and the GBA protein increases the duration of time in which GBA is found in a cell of the subject or the subject by about 2-fold, about 3-fold, about 4-fold, about 5-fold, about 6-fold, about 7-fold, about 8-fold, about 9-fold, about 10, about 11-fold, about 12-fold, about 13-fold, about 14-fold, about 15-fold, )about 16-fold, about 17-fold, about 18-fold, about 19-fold, or about 20-fold.

Embodiment II-72. The method of any one of embodiments II-67 to II-71, wherein the co-expression of the enhancer protein increases the coefficient of variation (CV %) of GBA in a tissue of the subject or the subject by about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.7-fold, about 2.8-fold, about 2.9-fold, or about 3-fold.

Embodiment II-73. The method of any one of embodiments II-67 to II-72, wherein the co-expression of the leader protein and the GBA protein reduces the degradation of GBA by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment II-74. The method of any one of embodiments II-67 to II-73, wherein the co-expression of the leader protein and the GBA protein reduces the concentration of GBA effective in producing 50% of the maximal response (EC₅₀).

Embodiment II-75. The method of any one of embodiments II-67 to II-74, wherein the vector system comprises one or more polynucleotides encoding adenovirus genes E4, E2A, VA, and a Cap protein of AAV.

Embodiment II-76. The method of any one of embodiments II-67 to II-75, wherein the vector system is administered via a lipid nanoparticle (LNP).

Embodiment II-77. The method of embodiment II-76, wherein the LNP comprises a PEGylated lipid, a cholesterol, and one or more ionizable lipids.

Embodiment II-78. The method of embodiment II-76, wherein the LNP comprises about 0.5% to about 2% PEGylated lipid, about 35% to about 45% cholesterol, and about 5% to about 65% one or more ionizable lipids.

Embodiment II-79. The method of embodiment II-76, wherein the LNP comprises DMG-PEG(2000), cholesterol, DOPC and DLin-KC2-DMA in a ratio of about 1% DMG-PEG(2000), to about 40% cholesterol, to about 10% DOPC and about 50% DLin-KC2-DMA.

Embodiment II-80. The method of any one of embodiments II-67 to II-79, wherein the system is delivered intramuscularly or subcutaneously.

Embodiment II-81. A method of expressing a target protein in a subject in need thereof, comprising administering to the subject a vector system comprising one or more vectors, the one or more vectors, comprising:
a) a first polynucleotide encoding a target protein; and
b) a second polynucleotide encoding a picornavirus leader (L) protein with an amino acid sequence selected from the group consisting of SEQ ID NOS: 1-6, and 24, or an amino acid sequence with at least 70%, at least 75%, at least 80%, at least 85%, at least 90%, at least 95%, at least 96%, at least 97%, at least 98%, or at least 99% sequence identity thereto; wherein the first polynucleotide encoding the target protein and the second polynucleotide encoding the L protein are operatively linked to one or more promoters; and wherein the target protein and the L protein are co-expressed.

Embodiment II-82. The method of embodiment II-81, wherein the first polynucleotide encodes a variable heavy chain sequence of Table 8, or an amino acid sequence with at least 70%, at least 75%, at least 80%, at least 85%, at least 90%, at least 95%, at least 96%, at least 97%, at least 98%, or at least 99% sequence identity thereto; and/or a variable light chain sequence of Table 8 or an amino acid sequence with at least 70%, at least 75%, at least 80%, at least 85%, at least 90%, at least 95%, at least 96%, at least 97%, at least 98%, or at least 99% sequence identity thereto.

Embodiment II-83. The method of embodiment II-81, wherein the first polynucleotide encodes protein sequence of Table 9, or an amino acid sequence with at least 70%, at least 75%, at least 80%, at least 85%, at least 90%, at least 95%, at least 96%, at least 97%, at least 98%, or at least 99% sequence identity thereto.

Embodiment II-84. The method of any one of embodiments II-81 to II-83, wherein the co-expression of the leader protein and the target protein reduces the expression level of the target protein in a cell or a subject by about 10%, about 20%, about 30%, about 40%, about 50%, about 60%, about 70%, about 80%, or about 90%.

Embodiment II-85. The method of any one of embodiments II-81 to II-84, wherein the co-expression of the leader protein and the target protein increases the activity of the target protein in a cell of the subject or the subject by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment II-86. The method of any one of embodiments II-81 to II-85, wherein the co-expression of the leader protein and the target protein increases the duration of time in which the target protein is found in a cell of the subject or the subject by about 2-fold, about 3-fold, about 4-fold, about 5-fold, about 6-fold, about 7-fold, about 8-fold, about 9-fold, about 10, about 11-fold, about 12-fold, about 13-fold, about 14-fold, about 15-fold, about 16-fold, about 17-fold, about 18-fold, about 19-fold, or about 20-fold.

Embodiment II-87. The method of any one of embodiments II-81 to II-85, wherein the co-expression of the leader protein and the target protein increases the coefficient of variation (CV %) of the target protein in the tissue of the subject or the subject by about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.7-fold, about 2.8-fold, about 2.9-fold, or about 3-fold.

Embodiment II-88. The method of any one of embodiments II-81 to II-87, wherein the co-expression of the leader protein and the target protein reduces the degradation of the target protein by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment II-89. The method of any one of embodiments II-81 to II-88, wherein the co-expression of the leader protein and the target protein reduces the EC₅₀ of target by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment II-90. The method of any one of embodiments II-81 to II-89, wherein the vector system comprises one or more polynucleotides encoding adenovirus genes E4, E2A, VA, and a Cap protein of AAV.

Embodiment II-91. The method of any one of embodiments II-81 to II-90, wherein the vector system is administered via a lipid nanoparticle (LNP).

Embodiment II-92. The method of any one of embodiments II-81 to II-91, wherein the LNP comprises a PEGylated lipid, a cholesterol, and one or more ionizable lipids.

Embodiment II-93. The method of embodiment II-92, wherein the LNP comprises about 0.5% to about 2% PEGylated lipid, about 35% to about 45% cholesterol, and about 5% to about 65% one or more ionizable lipids.

Embodiment II-94. The method of embodiment II-92, wherein the LNP comprises DMG-PEG(2000), cholesterol, DOPC and DLin-KC2-DMA in a ratio of about 1% DMG-PEG(2000), to about 40% cholesterol, to about 10% DOPC and about 50% DLin-KC2-DMA.

Embodiment II-95. The method of any one of embodiments II-81 to II-94, wherein the system is delivered intramuscularly or subcutaneously.

Embodiment II-96. A vector system for use in a method according to any preceding embodiment.

### EXAMPLES

### Materials and Methods for In Vitro Studies

Construction of DNA Molecules

All assemblies were made into a plasmid backbone capable of propagation in E. coli comprising a promoter controlling a high copy number origin of replication (ColE1) followed by a terminator (rrnB T1 and T2 terminator). This is followed by a promoter controlling an antibiotic resistance gene which is isolated from the rest of the vector by a second terminator (transcription terminator from phage lambda). The genes comprising elements of the backbone were synthesized by phosphoramidite chemistry.

Structure genes used for the construction of the plasmids were synthesized by phosphoramidite chemistry, chemistry, amplified and cloned into the vector described above using an isothermal assembly reaction such as NEB HI-FI or Gibson Assembly using the primers listed in Table 2. Select amino acid sequences comprised by the illustrative constructs employed in these examples are provided in Table 3.

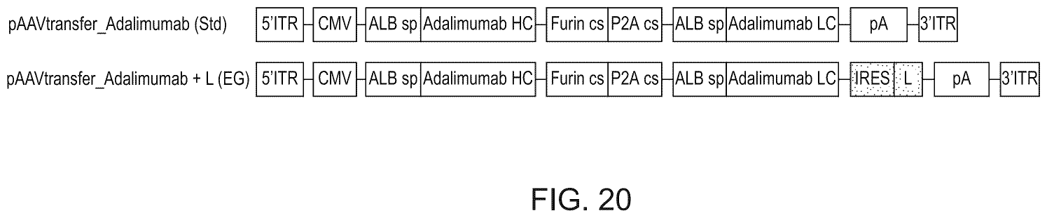

TABLE 2
Construct design

| | | | Used in |
|---|---|---|---|
| Construct | Schematic | Primer | Example |
| EG1 | FIG. 2A | 1a.) | 1 |
| | | gcccgggatccaccggtcgccacca | |
| | | tggtgagcaagggcgaggagc | |
| | | (SEQ ID NO: 25) | |
| | | 1b.) | |
| | | agatggctggcaactagaaggcaca | |
| | | gttacttgtacagctcgtccatgcc | |
| | | gag | |
| | | (SEQ ID NO: 26) | |
| | | 2a.) | |
| | | cactctcggcatggacgagctgtac | |
| | | aagtaactgtgccttctagttgcca | |
| | | gccatctgt | |
| | | (SEQ ID NO: 27) | |
| | | 2b.) | |
| | | cagctcctcgcccttgctcaccatg | |
| | | gtggcgaccggtggatccc | |
| | | (SEQ ID NO: 28) | |
| EG2 | FIG. 2B | 1a.) | 2 |
| | | cggccagtaacgttagggggggggg | |
| | | attacttgtacagctcgtccatgcc | |
| | | gag | |
| | | (SEQ ID NO: 29) | |
| | | 1b.) | |
| | | cggtaccgcgggcccgggatccacc | |
| | | ggtcgccaccatggtgagcaagggc | |
| | | gaggagc | |
| | | (SEQ ID NO: 30) | |
| | | 2a.) | |
| | | cactctcggcatggacgagctgtac | |
| | | aagtaactgtgccttctagttgcca | |
| | | gccatctgt | |
| | | (SEQ ID NO: 27) | |
| | | 2b.) | |
| | | cagctcctcgcccttgctcaccatg | |
| | | gtggcgaccggtggatccc | |
| | | (SEQ ID NO: 28) | |
| | | 3a.) | |
| | | ctcggcatggacgagctgtacaagt | |
| | | aatcccccccccctaacgttactgg | |
| | | (SEQ ID NO: 31) | |
| | | 3b.) | |
| | | acgggggaggggcaaacaacagatg | |
| | | gctggcaactagaaggcacagctgt | |
| | | aactcgaaaacgacttccatgtcta | |
| | | attcgg | |
| | | (SEQ ID NO: 32) | |
| EG3 | FIG. 2C | 1a.) | 1 |
| + | | cgcgggcccgggatccaccggtcgc | |
| EG4 | | caccATGAACACCATCAATATTGCC | |
| | | AAGAACGACTTTTCTGACATCG | |
| | | (SEQ ID NO: 33) | |
| | | 1b.) | |
| | | agatggctggcaactagaaggcaca | |
| | | gttagggTCAGGCAAATGCGAAATC | |
| | | GGACTCCAG | |
| | | (SEQ ID NO: 34) | |
| | | 2a.) | |
| | | CCTGGAGTCCGATTTCGCATTTGCC | |
| | | TGAccctaactgtgccttctagttg | |
| | | cca | |
| | | gccatctgt | |
| | | (SEQ ID NO: 35) | |
| | | 2b.) | |
| | | CGTTCTTGGCAATATTGATGGTGTT | |
| | | CATggtggcgaccggtggatcccgg | |
| | | gcc | |
| | | (SEQ ID NO: 36) | |
| | | 3a) | |
| | | accttggccgactctggtaatgGT | |
| | | AATACGACTCACTATAGGaaaaa | |
| | | (SEQ ID NO: 37) | |
| | | 3b.agtcagtgagcgaggaagccC | |
| | | AAAAAACCCCTCAAGACCCGTTTA | |
| | | (SEQ ID NO: 38) | |
| | | 4a) | |
| | | AAACGGGTCTTGAGGGGTTTTTT | |
| | | Gggcttcctcgctcactgac | |
| | | (SEQ ID NO: 39) | |
| | | 4b) | |
| | | TAGTGAGTCGTATTACcattacc | |
| | | agagtcggccaaggt | |
| | | (SEQ ID NO: 40) | |
| EG5 | FIG. 2D | 1a) | 2 |
| | | gcccgggatccaccggtcgccacct | |
| | | cgccaccatgaggactctgaacacc | |
| | | tctgccatgg | |
| | | (SEQ ID NO: 41) | |
| | | 1b) | |
| | | CTTTTCGAACTGCGGGTGGCTCCAG | |
| | | AGCGGCCGCGTtccCGTggttgggt | |
| | | gctgaccgttttgtgtg | |
| | | (SEQ ID NO: 42) | |
| | | 2a) | |
| | | ACGCGGCCGCTCTGGAGCCACCCGC | |
| | | AGTTCGAAAAGtaaagcggccgcga | |
| | | ctctagatca | |
| | | (SEQ ID NO: 43) | |
| | | 2b) | |
| | | gtgttcagagtcctcatggtggcga | |
| | | ggtggcgacc | |
| | | (SEQ ID NO: 44) | |
| EG6 | FIG. 2E | 1a.) | 2 |
| | | atccaccggtcgccaccatgaggac | |
| | | tctgaacacctctgccatgg | |
| | | (SEQ ID NO: 45) | |
| | | 1b.) | |
| | | tgtggtatggctgattatgatttac | |
| | | tgtaactcgaaaacgacttccatgt | |
| | | ctaattcggg | |
| | | (SEQ ID NO: 46) | |
| | | 2a.) | |
| | | gttttcgagttacagtaaatcataa | |
| | | tcagccataccacatttgtagaggt | |
| | | tttacttgct | |
| | | (SEQ ID NO: 47) | |
| | | 2b.) | |
| | | tggcagaggtgttcagagtcctcat | |
| | | ggtggcgaccggtgg | |
| | | (SEQ ID NO: 48) | |
| EG7 | FIG. 2F | | 2 |
| EG8 | FIG. 2G | 1a.) | 2, 5 |
| | | atccaccggtcgccaccatgaggac | |
| | | tctgaacacctctgccatgg | |
| | | (SEQ ID NO: 45) | |
| | | 1b.) | |
| | | cggccagtaacgttagggggggggg | |
| | | attacttgtacagctcgtccatgcc | |
| | | gag | |
| | | (SEQ ID NO: 29) | |
| | | 2a.) | |
| | | ctcggcatggacgagctgtacaagt | |
| | | aatcccccccccctaacgttactgg | |
| | | (SEQ ID NO: 31) | |
| | | 2b.) | |
| | | tggcagaggtgttcagagtcctcat | |
| | | ggtggcgaccggtgg | |
| | | (SEQ ID NO: 48) | |
| EG9 | FIG. 2H | 1a) | 8 |
| | | CACCATCACCATCACCATGTTatgg | |
| | | ccacaaccatggaacaagaga | |
| | | ctt | |
| | | (SEQ ID NO: 49) | |
| | | 1b) | |
| | | tcttgatgagctgttcttccaggag | |
| | | gataaagttgttcatggtggcgacc | |
| | | ggtggatccc | |
| | | (SEQ ID NO: 50) | |
| | | 2a) | |
| | | cgggcccgggatccaccggtcgcca | |
| | | ccatgaacaactttatcctcctgga | |
| | | agaacagctc | |
| | | (SEQ ID NO: 51) | |
| | | 2b) | |
| | | aagtctcttgttccatggttgtggc | |
| | | catAACATGGTGATGGTGATGGTG | |
| | | (SEQ ID NO: 52) | |
| EG10 | FIG. 2I | 1a.) | 2 |
| + | | gtgttcagagtcctcatggtggcga | |
| EG11 | | ggtggcgacc | |
| | | (SEQ ID NO: 44) | |
| | | 1b.) | |
| | | CTCTCGGCATGGACGAGCTGTACAAG | |
| | | (SEQ ID NO: 53) | |
| | | 2a.) | |
| | | ttaCTTGTACAGCTCGTCCATGCCGA | |
| | | GAG | |
| | | (SEQ ID NO: 54) | |
| | | 2b.) | |
| | | gcccgggatccaccggtcgccacct | |
| | | cgccaccatgaggactctgaacacc | |
| | | tctgccatgg | |
| | | (SEQ ID NO: 41) | |
| | | 3a) | |
| | | tgcgcgcaagtctcttgttccatgg | |
| | | ttgtggccatggtggcgaccggtgg | |
| | | atccc | |
| | | (SEQ ID NO: 55) | |
| | | 3b) | |
| | | cccgaattagacatggaagtcgttt | |
| | | tcgagttacag | |
| | | (SEQ ID NO: 56) | |
| | | 4a) | |
| | | gggatccaccggtcgccaccatggc | |
| | | cacaaccatggaacaagagacttg | |
| | | (SEQ ID NO: 57) | |
| | | 4b) | |
| | | ctgtaactcgaaaacgacttccatg | |
| | | tctaattcggg | |
| | | (SEQ ID NO: 58) | |
| EG12 | FIG. 2J | 1a) | 3 |
| + | | tctcttgttccatggttgtggccat | |
| EG4 | | ggtggcgaccggtgg | |
| | | (SEQ ID NO: 59) | |
| | | 1b) | |
| | | acgtggttttcctttgaaaaacacg | |
| | | atgataaatgaggactctgaacacc | |
| | | tctgccatgg | |
| | | (SEQ ID NO: 60) | |
| | | 2a) | |
| | | gcagaggtgttcagagtcctcattt | |
| | | atcatcgtgtttttcaaaggaaaac | |
| | | cacg | |
| | | (SEQ ID NO: 61) | |
| | | 2b) | |
| | | agtcgttttcgagttacagtaatcc | |
| | | cccccccctaacgttactgg | |
| | | (SEQ ID NO: 62) | |
| | | 3a) | |
| | | ccagtaacgttaggggggggggatt | |
| | | actgtaactcgaaaacgacttccat | |
| | | gt | |
| | | (SEQ ID NO: 63) | |
| | | 3b) | |
| | | ccaccggtcgccaccatggccacaa | |
| | | ccatggaacaagag | |
| | | (SEQ ID NO: 64) | |
| EG10 | FIG. 2K | 1a.) | 2, 3, 4 |
| | | gtgttcagagtcctcatggtggcga | |
| | | ggtggcgacc | |
| | | (SEQ ID NO: 44) | |
| | | 1b.) | |
| | | CTCTCGGCATGGACGAGCTGTACAAG | |
| | | (SEQ ID NO: 53) | |
| | | 2a.) | |
| | | ttaCTTGTACAGCTCGTCCATGCCGA | |
| | | GAG | |
| | | (SEQ ID NO: 54) | |
| | | 2b.) | |
| | | gcccgggatccaccggtcgccacct | |
| | | cgccaccatgaggactctgaacacc | |
| | | tctgccatgg | |
| | | (SEQ ID NO: 41) | |
| EG13 | FIG. 2L | 1a.) | 11 |
| | | cgggcccgggatccaccggtcgcca | |
| | | ccatgaacaactttatcctcctgga | |
| | | agaacagctc | |
| | | (SEQ ID NO: 51) | |
| | | 1b.) | |
| | | GATGGTGTCCCCCGCCACCTCCGCC | |
| | | ACCTCCaagtcctgattctgcaatt | |
| | | tcagcc | |
| | | agtt | |
| | | (SEQ ID NO: 65) | |
| | | 2a.) | |
| | | aattgcagaatcaggacttGGAGGT | |
| | | GGCGGAGGTGGCGGGGGACACCATC | |
| | | ACCATCACCATGTTTAAtccccccc | |
| | | ccctaacgttactgg | |
| | | (SEQ ID NO: 66) | |
| | | 2b.) | |
| | | tcttgatgagctgttcttccaggag | |
| | | gataaagttgttcatggtggcgacc | |
| | | ggtggatccc | |
| | | (SEQ ID NO: 50) | |
| EG14 | FIG. 2M | 1a.) | 11 |
| | | ttataggcggacagcagcagggtca | |
| | | gcaccatggtggcgaggtggcgacc | |
| | | (SEQ ID NO: 67) | |
| | | 1b.) | |
| | | CGGCCGCTCGATTACAAGGATGACG | |
| | | ACGATAAGGTTTAAagcggccgcga | |
| | | ctctagatca | |
| | | (SEQ ID NO: 68) | |
| | | 2a.) | |
| | | TAAACCTTATCGTCGTCATCCTTGT | |
| | | AATCGAGCGGCCGCGTtgtagggcc | |
| | | catgggggcg | |
| | | (SEQ ID NO: 69) | |
| | | 2b.) | |
| | | gcgggcccgggatccaccggtcgcc | |
| | | acctcgccaccatggtgctgaccct | |
| | | gctgctgtcc | |
| | | (SEQ ID NO: 70) | |
| EG15 | FIG. 2N | 1a) | 7 |
| | | ccctgtcttcatggggcgagtatat | |
| | | gaccccagggccGGAGGTGGCGGAG | |
| | | GTGGC | |
| | | (SEQ ID NO: 71) | |
| | | 1b) | |
| | | ggagggtcagcagggtcagcctgga | |
| | | ggccatggtggcgaccggtggatcc | |
| | | (SEQ ID NO: 72) | |
| | | 2a) | |
| | | cggtaccgcgggcccgggatccacc | |
| | | ggtcgccaccatggcctccaggctg | |
| | | accctg | |
| | | (SEQ ID NO: 73) | |
| | | 2b) | |
| | | TGGTGTCCCCCGCCACCTCCGCCAC | |
| | | CTCCggccctggggtcatatactcg | |
| | | cc | |
| | | (SEQ ID NO: 74) | |
| EG16 | FIG. 20 | 1a) | 7 |
| | | ccctgtcttcatggggcgagtatat | |
| | | gaccccagggccGGAGGTGGCGGAG | |
| | | GTGGC | |
| | | (SEQ ID NO: 71) | |
| | | 1b) | |
| | | ggagggtcagcagggtcagcctgga | |
| | | ggccatggtggcgaccggtggatcc | |
| | | (SEQ ID NO: 72) | |
| | | 2a) | |
| | | cggtaccgcgggcccgggatccacc | |
| | | ggtcgccaccatggcctccaggctg | |
| | | accctg | |
| | | (SEQ ID NO: 73) | |
| | | 2b) | |
| | | TGGTGTCCCCCGCCACCTCCGCCAC | |
| | | CTCCggccctggggtcatatactcg | |
| | | cc | |
| | | (SEQ ID NO: 74) | |
| EG17 | FIG. 2P | 1a.) | 8, 9, 10 |
| | | cgggcccgggatccaccggtcgcca | |
| | | ccatgaacaactttatcctcctgga | |
| | | agaacagctc | |
| | | (SEQ ID NO: 51) | |
| | | 1b.) | |
| | | GATGGTGTCCCCCGCCACCTCCGCC | |
| | | ACCTCCaagtcctgattctgcaatt | |
| | | tcagcc | |
| | | agtt | |
| | | (SEQ ID NO: 65) | |
| | | 2a.) | |
| | | tcttgatgagctgttcttccaggag | |
| | | gataaagttgttcatggtggcgacc | |
| | | ggtggatccc | |
| | | (SEQ ID NO: 50) | |
| | | 2b.) | |
| | | aattgcagaatcaggacttGGAGGT | |
| | | GGCGGAGGTGGCGGGGGACACCATC | |
| | | ACCATCACCATGTTTAAtccccccc | |
| | | ccctaacgttactgg | |
| | | (SEQ ID NO: 66) | |
| EG18 | FIG. 2Q | 1a) | 5 |
| | | aattgcagaatcaggacttGGAGGT | |
| | | GGCGGAGGTGGCGGGGGACACC | |
| | | (SEQ ID NO: 75) | |
| | | 1b) | |
| | | tcttgatgagctgttcttccaggag | |
| | | gataaagttgttcatggtggcgacc | |
| | | ggtggatccc | |
| | | (SEQ ID NO: 50) | |
| | | 2a) | |
| | | cgggcccgggatccaccggtcgcca | |
| | | ccatgaacaactttatcctcctgga | |
| | | agaacagctc | |
| | | (SEQ ID NO: 51) | |
| | | 2b) | |
| | | GATGGTGTCCCCCGCCACCTCCGCC | |
| | | ACCTCCaagtcctgattctgcaatt | |
| | | tcagcc | |
| | | agtt | |
| | | (SEQ ID NO: 65) | |
| EG19 | FIG. 2R | 1a) | 5 |
| | | cataatcagccataccacatttgta | |
| | | gaggttttacttgc | |
| | | (SEQ ID NO: 76) | |
| | | 1b) | |
| | | taCTTGTACAGCTCGTCCATGCCGAGAG | |
| | | (SEQ ID NO: 77) | |
| | | 2a) | |
| | | CTCTCGGCATGGACGAGCTGTACAAGta | |
| | | (SEQ ID NO: 78) | |
| | | 2b) | |
| | | gcaagtaaaacctctacaaatgtgg | |
| | | tatggctgattatg | |
| | | (SEQ ID NO: 79) | |
| EG20 | FIG. 2S | 1a) | 5 |
| | | tcctctctgcttctagaataaatca | |
| | | taatcagccataccacatttgtaga | |
| | | ggttttacttgct | |
| | | (SEQ ID NO: 80) | |
| | | 1b) | |
| | | tgtcatgaatcagtaggtccgcaaa | |
| | | gtaaccagcgtagtgCTTGTACAGC | |
| | | TCGTCCATGCCGAGAG | |
| | | (SEQ ID NO: 81) | |
| | | 2a) | |
| | | actttgcggacctactgattcatga | |
| | | cattgagacaaatccagggatgaac | |
| | | tttctacgtaagatagtgaaaaatt | |
| | | (SEQ ID NO: 82) | |
| | | 2b) | |
| | | acctctacaaatgtggtatggctga | |
| | | ttatgatttattctagaagcagaga | |
| | | ggaatctttg | |
| | | (SEQ ID NO: 83) | |
| EG21 | FIG. 2T | 1a) | 5 |
| | | gctggttactttgcggacctactga | |
| | | ttcatgacattgagacaaatccagg | |
| | | gggattcggacaccaaaacaaagcg | |
| | | gtgtacactg | |
| | | (SEQ ID NO: 84) | |
| | | 1b) | |
| | | aaacctctacaaatgtggtatggct | |
| | | gattatgatttgttccatggcttct | |
| | | tcttcgtaggcatacaagtc | |
| | | (SEQ ID NO: 85) | |
| | | 2a) | |
| | | tgtctcaatgtcatgaatcagtagg | |
| | | tccgcaaagtaaccagcgtagtgCT | |
| | | TGTACAGCTCGTCCATGCCGAGAGT | |
| | | GATCCC | |
| | | (SEQ ID NO: 86) | |
| | | 2b) | |
| | | gagacttgtatgcctacgaagaaga | |
| | | agccatggaacaaatcataatcagc | |
| | | cataccacatttgtagaggttttac | |
| | | ttgct | |
| | | (SEQ ID NO: 87) | |
| EG22 | FIG. 2U | 1a) | 4 |
| | | catggcagaggtgttcagagtcctc | |
| | | atggtggcgaccggtggattcacga | |
| | | cacctgaaatggaagaaaaaaac | |
| | | (SEQ ID NO: 88) | |
| | | 1b) | |
| | | attaccgccatgcattagttattag | |
| | | gctccggtgcccgtcagtgggcaga | |
| | | gcg | |
| | | (SEQ ID NO: 89) | |
| | | 2a) | |
| | | agtttttttcttccatttcaggtgt | |
| | | cgtgaatccaccggtcgccaccatg | |
| | | aggactctgaacacctc | |
| | | (SEQ ID NO: 90) | |
| | | 2b) | |
| | | gtgcgctctgcccactgacgggcac | |
| | | cggagcctaataactaatgcatggc | |
| | | ggtaat | |
| | | (SEQ ID NO: 91) | |
| EG23 | FIG. 2V | 1a) | 4 |
| | | gaggccgaggccgcctcggcctctg | |
| | | agctaatccaccggtcgccaccatg | |
| | | aggactctgaacacctc | |
| | | (SEQ ID NO: 92) | |
| | | 1b) | |
| | | ataaccgtattaccgccatgcatta | |
| | | gttattaggtgtggaaagtccccag | |
| | | gctccccagcaggcaga | |
| | | (SEQ ID NO: 93) | |
| | | 2a) | |
| | | ttcagagtcctcatggtggcgaccg | |
| | | gtggattagctcagaggccgaggcg | |
| | | gccteggcctct | |
| | | (SEQ ID NO: 94) | |
| | | 2b) | |
| | | tctgcctgctggggagcctggggac | |
| | | tttccacacctaataactaatgcat | |
| | | ggcggtaatacggtta | |
| | | (SEQ ID NO: 95) | |
| EG24 | FIG. 2W | 1a) | 6 |
| | | GGAGGTGGCGGAGGTGGCGGGGGAC | |
| | | ACCATCACCATCA | |
| | | (SEQ ID NO: 96) | |
| | | 1b) | |
| | | AGACAACGCTGGCCTTTTCCAGAGG | |
| | | CGACCTCTGCATggtggcgaccggt | |
| | | ggatcccgggcc | |
| | | cg | |
| | | (SEQ ID NO: 97) | |
| | | 2a) | |
| | | cgggcccgggatccaccggtcgcca | |
| | | ccATGCAGAGGTCGCCTCTGGAAAA | |
| | | GGCCAGCGTTGTCTC | |
| | | (SEQ ID NO: 98) | |
| | | 2b) | |
| | | CCCCGCCACCTCCGCCACCTCCAAG | |
| | | CCTTGTATCTTGCACCTCTTCTTCT | |
| | | GTCTCC | |
| | | (SEQ ID NO: 99) | |
| EG25 | FIG. 2X | 1a) | 6 |
| | | GGAGGTGGCGGAGGTGGCGGGGGAC | |
| | | ACCATCACCATCA | |
| | | (SEQ ID NO: 96) | |
| | | 1b) | |
| | | AGACAACGCTGGCCTTTTCCAGAGG | |
| | | CGACCTCTGCATggtggcgaccggt | |
| | | ggatcccgggcc | |
| | | cg | |
| | | (SEQ ID NO: 97) | |
| | | 2a) | |
| | | cgggcccgggatccaccggtcgcca | |
| | | ccATGCAGAGGTCGCCTCTGGAAAA | |
| | | GGCCAGCGTTGTCTC | |
| | | (SEQ ID NO: 98) | |
| | | 2b) | |
| | | CCCCGCCACCTCCGCCACCTCCAAG | |
| | | CCTTGTATCTTGCACCTCTTCTTCT | |
| | | GTCTCC | |
| | | (SEQ ID NO: 99) | |

TABLE 3
Illustrative amino acid sequences
comprised by some constructs

| Descrip- | Illustrative | |
|---|---|---|
| tion | constructs | Amino acid sequence |
| DRD1-GFP | EG7, EG8, | MRTLNTSAMDGTGLVVERD |
| | EG10, EG12, | FSVRILTACFLSLLILSTL |
| | EG10, EG19, | LGNTLVCAAVIRFRHLRSK |
| | EG20, EG21, | VTNFFVISLAVSDLLVAVL |
| | EG22, EG23 | VMPWKAVAEIAGFWPFGSF |
| | | CNIWVAFDIMCSTASILNL |
| | | CVISVDRYWAISSPFRYER |
| | | KMTPKAAFILISVAWTLSV |
| | | LISFIPVQLSWHKAKPTSP |
| | | SDGNATSLAETIDNCDSSL |
| | | SRTYAISSSVISFYIPVAI |
| | | MIVTYTRIYRIAQKQIRRI |
| | | AALERAAVHAKNCQTTTGN |
| | | GKPVECSQPESSFKMSFKR |
| | | ETKVLKTLSVIMGVFVCCW |
| | | LPFFILNCILPFCGSGETQ |
| | | PFCIDSNTFDVFVWFGWAN |
| | | SSLNPIIYAFNADFRKAFS |
| | | TLLGCYRLCPATNNAIETV |
| | | SINNNGAAMFSSHHEPRGS |
| | | ISKECNLVYLIPHAVGSSE |
| | | DLKKEEAAGIARPLEKLSP |
| | | ALSVILDYDTDVSLEKIQP |
| | | ITQNGQHPTGGGGSGGGGS |
| | | GGGGSMVSKGEELFTGVVP |
| | | ILVELDGDVNGHKFSVSGE |
| | | GEGDATYGKLTLKFICTTG |
| | | KLPVPWPTLVTTLTYGVQC |
| | | FARYPDHMKQHDFFKSAMP |
| | | EGYVQERTIFFKDDGNYKT |
| | | RAEVKFEGDTLVNRIELKG |
| | | IDFKEDGNILGHKLEYNYN |
| | | SHKVYITADKQKNGIKVNF |
| | | KTRHNIEDGSVQLADHYQQ |
| | | NTPIGDGPVLLPDNHYLST |
| | | QSKLSKDPNEKRDHMVLLE |
| | | FVTAAGITLGMDELYK |
| | | (SEQ ID NO: 12) |
| GFP | EG1, EG2, | MVSKGEELFTGVVPILVEL |
| | EG3, EG7, | DGDVNGHKFSVSGEGEGDA |
| | EG8, EG10, | TYGKLTLKFICTTGKLPVP |
| | EG12, EG10, | WPTLVTTLTYGVQCFARYP |
| | EG19, EG20, | DHMKQHDFFKSAMPEGYVQ |
| | EG21, EG22, | ERTIFFKDDGNYKTRAEVK |
| | EG23 | FEGDTLVNRIELKGIDFKE |
| | | DGNILGHKLEYNYNSHKVY |
| | | ITADKQKNGIKVNFKTRHN |
| | | IEDGSVQLADHYQQNTPIG |
| | | DGPVLLPDNHYLSTQSKLS |
| | | KDPNEKRDHMVLLEFVTAA |
| | | GITLGMDELYK |
| | | (SEQ ID NO: 13) |
| DRD1-Strep | EG5, EG6 | MRTLNTSAMDGTGLVVERD |
| | | FSVRILTACFLSLLILSTL |
| | | LGNTLVCAAVIRFRHLRSK |
| | | VTNFFVISLAVSDLLVAVL |
| | | VMPWKAVAEIAGFWPFGSF |
| | | CNIWVAFDIMCSTASILNL |
| | | CVISVDRYWAISSPFRYER |
| | | KMTPKAAFILISVAWTLSV |
| | | LISFIPVQLSWHKAKPTSP |
| | | SDGNATSLAETIDNCDSSL |
| | | SRTYAISSSVISFYIPVAI |
| | | MIVTYTRIYRIAQKQIRRI |
| | | AALERAAVHAKNCQTTTGN |
| | | GKPVECSQPESSFKMSFKR |
| | | ETKVLKTLSVIMGVFVCCW |
| | | LPFFILNCILPFCGSGETQ |
| | | PFCIDSNTFDVFVWFGWAN |
| | | SSLNPIIYAFNADFRKAFS |
| | | TLLGCYRLCPATNNAIETV |
| | | SINNNGAAMFSSHHEPRGS |
| | | ISKECNLVYLIPHAVGSSE |
| | | DLKKEEAAGIARPLEKLSP |
| | | ALSVILDYDTDVSLEKIQP |
| | | ITQNGQHPTTGTRPLWSHP |
| | | QFEK |
| | | (SEQ ID NO: 14) |
| ITK | EG9, EG17, | MNNFILLEEQLIKKSQQKR |
| | EG18 | RTSPSNFKVRFFVLTKASL |
| | | AYFEDRHGKKRTLKGSIEL |
| | | SRIKCVEIVKSDISIPCHY |
| | | KYPFQVVHDNYLLYVFAPD |
| | | RESRQRWVLALKEETRNNN |
| | | SLVPKYHPNFWMDGKWRCC |
| | | SQLEKLATGCAQYDPTKNA |
| | | SKKPLPPTPEDNRRPLWEP |
| | | EETVVIALYDYQTNDPQEL |
| | | ALRRNEEYCLLDSSEIHWW |
| | | RVQDRNGHEGYVPSSYLVE |
| | | KSPNNLETYEWYNKSISRD |
| | | KAEKLLLDTGKEGAFMVRD |
| | | SRTAGTYTVSVFTKAVVSE |
| | | NNPCIKHYHIKETNDNPKR |
| | | YYVAEKYVFDSIPLLINYH |
| | | QHNGGGLVTRLRYPVCFGR |
| | | QKAPVTAGLRYGKWVIDPS |
| | | ELTFVQEIGSGQFGLVHLG |
| | | YWLNKDKVAIKTIREGAMS |
| | | EEDFIEEAEVMMKLSHPKL |
| | | VQLYGVCLEQAPICLVFEF |
| | | MEHGCLSDYLRTQRGLFAA |
| | | ETLLGMCLDVCEGMAYLEE |
| | | ACVIHRDLAARNCLVGENQ |
| | | VIKVSDFGMTRFVLDDQYT |
| | | SSTGTKFPVKWASPEVFSF |
| | | SRYSSKSDVWSFGVLMWEV |
| | | FSEGKIPYENRSNSEVVED |
| | | ISTGFRLYKPRLASTHVYQ |
| | | IMNHCWKERPEDRPAFSRL |
| | | LRQLAEIAESGLGGGGGGG |
| | | GHHHHHHV |
| | | (SEQ ID NO: 15) |
| C1 Inhibitor | EG15, EG16 | MASRLTLLTLLLLLLAGDR |
| | | ASSNPNATSSSSQDPESLQ |
| | | DRGEGKVATTVISKMLFVE |
| | | PILEVSSLPTTNSTTNSAT |
| | | KITANTTDEPTTQPTTEPT |
| | | TQPTIQPTQPTTQLPTDSP |
| | | TQPTTGSFCPGPVTLCSDL |
| | | ESHSTEAVLGDALVDFSLK |
| | | LYHAFSAMKKVETNMAFSP |
| | | FSIASLLTQVLLGAGENTK |
| | | TNLESILSYPKDFTCVHQA |
| | | LKGFTTKGVTSVSQIFHSP |
| | | DLAIRDTFVNASRTLYSSS |
| | | PRVLSNNSDANLELINTWV |
| | | AKNTNNKISRLLDSLPSDT |
| | | RLVLLNAIYLSAKWKTTFD |
| | | PKKTRMEPFHFKNSVIKVP |
| | | MMNSKKYPVAHFIDQTLKA |
| | | KVGQLQLSHNLSLVILVPQ |
| | | NLKHRLEDMEQALSPSVFK |
| | | AIMEKLEMSKFQPTLLTLP |
| | | RIKVTTSQDMLSIMEKLEF |
| | | FDFSYDLNLCGLTEDPDLQ |
| | | VSAMQHQTVLELTETGVEA |
| | | AAASAISVARTLLVFEVQQ |
| | | PFLFVLWDQQHKFPVFMGR |
| | | VYDPRA |
| | | (SEQ ID NO: 16) |
| T7 RNA | EG4 | MNTINIAKNDFSDIELAAI |
| polymerase | | PFNTLADHYGERLAREQLA |
| | | LEHESYEMGEARFRKMFER |
| | | QLKAGEVADNAAAKPLITT |
| | | LLPKMIARINDWFEEVKAK |
| | | RGKRPTAFQFLQEIKPEAV |
| | | AYITIKTTLACLTSADNTT |
| | | VQAVASAIGRAIEDEARFG |
| | | RIRDLEAKHFKKNVEEQLN |
| | | KRVGHVYKKAFMQVVEADM |
| | | LSKGLLGGEAWSSWHKEDS |
| | | IHVGVRCIEMLIESTGMVS |
| | | LHRQNAGVVGQDSETIELA |
| | | PEYAEAIATRAGALAGISP |
| | | MFQPCVVPPKPWTGITGGG |
| | | YWANGRRPLALVRTHSKKA |
| | | LMRYEDVYMPEVYKAINIA |
| | | QNTAWKINKKVLAVANVIT |
| | | KWKHCPVEDIPAIEREELP |
| | | MKPEDIDMNPEALTAWKRA |
| | | AAAVYRKDKARKSRRISLE |
| | | FMLEQANKFANHKAIWFPY |
| | | NMDWRGRVYAVSMFNPQGN |
| | | DMTKGLLTLAKGKPIGKEG |
| | | YYWLKIHGANCAGVDKVPF |
| | | PERIKFIEENHENIMACAK |
| | | SPLENTWWAEQDSPFCFLA |
| | | FCFEYAGVQHHGLSYNCSL |
| | | PLAFDGSCSGIQHFSAMLR |
| | | DEVGGRAVNLLPSETVQDI |
| | | YGIVAKKVNEILQADAING |
| | | TDNEVVTVTDENTGEISEK |
| | | VKLGTKALAGQWLAYGVTR |
| | | SVTKRSVMTLAYGSKEFGF |
| | | RQQVLEDTIQPAIDSGKGL |
| | | MFTQPNQAAGYMAKLIWES |
| | | VSVTVVAAVEAMNWLKSAA |
| | | KLLAAEVKDKKTGEILRKR |
| | | CAVHWVTPDGFPVWQEYKK |
| | | PIQTRLNLMFLGQFRLQPT |
| | | INTNKDSEIDAHKQESGIA |
| | | PNFVHSQDGSHLRKTVVWA |
| | | HEKYGIESFALIHDSFGTI |
| | | PADAANLFKAVRETMVDTY |
| | | ESCDVLADFYDQFADQLHE |
| | | SQLDKMPALPAKGNLNLRD |
| | | ILESDFAFA |
| | | (SEQ ID NO: 17) |
| CFTR | EG24, EG25 | MQRSPLEKASVVSKLFFSW |
| | | TRPILRKGYRQRLELSDIY |
| | | QIPSVDSADNLSEKLEREW |
| | | DRELASKKNPKLINALRRC |
| | | FFWRFMFYGIFLYLGEVTK |
| | | AVQPLLLGRIIASYDPDNK |
| | | EERSIAIYLGIGLCLLFIV |
| | | RTLLLHPAIFGLHHIGMQM |
| | | RIAMFSLIYKKTLKLSSRV |
| | | LDKISIGQLVSLLSNNLNK |
| | | FDEGLALAHFVWIAPLQVA |
| | | LLMGLIWELLQASAFCGLG |
| | | FLIVLALFQAGLGRMMMKY |
| | | RDQRAGKISERLVITSEMI |
| | | ENIQSVKAYCWEEAMEKMI |
| | | ENLRQTELKLTRKAAYVRY |
| | | FNSSAFFFSGFFVVFLSVL |
| | | PYALIKGIILRKIFTTISF |
| | | CIVLRMAVTRQFPWAVQTW |
| | | YDSLGAINKIQDFLQKQEY |
| | | KTLEYNLTTTEVVMENVTA |
| | | FWEEGFGELFEKAKQNNNN |
| | | RKTSNGDDSLFFSNFSLLG |
| | | TPVLKDINFKIERGQLLAV |
| | | AGSTGAGKTSLLMMIMGEL |
| | | EPSEGKIKHSGRISFCSQF |
| | | SWIMPGTIKENIIFGVSYD |
| | | EYRYRSVIKACQLEEDISK |
| | | FAEKDNIVLGEGGITLSGG |
| | | QRARISLARAVYKDADLYL |
| | | LDSPFGYLDVLTEKEIFES |
| | | CVCKLMANKTRILVTSKME |
| | | HLKKADKILILHEGSSYFY |
| | | GTFSELQNLQPDFSSKLMG |
| | | CDSFDQFSAERRNSILTET |
| | | LHRFSLEGDAPVSWTETKK |
| | | QSFKQTGEFGEKRKNSILN |
| | | PINSIRKFSIVQKTPLQMN |
| | | GIEEDSDEPLERRLSLVPD |
| | | SEQGEAILPRISVISTGPT |
| | | LQARRRQSVLNLMTHSVNQ |
| | | GQNIHRKTTASTRKVSLAP |
| | | QANLTELDIYSRRLSQETG |
| | | LEISEEINEEDLKECLFDD |
| | | MESIPAVTTWNTYLRYITV |
| | | HKSLIFVLIWCLVIFLAEV |
| | | AASLVVLWLLGNTPLQDKG |
| | | NSTHSRNNSYAVIITSTSS |
| | | YYVFYIYVGVADTLLAMGF |
| | | FRGLPLVHTLITVSKILHH |
| | | KMLHSVLQAPMSTLNTLKA |
| | | GGILNRFSKDIAILDDLLP |
| | | LTIFDFIQLLLIVIGAIAV |
| | | VAVLQPYIFVATVPVIVAF |
| | | IMLRAYFLQTSQQLKQLES |
| | | EGRSPIFTHLVTSLKGLWT |
| | | LRAFGRQPYFETLFHKALN |
| | | LHTANWFLYLSTLRWFQMR |
| | | IEMIFVIFFIAVTFISILT |
| | | TGEGEGRVGIILTLAMNIM |
| | | STLQWAVNSSIDVDSLMRS |
| | | VSRVFKFIDMPTEGKPTKS |
| | | TKPYKNGQLSKVMIIENSH |
| | | VKKDDIWPSGGQMTVKDLT |
| | | AKYTEGGNAILENISFSIS |
| | | PGQRVGLLGRTGSGKSTLL |
| | | SAFLRLLNTEGEIQIDGVS |
| | | WDSITLQQWRKAFGVIPQK |
| | | VFIFSGTFRKNLDPYEQWS |
| | | DQEIWKVADEVGLRSVIEQ |
| | | FPGKLDFVLVDGGCVLSHG |
| | | HKQLMCLARSVLSKAKILL |
| | | LDEPSAHLDPVTYQIIRRT |
| | | LKQAFADCTVILCEHRIEA |
| | | MLECQQFLVIEENKVRQYD |
| | | SIQKLLNERSLFRQAISPS |
| | | DRVKLFPHRNSSKCKSKPQ |
| | | IAALKEETEEEVQDTRL |
| | | (SEQ ID NO: 18) |

Cell Lines—Culturing and Transfection

HEK293 cells were used to illustrate the application of the present systems, methods, and compositions in human eukaryotic cells. HEK293 adherent cells (CLS) were cultured in Dulbecco's Modified Eagle Medium high glucose (Gibco) supplemented with 10% Fetal Bovine Serum (Gibco) and 50,000 U Pen Strep (Gibco). HEK293 cells were grown to 80% confluency at 37° C. and 5% CO₂ before transiently transfecting using 293 fectin (ThermoFisher) according to manufacturer's instruction. Protein-expressing cells were harvested after 48h by detaching the cells using 0.5% trypsin solution for 5 min at 37° C. and scraping. Cells were pelleted (5,000×g, 15 min, 4° C.) and supernatant was discarded. Cell pellets were stored at −80° C. until further usage.

CHO-K1 cells are used to illustrate the application of the present systems, methods, and compositions in eukaryotic animal cells. CHO-K1 adherent cells (CLS) are cultured in F-12K medium (ATCC) supplemented with 10% Fetal Bovine Serum (Gibco). CHO-K1 cells are grown to 80% confluency at 37° C. and 5% CO₂ before transiently transfecting using Lipofectamine LTX (ThermoFisher) according to manufacturer's instruction. Protein-expressing cells are harvested after 48h by detaching the cells using 0.5% trypsin solution for 5 min at 37° C. and scraping. Cells are pelleted (5,000×g, 15 min, 4° C.) and supernatant is discarded. Cell pellets are stored at −80° C. until further usage.

SF9 cells are used to illustrate the application of the present systems, methods, and compositions in eukaryotic insect cells. SF9 suspension cells (CLS) are cultured in Grace's Insect Medium, supplemented (ThermoFisher) supplemented with 10% Fetal Bovine Serum (Gibco). SF9 cells are grown at 26° C. and 130 rpm before transiently transfecting using Cellfectin II (ThermoFisher) according to manufacturer's instruction. Protein expressing cells are harvested after 48h (5,000×g, 15 min, 4° C.) and supernatant is discarded. Cell pellets are stored at −80° C. until further usage.

### Example 1: Expression of the L Enhancer Protein with GFP Reduced Over-Expression of the GFP Protein

CMV Promoter System

To demonstrate the influence of the introduction of the viral nuclear pore blocking proteins during an expression, HEK293 cells were transfected with either EG1, EG2 or co-transfected with EG3 and EG4 constructs (see Table 2 and FIG. 2 for construct details). The expression of the viral pore blocking proteins resulted in controlled regulation of protein expression. Consequently, the obtained GFP signal was decreased. The reason for the controlled regulation of the gene of interest that is in tandem with the pore blocking proteins is the mode of action of the viral protein. Without being bound by theory, a possible mechanism for protein regulation is that by expressing pore blocking proteins, nuclear export of mRNA may be inhibited and as a consequence the translation of the target protein will be downregulated. After stabilizing, the pore blocking proteins will be degraded and mRNA transport will resume. This again leads to the expression of both the target protein and the enhancer protein, e.g., a pore blocking protein. This tightly controlled feedback ensures stabilization and permanent expression of the target protein and prevents the usual regulation of eukaryotic cells that leads to a shut-down of protein expression.

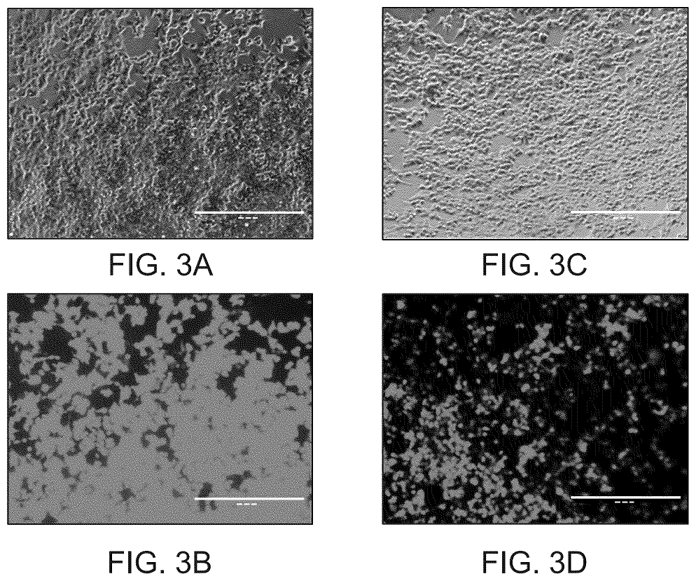

FIGS. 3A-3D show the effect on GFP expression in the absence and presence of the L-protein from ECMV as an illustrative enhancer protein according to the present disclosure. HEK293 cells were seeded at 0.05×10 6 cells/well in a 24 well plate and incubated at 37° C. and 5% CO₂ over night before transiently transfecting with either EG1 or EG2 as described above. GFP expression was monitored after 24 h and 48h using fluorescence microscopy. Images were taken using a CCD Camera (Amscope) and analysed with ISCapture (Amscope). This example demonstrates the improved regulation of target protein expression in an illustrative system comprising a target protein polynucleotide and an enhancer protein polynucleotide according to the present disclosure.

T7 Polymerase System

While EG2 uses the natural polymerases of the eukaryotic host, other viral polymerases like T7 can be used to initiate transcription outside of the nucleus. The viral polymerase is under control of a standard eukaryotic promoter and the corresponding mRNA will depend on nuclear export. In the cytosol, the viral polymerase is translated and then initiates transcription of the target protein polynucleotide and the enhancer protein polynucleotide. In some embodiments, as a consequence of the expression of the enhancer proteins, the nuclear transport of the viral polymerase will decrease. The stabilization of the system will lead to degradation of the enhancer proteins and mRNA transport of the viral polymerase will resume. Without being bound by theory, this feedback may prevent the usual regulation of the cell while overexpressing a recombinant protein. In some circumstances, using viral polymerase gives the advantage of higher expression levels on a cell to cell basis compared to the system using eukaryotic polymerases.

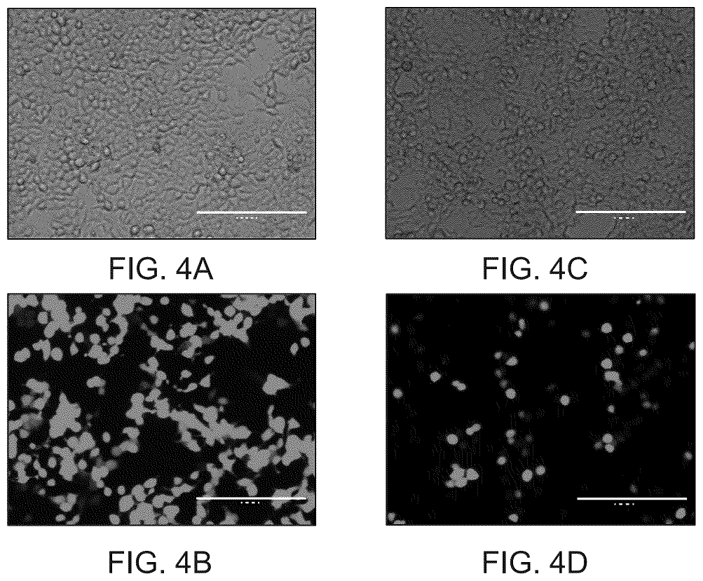

FIGS. 4A-4D show the successful expression of GFP in tandem with the L protein from ECMV from a T7 promoter when co-transfected with a T7 harboring vector. HEK293 cells were seeded at 0.05×10⁶ cells/well in a 24 well plate and incubated at 37° C. and 5% CO₂ over night before transiently transfecting with either EG1 or EG3 and EG4 as described above. GFP expression was monitored after 24 h and 48h using fluorescence microscopy. Images were taken using a CCD Camera (Amscope) and analyzed with ISCapture (Amscope). This example demonstrates the successful use of T7 as an illustrative viral polymerase in tandem with GFP as target protein and the L-protein of ECMV as enhancer protein. Similar to the example above, the introduction of the L-protein led to a tighter regulation of expression and therefore an overall reduction in over-expression.

### Example 2: Co-Expression of the L Enhancer Protein with DRD1-GFP LED to Improved Expression and Localization of the DRD1 Membrane Protein

DRD1 was used as to illustrate the application of the disclosed systems and methods to the co-expression of a membrane protein as target protein in combination with pore blocking proteins as enhancer proteins in order to yield a high density of active membrane receptors. DRD1 is a G-protein-coupled receptor and is known to be difficult to express using the academic standard. To visualize the correct translocation into the outer membrane of the cells, DRD1-GFP fusions (EG8) were used in the present system. To illustrate the problem with GPCRs in academic and industrial settings, the academic standard (EG10) was used as a control.

Improved Membrane Protein Expression and Membrane Localization

DRD1-GFP fusions were expressed in HEK293 cells. HEK293 cells were seeded at 0.05×10⁶ cells/well in a 24 well plate and incubated at 37° C. and 5% CO₂ over night before transiently transfecting with either EG10 or EG8 as described above. DRD1-GFP expression was monitored after 24 h and 48h using fluorescence microscopy. Images were taken using a CCD Camera (Amscope) and analyzed with ISCapture (Amscope).

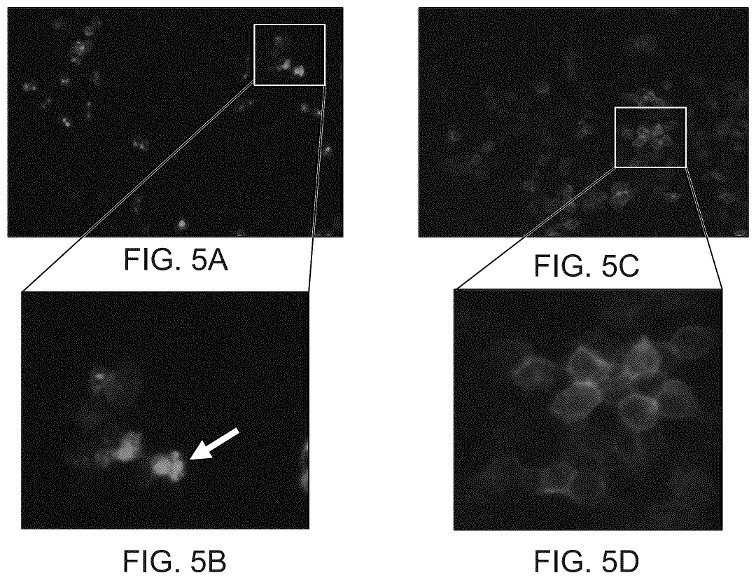

FIGS. 5A-5D demonstrate that EG10 fails to correctly translocate the expressed receptor. Without being bound by theory, it is believed that as a consequence of the overexpression of the human DRD1 receptor in human cells with the EG10 construct, the cells start to degrade or control the expressed target protein. This form of regulation results in the formation of denatured protein as inclusion bodies (FIG. 5B, red arrow). The control of expression of membrane proteins by the cells in this way may result in inactive and misfolded protein and consequently in unusable, poor quality expressed protein. In contrast, the co-expression of the target membrane protein with illustrative enhancer proteins resulted in correctly translocated DRD1-GFP, as can be seen by the correct insertion into the membrane and the absence of inclusion bodies (FIG. 5C-5D). This example demonstrates that the co-expression of an illustrative enhancer protein (the L-protein of ECMV) in conjunction with an illustrative target membrane protein (DRD1) resulted in improved expression and localization of the membrane protein. Without being bound by theory, it is believed that the present system produces tight regulation of target protein expression, thereby bypassing the normal regulation of the cell that would result in degradation of the expressed membrane protein. Thus, the present system is suitable for high yield expression and purification of GPCRs.

Separate Expression of the Target Protein and the Enhancer Protein

Furthermore, to illustrate that the enhancer protein can be located on a separate DNA molecule, DRD1-GFP (EG10) constructs are co-expressed with the L-protein from ECMV (EG11) under the control of a separate promoter on a separate vector. HEK293 cells are seeded at 0.05×10⁶ cells/well in a 24 well plate and incubated at 37° C. and 5% CO₂ over night before transiently transfecting with EG10 and EG11 as described above. DRD1-GFP expression is monitored after 24 h and 48h using fluorescence microscopy. Images are taken using a CCD Camera (Amscope) and analyzed with ISCapture (Amscope).

Functional Activity of the Membrane Protein

In addition to the illustration of the correctly translocated GPCR, activity tests were performed using a DRD1-Strep fusion. The smaller strep-tag ensures that the correct interaction with the cytosolic located G-protein is intact and a functional assay can be performed. Upon binding of dopamine, DRD1 releases the heterotrimeric G-protein to its Ga subunit and its Gβγ complex. In the resting state, Ga binds GDP but upon activation exchanges GTP for GDP. The Ga-GTP complex interacts with adenylate cyclase (AC), resulting in activation of AC activity and as consequence increasing cAMP levels. Changes in intracellular cAMP can be measured by standard cAMP assays. Again, the academic and industry standard (EG5) was compared to the same target protein in co-expression with the L-protein of ECMV.

DRD1-Strep fusions are expressed in HEK293 cells. HEK293 cells are seeded at 5,000 cells/well in a 96 well white clear bottom plates and incubated at 37° C. and 5% CO₂ over night before transiently transfecting with either EG5 or EG6 as described above. Protein is expressed for 48h and DRD1 activity is analyzed using the cAMP (glo) assay (Promega) according to manufacturer's instruction. In detail, after 48h cells are washed with sterile PBS pH 7.2 and cells are incubated for 2h with 20 μl dopamine substrate concentrations ranging from 1 mM-1 μM at 37° C. As non-induced control, cells are incubated with 20 μl PBS pH 7.2. After incubation, cells are washed with PBS pH 7.2 and 20 μl lysis buffer is added. Lysis is performed for 15 min at room temperature (RT) with shaking. Following, 40 μl detection solution is added and cells are incubated for 20 min at RT with shaking. Reactions are stopped using 80 μl Kinase-Glo Reagent incubated for 15 min at RT before analyses. Luminescence is measured using a plate reader (Synergy LX (BioTek)) and data are analyzed using standard analysis programs.

### Example 3: T7 Viral Promoter Successfully Used to Initiate Transcription of the DRD1-GFP and L Enhancer Protein Construct Outside of the Nucleus

For this example, DRD1-GFP was selected as an illustrative difficult to express target membrane protein in combination with a T7 promoter to demonstrate that viral polymerases like T7 can be used to initiate transcription outside of the nucleus. As in Example 1, the viral polymerase was under control of a standard eukaryotic promoter and the corresponding mRNA relied on nuclear export.

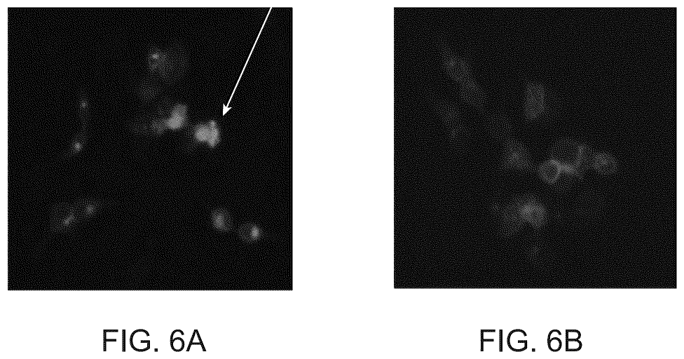

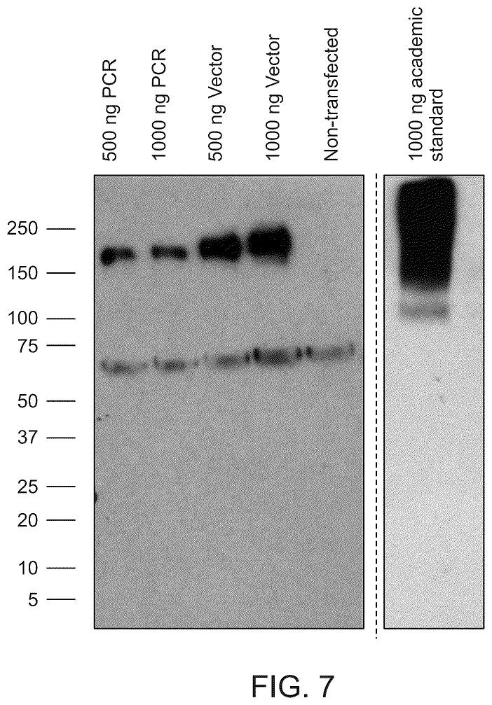

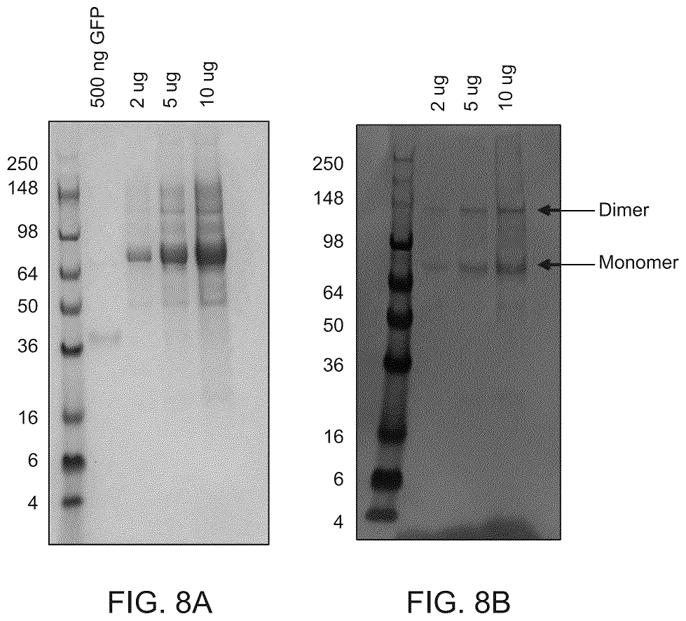

FIGS. 6A-6B demonstrate the successful expression of DRD1-GFP in tandem with the L protein from ECMV from a T7 promoter when co-transfected with a T7 harboring vector. HEK293 cells were seeded at 0.05×10⁶ cells/well in a 24 well plate and incubated at 37° C. and 5% CO₂ over night before transiently transfecting with either EG10 or EG12 and EG4 as described above. DRD1-GFP expression was monitored after 24 h and 48h using fluorescence microscopy. Images were taken using a CCD Camera (Amscope) and analyzed with ISCapture (Amscope). This example demonstrates the successful use of T7 as viral polymerase in tandem with DRD1-GFP as target protein and the L-protein of ECMV as enhancer protein.

### Example 4: Expression of DRD1-GFP and the L Enhancer Protein is Compatible with Different Mammalian Promoters

Systems, methods, and compositions according to the present disclosure are compatible with a wide variety of mammalian promoters. To demonstrate the compatibility of the co-expression of the target protein and the enhancer protein from different promoters, DRD1-GFP was used as an illustrative target protein. As described in Example 2, the correct expression and translocation of DRD1-GFP can be easily detected by fluorescence microscopy. Beside the CMV promoter (EG10), EF1-α (EG22) and SV40 (EG23) are used followed by the identical DRD1-GFP IRES L assembly.

DRD1-GFP fusions under the control of different mammalian promoters are expressed in HEK293 cells. HEK293 cells are seeded at 0.05×10⁶ cells/well in a 24 well plate and incubated at 37° C. and 5% CO₂ over night before transiently transfecting with either EG8, EG22 or EG23 as described above. DRD1-GFP expression is monitored after 24h and 48h using fluorescence microscopy. Images are taken using a CCD Camera (Amscope) and analyzed with ISCapture (Amscope).

### Example 5: Any of the Tested L Enhancer Proteins May be Used to Successfully Enhance DRD1-GFP Expression and Localization

Without being bound by theory, one mechanism that may be used to regulate expression of a recombinantly inserted target protein polynucleotide is the introduction of a pore blocking protein. To demonstrate that natural or synthetic pore blocking proteins can be exchanged with each other in one embodiment of the present system while still retaining the benefits of controlling the cell regulation, the Leader protein of ECMV (EG10), the Leader protein of Theiler's virus (EG19), the 2A protease of Polio virus (EG21) and the M protein of vesicular stomatitis virus (EG20) were cloned in tandem with DRD1-GFP as the illustrative target protein. As described in Example 2, the correct expression and translocation of DRD1-GFP can be easily detected by fluorescence microscopy.

DRD1-GFP fusions in tandem with different enhancer proteins are expressed in HEK293 cells. HEK293 cells are seeded at 0.05×10⁶ cells/well in a 24 well plate and incubated at 37° C. and 5% CO₂ over night before transiently transfecting with either EG8, EG19, EG20 or EG21 as described above. DRD1-GFP expression is monitored after 24h and 48h using fluorescence microscopy. Images are taken using a CCD Camera (Amscope) and analyzed with ISCapture (Amscope).

### Example 6: Expression of L Enhancer Protein Improves the Expression of Cystic Fibrosis Transmembrane Conductance Regulator (CFTR)

CFTR was used as an additional example to demonstrate that the co-expression of a membrane protein as target protein in combination with pore blocking proteins as enhancer proteins yielded a high density of active ion-channel. CFTR is a transmembrane transporter of the ABC-transporter class that conducts chloride ions across epithelial cell membranes. CFTR is known to express in a heterogenous manner when using the academic standard. Heterogeneity increases the difficulty in purifying or analyzing the ABC transporter. To demonstrate the improvement of homogeneity, CFTR was either cloned into the backbone of an illustrative system (EG25) or was used as a PCR product. As comparison, the academic standard (EG24) was used alongside as a control.

CFTR constructs were expressed in HEK293 cells. HEK293 cells were seeded at 0.3×10⁶ cells/well in a 6 well plate and incubated at 37° C. and 5% CO₂ over night before transiently transfecting with either EG25, the PCR-product of EG25 or EG24 as described above. CFTR expression was monitored after 24 h and 48h using microscopy. Cells were harvested and lysed after 48h using RIPA buffer (CellGene). Lysate was cleared and analyzed by SDS-PAGE (6-12% BOLT, ThermoFisher) followed by Westernblot (NC membrane, ThermoFisher) using anti-CFTR (Abcam, 2n d anti-mouse-hrp).

FIG. 6 demonstrates the impact of the co-expression of the L-protein with the CFTR. Whereas the academic standard produced a wide band on the Western blot, transcription and translation based on the EG25 construct resulted in defined bands demonstrating a highly homogenous expression of the ABC-transporter. Additionally, this example demonstrates that the expression system can be delivered into the cell as a vector or as a PCR product.

### Example 7: Production of C1 Esterase Inhibitor (C1-Inh) Protein, Used as an Example Protein Requiring Post-Translational Modifications

C1-Inh is used as an illustrative target protein to exemplify the application of the disclosed systems for difficult to express secreted proteins yielding the correct post-translational modifications. C1-Inh is a protease inhibitor belonging to the serpin superfamily. As a secreted protein C1-Inh is highly glycosylated and therefore proofs to be a difficult target for recombinant expression. To demonstrate that the system can produce correctly glycosylated C1-Inh in high yields, C1-Inh-his fusion are expressed using the presented system (EG16). As comparison, the academic and industrial standard (EG15) is used alongside.

C1-Inh-his fusions are expressed in HEK293 cells. HEK293 cells are seeded at 4.9×10⁶ cells in a T175 flask and incubated at 37° C. and 5% CO₂ over night before transiently transfecting with either EG15 or EG16 as described above. C1-Inh-his expression is monitored after 24 h and 48h using microscopy. Supernatant containing protein is harvested after 48h and supernatant is cleared by filtration (22 um, nitrocellulose). To purify C1-Inh, His-resin (GE Healthcare HisTrap) is equilibrated with 20 mM Tris pH 7.5, 50 mM NaCl prior to adding to the supernatant. Supernatant is incubated with the resin for 2 h at 4° C. with shaking. Resin is settled and washed with 5 CV 20 mM Tris pH 7.5, 50 mM NaCl and protein was eluted with 3 CV 20 mM Tris pH 7.5, 50 mM NaCl, 500 mM Imidazole. Purification is analyzed by SDS-PAGE (6-12% BOLT, ThermoFisher) and protein containing fractions are pooled and concentrated. Protein is further polished by size-exclusion chromatography (SEC) (Superdex 200, ThermoFisher) and fraction are analyzed by SDS-PAGE (6-12% BOLT, ThermoFisher). Protein containing fractions are pooled and send for analysis regarding glycosylation pattern and sequence analysis.

### Example 8: Production of IL2 Inducible T Cell Kinase (ITK), Used as an Example Protein that is Difficult to Solubilize

ITK was used as an illustrative target protein to exemplify the application of the disclosed systems for difficult to express soluble proteins. ITK is a member of the TEC family of kinases and is believed to play a role in T-cell proliferation and differentiation in T-cells. Also, ITK was used to demonstrate the consistency in enzyme activity in between batches and the scalability of the methods disclosed herein. ITK was expressed in 3×10 ml, 100 ml, and 1000 ml growth medium. Additionally, an ITK-L-his protein fusion construct (EG9) was used to demonstrate that enhancer proteins can be fused to the recombinantly expressed target proteins without losing the ability to control the regulation. ITK-his fusions were expressed in the presented system (EG17) and in the academic and industrial standard (EG18) as comparison.

ITK-his and ITK-L-his fusions were expressed in HEK293 cells. HEK293 cells were seeded at 2×10⁶ cells/ml in 10 ml, 100 ml or 1000 ml Expi293 medium and incubated at 37° C., 120 rpm and 5% CO₂ over night before transiently transfecting with either EG9, EG17 or EG18 as described above. Cells were harvested after 48h (5,000×g, 15 min, 4 C) and cell pellets were stored at −80° C. until further usage. To purify ITK, cells were resuspended in lysis buffer (40 mM Tris, 7.5; 20 mM MgCl₂; 0.1 mg/ml BSA; 50 μM DTT; and 2 mM MnCl₂, protease inhibitor, DNAse) and lysed by sonication (2 min, 10 s ON, 10 s OFF, 40% Amplitude) and crude cell extract was cleared (100,000×g, 45 min, 4 C). His-resin (GE Healthcare HisTrap) was equilibrated with wash buffer (40 mM Tris, 7.5; 20 mM MgCl2; 0.1 mg/ml BSA; 50 μM DTT; and 2 mM MnCl2) prior to adding to the cleared lysate. Lysate was incubated with the resin for 2 h at 4° C. with shaking. Resin was settled and washed with 5 CV wash buffer and proteins were eluted with 3 CV elution buffer (wash buffer+300 mM imidazole).

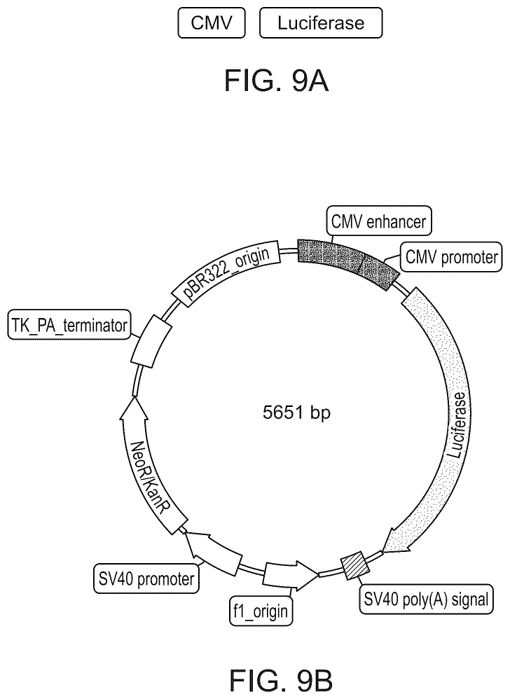

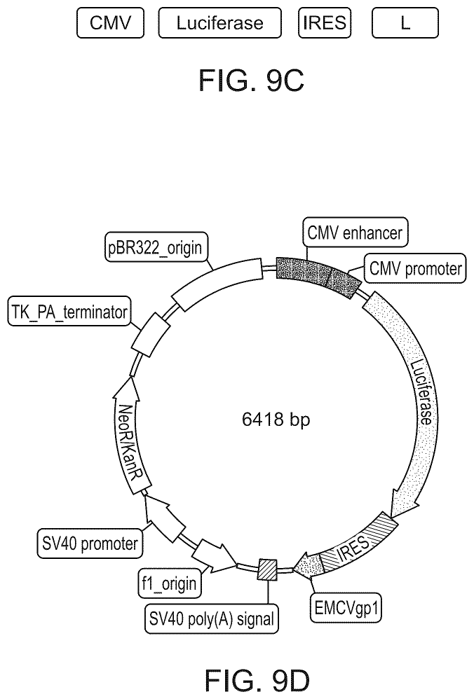

Purification was analyzed by SDS-PAGE (6-12% BOLT, ThermoFisher). FIG. 9A shows the SDS-PAGE results of His-tag purification of ITK. From left to right, the lanes show: protein ladder (Seeblue2 plus prestained), 500 ng GFP, 2 pg ITK, 5 pg ITK, and 10 pg ITK. The SDS-PAGE gel was then analyzed by non-reducing Western blot. Protein was transferred to a nitrocellulose membrane, bound with primary mouse-anti-His antibody and secondary anti-mouse-HRP antibody, and then visualized using NBT/BCIP solution. FIG. 9B shows the results of the Western blot analysis, with ITK protein monomers and dimers indicated by arrows.

After SDS-PAGE analysis, and protein containing fractions are pooled and concentrated. Protein is further purified by size-exclusion chromatography (SEC) (Superdex 200, ThermoFisher) and fractions are analyzed by SDS-PAGE (6-12% BOLT, ThermoFisher). Protein containing fractions are pooled and sent for analysis regarding phosphorylation pattern and sequence analysis.

ITK activity is analyzed using the ADP-Glo Kinase assay (Promega) according to manufacturer's instruction. E4Y1 is used as ITK substrate. ITK concentrations are varied from 0.1-500 ng. To compare the quality of the recombinantly expressed ITK with a standard available ITK, the ITK Kinase enzyme system (Promega) is used. ITK, substrate and ATP are diluted to working concentrations in wash buffer. ITK is mixed with substrate and ATP and incubated for 60 min at room temperature (RT). ADP-Glo reagent is added and reaction is incubated for 40 min at RT. The reaction is stopped by adding Kinase detection reagent and incubated for 30 min at RT. Luminescence is measured using a plate reader (Synergy LX (BioTek)) and data are analyzed using standard analysis programs.

### Example 9: Production of IL2 Inducible T Cell Kinase (ITK) was Compatible with CHO-K1 Cells

To demonstrate the compatibility of embodiments of the present system with other eukaryotic cell lines, the experiment of Example 7 is repeated using CHO cells instead of HEK293. ITK-his is expressed in either the presented system (EG17) or the industrial and academic standard (EG18).

ITK-his fusions are expressed in CHO-K1 cells. CHO-K1 cells are seeded at 2×10⁶ cells/ml 100 ml and incubated at 37° C., 120 rpm and 5% CO₂ over night before transiently transfecting with either EG17 or EG18 as described above. Cells were harvested after 48h (5,000×g, 15 min, 4 C) and cell pellets are stored at −80° C. until further usage. To purify ITK, cells are resuspended in lysis buffer (40 mM Tris, 7.5; 20 mM MgCl₂; 0.1 mg/ml BSA; 50 μM DTT; and 2 mM MnCl₂, protease inhibitor, DNAse) and lysed by sonication (2 min, 10 s ON, 10 s OFF, 40% Amplitude) and crude cell extract is cleared (100,000×g, 45 min, 4 C). His-resin (GE Healthcare HisTrap) is equilibrated with wash buffer (40 mM Tris, 7.5; 20 mM MgCl2; 0.1 mg/ml BSA; 50 μM DTT; and 2 mM MnCl2) prior to adding to the cleared lysate. Lysate is incubated with the resin for 2 h at 4° C. with shaking. Resin is settled and washed with 5 CV wash buffer and proteins was eluted with 3 CV elution buffer (wash buffer+300 mM imidazole). Purification is analyzed by SDS-PAGE (6-12% BOLT, ThermoFisher) and protein containing fractions are pooled and concentrated. Protein is further polished by size-exclusion chromatography (SEC) (Superdex 200, ThermoFisher) and fraction were analyzed by SDS-PAGE (6-12% BOLT, ThermoFisher). Protein containing fractions are pooled and send for analysis regarding phosphorylation pattern and sequence analysis.

### Example 10: Production of IL2 Inducible T Cell Kinase (ITK) was Compatible with Sf9 Cells

To demonstrate the compatibility of the presented system with other eukaryotic cell lines, the experiment of Example 7 is repeated using Sf9 cells instead of HEK293. ITK-his is expressed in either the presented system (EG17) or the industrial and academic standard (EG18).

ITK-his fusions are expressed in CHO-K1 cells. CHO-K1 cells are seeded at 2×10⁶ cells/ml 100 ml and incubated at 26° C. and 130 rpm over night before transiently transfecting with either EG17 or EG18 as described above. Cells were harvested after 48h (5,000×g, 15 min, 4 C) and cell pellets are stored at −80° C. until further usage. To purify ITK, cells are resuspended in lysis buffer (40 mM Tris, 7.5; 20 mM MgCl₂; 0.1 mg/ml BSA; 50 μM DTT; and 2 mM MnCl₂, protease inhibitor, DNAse) and lysed by sonication (2 min, 10 s ON, 10 s OFF, 40% Amplitude) and crude cell extract is cleared (100,000×g, 45 min, 4 C). His-resin (GE Healthcare HisTrap) is equilibrated with wash buffer (40 mM Tris, 7.5; 20 mM MgCl2; 0.1 mg/ml BSA; 50 μM DTT; and 2 mM MnCl2) prior to adding to the cleared lysate. Lysate is incubated with the resin for 2 h at 4° C. with shaking. Resin is settled and washed with 5 CV wash buffer and proteins was eluted with 3 CV elution buffer (wash buffer+300 mM imidazole). Purification is analyzed by SDS-PAGE (6-12% BOLT, ThermoFisher) and protein containing fractions are pooled and concentrated. Protein is further polished by size-exclusion chromatography (SEC) (Superdex 200, ThermoFisher) and fraction are analyzed by SDS-PAGE (6-12% BOLT, ThermoFisher). Protein containing fractions are pooled and sent for analysis of phosphorylation pattern and sequence analysis.

### Example 11: Materials and Methods of In Vivo Studies

### Construction of DNA Molecules

All assemblies were made into a plasmid backbone capable of propagation in E. coli comprising a promoter controlling a high copy number origin of replication (ColE1) followed by a terminator (rrnB T1 and T2 terminator). This is followed by a promoter controlling an antibiotic resistance gene which is isolated from the rest of the vector by a second terminator (transcription terminator from phage lambda). The genes comprising elements of the backbone were synthesized by phosphoramidite chemistry.

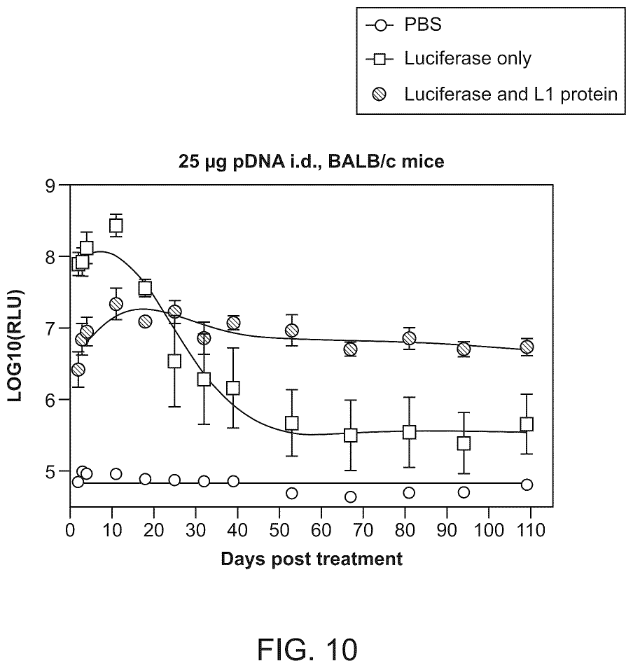

Structure genes used for the construction of the plasmids were synthesized by phosphoramidite chemistry, and cloned into the vector described above using restriction digest and golden gate assembly via Esp3I restriction with assembly site over GATG for the 3′ region and TAAG for the 5′ region. See FIG. 10.

The construct shown in F1G. 10A was synthesized and cloned using ESP3I with assembly sites GATG for the 3′ and TAAG for the 5′ into an adapted version of pVax1 to obtain a plasmid shown in FIG. 10B.

The construct shown in F1G. 10C was synthesized and cloned using ESP3I with assembly sites GATG for the 3′ and TAAG for the 5′ into an adapted version of pVax1 to obtain a plasmid shown in FIG. 13D.

TABLE 7
Nucleic acid sequences and amino acid
sequences of inserts

| | | | | SEQ |
|---|---|---|---|---|
| Gene name | Type of | | | ID |
| in plasmid | sequence | Sequence | Features | NO.: |
| Luciferase | Amino acid | MEDAKNIKKGPAPFYPLEDG | | 19 |
| in plasmid | | TAGEQLHKAMKRYALVPGTI | | |
| shown in | | AFTDAHIEVDITYAEYFEMS | | |
| FIG. 10B | | VRLAEAMKRYGLNTNHRIVV | | |
| | | CSENSLQFFMPVLGALFIGV | | |
| | | AVAPANDIYNERELLNSMGI | | |
| | | SQPTVVFVSKKGLQKILNVQ | | |
| | | KKLPIIQKIIIMDSKTDYQG | | |
| | | FQSMYTFVTSHLPPGFNEYD | | |
| | | FVPESFDRDKTIALIMNSSG | | |
| | | STGLPKGVALPHRTACVRFS | | |
| | | HARDPIFGNQIIPDTAILSV | | |
| | | VPFHHGFGMFTTLGYLICGF | | |
| | | RVVLMYRFEEELFLRSLQDY | | |
| | | KIQSALLVPTLFSFFAKSTL | | |
| | | IDKYDLSNLHEIASGGAPLS | | |
| | | KEVGEAVAKRFHLPGIRQGY | | |
| | | GLTETTSAILITPEGDDKPG | | |
| | | AVGKVVPFFEAKVVDLDTGK | | |
| | | TLGVNQRGELCVRGPMIMSG | | |
| | | YVNNPEATNALIDKDGWLHS | | |
| | | GDIAYWDEDEHFFIVDRLKS | | |
| | | LIKYKGYQVAPAELESILLQ | | |
| | | HPNIFDAGVAGLPDDDAGEL | | |
| | | PAAVVVLEHGKTMTEKEIVD | | |
| | | YVASQVTTAKKLRGGVVFVD | | |
| | | EVPKGLTGKLDARKIREILI | | |
| | | KAKKGGKIAV* | | |
| | Nucleic acid | GACATTGATTATTGACTAGT | Underlined | 20 |
| | | TATTAATAGTAATCAATTAC | CMV | |
| | | GGGGTCATTAGTTCATAGCC | enhancer | |
| | | CATATATGGAGTTCCGCGTT | Bold CMV | |
| | | ACATAACTTACGGTAAATGG | promoter | |
| | | CCCGCCTGGCTGACCGCCCA | Italics | |
| | | ACGACCCCCGCCCATTGACG | Luciferase | |
| | | TCAATAATGACGTATGTTCC | gene | |
| | | CATAGTAACGCCAATAGGGA | | |
| | | CTTTCCATTGACGTCAATGG | | |
| | | GTGGACTATTTACGGTAAAC | | |
| | | TGCCCACTTGGCAGTACATC | | |
| | | AAGTGTATCATATGCCAAGT | | |
| | | ACGCCCCCTATTGACGTCAA | | |
| | | TGACGGTAAATGGCCCGCCT | | |
| | | GGCATTATGCCCAGTACATG | | |
| | | ACCTTATGGGACTTTCCTAC | | |
| | | TTGGCAGTACATCTACGTAT | | |
| | | TAGTCATCGCTATTACCATG | | |
| | | GTGATGCGGTTTTGGCAGTA | | |
| | | CATCAATGGGCGTGGATAGC | | |
| | | GGTTTGACTCACGGGGATTT | | |
| | | CCAAGTCTCCACCCCATTGA | | |
| | | CGTCAATGGGAGTTTGTTTT | | |
| | | GGCACCAAAATCAACGGGAC | | |
| | | TTTCCAAAATGTCGTAACAA | | |
| | | CTCCGCCCCATTGACGCAAA | | |
| | | TGGGCGGTAGGCGTGTACGG | | |
| | | TGGGAGGTCTATATAAGCAG | | |
| | | AGCTATGGAAGATGCCAAAA | | |
| | | ACATTAAGAAGGGCCCAGCG | | |
| | | CCATTCTACCCACTCGAAGA | | |
| | | CGGGACCGCCGGCGAGCAGC | | |
| | | TGCACAAAGCCATGAAGCGC | | |
| | | TACGCCCTGGTGCCCGGCAC | | |
| | | CATCGCCTTTACCGACGCAC | | |
| | | ATATCGAGGTGGACATTACC | | |
| | | TACGCCGAGTACTTCGAGAT | | |
| | | GAGCGTTCGGCTGGCAGAAG | | |
| | | CTATGAAGCGCTATGGGCTG | | |
| | | AATACAAACCATCGGATCGT | | |
| | | GGTGTGCAGCGAGAATAGCT | | |
| | | TGCAGTTCTTCATGCCCGTG | | |
| | | TTGGGTGCCCTGTTCATCGG | | |
| | | TGTGGCTGTGGCCCCAGCTA | | |
| | | ACGACATCTACAACGAGCGC | | |
| | | GAGCTGCTGAACAGCATGGG | | |
| | | CATCAGCCAGCCCACCGTCG | | |
| | | TATTCGTGAGCAAGAAAGGG | | |
| | | CTGCAAAAGATCCTCAACGT | | |
| | | GCAAAAGAAGCTACCGATCA | | |
| | | TACAAAAGATCATCATCATG | | |
| | | GATAGCAAGACCGACTACCA | | |
| | | GGGCTTCCAAAGCATGTACA | | |
| | | CCTTCGTGACTTCCCATTTG | | |
| | | CCACCCGGCTTCAACGAGTA | | |
| | | CGACTTCGTGCCCGAGAGCT | | |
| | | TCGACCGGGACAAAACCATC | | |
| | | GCCCTGATCATGAACAGTAG | | |
| | | TGGCAGTACCGGATTGCCCA | | |
| | | AGGGCGTAGCCCTACCGCAC | | |
| | | CGCACCGCTTGTGTCCGATT | | |
| | | CAGTCATGCCCGCGACCCCA | | |
| | | TCTTCGGCAACCAGATCATC | | |
| | | CCCGACACCGCTATCCTCAG | | |
| | | CGTGGTGCCATTTCACCACG | | |
| | | GCTTCGGCATGTTCACCACG | | |
| | | CTGGGCTACTTGATCTGCGG | | |
| | | CTTTCGGGTCGTGCTCATGT | | |
| | | ACCGCTTCGAGGAGGAGCTA | | |
| | | TTCTTGCGCAGCTTGCAAGA | | |
| | | CTATAAGATTCAATCTGCCC | | |
| | | TGCTGGTGCCCACACTATTT | | |
| | | AGCTTCTTCGCTAAGAGCAC | | |
| | | TCTCATCGACAAGTACGACC | | |
| | | TAAGCAACTTGCACGAGATC | | |
| | | GCCAGCGGCGGGGCGCCGCT | | |
| | | CAGCAAGGAGGTAGGTGAGG | | |
| | | CCGTGGCCAAACGCTTCCAC | | |
| | | CTACCAGGCATCCGCCAGGG | | |
| | | CTACGGCCTGACAGAAACAA | | |
| | | CCAGCGCCATTCTGATCACC | | |
| | | CCCGAAGGGGACGACAAGCC | | |
| | | TGGCGCAGTAGGCAAGGTGG | | |
| | | TGCCCTTCTTCGAGGCTAAG | | |
| | | GTGGTGGACTTGGACACCGG | | |
| | | TAAGACACTGGGTGTGAACC | | |
| | | AGCGCGGCGAGCTGTGCGTC | | |
| | | CGTGGCCCCATGATCATGAG | | |
| | | CGGCTACGTTAACAACCCCG | | |
| | | AGGCTACAAACGCTCTCATC | | |
| | | GACAAGGACGGCTGGCTGCA | | |
| | | CAGCGGCGACATCGCCTACT | | |
| | | GGGACGAGGACGAGCACTTC | | |
| | | TTCATCGTGGACCGGCTGAA | | |
| | | GAGCCTGATCAAATACAAGG | | |
| | | GCTACCAGGTAGCCCCAGCC | | |
| | | GAACTGGAGAGCATCCTGCT | | |
| | | GCAACACCCCAACATCTTCG | | |
| | | ACGCCGGGGTCGCCGGCCTG | | |
| | | CCCGACGACGATGCCGGCGA | | |
| | | GCTGCCCGCCGCAGTCGTCG | | |
| | | TGCTGGAACACGGTAAAACC | | |
| | | ATGACCGAGAAGGAGATCGT | | |
| | | GGACTATGTGGCCAGCCAGG | | |
| | | TTACAACCGCCAAGAAGCTG | | |
| | | CGCGGTGGTGTTGTGTTCGT | | |
| | | GGACGAGGTGCCTAAAGGAC | | |
| | | TGACCGGCAAGTTGGACGCC | | |
| | | CGCAAGATCCGCGAGATTCT | | |
| | | CATTAAGGCCAAGAAGGGCG | | |
| | | GCAAGATCGCcGTGTAA | | |
| L1 in | Amino acid | MATTMEQETCAHSLTFEECP | | 21 |
| plasmid | | KCSALQYRNGFYLLKYDEEW | | |
| shown in | | YPEELLTDGEDDVFDPELDM | | |
| FIG. 10D, | | EVVFELQ* | | |
| 13B | | | | |
| | Nucleic acid | ATGGCCACAACCATGGAACA | | 22 |
| | | AGAGACTTGCGCGCACTCTC | | |
| | | TCACTTTTGAGGAATGCCCA | | |
| | | AAATGCTCTGCTCTACAATA | | |
| | | CCGTAATGGATTTTACCTGC | | |
| | | TAAAGTATGATGAAGAATGG | | |
| | | TACCCAGAGGAGTTATTGAC | | |
| | | TGATGGAGAGGATGATGTCT | | |
| | | TTGATCCCGAATTAGACATG | | |
| | | GAAGTCGTTTTCGAGTTACA | | |
| | | GTAA | | |
| IRES shown | Nucleic acid | CCCCCCCCCCTAACGTTACT | | 23 |
| in FIG. 10D, | | GGCCGAAGCCGCTTGGAATA | | |
| 13B | | AGGCCGGTGTGCGTTTGTCT | | |
| | | ATATGTTATTTTCCACCATA | | |
| | | TTGCCGTCTTTTGGCAATGT | | |
| | | GAGGGCCCGGAAACCTGGCC | | |
| | | CTGTCTTCTTGACGAGCATT | | |
| | | CCTAGGGGTCTTTCCCCTCT | | |
| | | CGCCAAAGGAATGCAAGGTC | | |
| | | TGTTGAATGTCGTGAAGGAA | | |
| | | GCAGTTCCTCTGGAAGCTTC | | |
| | | TTGAAGACAAACAACGTCTG | | |
| | | TAGCGACCCTTTGCAGGCAG | | |
| | | CGGAACCCCCCACCTGGCGA | | |
| | | CAGGTGCCTCTGCGGCCAAA | | |
| | | AGCCACGTGTATAAGATACA | | |
| | | CCTGCAAAGGCGGCACAACC | | |
| | | CCAGTGCCACGTTGTGAGTT | | |
| | | GGATAGTTGTGGAAAGAGTC | | |
| | | AAATGGCTCTCCTCAAGCGT | | |
| | | ATTCAACAAGGGGCTGAAGG | | |
| | | ATGCCCAGAAGGTACCCCAT | | |
| | | TGTATGGGATCTGATCTGGG | | |
| | | GCCTCGGTGCACATGCTTTA | | |
| | | CATGTGTTTAGTCGAGGTTA | | |
| | | AAAAAACGTCTAGGCCCCCC | | |
| | | GAACCACGGGGACGTGGTTT | | |
| | | TCCTTTGAAAAACACGATGA | | |
| | | TAAT | | |

### Cell Lines—Culturing and Transfection

HEK293 cells were used to validate constructs in vitro before injecting them into animal. HEK293 adherent cells (CLS) were cultured in Dulbecco's Modified Eagle Medium high glucose (Gibco) supplemented with 10% Fetal Bovine Serum (Gibco) and 50,000 U Pen Strep (Gibco). HEK293 cells were grown to 80% confluency at 37° C. and 5% CO₂ before transiently transfecting using 293 fectin (ThermoFisher) according to manufacturer's instruction. Protein-expressing cells were harvested after 48h by detaching the cells using 0.5% trypsin solution for 5 min at 37° C. and scraping. Cells were pelleted (5,000×g, 15 min, 4° C.) and supernatant was discarded. Cell pellets were stored at −80° C. until further usage.

HEK293 suspension adapted cells were cultured in Expi293 serum free medium (Gibco). HEK293 cells seeded at 3.0×10\hat{ }6 cell/ml at 37° C., 5% CO₂ and 120 rpm before transiently transfecting using ExpiFectamin 293 (Gibco) according to manufacturer's instruction. Depending on the example, for protein-expression, cells were harvested after 48h-72h by pelleting the cells (5,000×g, 15 min, 4° C.) and supernatant was discarded. Cell pellets were stored at −80° C. until further usage. For secreted proteins, the supernatant was collected after 96h and cleared by centrifugation (5,000×g, 15 min, 4° C.). The supernatant was immediately used for further analysis or purification.

### Animals

For animal studies, BALB/c mice (Charles River Laboratories) and wild type mice were used. A corresponding disease model could also be used. Age is indicated in the examples. Animals of the same sex were group housed in polycarbonate cages containing appropriate bedding. Mice were identified with by either visible tattoos on the tail or by implantation of an electronic identification chip. Mice were allowed acclimation for at least 5 days prior to treatment to accustom the animals to the laboratory environment. Housing was set-up as described in the Guide for the Care and Use of Laboratory Animals with social housing and a chewing object for animal environmental enrichment. The targeted environmental conditions were a temperature of 19 to 25° C., a humidity between 30% to 70% and a light cycle of 12h light and 12h dark. Food (Lab Diet Certified CR Rodent Diet 5CR4) was provided ad libitum in form of pellets. Water was provided freely available in form of municipal tap water, treated by reverse osmosis and ultraviolet irradiation.

### Example 12: The In Vivo Expression of the L Enhancer Protein with Luciferase Increases the Stability and Duration of Luciferase Expression

To evaluate the expression of firefly luciferase in vivo, groups of 4 mice each of 6-8 weeks old female BALB/c mice were used. The groups were once injected intradermally with 50 μl of 25 μg in PBS (137 mM NaCl, 2.7 mM KCl, 10 mM Na₂HPO₄, 1.8 mM KH₂PO₄ pH 7.4), of (a) the plasmid encoding luciferase shown in FIG. 9B or (b) the plasmid encoding luciferase sequential with an enhancer protein shown in FIG. 9D. For baseline measurements, 4 mice were injected intradermally with 50 μl PBS only. The volume of the dose was administered using a syringe/needle within the demarcated area of the lumbar or sacral region of the dorsum.

Animals were randomized in groups and bodyweight was recorded on day 1 and then bi-weekly until the end of the study. Adverse events (RM, SD, RD) were recorded according to good laboratory standards. Any individual animal with a single observation of >30% body weight loss or three consecutive measurements of >25% body weight loss was euthanized.

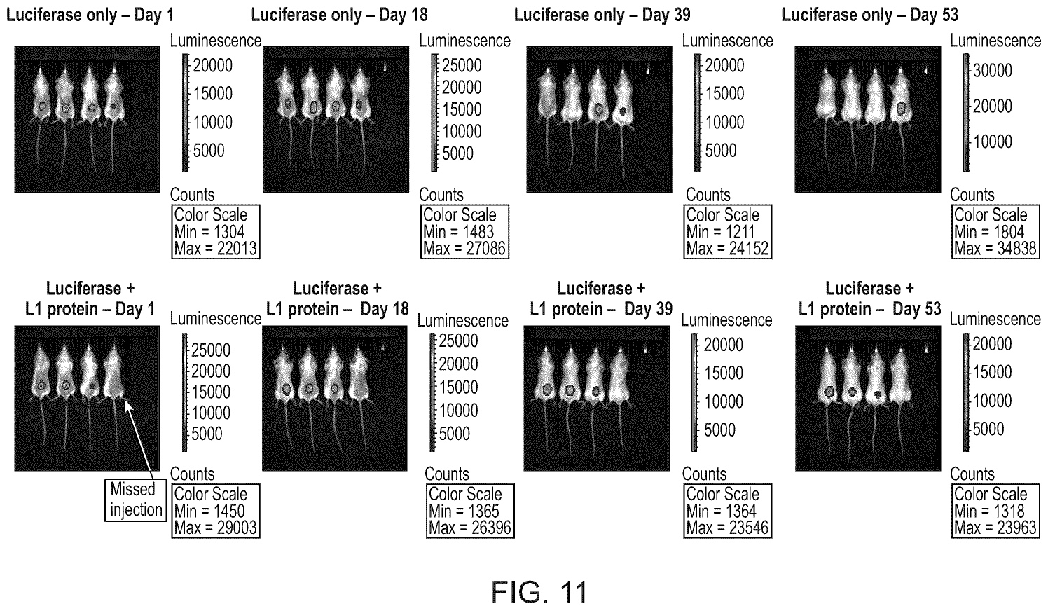

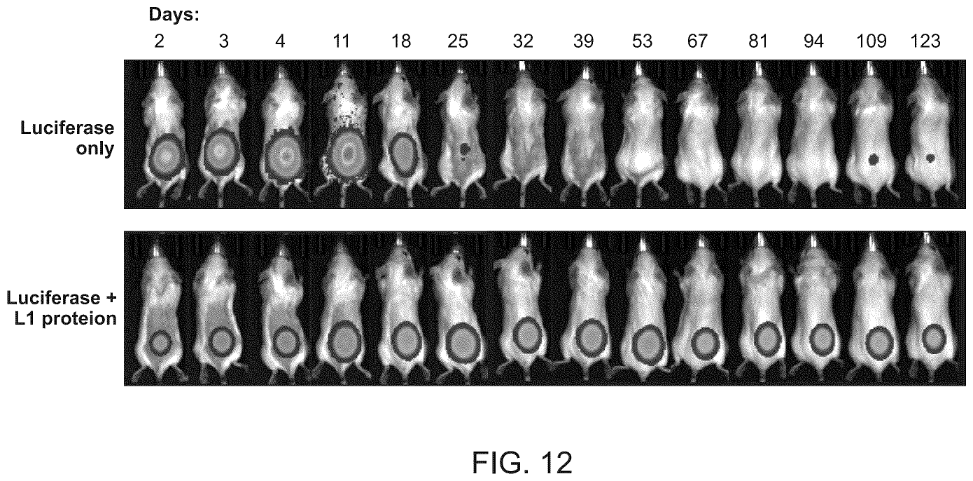

The expression of luciferase was measured by bioluminescence imaging of the whole animal on days 2 (24 hrs post injection), 3 (48 hrs post injection), 4 (72 hrs post injection), 11, 18, 25, 32, 38, 53, 67. The endpoint of the study, when all animals were euthanized, was Day 67. Bioluminescence imaging was conducted under anesthesia. Dorsal images were captured 10 min post substrate injection of luciferin. Substrate was administered at 150 mg/kg i.p at 10 ml/kg based on recent body weights. See FIGS. 11 and 12.

These results show that when luciferase is expressed along with an enhancer protein, such as L1 protein of EMCV, then the in vivo expression of luciferase is maintained for longer. As shown in FIG. 10, the expression level of luciferase in the control animal is initially high and then reduces by 30 days post treatment. In contrast, the expression level of luciferase, when expressed in combination with the L1 protein, is maintained at a steady level for a longer period of time, even until 110 days post treatment. That is, there is less variation in the level of luciferase expression over time when luciferase is expressed along with L1 protein.

Further, the variation in the expression level of luciferase among animals expressing luciferase and L1 protein is less than among animals expressing only luciferase. This effect is especially evident over time, such as at the 53 day time point. See FIG. 11. While only 25% of the animals (1 out of 4) injected with the plasmid of FIG. 9B show detectable luciferase expression at 53 days after injection, 100% of the animals (3 out of 3) that were injected with the plasmid of FIG. 9D showed luciferase expression at the 53 day time point. Therefore, the disclosed methods reduce variability in expression levels of the target protein among animals, providing more reproducibility in the expression of the target protein, which has important applications in therapeutics.

Finally, FIG. 12 further demonstrates that the expression of luciferase in the presence of the enhancer protein results in more stable expression over a longer period of time. While the control mouse expressing only luciferase shows high levels of luciferase for a few days followed by tapering off of the expression down to a minimum, the mouse expressing luciferase in the presence of L1 protein shows detectable and stable luciferase expression for 123 days. This result demonstrates the superior advantages of the disclosed compositions and methods for in vivo expression of target proteins.

To demonstrate that the beneficial effect over time of reporter gene expression was not specific to either of the administration route or the injection site, the experiment was repeated using subcutaneous administration. In short, groups of four female BALB/c mice, 6-8 weeks old were used. The groups were injected once subcutaneously with 200 μl of 30 μg naked plasmid in PBS (137 mM NaCl, 2.7 mM KCl, 10 mM Na₂HPO₄, 1.8 mM KH₂PO₄ pH 7.4), with either the plasmid encoding luciferase shown in FIGS. 9A and 9B or the plasmid encoding luciferase sequential with an enhancer proteinshown in FIGS. 9C and 9D. For baseline measurements, four control mice were treated with only PBS. The dose was administered using a syringe-needle as a subcutaneous injection between the scapulae.

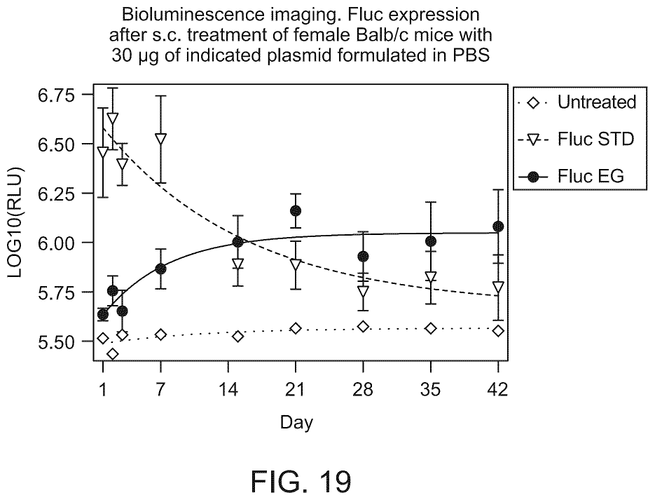

Mice were dosed on day 0, and the expression of luciferase was measured by bioluminescence imaging of the whole body of the animal on days 1, 2, 3, 7, 15, 21, 28, 35 and 42 (imaging data quantified in FIG. 19). For imaging, luciferin (i.e., the substrate of firefly luciferase) was injected into the intraperitoneal (i.p.) cavity of mice at the dose of 150 mg/kg at 10 ml/kg volume. The images were acquired 10 min after substrate administration under anesthesia. Mice were imaged in the prone positions to focus on the injection site.

FIG. 19 shows the results from bioluminescence imaging of Firefly luciferase (Fluc) after subcutaneous treatment of Balb/c mice with either the plasmid expressing either Firefly luciferase in combination with an enhancer protein(Fluc EG) or the same plasmid without the enhancer protein (Fluc Std). As described above, the addition of the enhancer proteinincreases the time that the reporter gene is functionally expressed. The enhancer proteinleads to stable expression over the whole period of time of the experiment, whereas without the enhancer protein, a loss of active luciferase expression can be detected within days. Surprisingly, the stabilizing effect of the enhancer protein is independent of the injection site. This result demonstrates the general applicability of the compositions and methods disclosed herein for in vivo expression of target proteins.

### Example 13: Expression of the L Enhancer Protein with Adalimumab Improves Adalimumab Protein Quality and Expression Level

To test whether adalimumab was expressed in HEK293T cells from a control plasmid comprising a nucleic acid sequence encoding adalimumab (EG140, FIG. 13A), and a plasmid comprising an enhancer gene in combination with the nucleic acid sequence encoding adalimumab (EG141, FIG. 13B), the following experiment was performed.

On Day 0, HEK293T cells were seeded on 24-well plates at 20,000 cells/well. On Day 1, the growth medium was changed to Opti-MEM (450 μl per well). Cells were then transfected using 0.5 μg plasmid and 1 μg PEI per well in 1:2 ratio, as per standard transfection procedure. A total of 6 replicates were used. On Day 3, the supernatant of the cell culture supernatant was collected for ELISA and cell fixation for immunofluorescence microscopy. Any remaining cellular debris was removed using centrifugation performed at 500×g for 5 minutes. The clear supernatant was then pipetted to new 1.5 ml Eppendorf tubes and stored at −20° C. until analysis by ELISA.

Cells were fixed using 10% neutral buffered formalin for 10 minutes at room temperature (RT), and permeabilized using 0.2% Triton X-100 for 10 minutes at room temperature. Cells were stained using DyLight488-labeled anti-human antibody (IgG Fc Cross-Adsorbed Goat anti-Human, DyLight® 488, Invitrogen—PISA510134 ANTI-HUMAN IGG-FC XMIN D488) at 1:500 dilution for 1 hour at RT, and washed. Cells were imaged using the FLoid fluorescence microscope.

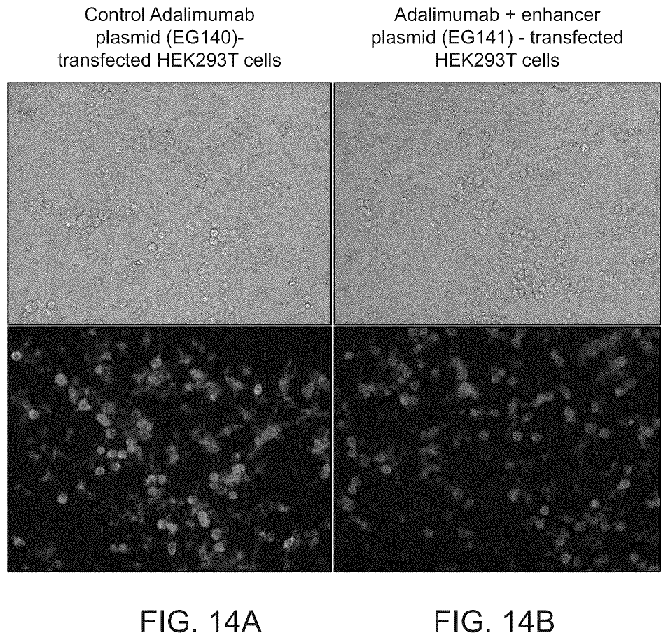

The immunofluorescence results are presented in FIG. 14. The results show that adalimumab was expressed from both the EG140 and EG141 plasmids in HEK293T cells. While the expression of adalimumab from either plasmid was readily detected, the expression of adalimumab in combination with the EMCV L1 protein (from EG141) was marginally lower than when adalimumab was expressed on its own (from EG140). Strikingly, when adalimumab is expressed in combination with the L1 enhancer protein, the intracellular distribution of the antibody is more uniform (FIG. 14B), as compared to in the absence of the enhancer protein (FIG. 14A). Moreover, intracellular foci indicating the presence of misfolded or unfolded protein seen upon expression of adalimumab alone are absent when adalimumab is expressed in combination with the L1 enhancer. These results further support that the presence of the L1 enhancer protein improves the expression quality and/or quantity of recombinant adalimumab antibody expressed in cells.

Further, it was tested whether adalimumab can be detected in the cell culture supernatant using the direct enzyme-linked immunosorbent assay (ELISA) method. For ELISA experiments, frozen cell culture supernatants were thawed and used for coating ELISA high-binding plates. Coating was performed using 2× dilution series of cell culture supernatants, 75 μl per well. For positive control, a dilution series of recombinant human anti-TNFa antibody (NBP2-62567, Novus Biologicals) was used instead. Coating was carried out overnight at 4° C.

The next day, the ELISA plate was washed 1× using PBS-T and blocked using EZ Block for 2 h at 37° C., 150 μl per well. The plate was washed and incubated with the secondary antibody (anti-human HRP labelled antibody, IgG (H+L) Goat anti-Human, HRP, Invitrogen—A18805 GTXHU IGG HRP AFFINITY) at a dilution of 1:2000, 1 hour at RT. The plate was washed 5× using PBS-T. TMB substrate was added (75 μl per well) and incubated 20 minutes, followed by adding the stop solution (75 μl per well). The absorbance at 450 nm was measured using the Biotek plate reader.

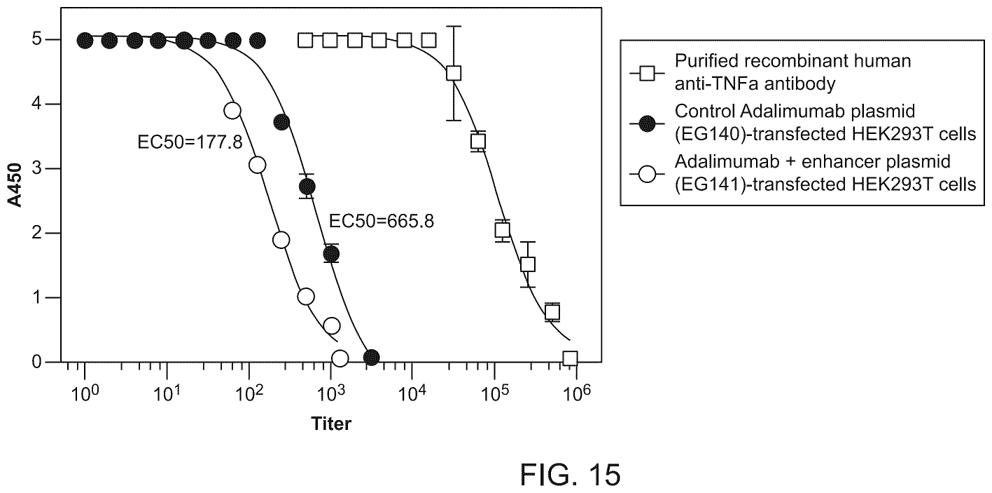

The results are presented in FIG. 15. As the ELISA plate was coated with cell culture supernatants derived from adalimumab-expressing HEK293T cells, the results suggest that adalimumab is readily detectable in the supernatant of both EG140- and EG141-transfected cells. The level of adalimumab upon expression of EG140 (control) is >3× higher than upon expression of EG141 (plasmid encoding adalimumab and L1 enhancer).

### Example 14: Expression of the L Enhancer Protein Significantly Improves Adalimumab Protein Quality and Expression Level

To detect whether the antibodies expressed and secreted from EG140- or EG141-transfected cells can specifically bind recombinant human TNF-alpha, the following experiment was performed.

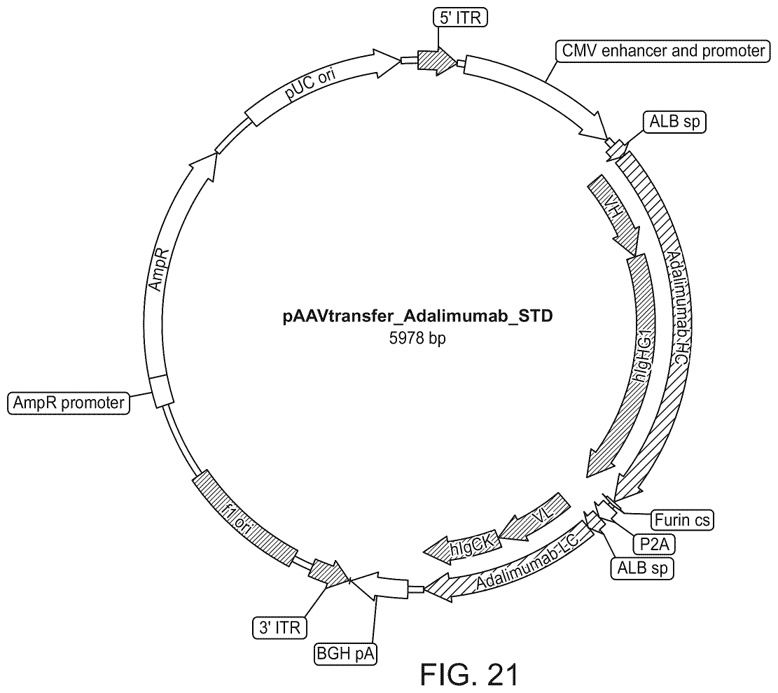

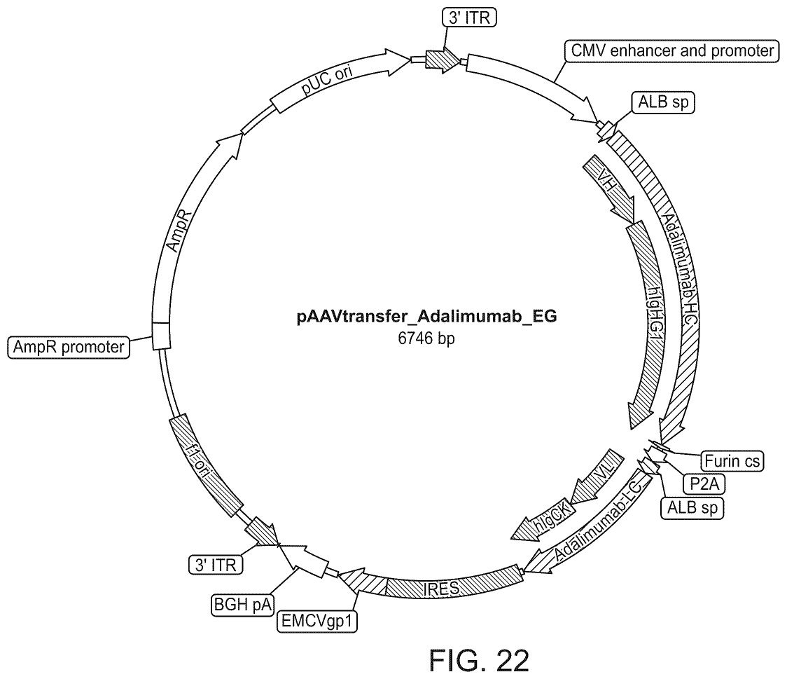

In addition to the plasmids EG140 and EG141, the same expression cassettes were cloned into an AAV transfer plasmid backbone, which differs from EG140 and EG141 backbone by a different polyadenylation signal and an expression cassette flanked by inverted terminal repeats (ITRs) for the purposes of generating recombinant AAV vectors for downstream in vivo use of AAVs in animals (FIG. 20). To test whether adalimumab from the AAV transfer plasmid without (FIG. 21) and with the enhancer protein(FIG. 22) expressed in HEK293T cells can specifically bind recombinant human TNF-alpha, the following experiments were performed.

Cells were grown and transfected as described in Example 13. However, in contrast to Experiment 13, the high binding ELISA plates were first coated with recombinant TNF-alpha (1 μg/ml, 75 μl per well) overnight at 4° C. On Day 2, the ELISA plates were washed with PBST and blocked with EZ Block reagent. Cell culture supernatant samples were diluted in Opti-MEM (1:256) and added to blocked wells (75 μl per well, 1 hour at 37° C.). The wells were washed 3× using PBST and the secondary antibody was added (anti-human IgG HRP) at 1:2000 dilution (75 μl per well, 1 hour at 37° C.). The wells were then washed 5× using PBST. 75 μl of 3,3′,5,5′-Tetramethylbenzidine (TMB) substrate was added to detect the bound antibodies. The reaction was stopped using 75 μl of the TMB Stop Solution, and the signal was read by measuring the absorbance at 450 nm using a Biotek microplate reader.

In some experiments the total concentration of secreted adalimumab was measured using quantitative ELISA. In those experiments, the ELISA was performed as described above, with the following modifications. Cell culture supernatant was pre-diluted in the EZ Block reagent and added to the pre-coated and blocked wells as samples. The positive control antibody, recombinant monoclonal adalimumab (Novus Biologicals NB001486) was diluted in the EZ Block reagent to the concentrations of 0, 0.1, 1, 10 and 100 ng/ml, and added to the pre-coated and blocked wells as a standard curve. In every other respect, the ELISA was performed as described above. After reading the absorbance at 450 nm, the absorbance values of samples were converted to total secreted adalimumab concentration in the unit of ng/ml using the absorbance values of the standard curve.

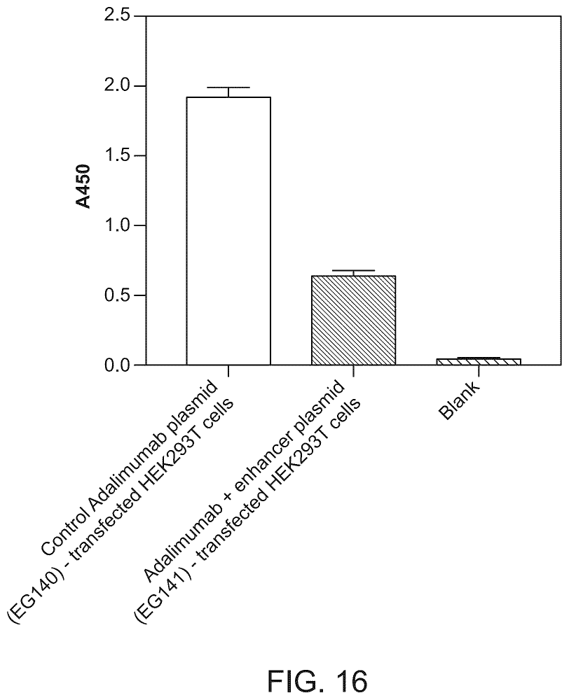

The results showed that cell culture supernatants from EG140-transfected cells contained approximately three times more TNF-alpha binding human antibodies as compared to EG141-transfected cells (FIG. 16).

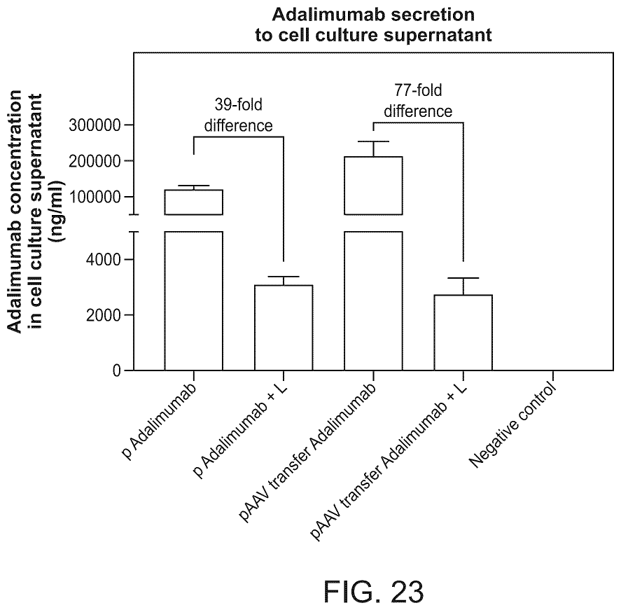

The results for the AAV transfer plasmid encoding adalimumab with (SEQ ID NOS: 243-272) and without the enhancer L protein (SEQ ID NOS: 217-242) is shown in FIG. 23, together with a repeated experiment of transfected plasmids encoding adalimumab without the enhancer protein (STD) and with the enhancer protein (EG). Secreted adalimumab concentration was quantified in ng/ml by quantitative ELISA. In both cases the presence of the enhancer protein reduced the total amount of adalimumab in the cell supernatant. Notably, the presence of adalimumab in a standard plasmid with the enhancer protein reduced the presence of adalimumab protein in the supernatant by 39-fold, while the enhancer proteinin the AAV transfer plasmid reduced the presence of adalimumab protein in the supernatant by 77-fold.

Analysis of adalimumab quality was tested by the level of TNF-alpha activation in the cell culture supernatant. Active adalimumab will block TNF-alpha. This experiment was performed using Luciferase TNF-alpha reporter cells.

The isolated cell culture supernatants were analyzed using a reporter cell designed to monitor the levels of bioactive TNF-alpha in samples by assessing the activation of NF-kappaB. The reporter HEK-Dual TNF-a cell line (Invivogen) was generated by transfecting the HEK293 cell with a NF-kappaB inducible secreted firefly luciferase. Upon TNF-alpha treatment, the NF-kappaB pathway is activated, which leads to the expression of secreted luciferase, which can be detected using a luciferase substrate. In the presence of TNF-alpha neutralizing antibodies in the cell culture supernatants of EG140- and EG141-transfected cells, or the same expression cassettes cloned into the AAV transfer plasmid backbone, NF-kappaB activation was predicted to be inhibited and luciferase signal reduced.

The experiment was performed as follows. The reporter cells were seeded on a 96-well plate (5000 cells per well) in DMEM+10% FBS. The next day, cell culture supernatants from EG140- and EG141-transfected cells were diluted in cell culture medium at indicated ratios and mixed with 1 ng/ml human recombinant TNF-alpha for 30 minutes at room temperature. The cell media of pre-seeded TNF-alpha reporter cells in 96-well plates was then replaced with the pre-incubated cell culture supernatant/TNF-alpha samples using 100 μl of mixture per well. The reporter cells were then incubated at 37° C. for 5 hours. The secreted luciferase signal was then detected using the Quanti-Luc Gold assay (Invivogen) according to manufacturer's instructions to detect TNF-alpha activation, measuring the luciferase activity using a microplate reader.

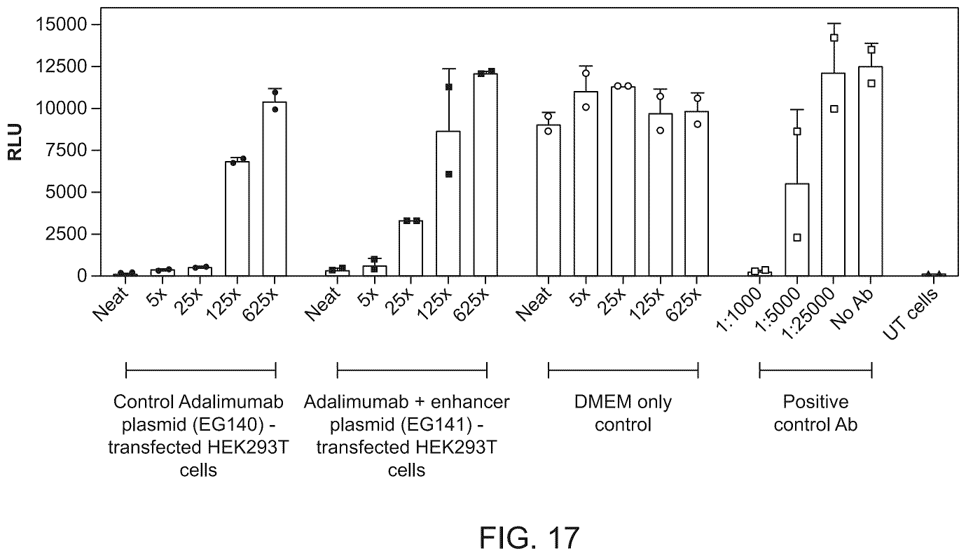

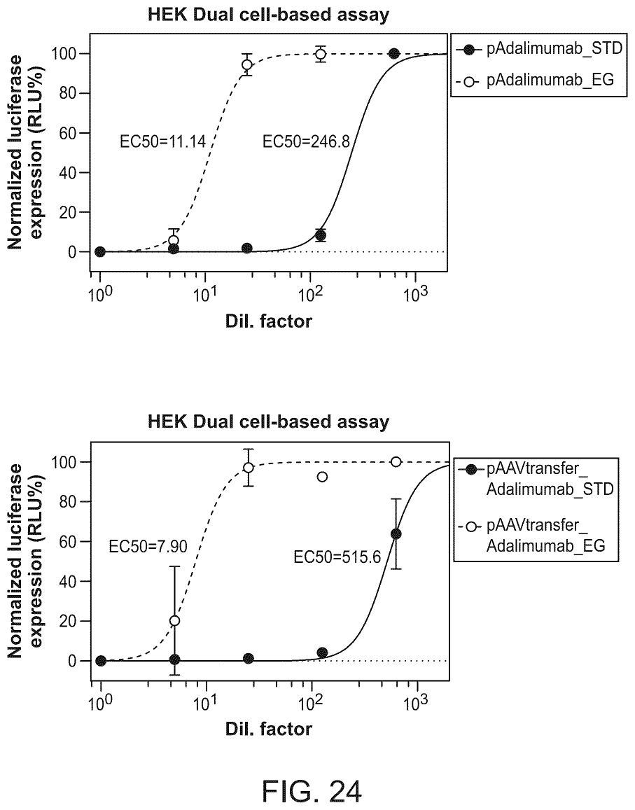

The results are presented in FIG. 17, FIG. 24 and Tables 4 and 5. While the cell culture supernatants isolated from both EG140- and EG141-transfected cells were able to inhibit TNF-alpha mediated activation of NF-kappaB driven Luciferase expression, the supernatants of EG140-transfected cells were about 3 times more active at suppressing TNF-alpha activation, as compared to the supernatants of EG141-transfected cells.

TABLE 4
Calculated EC₅₀ values of adalimumab constructs
with (EG) and without the enhancer protein (STD)

| | Construct | | |
|---|---|---|---|
| | pAdali- | pAdali- | |
| | mumab_ | mumab_ | |
| | STD | EG | |
| | (SEQ | (SEQ | Fold |
| | ID NOS: | ID NOS: | differ- |
| | 169-190) | 191-216) | ence |
| Secreted Adalimumab | 121.5 | 3.1 | 39.1X |
| concentration measured by | | | |
| anti-TNFα ELISA (μg/ml) | | | |
| Secreted Adalimumab biological | 246.8 | 11.14 | 22.2X |
| activity in HEK293 Dual reporter | | | |
| cells (EC50 of the titer) | | | |

TABLE 5
Calculated EC₅₀ values of adalimumab AAV constructs
with (EG) and without the enhancer protein (STD)

| | Construct | | |
|---|---|---|---|
| | pAAVtransfer_ | pAAVtransfer_ | |
| | Adalimumab_ | pAdalimumab_ | |
| | STD | EG | |
| | (SEQ | (SEQ | Fold |
| | ID NOS: | ID NOS: | differ- |
| | 217-242) | 243-272) | ence |
| Secreted Adalimumab | 213.2 | 2.5 | 77.3X |
| concentration measured by | | | |
| anti-TNFα ELISA (μg/ml) | | | |
| Secreted Adalimumab | 515.6 | 7.9 | 65.3X |
| biological activity in | | | |
| HEK293 Dual reporter | | | |
| cells (EC50 of the titer) | | | |

Additionally, to estimate the relative quality of secreted adalimumab, the total amount of secreted adalimumab in supernatants (in units of ng/ml, as measured by quantitative ELISA) was compared to the potency of the same supernatants to suppress TNF-alpha signaling (in EC₅₀ units, as measured by using the HEK Dual TNF-alpha reporter cells).

Interestingly, although the amount of the produced and secreted adalimumab for the standard system was higher (FIG. 24 and Tables 4 and 5) the relative quality of the produced adalimumab, as defined by the biological activity, was higher in constructs with the enhancer L protein. Namely, the activity with the enhancer protein was 22.2 fold higher in the standard construct transfected cells and 65.3 fold higher in the AAV construct transfected cells.

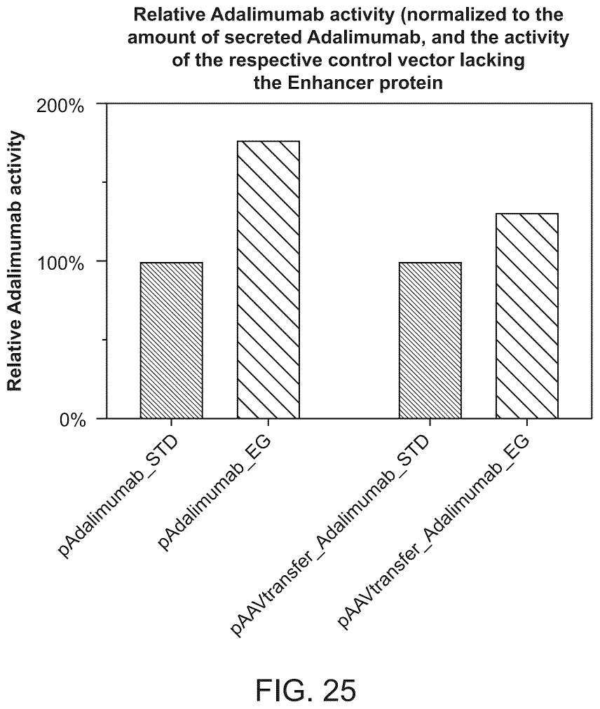

Notably, the relative percentage of biologically active adalimumab was further analyzed. FIG. 25 demonstrates that relative amount of active adalimumab with enhancer protein co-expression was higher than without. Importantly, the percentage of active expressed adalimumab was up to 60% higher with enhancer protein co-expression.

### Example 15: Expression of the L Enhancer Appears to Reduce the Number of Inactive Adalimumab Expression Products

To visualize the total amount of secreted antibodies in cell culture supernatants of HEK293T cells transiently transfected with EG140 and EG141 plasmids, the following experiment was performed. Cells were seeded into 15-cm cell culture dishes 4E6 cells per dish and transfected using 40 μg plasmid and 80 μg PEI. Antibodies were purified from cell culture supernatants using Protein A/G agarose resin (MPbio), as per manufacturer's instructions. Equal volumes of purified antibody were analyzed on SDS-Page and by western blotting (GenScript) as per manufacturer's instructions.

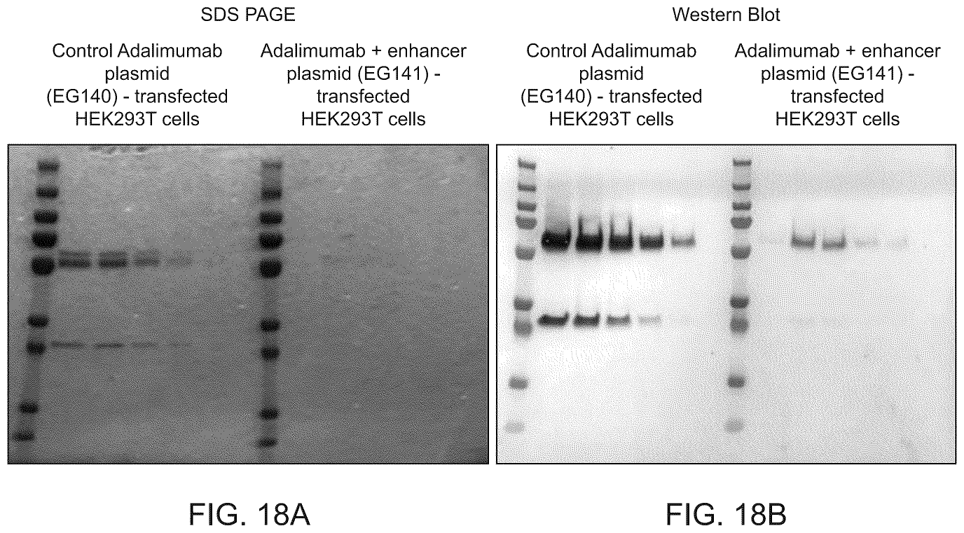

The SDS PAGE (FIG. 18A) and western blot (FIG. 18B) results indicate that purified adalimumab light and heavy chains were detected in cell culture supernatants of both EG140- and EG141-transfected HEK293T cells. The results show that amount of purified adalimumab expressed in control EG140-transfected cells is significantly greater than the purified adalimumab expressed in the presence of the L1 enhancer in EG141-transfected cells. Without being bound by a theory, it is thought that a large proportion of secreted adalimumab expressed by EG140-transfected cells is potentially non-functional due to misfolding and/or improper localization to intracellular foci, as compared to the secreted adalimumab expressed by EG141-transfected cells.

### Example 16: Expression of the L Enhancer Protein In Vivo Reduces Injection Site Dependent Effects

To evaluate the expression of adalimumab in vivo, groups of 6 female BALB/c mice, each 6-8 weeks, were used. The groups were once injected either via intramuscular (i.m.) or subcutaneous (s.c.) route with 50 μl of 2×10¹¹ genome copies of recombinant AAVs in PBS (137 mM NaCl, 2.7 mM KCl, 10 mM Na₂HPO₄, 1.8 mM KH₂PO₄ pH 7.4). The used recombinant AAV vectors were produced using either (a) the plasmid encoding adalimumab shown in FIG. 21 or (b) the plasmid encoding adalimumab with an enhancer protein shown in FIG. 22. The recombinant AAV vectors were produced by VectorBuilder Inc. by triple-transfecting the AAV transfer plasmid together with the helper plasmid (encoding adenovirus genes E4, E2A and VA) and the Rep-Cap plasmid (encoding the Cap proteins of AAV serotype 8, AAV8) into HEK293T packaging cells. Similarly, any replication genes that are sufficient for viral genome replication and packaging, and any viral capsid genes that are sufficient for viral capsid formation may be used in place of Rep-Cap. Two days after transfection, the cells were harvested, concentrated by PEG and purified by CsCl gradient purification.

The pharmacokinetics of adalimumab in AAV8-treated mice were compared to that of mice treated with recombinantly produced adalimumab protein as a control, which mimics the current standard of care of anti-TNFa therapies. In the control group, 3 mice were injected subcutaneously with 50 μl of 100 μg recombinantly produced adalimumab in PBS.

Animals were randomized in groups and body weight was recorded on day 1 and then bi-weekly until the end of the study. Adverse events (RM, SD, RD) were recorded according to good laboratory standards. Any individual animal with a single observation of >20% body weight loss, wounds that inhibited normal physiological function such as eating, drinking and mobility, or clinical observation of prostration, seizure and haemorrhages, were euthanized.

Whole blood was collected by submandibular vein and processed to collect serum for analysis. Blood was collected on Day 0 (prior to dosing), Day 3, Day 7, Day 14, Day 21, Day 28 and Day 42. Serum was analyzed for adalimumab concentration by quantitative ELISA.

The quantitative ELISA was performed as described in the Example 14 above, by using mouse serum samples (pre-diluted in the EZ Block reagent) as samples. The positive control antibody, recombinant monoclonal adalimumab (Novus Biologicals NB001486) was diluted in the EZ Block reagent to the concentrations of 0, 0.1, 1, 10 and 100 ng/ml, was used as a standard curve. After reading the absorbance at 450 nm, the absorbance values of samples were converted to total secreted adalimumab concentration in serum in the units of pg/ml using the absorbance values of the standard curve.

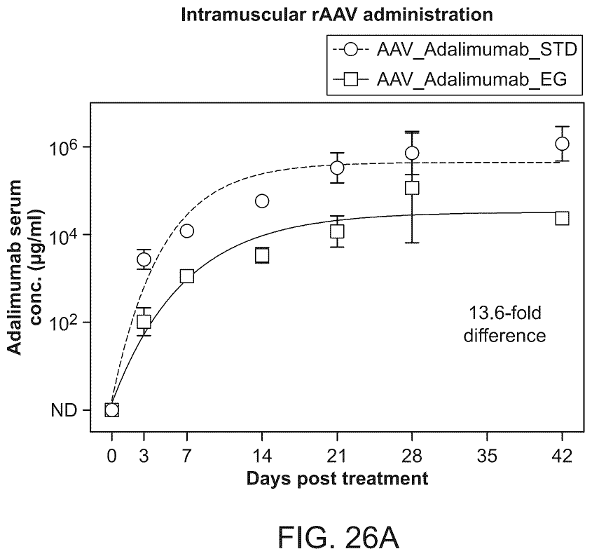

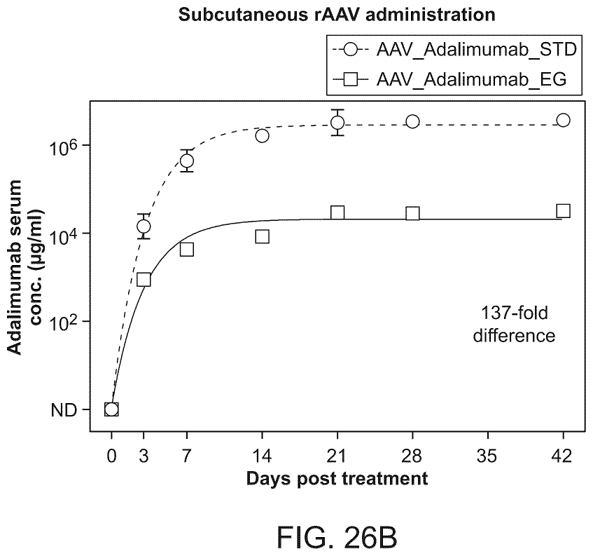

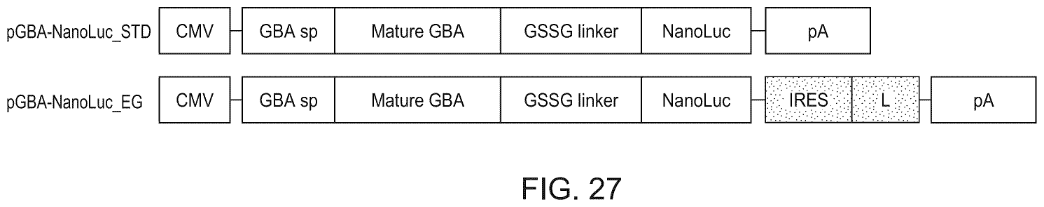

The results of the ELISA experiments are shown FIGS. 26A and 26B. The straight dotted line threshold shows adalimumab concentration of 5 pg/ml, above which a therapeutic effect of adalimumab is observed in rheumatoid arthritis models. FIG. 26A shows the result of intramuscular injection of AAVs with (EG) and without (STD) the enhancer protein. FIG. 26B shows the subcutaneous injection of the same materials. As described above regarding other experiments presented herein, the system without the enhancer protein (STD) showed a higher concentration of adalimumab in the blood serum compared to the system with the enhancer protein (EG).

Notably, the concentration of adalimumab without the enhancer protein co-expression was highly dependent on the site of injection. In total, a 10-fold difference between the injection sites could be observed. This might be highly challenging for specific gene therapies where the injection site can't be varied because relatively small deviations in treatment administration could lead to large changes in the steady state serum levels of the therapeutic transgene.

In contrast, the system with the enhancer protein (EG) showed bloods stream dosing independent of the injection site. These data demonstrate that the enhancer protein ensured similar protein expression levels regardless of the cell type during therapy, an important result for achieving robust and reproducible therapeutic effects. Importantly, in both intramuscular and subcutaneous administration, an identical level of adalimumab was found in the blood serum of treated mice, both over the required therapeutic concentration. Surprisingly, the addition of the enhancer protein ensured that that adalimumab was expressed in a stable and constant level independent of the injection site.

### Example 17: Expression of the L Enhancer Protein Increases Relative the Activity of Secreted Glucosylceramidase (GBA) In Vitro

To test Glucosylceramidase (GBA) expression with and without the enhancer L protein, in HEK293T cells, the following experiment was performed.

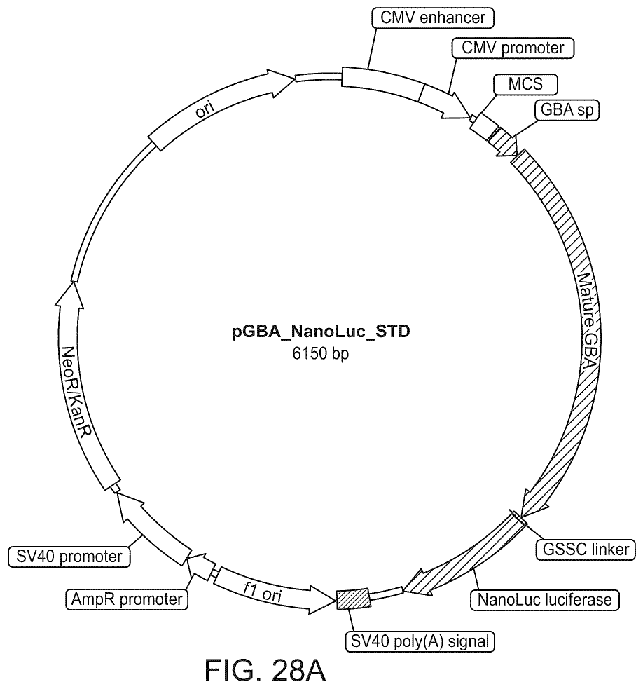

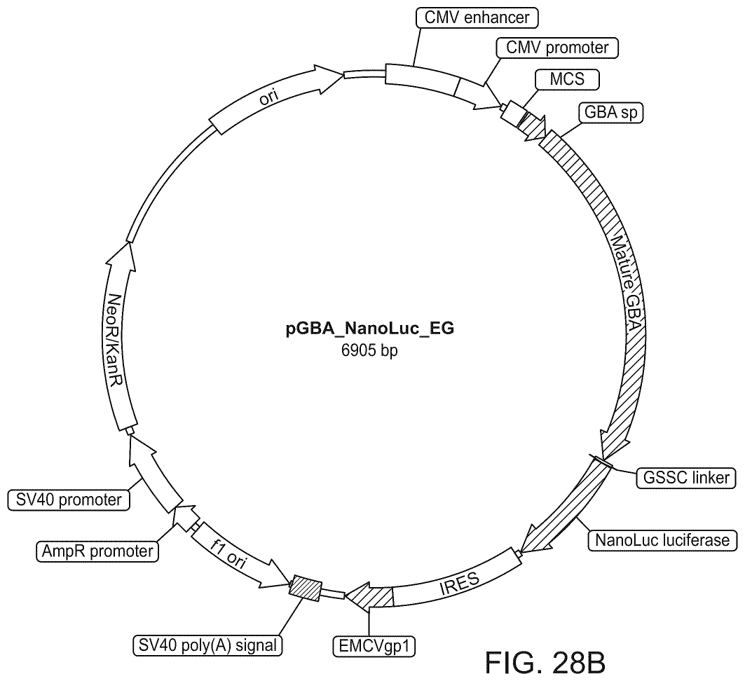

HEK293T cells were transfected with a control plasmid expressing GBA-NanoLuc fusion protein (FIG. 28A) and with a plasmid co-expressing the enhancer protein L with the GBA-NanoLuc chimera (FIG. 28B). On Day 0, HEK 293T cells were seeded to 24-well cell culture microplates in 500 μl DMEM containing 10% FBS. On Day 1, the cells were transfected with respective pGBA-NanoLuc_STD (SEQ ID NOS: 273-296) and pGBA-NanoLuc_EG (SEQ ID NOS: 297-324) plasmids using Mirus TransIT-Lenti transfection reagent according to manufacturer's instructions. Each well was treated with complexes made using 0.2 pg plasmid, 0.6 μl Mirus TransIT-Lenti reagent and 50 μl of Opti-MEM medium. On Day 3, the transfected cells and cell culture supernatants were assayed for the activity of NanoLuc luciferase and GBA, and processed for western blotting.

For the NanoLuc assay, cell culture supernatants were collected in microcentrifuge tubes and cleared of cell debris by centrifugation. The cells, left adherent onto the 24-well plates, were lysed using 250 μl of 0.2% Triton X-100 in PBS at room temperature. 50 μl of cleared cell culture supernatants or 50 μl of cell lysate was loaded to opaque black 96-well microplates. 50 μl of fresh 1× Nano-Glo assay reagent in the respective assay buffer (Promega) was added to each well, incubated 2-5 minutes, followed by luminescence measurement using a Biotex Synergy LX plate reader. Cell lysates were further analysed for their protein concentration using the A660 reagent (Thermo Scientific) according to the manufacturer's instructions. Briefly, 40 μl of samples or standards were mixed with 150 μl A660 assay reagent, the absorbance at 660 nm was measured using a Biotek Synergy LX plate reader, and protein concentration was quantified based on the standard curve. The luminescence of cell lysates was normalized per pg cell lysate protein (i.e., yielding luminescence values in units of relative light unit (RLU)/pg protein) while the luminescence of cell culture supernatants was normalized using the volume of supernatant used in the assay (i.e., yielding luminescence values in units of RLU/ml supernatant).

To determine GBA activity, the cells were seeded and transfected with respective plasmids as for the NanoLuc assay above. On Day 3, cell culture supernatant was collected, cleared from cell debris by centrifugation, and kept for assaying secreted GBA activity. Adherent cells were detached from the plate using 500 μl PBS and pelleted by centrifugation. The cell pellet was lysed using 1×GBA Assay Buffer (0.1 M sodium citrate, 0.2 M sodium phosphate, 0.25% Triton X-100, 0.25% sodium taurocholate, 1.25 mM EDTA, 5 mM DTT), pre-equilibrated to 37° C. prior for lysis. The protein concentration of the cell lysate was determined using the Pierce A660 protein assay per the manufacturer's instructions, after which the cell lysate was diluted to the final protein concentration of 125 pg/ml using 1×GBA Assay Buffer. 40 μl of pre-diluted cell lysate was pipetted to individual wells of a clear 96-well plate in duplicate. 20 μl of cell culture supernatants, as collected earlier, were added to individual wells of a clear 96-well plate in duplicate, and 20 μl of 2×GBA Assay Buffer was added to each well that contained cell culture supernatant. Thereafter, 20 μl of Assay Substrate (6 mM 4-MU-beta-D-glucopuranoside prepared in 1×GBA Assay Buffer) was added to each well that contained cell lysate or cell culture supernatant. In adjacent wells, a calibration curve using 4-methyl-umbelliferone was prepared in 1×GBA Assay Buffer. The samples were incubated in the presence of Assay Substrate at 37° C. for 30 minutes-4 hours, followed by the addition of 100 μl of Stop Solution (0.5 M Glycine, 0.3 M NaOH at pH10). Thereafter, the fluorescence of each sample and standard was measured at Excitation/Emission wavelengths of 360/445 nm using a Biotek Synergy LX plate reader. GBA activity in cell lysate samples was calculated using the following equation: Activity=[B/(T×V×P)]×D=pmol/min/mg=μU/mg, where B is converted 4-MU amount as calculated using the standard curve (pmol), T is the reaction time (min), V is the sample volume (ml), P is the initial protein sample concentration, and D is the sample dilution factor (if applicable). GBA activity in cell culture supernatants was calculated using the following equation: Activity=[B/(T×V)]×D=pmol/min/ml=μU/ml, where B is converted 4-MU amount as calculated using the standard curve (pmol), T is the reaction time (min), V is the sample volume (ml), and D is the sample dilution factor (if applicable).

For western blotting, the transfected cells were harvested by scraping and pelleted by centrifugation. The cell pellet was lysed using 100 μl RIPA buffer (Cell Signalling technologies), supplemented with 1× protease inhibitor cocktail and 10 U of universal nuclease, incubated on ice for 10 minutes, followed by 10 seconds of sonication. The lysed samples were centrifuged at 14,000 g for 10 minutes, and 80 μl of supernatant collected for analysis. The protein concentration of each sample was determined using the Pierce A660 assay as per manufacturer's instructions. 40 pg of cell lysate was loaded in each well of a 4-12% Bis-Tris MES gel and continue with western blotting as described earlier in Example 15. For detecting GBA, rabbit anti-GBA antibody (Abcam ab128879) was used at 1:500 dilution overnight.

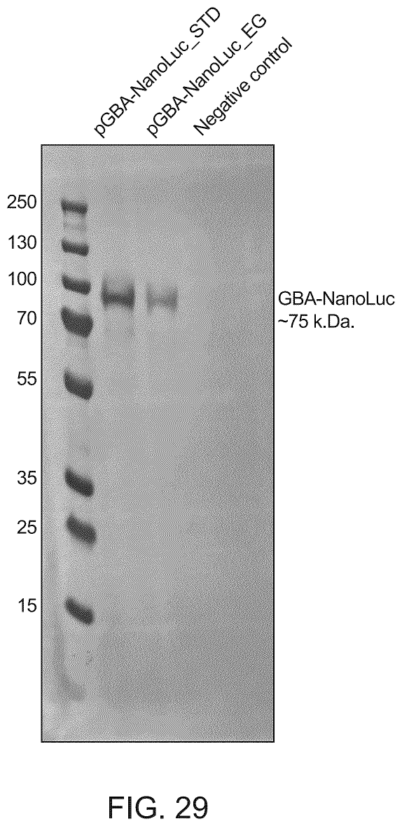

Western blotting revealed that the designed pGBA-NanoLuc_STD and pGBA-NanoLuc_EG constructs, when expressed in HEK 293T cells, are detected as a single band of the correct predicted size for pGBA-NanoLuc protein chimera of approx. 75 kDa. The band of the pGBA-NanoLuc_STD construct was somewhat stronger than that of the pGBA-NanoLuc_EG construct (FIG. 29), which corresponds to earlier observations with expressed secreted proteins as presented in Example 15.

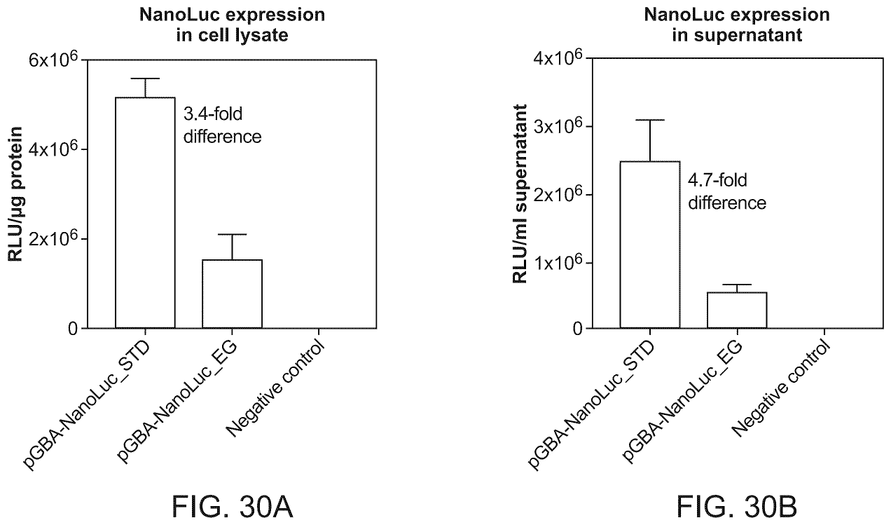

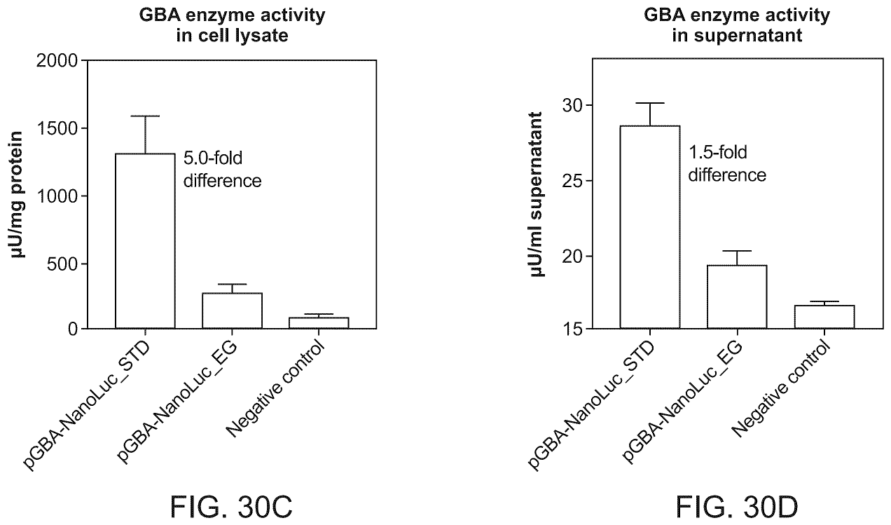

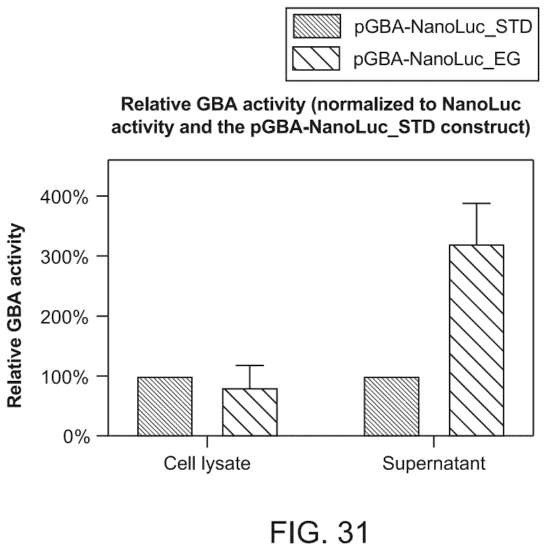

By using NanoLuc-tagged GBA protein during analysis, it was possible to differentiate between the total expression of the protein of interest (NanoLuc reporter readout) and the expression of functionally active protein of interest (GBA enzymatic activity). The total amount of both the reporter protein (NanoLuc) and the enzymatic activity (GBA) were higher in the case of the pGBA-NanoLuc_STD construct (FIGS. 30A-30D), consistent with the western blot results above. However, the relative quality of the produced protein was much higher in case of the pGBA-NanoLuc_EG construct, when the enhancer protein was co-expressed (FIG. 31). The relative quality of the expressed GBA protein was estimated by first normalizing the GBA enzymatic activity using the NanoLuc reporter signal (i.e., yielding the specific GBA activity GBA_S=GBA activity/NanoLuc signal), and then by comparing the GBA_S of the two different constructs. This analysis revealed that in cell lysates, the specific GBA activity was similar between the pGBA-NanoLuc_STD and the pGBA-NanoLuc_EG construct.

Notably, however, in cell supernatants, the specific GBA activity in the presence of the enhancer protein (i.e., the pGBA-NanoLuc_EG construct) was approximately 300% higher than that in the absence of the enhancer L protein (i.e., the pGBA-NanoLuc_STD construct) (FIG. 31). Given that endogenous GBA is a secreted protein fully active in the supernatant, these data support the increased quality of the protein when co-expressed with the enhancer protein L, as presented herein.

### Example 18: Expression of the L Enhancer Protein Improves the Uniformity of In Vivo Glucosylceramidase (GBA) Expression

To evaluate the expression of GBA in vivo, the following experiments were performed. Both of pGBA-NanoLuc_STD (without the enhancer protein L, SEQ ID NOS: 273-296) and pGBA-NanoLuc_EG (with the enhancer protein L, SEQ ID NOS: 297-324) plasmids were formulated into lipid nanoparticles (LNPs) for in vivo delivery as followed. 300 pg plasmid was dissolved in 2.6 ml of encapsulation buffer (EB, 25 mM sodium acetate at pH4). The LNP lipid mixture was prepared in 2.6 ml of 100% EtOH, containing 1% DMG-PEG(2000), 39% cholesterol, 10% DOPC and 50% DLin-KC2-DMA ionizable lipid, with the final total lipid concentration of 4 mM. Equal volumes (2.6 ml each) of plasmid in EB and lipid in EtOH were combined by rapid mixing. Immediately after mixing, 5.2 ml of neutrbiosimilarffer (NB, 300 mM NaCl+20 mM sodium citrate at pH6) was added to the lipid/plasmid mixture and mixed rapidly and incubated at 37° C. for 30 minutes. The mixture was diafiltrated against PBS using Amicon Ultra 15-ml 100 kDa MWCO spin filters. Encapsulation efficiency and total concentration of loaded plasmid were determined using the SYBRSafe encapsulation efficiency assay, as followed. 5 μl of plasmid/LNPs were mixed with 1×SYBRSafe DNA binding dye either in TE buffer (to detect the amount of plasmid not loaded into LNPs) or in TE buffer containing 1% Triton X-100 (to detect the total amount of plasmid, i.e., both loaded and not loaded plasmid in LNPs). For plasmid DNA absolute amount calculation, a standard curve was built using known amounts of reference naked plasmid DNA, mixed in 1×SYBRSafe DNA binding dye either in TE buffer or in TE buffer containing 1% Triton X-100, respectively. Samples or standards were incubated for 5 min and the fluorescence was read using the Biotek Synergy LX fluorescence microplate reader using the filter set for FITC. Encapsulation efficiency was calculated using the equation: Loading_Efficiency=(Plasmid_total−Plasmid_non-loaded)/Plasmid_total×100%.

Loading efficiency was >90%. Plasmid/LNPs were diluted using PBS to the equivalent plasmid concentration of 30 pg plasmid per 200 μl PBS. Female Balb/c mice were anaesthetized and dosed individually with a fixed volume of 200 μl for subcutaneous injection between the scapulae, N=3 per group. At indicated time points, whole-body bioluminescence imaging (BLI) was performed by injecting the mice intraperitoneally (i.p.) with Nano-Glo® In Vivo Substrate, FFz, and imaged in the prone and supine positions. The prone position focused on the injection site and the supine position focused on the liver area. Luminescence values were quantified, tabulated and plotted.

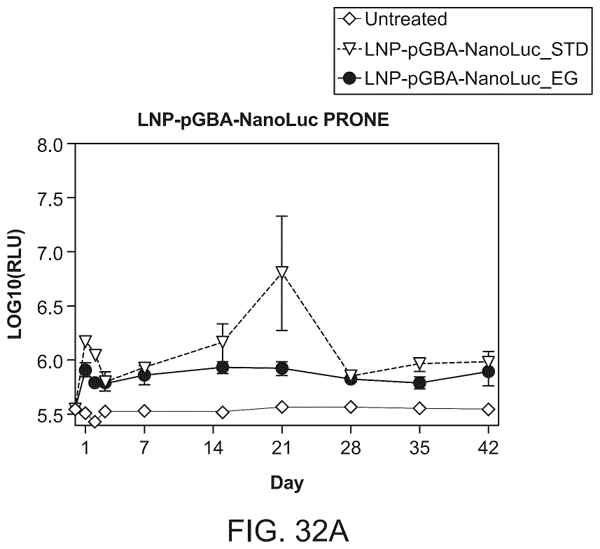

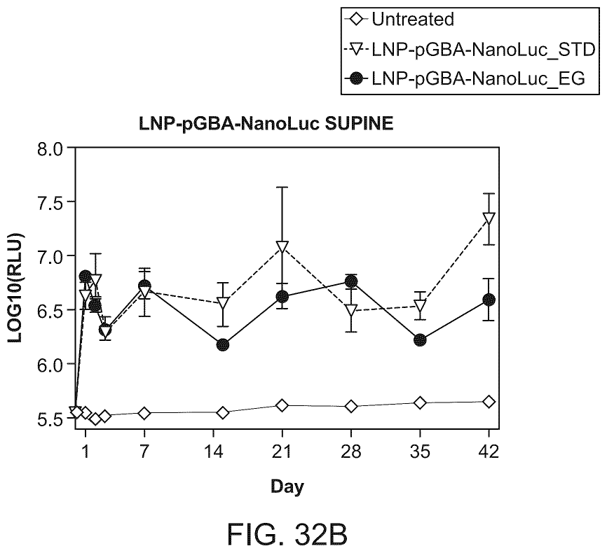

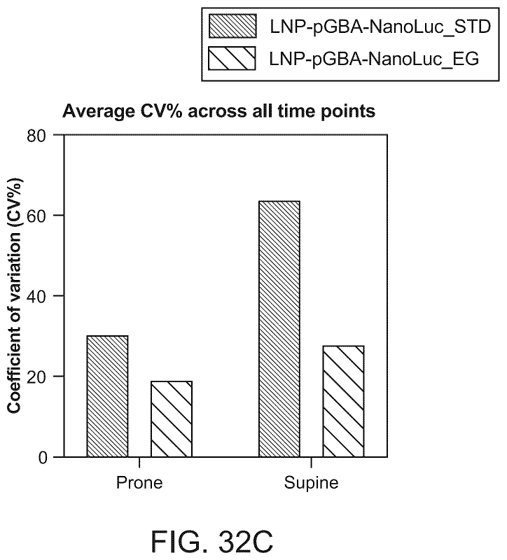

Bioluminescence imaging (BLI) results indicated that the detected luminescence signal was relatively equal if not marginally higher in the LNP-pGBA-NanoLuc_STD group as compared to the LNP-pGBA-NanoLuc_EG group (FIGS. 32A-C and Table 6), consistent with the in vitro experiments. Strong signal was detected both at the prone position BLI (i.e., at the site of injection) as well as the supine position (i.e., at the site of expressed GBA-NanoLuc accumulation, which is likely the liver, indicating the in vivo produced protein is secreted and accumulates in target tissues).

TABLE 6
Average coefficient of variation of LNP/vectors with (EG) and
without enhancer protein (STD) as imaged in mice
Average coefficient of variation (CV %) across all time points

| | LNP-pGBA-NanoLuc_STD | LNP-pGBA-NanoLuc_EG |
|---|---|---|
| | (SEQ ID NOS: 273-296) | (SEQ ID NOS: 297-324) |
| Prone | 30.5% | 19.1% |
| Supine | 63.9% | 27.8% |

The BLI signals in the LNP-pGBA-NanoLuc_STD group were more variable both across time as well as between individual animals, as measured at the same time point when compared to LNP-pGBA-NanoLuc_EG group. This was investigated further by quantifying the coefficient of variation (CV %) of the signal at each measurement time point, and then calculating the average CV % for each treatment group. The CV % was defined as the standard deviation of the signal divided by the signal average. This analysis revealed that the average CV % was higher in the absence of the Enhancer protein (FIG. 32 C and Table 6.)). Whereas in the prone position (i.e., the site of LNP administration) the difference in CV % was relatively small, in the prone position (i.e., the site of secreted GBA-NanoLuc protein accumulation) the difference was approximately 2-fold. This suggests that in the presence of the enhancer protein L, using vectorized secreted proteins in vivo will be more uniform and robust as compared to the absence of the enhancer protein L.

1. A method of expressing a target protein in a subject in need thereof, comprising administering to the subject a system comprising:
a) a first polynucleotide encoding the target protein; and
b) a second polynucleotide encoding an enhancer protein wherein:
the enhancer protein is an inhibitor of nucleocytoplasmic transport (NCT).

2. (canceled)

3. The method of claim 1, wherein the NCT inhibitor is a viral protein.

4. The method of claim 3, wherein the NCT inhibitor is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

5. The method of claim 4, wherein the NCT inhibitor is a picornavirus leader (L) protein or a functional variant thereof.

6. The method of claim 4, wherein the NCT inhibitor is a picornavirus 2A protease or a functional variant thereof.

7-11. (canceled)

12. The method of claim 4, wherein the NCT inhibitor is a rhabdovirus matrix (M) protein or a functional variant thereof.

13. The method of claim 5, wherein the L protein is the L protein of Theiler's virus or a functional variant thereof.

14. The method of claim 5, wherein the L protein shares at least 90% identity to SEQ ID NO: 1.

15. The method of claim 5, wherein the L protein is the L protein of Encephalomyocarditis virus (EMCV) or a functional variant thereof.

16. The method of claim 5, wherein the L protein shares at least 90% identity to SEQ ID NO: 2.

17. The method of claim 5, wherein the L protein is selected from the group consisting of the L protein of poliovirus, the L protein of HRV16, the L protein of mengo virus, and the L protein of Saffold virus 2 or a functional variant thereof.

18-32. (canceled)

33. The method of claim 1, wherein the target protein is a therapeutic protein.

34. The method of claim 1, wherein the target protein is an immunogenic protein.

35. The method of claim 1, wherein the target protein is an antibody, a nanobody, a receptor, a bi-specific T-cell engager (BiTE), a growth factor, a hormone, an enzyme, an immunomodulatory protein, an antigen, a structural protein, a blood protein, an anti-microbial polypeptide, an anti-viral polypeptide_, a tumor suppressor, a transcription factor, or a translation factor.

36-37. (canceled)

38. The method of claim 1, wherein the method elicits an immune response in the subject.

39. A method of treating a disease in a subject, wherein the disease is caused by, correlated with, or associated with a target protein, comprising administering to the subject a system comprising:
a) a first polynucleotide encoding the target protein; and
b) a second polynucleotide encoding an enhancer protein wherein the enhancer protein is an inhibitor of nucleocytoplasmic transport (NCT).

40-47. (canceled)

48. The method of claim 1, wherein the enhancer protein increases the activity of the target protein in the subject.

49. The method of claim 1, wherein the enhancer protein lowers the expression level of the target protein in the subject.

50. The method of claim 1, wherein the enhancer protein increases the uniformity of expression of the target protein at the injection site of the subject.

51. The method of claim 1, wherein the enhancer protein increases the duration of active target protein in a cell of the subject or the subject.

52-61. (canceled)

62. The method of claim 1, wherein the system is administered via a lipid nanoparticle (LNP).

63. The method of claim 62, wherein the LNP comprises a PEGylated lipid, a cholesterol, and one or more ionizable lipids.

64-65. (canceled)

66. The method of claim 1, wherein the system is delivered intramuscularly or subcutaneously.

67-98. (canceled)

99. A composition comprising a vector system comprising one or more vectors, the one or more vectors, comprising:
a) a first polynucleotide encoding an amino acid sequence from Table 8 or Table 9; and
b) a second polynucleotide encoding a picornavirus leader (L) protein with an amino acid sequence selected from the group consisting of SEQ ID NOS: 1-6, and 24, or an amino acid sequence with at least 70%, at least 75%, at least 80%, at least 85%, at least 90%, at least 95%, at least 96%, at least 97%, at least 98%, or at least 99% sequence identity thereto;
wherein the first polynucleotide encoding the amino acid sequence from Table 8 or Table 9, and the second polynucleotide encoding the L protein are operatively linked to one or more promoters; and wherein the amino acid sequence from Table 8 or Table 9 and the L protein are co-expressed.

100. The composition of claim 99, wherein the first polynucleotide encodes a GBA amino acid sequence of SEQ ID NO: 406.

101-103. (canceled)

104. The method of claim 1, wherein the enhancer protein increases the expression level of the target protein in the subject.
